SEQUENCE LISTING (143 sequences)

SEQ ID NO: 1 - DNA, 2053 bp, Homo sapiens
  source 1..2053: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 2 - protein, 287 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 3 - DNA, 6607 bp, Mus musculus
  source 1..6607: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 4 - protein, 287 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 5 - DNA, 2110 bp, Homo sapiens
  source 1..2110: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtgaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 6 - protein, 306 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 7 - DNA, 6664 bp, Mus musculus
  source 1..6664: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggagatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 8 - protein, 306 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGDQQSGYGKVSRRGGHQNSYKPY

SEQ ID NO: 9 - DNA, 2200 bp, Homo sapiens
  source 1..2200: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaagggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 10 - protein, 336 aa, Homo sapiens
MSEEQFGGDGAAAAATAAVGGSAGEQEGAMVAATQGAAAAAGSGAGTGGGTASGGTEGGSAESEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 11 - DNA, 6754 bp, Mus musculus
  source 1..6754: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaagggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 12 - protein, 336 aa, Mus musculus
MSEEQFGGDGAAAAATAAVGGSAGEQEGAMVAAAAQGPAAAAGSGSGGGGSAAGGTEGGSAEAEGAKIDASKNEEDEGKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 13 - DNA, 2257 bp, Homo sapiens
  source 1..2257: mol_type = other DNA
cttccgtcggccattttaggtggtccgcggcggcgccattaaagcgaggaggaggcgagagcggccgccgctggtgcttattcttttttagtgcagcgggagagagcgggagtgtgcgccgcgcgagagtgggaggcgaagggggcaggccagggagaggcgcaggagcctttgcagccacgcgcgcgccttccctgtcttgtgtgcttcgcgaggtagagcgggcgcgcggcagcggcggggattactttgctgctagtttcggttcgcggcagcggcgggtgtagtctcggcggcagcggcggagacactagcactatgtcggaggagcagttcggcggggacggggcggcggcagcggcaacggcggcggtaggcggctcggcgggcgagcaggagggagccatggtggcggcgacacagggggcagcggcggcggcgggaagcggagccgggaccgggggcggaaccgcgtctggaggcaccgaagggggcagcgccgagtcggagggggcgaagattgacgccagtaagaacgaggaggatgaaggccattcaaactcctccccacgacactctgaagcagcgacggcacagcgggaagaatggaaaatgtttataggaggccttagctgggacactacaaagaaagatctgaaggactacttttccaaatttggtgaagttgtagactgcactctgaagttagatcctatcacagggcgatcaaggggttttggctttgtgctatttaaagaatcggagagtgtagataaggtcatggatcaaaaagaacataaattgaatgggaaggtgattgatcctaaaagggccaaagccatgaaaacaaaagagccggttaaaaaaatttttgttggtggcctttctccagatacacctgaagagaaaataagggagtactttggtggttttggtgaggtggaatccatagagctccccatggacaacaagaccaataagaggcgtgggttctgctttattacctttaaggaagaagaaccagtgaagaagataatggaaaagaaataccacaatgttggtcttagtaaatgtgaaataaaagtagccatgtcgaaggaacaatatcagcaacagcaacagtggggatctagaggaggatttgcaggaagagctcgtggaagaggtggtggccccagtcaaaactggaaccagggatatagtaactattggaatcaaggctatggcaactatggatataacagccaaggttacggtggttatggaggatatgactacactggttacaacaactactatggatatggtgattatagcaaccagcagagtggttatgggaaggtatccaggcgaggtggtcatcaaaatagctacaaaccatactaaattattccatttgcaacttatccccaacaggtggtgaagcagtattttccaatttgaagattcatttgaaggtggctcctgccacctgctaatagcagttcaaactaaattttttgtatcaagtccctgaatggaagtatgacgttgggtccctctgaagtttaattctgagttctcattaaaagaaatttgctttcattgttttatttcttaattgctatgcttcagaatcaatttgtgttttatgccctttcccccagtattgtagagcaagtcttgtgttaaaagcccagtgtgacagtgtcatgatgtagtagtgtcttactggttttttaataaatccttttgtataaaaatgtattggctcttttatcatcagaataggaaaaattgtcatggattcaagttattaaaagcataagtttggaagacaggcttgccgaaattgaggacatgattaaaattgcagtgaagtttgaaatgtttttagcaaaatctaatttttgccataatgtgtcctccctgtccaaattgggaatgacttaatgtcaatttgtttgttggttgttttaataatacttccttatgtagccattaagatttatatgaatattttcccaaatgcccagtttttgcttaatatgtattgtgctttttagaacaaatctggataaatgtgcaaaagtacccctttgcacagatagttaatgttttatgcttccattaaataaaaaggacttaaaatctgttaattataatagaaatgcggctagttcagagagatttttagagctgtggtggacttcatagatgaattcaagtgttgagggaggattaaagaaatatataccgtgtttatgtgtgtgtgctt

SEQ ID NO: 14 - protein, 355 aa, Homo sapiens
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 15 - DNA, 6811 bp, Mus musculus
  source 1..6811: mol_type = other DNA
ccattttaggtggtccgcggcggcgccattaaagcgaggaggaggcgagagtggccgccgctgctacttcattcttttttttttcagtgcagccggggagagcgagagagcgcgctgcgcgagagtgggaggcgaggggggcaggccggggagaggcgcaggagcccttgcagccacgcgcgcgccttgtctagggtgcctcgcgaggtagagcgggcatcgcgcggcggcggcggggattactttgctgctagtttcggttcgcggcggcggcggcgtcggcgggtgtcgtcttcggcggcggcagtagcactatgtcggaggagcagttcggaggggacggggcggcggcggcggcaacggcggcggtaggcggctcggcgggcgagcaggagggagccatggtggcggcggcggcgcaggggccggcggcggcggcgggaagcgggagcggcggcggcggctctgcggccggaggcaccgaaggaggcagcgccgaggcagagggagccaagatcgacgccagtaagaacgaggaggatgaaggccattcaaactcctccccacgacacactgaagcagcggcggcacagcgggaagaatggaaaatgtttataggaggccttagctgggacaccacaaagaaagatctgaaggactacttttccaaatttggtgaagttgtagactgcactctgaagttagatcctatcacagggcgatcaaggggttttggctttgtgctatttaaagagtcggagagtgtagataaggtcatggatcagaaagaacataaattgaatgggaaagtcattgatcctaaaagggccaaagccatgaaaacaaaagagcctgtcaaaaaaatttttgttggtggcctttctccagacacacctgaagaaaaaataagagagtactttggtggttttggtgaggttgaatccatagagctccctatggacaacaagaccaataagaggcgtgggttctgttttattacctttaaggaagaggagccagtgaagaagataatggaaaagaaataccacaatgttggtcttagtaaatgtgaaataaaagtagccatgtcaaaggaacagtatcagcagcagcagcagtggggatctagaggagggtttgcaggcagagctcgcggaagaggtggaggccccagtcaaaactggaaccagggatatagtaactattggaatcaaggctatggcaactatggatataacagccaaggttacggaggttatggaggatatgactacactggttacaacaactactatggatatggtgattatagcaatcagcagagtggttatgggaaagtatccaggcgaggtggacatcaaaatagctacaaaccatactaaattattccatttgcaacttatccccaacaggtggtgaagcagtattttccaatttgaagattcatttgaaggtggctcctgccacctgctaatagcagttcaaactaaattttttctatcaagttcctgaatggaagtatgacgttgggtccctctgaagtttaattctgagttctcattaaaagaatttgctttcattgttttatttcttaattgctatgcttcagtatcaatttgtgttttatgccccccctcccccccagtattgtagagcaagtcttgtgttaaaaaaagcccagtgtgacagtgtcatgatgtagtagtgtcttactggttttttaataaatccttttgtataaaaatgtattggctcttttatcatcagaataggaggaagtgaaatactacaaatgtttgtcttggattcaagtcactagaagcataaatttgaggggataaaaacaacggtaaactttgtctgaaagagggcatggttaaaaatgtagtgaattttaaatgtttttagcaaaatttgattttgcccaagaatccctgtctgaattggaaatgacttaatgtagtcaatgtgcttgttggttgtcttaatattacttctgtagccattaagttttatgagtaacttcccaaatacccacgtttttctttatatgtattgtgctttttaaaaacaaatctggaaaaatgggcaagaacatttgcagacaattgtttttaagcttccattaaataaaaaaaatgtggacttaaggaaatctattaatttaaatagaactgcagctagtttagagagtatttttttcttaaagctttggtgtaattagggaagattttaaaaaatgcatagtgtttatttgtatgtgtgctctttttttaagtcaatttttggggggttggtctgttaactgagtctaggatttaaaggtaagatgttcctagaaatcttgtcatcccaaaggggcgggcgctaaggtgaaacttcagggttcagtcagggtcactgctttatgtgtgaaatcactcaaattggtaagtctcttatgttagcattcaggacattgatttcaacttggatggacaatttatagttactactgaattgtgtgttaatgtgttcagtcctggtaagttttcagtttgatcagttagttggaagcagacttgaagagctgttagtcacgtgagccatgggtgcagtcgatctgtggtcagatgcctgagtctgtgatagtgaattgtgtctaaagacattttaatgataaaagtcagtgctgtaaagttgaaagttcatgagagacatacaatgagggctgcagcccatttttaaaaacattataatacaaaagtatgcacatttgtttacatatccctgcctttgtattacagtggcaggtttgtgtacttaaactgggaaagcctcagatctatgattacctggcctatcatagaaagtgtctaaataaatcactctgtcaattgaatacattagtattagctagcatacttcattatgcctgttttccataaataccacaccaaaaacttgcttggggcagtttgagcctagttcatgagctgctatcagattggtcttgatcctatataataggccaaatgtctgtaaacagctgtgctggtggaatgtagaaagtcactgcactcagattcaacttcctgattggaagtcatcacagtgtgattaaacattttcacaaagaatagtagataaataacttggtttttaatgttaactttgtttccattaagtcacatttaaaaacttatcctcacgcctacctgagttaattatctgttgacctagatatctttctggccactcactgacttatttcttgaacttttgccatttgcataaatcttgtcagctttgttcttgattatgcattgtccaggctgagctagttgtctttccaggaatccctttgtctctgaattaggtcctttgtttcctaaatcatcctgcttgtttggcacaagtcttcccaggccagtgagacctccgtgtcctctcagcaccataggggtaggtaaccctggttaggctggacaggggtttgctgagggagtttgttcatttgaatctaggtcttacatgacgtctttcaaatagggtttttaccttgacactaaactgtccagtctaagcagttctgcaaaatgtgagggaattatgaacttcttcctgcagtgggtttttatggttttggtttgttttttgttgttttggttctttgttgagccctggacaaaaacttccctagttctggtttctacaatttaaattaaaaacagaattcatcttagaatttttcaccctcttccccaactattctaatcaatcttaagtatgcccttcatcttttttccttcctaaggcttttactgatagtgtaattccgtactcttcaaccctgggaaggctgaagtggattcttgagctcatttcaaggctgacctgggtgttggcaagaacccagcttagaacaaacacatgcaaggccatcttaccttacatcctgttgcttggacttcttcctgctcaaagtttttagtggatgctaagtgatctttgcttccactgaggagtggaacactttagaatgaacctctagatagatatttttattgtctggtgagggttactggagtttcccaccctgcctgaagggtgaatctggcttacagtgttctcatctcaaagggaagaaggcagatggctgtgtccagagagagccatcacagtttgcttcagagacactagaatgggctggaagatctagtggtcttaatcagacttgaaacctggcctttcttcattacccatatgtctaccagtacttgggctaacacttaagccattagggcctttgtaggggtgttttgagaccccctccatgctaacaaatatacaggtttcttaacatttgctcataaacttgtaaagcttactttctcttaatccaccccacatttaacaagccctggtacttagaatttcagaagagtaatggcaggtaggtgtgtgtgtgtgtgtgtgtgtgtgtgtgtgtgtgtgtgtgtgagagagagagagagagagagagagagagagagagagagagagagagagagagagaagtttgtggaaaatcaggtaatgacagctcatccttttagaattgtacttcagaatagaaacatttggtgggctgttaggtagctttgattacttgtgggtagacctgctagtattgccagtcctcaagcaatgagctttctgtatcttgtttactagatatatactaccaggtgagtcatttcctggggttctgttttcttttaaaatctttccctaaacttaatatgtattaaaaagtctggcttttcagtccattctttgtgcactgggatggcaattgcttcattatatgacaattgctgttcccaagtcagaattcagtgtgctgatttgacatcagttcgtcccgaataagttcctgttaccaggatttacattcagcacattagaaacttgttggtgtgcttttattcttggagcattttccttagactaccttccactttgagtgctctgtttaggatgttgaggtgttaggattcttgacagccagaaagactgaacccactatctgggcacagtgttcgtgttgctctataaatgtatgctttttttgatttggggttgttttacctacattgtcaaactagatccatgcttaacagtgataatgaaggctttttgtttgttttgtttgtgggtcctccccccccccccaagacagggtttctctgtaggctgtcctagaacttgttcttttttaaccaaaatttggcaaggctgaaaatggaatcctataatcaatgctggccacattaaagttaatagttgagaagtcttgtctgaatttccttgggcaaaaagattctagccagttcaataccctgttgtgcaaattcaatttgctgttataatttgctctcagttatcagttggaaggaggttaattctaatgtacttggaagaggcctgtagaccatctataactgcatcagttgtacagcgttgttgcctgggattctctagttcacataaactcccaagtcttagccgtggtgatggctacagtgtggaagatggtgagcattctagtgagtatcgcgatgacggcagtaaagagcagcaggcagccgtggctgggctcactgaccgtggctgtaagttacggaggcagcacacacttctgtacacacctctcatcagttaccggagtcattgcattgcggactaactggctgactcaagttgtcttgctactgaagtcttgagttggtctcatgcatttaccctgttgacttgagcaccttaaagtcgaaaggatgtctggttgtggctttattgtaaacagccttaggtaaagaggggagtatatcggttaggaaggtgaaaaatgatacttccaagttcagtgggaaaccctgggtttatcccccagcttaagaaagaatgcctaacaatgtttcagaattagattctgtggaaggtgagggtgttagaacagtccaaatttgttattgtagacttgcagtgggaggaatttttaaatatacagatcagtcgacactcattaacttcactgataaaggtggaaacggatgtggcaacacttctaagttcatttgtatatgtttgtaatttgattggttgtattctgttgcactctagaatttgaaggcaaggttacctctgctttttaatttttttttttttaaagaaagaaaaaacactgaaagaaacttcaaaagatctgttaatgctaatacctgaatgtggcatttaacatgtcatggaaactgctttgaataaatacttgagaaaaggaatgaaataattgccgtttttgttgttgagtgaatgggtgtggtttaatgagcgtaatcatttttataaaacagctgtgagactgaagtggaatccttattaaatgtggaaaatggcctttgaggattacagtagagattcaactaagagagtaaataaagcttgaaactaattcgttgtaaattgcttctacaatcattgctctatatagcatgctattgccaatcagttttatgtattaagacctatcagcatgtcttttttaggttgacctcattttaaattataagatgctctctgtaccgttttaacatttccaggatttattctttctaggcaaattccactggactgtttccattgtagaagcttccttatagattcttcaaatgaagcttacagtgtgctttcttggggttttgatttgcactaaattttattttctgaaagatcacttatgtttataatgtagtgctttgtcttaacaattaaactttccagcactcatgca

SEQ ID NO: 16 - protein, 355 aa, Mus musculus
MSEEQFGGDGAAAAATAAVGGSAGEQEGAMVAAAAQGPAAAAGSGSGGGGSAAGGTEGGSAEAEGAKIDASKNEEDEGHSNSSPRHTEAAAAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 17 - DNA, 921 bp, synthetic construct
  source 1..921: mol_type = other DNA
atgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactga

SEQ ID NO: 18 - DNA, 328 bp, synthetic construct
  source 1..328: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagc

SEQ ID NO: 19 - DNA, 82 bp, synthetic construct
  source 1..82: mol_type = other DNA
gtgagtatctcagggatccagacatggggatatgggaggtgcctctgatcccagggctcactgtgggtctctctgttcacag

SEQ ID NO: 20 - DNA, 424 bp, synthetic construct
  source 1..424: mol_type = other DNA
ggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcccgcggaacaggtgagtatctcagggatccagacatggggatatgggaggtgcctctgatcccagggctcactgtgggtctctctgttcacaggtt

SEQ ID NO: 21 - DNA, 724 bp, synthetic construct
  source 1..724: mol_type = other DNA
gccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgcccccgggtcacc

SEQ ID NO: 22 - DNA, 577 bp, synthetic construct
  source 1..577: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 23 - DNA, 127 bp, synthetic construct
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 24 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 25 - DNA, 135 bp, synthetic construct
  source 1..135: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 26 - DNA, 141 bp, synthetic construct
  source 1..141: mol_type = other DNA
aaaaagtacctcaataataaatacagaacttctcctttcaacctcttccatcacatcaacacctatgaagacaatgggtttctgattgtggatctctgctgctggaaaggatttgagtttgtttataattacttatattta

SEQ ID NO: 27 - DNA, 893 bp, synthetic construct
  source 1..893: mol_type = other DNA
gcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaata

SEQ ID NO: 28 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 29 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 30 - DNA, 106 bp, synthetic construct
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 31 - DNA, 3017 bp, synthetic construct
  source 1..3017: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagatatagctagtcgacaaaaagtacctcaataataaatacagaacttctcctttcaacctcttccatcacatcaacacctatgaagacaatgggtttctgattgtggatctctgctgctggaaaggatttgagtttgtttataattacttatatttagttaccggtcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcccgcggaacaggtgagtatctcagggatccagacatggggatatgggaggtgcctctgatcccagggctcactgtgggtctctctgttcacaggttccttaagggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgacgcgttaatgaggtacctcgaggatctttttccctctgccaaaaattatggggacatcatgaagccccttgagcatctgacttctggctaataaaggaaatttattttcattgcaatagtgtgttggaattttttgtgtctctcactcgcatgcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaatagactagtcgatccagacatgataagatacattgatgagtttggacaaaccacaactagaatgcagtgaaaaaaatgctttatttgtgaaatttgtgatgctattgctttatttgtaaccattataagctgcaataaacaagttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 32 - DNA, 3314 bp, synthetic construct
  source 1..3314: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagatatagctagtcgacaaaaagtacctcaataataaatacagaacttctcctttcaacctcttccatcacatcaacacctatgaagacaatgggtttctgattgtggatctctgctgctggaaaggatttgagtttgtttataattacttatatttagttaccggtgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatcgccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtggatccctccctggggacagcccctcctggctagtcacaccctgtaggctcctctatataacccaggggcacaggggctgcccccgggtcaccttaagggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgacgcgttaatgaggtacctcgaggatctttttccctctgccaaaaattatggggacatcatgaagccccttgagcatctgacttctggctaataaaggaaatttattttcattgcaatagtgtgttggaattttttgtgtctctcactcgcatgcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaatagactagtcgatccagacatgataagatacattgatgagtttggacaaaccacaactagaatgcagtgaaaaaaatgctttatttgtgaaatttgtgatgctattgctttatttgtaaccattataagctgcaataaacaagttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 33 - DNA, 3600 bp, synthetic construct
  source 1..3600: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagatatagctagtcgacaaaaagtacctcaataataaatacagaacttctcctttcaacctcttccatcacatcaacacctatgaagacaatgggtttctgattgtggatctctgctgctggaaaggatttgagtttgtttataattacttatatttagttaccggtcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcccgcggaacaggtgagtatctcagggatccagacatggggatatgggaggtgcctctgatcccagggctcactgtgggtctctctgttcacaggttccttaagggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcggtacctcgaggatctttttccctctgccaaaaattatggggacatcatgaagccccttgagcatctgacttctggctaataaaggaaatttattttcattgcaatagtgtgttggaattttttgtgtctctcactcgcatgcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaatagactagtcgatccagacatgataagatacattgatgagtttggacaaaccacaactagaatgcagtgaaaaaaatgctttatttgtgaaatttgtgatgctattgctttatttgtaaccattataagctgcaataaacaagttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 34 - DNA, 3169 bp, synthetic construct
  source 1..3169: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagatatagctagtcgacaaaaagtacctcaataataaatacagaacttctcctttcaacctcttccatcacatcaacacctatgaagacaatgggtttctgattgtggatctctgctgctggaaaggatttgagtttgtttataattacttatatttagttaccggtccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgaccttaagggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgacgcgttaatgaggtacctcgaggatctttttccctctgccaaaaattatggggacatcatgaagccccttgagcatctgacttctggctaataaaggaaatttattttcattgcaatagtgtgttggaattttttgtgtctctcactcgcatgcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaatagactagtcgatccagacatgataagatacattgatgagtttggacaaaccacaactagaatgcagtgaaaaaaatgctttatttgtgaaatttgtgatgctattgctttatttgtaaccattataagctgcaataaacaagttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 35 - DNA, 2921 bp, synthetic construct
  source 1..2921: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagatatagctagtcgacaaaaagtacctcaataataaatacagaacttctcctttcaacctcttccatcacatcaacacctatgaagacaatgggtttctgattgtggatctctgctgctggaaaggatttgagtttgtttataattacttatatttagttaccggtcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcccttaagggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgacgcgttaatgaggtacctcgaggatctttttccctctgccaaaaattatggggacatcatgaagccccttgagcatctgacttctggctaataaaggaaatttattttcattgcaatagtgtgttggaattttttgtgtctctcactcgcatgcttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaatagactagtcgatccagacatgataagatacattgatgagtttggacaaaccacaactagaatgcagtgaaaaaaatgctttatttgtgaaatttgtgatgctattgctttatttgtaaccattataagctgcaataaacaagttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 36 - DNA, 1987 bp, synthetic construct
  source 1..1987: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtacctagaccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagccgcggaacggccgtgccaccatgtctgaggaacagtttggtggtgatggggctgctgctgcagctacagctgctgttggaggatctgctggggaacaagagggtgccatggttgctgctacacaaggtgctgcagctgctgctggtagtggtgctggaacaggtggtggaacagccagtggtggcacagaaggaggctctgctgaatctgaaggggccaagattgatgccagcaagaatgaggaagatgagggccacagcaacagctccccaagacactctgaagctgccacagctcagagggaagagtggaagatgttcattggaggcctgagctgggacaccaccaagaaggacctgaaggactacttcagcaagtttggagaagtggtggactgcaccctgaagctggaccctatcacaggcagaagcagaggctttggctttgtgctgttcaaagaatctgagtctgtggacaaagtgatggaccagaaagaacacaagctgaatgggaaagtgattgaccccaagagggccaaagccatgaagaccaaagagcctgtcaagaagatctttgttggagggctgtcccctgacacacctgaggaaaagatcagagagtactttggaggatttggagaggtggaatccattgagctgcccatggacaacaagaccaacaagagaagaggcttctgcttcatcaccttcaaagaggaagaaccagtcaagaaaatcatggaaaagaaataccacaatgtgggcctgagcaagtgtgaaatcaaggtggccatgagcaaagagcagtaccagcaacaacagcagtggggctccagaggaggttttgctggcagagctagaggcagaggtggtgaccagcagtctggctatggcaaggtgtccagaagaggtggacatcagaacagctacaagccctactgatgaagcggccatcctcgagggtaccgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 37 - protein, 240 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLP

SEQ ID NO: 38 - protein, 12 aa, synthetic construct
QQVSIEAIQEVE

SEQ ID NO: 39 - protein, 75 aa, synthetic construct
MLPRPPKVTKEEHFQLHHQMHYSQQITVSLAQGYERTSSPKPRFKSYAYTQAAYVTTSDPTRSPFPSQHLEAPED

SEQ ID NO: 40 - protein, 9 aa, synthetic construct
KSFGSSLME

SEQ ID NO: 41 - protein, 111 aa, synthetic construct
SEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHR

SEQ ID NO: 42 - protein, 109 aa, synthetic construct
VLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQD

SEQ ID NO: 43 - protein, 109 aa, synthetic construct
LKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQ

SEQ ID NO: 44 - protein, 47 aa, synthetic construct
QPDLAPGLTTIGASPTQTVTLVTQPVVTKETAISKLEMPSSLMLEVP

SEQ ID NO: 45 - protein, 106 aa, synthetic construct
RLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHE

SEQ ID NO: 46 - protein, 72 aa, synthetic construct
AHRDFGPASQHFLSTSVQGPWERAISPNKVPYYINHETQTTCWDHPKMTELYQSLADLNNVRFSAYRTAMKL

SEQ ID NO: 47 - protein, 248 aa, synthetic construct
RRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYC

SEQ ID NO: 48 - protein, 194 aa, synthetic construct
TPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 49 - protein, 140 aa, synthetic construct
TPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 50 - protein, 35 aa, synthetic construct
MENSNGSYLNDSISPNESIDDEHLLIQHYCQSLNQ

SEQ ID NO: 51 - protein, 3685 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 52 - protein, 1337 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPR

SEQ ID NO: 53 - protein, 1191 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 54 - protein, 1283 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQQPDLAPGLTTIGASPTQTVTLVTQPVVTKETAISKLEMPSSLMLEVPTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLES

SEQ ID NO: 55 - DNA, 720 bp, synthetic construct
  source 1..720: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcct

SEQ ID NO: 56 - DNA, 36 bp, synthetic construct
  source 1..36: mol_type = other DNA
caacaagtgagcattgaagccatccaggaagtggaa

SEQ ID NO: 57 - DNA, 225 bp, synthetic construct
  source 1..225: mol_type = other DNA
atgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagac

SEQ ID NO: 58 - DNA, 225 bp, synthetic construct; identical to SEQ ID NO: 57
  source 1..225: mol_type = other DNA

SEQ ID NO: 59 - DNA, 333 bp, synthetic construct
  source 1..333: mol_type = other DNA
agtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacataga

SEQ ID NO: 60 - DNA, 327 bp, synthetic construct
  source 1..327: mol_type = other DNA
gttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagac

SEQ ID NO: 61 - skipped

SEQ ID NO: 62 - DNA, 327 bp, synthetic construct
  source 1..327: mol_type = other DNA
ctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacag

SEQ ID NO: 63 - DNA, 12 bp, synthetic construct
  source 1..12: mol_type = other DNA
caaacccttgaa

SEQ ID NO: 64 - DNA, 141 bp, synthetic construct
  source 1..141: mol_type = other DNA
cagcctgacctagctcctggactgaccactattggagcctctcctactcagactgttactctggtgacacaacctgtggttactaaggaaactgccatctccaaactagaaatgccatcttccttgatgttggaggtacct

SEQ ID NO: 65 - skipped

SEQ ID NO: 66 - DNA, 315 bp, synthetic construct
  source 1..315: mol_type = other DNA
agactccaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaa

SEQ ID NO: 67 - DNA, 216 bp, synthetic construct
  source 1..216: mol_type = other DNA
gcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactc

SEQ ID NO: 68 - DNA, 744 bp, synthetic construct
  source 1..744: mol_type = other DNA
cgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgc

SEQ ID NO: 69 - DNA, 582 bp, synthetic construct
  source 1..582: mol_type = other DNA
actccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactcccgttactctgatcaacttctggccagtagattctgcgcctgcctcgtcccctcagctttcacacgatgatactcattcacgcattgaacattatgctagcaggctagcagaaatggaaaacagcaatggatcttatctaaatgatagcatctctcctaatgagagcatagatgatgaacatttgttaatccagcattactgccaaagtttgaaccaggactcccccctgagccagcctcgtagtcctgcccagatcttgatttccttagagagtgaggaaagaggggagctagagagaatcctagcagatcttgaggaagaaaacaggaatctgcaagcagaatatgaccgtctaaagcagcagcacgaacataaaggcctgtccccactgccgtcccctcctgaaatgatgcccacctctccccagagtccccgg

SEQ ID NO: 70 - DNA, 12 bp, synthetic construct
  source 1..12: mol_type = other DNA
gagacccttgaa

SEQ ID NO: 71 - skipped

SEQ ID NO: 72 - DNA, 720 bp, synthetic construct
  source 1..720: mol_type = other DNA
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccc

SEQ ID NO: 73 - DNA, 36 bp, synthetic construct
  source 1..36: mol_type = other DNA
cagcaagtgtccattgaggccattcaagaggttgag

SEQ ID NO: 74 - DNA, 225 bp, synthetic construct
  source 1..225: mol_type = other DNA
atgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggac

SEQ ID NO: 75 - DNA, 27 bp, synthetic construct
  source 1..27: mol_type = other DNA
aagagctttggcagcagcctgatggaa

SEQ ID NO: 76 - DNA, 333 bp, synthetic construct
  source 1..333: mol_type = other DNA
tctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacaga

SEQ ID NO: 77 - DNA, 327 bp, synthetic construct
  source 1..327: mol_type = other DNA
gtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggac

SEQ ID NO: 78 - skipped

SEQ ID NO: 79 - DNA, 327 bp, synthetic construct
  source 1..327: mol_type = other DNA
ctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccag

SEQ ID NO: 80 - DNA, 141 bp, synthetic construct
  source 1..141: mol_type = other DNA
caacctgatcttgcccctggcctgaccacaattggagcctctccaacacagactgtgaccctggttacccagccagtggtcaccaaagagacagccatcagcaaactggaaatgcccagctctctgatgctggaagtcccc

SEQ ID NO: 81-82 - skipped (2 entries)

SEQ ID NO: 83 - DNA, 12 bp, synthetic construct
  source 1..12: mol_type = other DNA
cagacactggaa

SEQ ID NO: 84 - DNA, 318 bp, synthetic construct
  source 1..318: mol_type = other DNA
aggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgag

SEQ ID NO: 85 - DNA, 216 bp, synthetic construct
  source 1..216: mol_type = other DNA
gcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctc

SEQ ID NO: 86 - DNA, 744 bp, synthetic construct
  source 1..744: mol_type = other DNA
agaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgc

SEQ ID NO: 87 - DNA, 588 bp, synthetic construct
  source 1..588: mol_type = other DNA
accccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatga

SEQ ID NO: 88 - DNA, 426 bp, synthetic construct
  source 1..426: mol_type = other DNA
accccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatga

SEQ ID NO: 89 - DNA, 12 bp, synthetic construct
  source 1..12: mol_type = other DNA
garacactggaa

SEQ ID NO: 90 - skipped

SEQ ID NO: 91 - DNA, 4017 bp, synthetic construct
  source 1..4017: mol_type = other DNA
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatga

SEQ ID NO: 92 - DNA, 3573 bp, synthetic construct
  source 1..3573: mol_type = other DNA
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacc

SEQ ID NO: 93 - DNA, 3855 bp, synthetic construct
  source 1..3855: mol_type = other DNA
atgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatga

SEQ ID NO: 94 - DNA, 4734 bp, synthetic construct
  source 1..4734: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatgactcgagaggcctaataaagagctcagatgcatcgatcagagtgtgttggttttttgtgtgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 95 - DNA, 4364 bp, synthetic construct
  source 1..4364: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaaggtgagtatctcagggatccagacatggggatatgggaggtgcctctgatcccagggctcactgtgggtctctctgttcacaggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacctgatgagtcgacaggcctaataaagagctcagatgcatcgatcagagtgtgttggttttttgtgtggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 96 - DNA, 4560 bp, synthetic construct
  source 1..4560: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtcttgatgagtcgacaggcctaataaagagctcagatgcatcgatcagagtgtgttggttttttgtgtggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 97 - DNA, 600 bp, synthetic construct
  source 1..600: mol_type = other DNA
agccagcctcagtttcccctccactcagtccctaggaggaaggggcgcccaagcgcgggtttctggggttagactgccctccattgcaattggtccttctcccggcctctgcttcctccagctcacagggtatctgctcctcctggagccacaccttggttccccgaggtgccgctgggactcgggtaggggtgagggcccagggggcacagggggagccgagggccacaggaagggctggtggctgaaggagactcaggggccaggggacggtggcttctacgtgcttgggacgttcccagccaccgtcccatgttcccggcggggggccagctgtccccaccgccagcccaactcagcacttggtcagggtatcagcttggtgggggggcgtgagcccagcccctggggcggctcagcccatacaaggccatggggctgggcgcaaagcatgcctgggttcagggtgggtatggtgcgggagcagggaggtgagaggctcagctgccctccagaactcctccctggggacaacccctcccagccaatagcacagcctaggtccccctatataaggccacggctgctggcccttccttt

SEQ ID NO: 98 - DNA, 600 bp, synthetic construct
  source 1..600: mol_type = other DNA
ctgaggctcagggctagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccct

SEQ ID NO: 99 - DNA, 240 bp, synthetic construct
  source 1..240: mol_type = other DNA
ctgcagacatgcttgctgcctgccctggcgtgccctggcgaggcttgccgtcacaggacccccgctggctgactcaggggcgcaggctcttgcgggggagctggcctcccgcccccacggccacgggccctttcctggcaggacagcgggatcttgcagctgtcaggggaggggatgacgggggactgatgtcaggaggggatacaaatagtgccgaacaaggaccggattagatctacc

SEQ ID NO: 100 - DNA, 600 bp, synthetic construct
  source 1..600: mol_type = other DNA
ggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagc

SEQ ID NO: 101 - DNA, 600 bp, synthetic construct
  source 1..600: mol_type = other DNA
agaaacctgtggtctagaggcggggcggggccgatggaggcaacgcacgcccccgcaggcgcccaggccacgccctctgccgcagcattcggtgaaacctgcgttccgagaacttctgaaaactttatctgggggccttcgagaaggctcagacagtaagggtgcatgctgccaatcctgaggagctgagttcgatccctgagaccttcagggtggacagagacggactcccacatgttgttttctgacttctacatgtgtccagtcatacatacacaaatatggaataaacagatggctcatcaggtaagagtgctggctgcttttgcagaggacccaggttcgatttccagaacccacatgtcggctcaaaatcatctgtaattccagttccagggagatccagcactttcttccagggcctccacagacacacataaaataaagataaaaatctccaaaaaatattgttttaataattacaacctgaagaccttgcacaactattcctggctgagaagatggtaagggcgctagctgccaagcttgacagcctgagtttcatctccaagaaccatgaaaactgactcctgggaatta

SEQ ID NO: 102 - DNA, 600 bp, synthetic construct
  source 1..600: mol_type = other DNA
ggaagcagaaggccaacattcctcccaagggaaactgaggctcagagttaaaacccaggtatcagtgatatgcatgtgccccggccagggtcactctctgactaaccggtacctaccctacaggcctacctagagactcttttgaaaggatggtagagacctgtccgggctttgcccacagtcgttggaaacctcagcattttctaggcaacttgtgcgaataaaacacttcgggggtccttcttgttcattccaataacctaaaacctctcctcggagaaaatagggggcctcaaacaaacgaaattctctagcccgctttccccaggataaggcaggcatccaaatggaaaaaaaggggccggccgggggtctcctgtcagctccttgccctgtgaaacccagcaggcctgcctgtcttctgtcctcttggggctgtccaggggcgcaggcctcttgcgggggagctggcctccccgccccctcgcctgtggccgcccttttcctggcaggacagagggatcctgcagctgtcaggggaggggcgccggggggtgatgtcaggagggctacaaatagtgcagacagctaaggggctcc

SEQ ID NO: 103 - DNA, 601 bp, synthetic construct
  source 1..601: mol_type = other DNA
ggggtgatgtgtgtcagatctctggattgggggagcttcaaagtgggaaagaaaatggagttcaaatgtggggcttattttccatccctacctggagcccatgactcctcccggctcacctgaccacagggctacctcccctgagcttaagcatcaaggcttagtagtctgagttaagdaacccataaatggggtgcattgtggcaggtcagcaatcgtgtgtccaggtgggcagaactggggagacctttcaaacaggtaaatcttgggaagtacagaccagcagtctgcaaagcagtgacctttggcccagcacagcccttccgtgagccttggagccagttgggaggggcagacagctggggatactctccatatacggcctggtccggtcctagctacctgggccagggccagtcctctccttctttggtcagtgcaggagacccgggcggggacccaggctgagaaccagccgaaggaagggactctagtgcccgacacccaaatatggcttgggaagggcagcaacattccttcggggcggtgtggggagagctcccgggactatataaaaacctgtgcaaggggacaggcggtc

SEQ ID NO: 104 - DNA, 770 bp, synthetic construct
  source 1..770: mol_type = other DNA
ctagaagctgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcac

SEQ ID NO: 105 - DNA, 714 bp, synthetic construct
  source 1..714: mol_type = other DNA
ccactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggatccactacgggtctatgctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggaccactacgggtctaggctgcccatgtaaggaggcaaggcctggggacacccgagatgcctggttataattaaccccaacacctgctgccccccccccccaacacctgctgcctgagcctgagcggttaccccaccccggtgcctgggtcttaggctctgtacaccatggaggagaagctcgctctaaaaataaccctgtccctggtcctccctggggacagcccctcctggctagtcacaccctgtaggctcctctatataacccaggggcacaggggctgcccccgggtcac

SEQ ID NO: 106 - DNA, 383 bp, synthetic construct
  source 1..383: mol_type = other DNA
cgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattc

SEQ ID NO: 107 - DNA, 865 bp, synthetic construct
  source 1..865: mol_type = other DNA
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 108 - DNA, 1725 bp, synthetic construct
  source 1..1725: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 109 - DNA, 251 bp, synthetic construct
  source 1..251: mol_type = other DNA
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 110 - DNA, 212 bp, synthetic construct
  source 1..212: mol_type = other DNA
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 111 - DNA, 82 bp, synthetic construct; identical to SEQ ID NO: 19
  source 1..82: mol_type = other DNA

SEQ ID NO: 112 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 113 - DNA, 97 bp, synthetic construct
  source 1..97: mol_type = other DNA
gtaagtttagtctttttgtcttttatttcaggtcccggatccggtggtggtgcaaatcaaagaactgctcctcagtggatgttgcctttacttctag

SEQ ID NO: 114 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 115 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 116 - protein, 738 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 117 - protein, 736 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 118 - protein, 737 aa, synthetic construct
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPGQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 119 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 120 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 121 - DNA, 22 bp, synthetic construct
  source 1..22: mol_type = other DNA
ggctttgtgctgttcaaagaat

SEQ ID NO: 122 - DNA, 18 bp, synthetic construct
  source 1..18: mol_type = other DNA
atggctttggccctcttg

SEQ ID NO: 123 - DNA, 17 bp, synthetic construct
  source 1..17: mol_type = other DNA
agctgaatgggaaagtg

SEQ ID NO: 124 - DNA, 23 bp, synthetic construct
  source 1..23: mol_type = other DNA
aagggacctttaccagtgatgtg

SEQ ID NO: 125 - DNA, 21 bp, synthetic construct
  source 1..21: mol_type = other DNA
acttactctcgccttcctcgg

SEQ ID NO: 126 - DNA, 15 bp, synthetic construct
  source 1..15: mol_type = other DNA
cagcaaaggaattca

SEQ ID NO: 127 - DNA, 335 bp, synthetic construct
  source 1..335: mol_type = other DNA
agaggccgtccgccctcggcaccatcctcacgacacccaaatatggcgacgggtgaggaatggtggggagttatttttagagcggtgaggaaggtgggcaggcagcaggtgttggcgctccatatttggcgggagttatttttagagcggaggaatggtggacacccaaatatggcgacggttcctcacccgtcgctaaaaataactccgtgtccgccctcggccggggccgcattcctgggggccgggcggtgctcccgcccgcctcgataaaaggctccggggccggcggcggcccacgagctacccggaggagcgggaggcgccaagcggaa

SEQ ID NO: 128 - DNA, 328 bp, synthetic construct
  source 1..328: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctcggccggggccgcattcctgggggccgggcggtgctcccgcccgcctcgataaaaggctccggggccggcggcggcccacgagctacccggaggagcgggaggcgccaagc

SEQ ID NO: 129 - DNA, 4361 bp, synthetic construct
  source 1..4361: mol_type = other DNA
agaggccgtccgccctcggcaccatcctcacgacacccaaatatggcgacgggtgaggaatggtggggagttatttttagagcggtgaggaaggtgggcaggcagcaggtgttggcgctccatatttggcgggagttatttttagagcggaggaatggtggacacccaaatatggcgacggttcctcacccgtcgctaaaaataactccgtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatga

SEQ ID NO: 130 - DNA, 4734 bp, synthetic construct
  source 1..4734: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttggcgctccatatttggcgggagttatttttagagcggaggaatggtggacacccaaatatggcgacggttcctcacccgtcgctaaaaataactccgtgtccgccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacccctgtgacactgatcaatttctggccagtggactctgcccctgcctcaagtccacagctgtcccatgatgacacccacagcagaattgagcactatgcctccagactggcagagatggaaaacagcaatggcagctacctgaatgatagcatcagccccaatgagagcattgatgatgagcatctgctgatccagcactactgtcagtccctgaaccaggactctccactgagccagcctagaagccctgctcagatcctgatcagccttgagtctgaggaaaggggagagctggaaagaatcctggcagatcttgaggaagagaacagaaacctgcaggcagagtatgacaggctcaaacagcagcatgagcacaagggactgagccctctgccttctcctcctgaaatgatgcccacctctccacagtctccaaggtgatgactcgagaggcctaataaagagctcagatgcatcgatcagagtgtgttggttttttgtgtgccagggtaatgggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 131 - DNA, 3920 bp, synthetic construct
  source 1..3920: mol_type = other DNA
ggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacctgatga

SEQ ID NO: 132 - DNA, 4284 bp, synthetic construct
  source 1..4284: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctcatatgcagggtaatggggatcctctagaggccgtccgccctcggcaccatcctcacgacacccaaatatggcgacgggtgaggaatggtggggagttatttttagagcggtgaggaaggtgggcaggcagcaggtgttgggggagttatttttagagcggggagttatttttagagcggaggaatggtggacacccaaatatggcgacggttcctcacggacacccaaatatggcgacgggccctcggccggggccgcattcctgggggccgggcggtgctcccgcccgcctcgataaaaggctccggggccggcggcggcccacgagctacccggaggagcgggaggcgccaagcggaattcgccaccatgctttggtgggaagaggtggaagattgctatgagagggaagatgtgcagaagaaaaccttcaccaaatgggtcaatgcccagttcagcaagtttggcaagcagcacattgagaacctgttcagtgacctgcaggatggcagaaggctgctggatctgctggaaggcctgacaggccagaagctgcctaaagagaagggcagcacaagagtgcatgccctgaacaatgtgaacaaggccctgagagtgctgcagaacaacaatgtggacctggtcaatattggcagcacagacattgtggatggcaaccacaagctgaccctgggcctgatctggaacatcatcctgcactggcaagtgaagaatgtgatgaagaacatcatggctggcctgcagcagaccaactctgagaagatcctgctgagctgggtcagacagagcaccagaaactaccctcaagtgaatgtgatcaacttcaccacctcttggagtgatggactggccctgaatgccctgatccacagccacagacctgacctgtttgactggaactctgttgtgtgccagcagtctgccacacagagactggaacatgccttcaacattgccagataccagctgggaattgagaaactgctggaccctgaggatgtggacaccacctatcctgacaagaaatccatcctcatgtacatcaccagcctgttccaggtgctgccccagcaagtgtccattgaggccattcaagaggttgagatgctgcccagacctcctaaagtgaccaaagaggaacacttccagctgcaccaccagatgcactactctcagcagatcacagtgtctctggcccagggatatgagagaacaagcagccccaagcctaggttcaagagctatgcctacacacaggctgcctatgtgaccacatctgaccccacaagaagcccatttccaagccagcatctggaagcccctgaggacaagagctttggcagcagcctgatggaatctgaagtgaacctggatagataccagacagccctggaagaagtgctgtcctggctgctgtctgctgaggatacactgcaggctcagggtgaaatcagcaatgatgtggaagtggtcaaggaccagtttcacacccatgagggctacatgatggacctgacagcccaccagggcagagtgggaaatatcctgcagctgggctccaagctgattggcacaggcaagctgtctgaggatgaagagacagaggtgcaagagcagatgaacctgctgaacagcagatgggagtgtctgagagtggccagcatggaaaagcagagcaacctgcacagagtgctcatggacctgcagaatcagaaactgaaagaactgaatgactggctgaccaagacagaagaaaggactaggaagatggaagaggaacctctgggaccagacctggaagatctgaaaagacaggtgcagcagcataaggtgctgcaagaggaccttgagcaagagcaagtcagagtgaacagcctgacacacatggtggtggttgtggatgagtcctctggggatcatgccacagctgctctggaagaacagctgaaggtgctgggagacagatgggccaacatctgtaggtggacagaggatagatgggtgctgctccaggacattctgctgaagtggcagagactgacagaggaacagtgcctgttttctgcctggctctctgagaaagaggatgctgtcaacaagatccataccacaggcttcaaggatcagaatgagatgctcagctccctgcagaaactggctgtgctgaaggctgacctggaaaagaaaaagcagtccatgggcaagctctacagcctgaagcaggacctgctgtctaccctgaagaacaagtctgtgacccagaaaactgaggcctggctggacaactttgctagatgctgggacaacctggtgcagaagctggaaaagtctacagcccagatcagccagcaacctgatcttgcccctggcctgaccacaattggagcctctccaacacagactgtgaccctggttacccagccagtggtcaccaaagagacagccatcagcaaactggaaatgcccagctctctgatgctggaagtccccacactggaaaggctgcaagaacttcaagaggccacagatgagctggacctgaagctgagacaggctgaagtgatcaaaggcagctggcagccagttggggacctgctcattgatagcctgcaggaccatctggaaaaagtgaaagccctgaggggagagattgcccctctgaaagaaaatgtgtcccatgtgaatgacctggccagacagctgaccacactgggaatccagctgagcccctacaacctgagcacccttgaggacctgaacaccaggtggaagctcctccaggtggcagtggaagatagagtcaggcagctgcatgaggcccacagagattttggaccagccagccagcactttctgtctacctctgtgcaaggcccctgggagagagctatctctcctaacaaggtgccctactacatcaaccatgagacacagaccacctgttgggatcaccccaagatgacagagctgtaccagagtctggcagacctcaacaatgtcagattcagtgcctacaggactgccatgaagctcagaaggctccagaaagctctgtgcctggacctgctttccctgagtgcagcttgtgatgccctggaccagcacaatctgaagcagaatgaccagcctatggacatcctccagatcatcaactgcctcaccaccatctatgataggctggaacaagagcacaacaatctggtcaatgtgcccctgtgtgtggacatgtgcctgaattggctgctgaatgtgtatgacacaggcagaacaggcaggatcagagtcctgtccttcaagacaggcatcatctccctgtgcaaagcccacttggaggacaagtacagatacctgttcaagcaagtggcctccagcacaggcttttgtgaccagagaaggctgggcctgctcctgcatgacagcattcagatccctagacagctgggagaagtggcttcctttggaggcagcaatattgagccatcagtcaggtcctgttttcagtttgccaacaacaagcctgagattgaggctgccctgttcctggactggatgagacttgagcctcagagcatggtctggctgcctgtgcttcatagagtggctgctgctgagactgccaagcaccaggccaagtgcaacatctgcaaagagtgccccatcattggcttcagatacagatccctgaagcacttcaactatgatatctgccagagctgcttctttagtggcagggttgccaagggccacaaaatgcactaccccatggtggaatactgcaccccaacaacctctggggaagatgttagagactttgccaaggtgctgaaaaacaagttcaggaccaagagatactttgctaagcaccccagaatgggctacctgcctgtccagacagtgcttgagggtgacaacatggaaacctgatgagtcgacaggcctaataaagagctcagatgcatcgatcagagtgtgttggttttttgtgtggctagctgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 133 - protein, 1197 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 134 - protein, 1192 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETDTM

SEQ ID NO: 135 - protein, 1325 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDQPDLAPGLTTIGASPTQTVTLVTQPVVTKETAISKLEMPSSLMLEVPTHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMET

SEQ ID NO: 136 - protein, 1392 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEDTM

SEQ ID NO: 137 - protein, 1471 aa, synthetic construct
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDTLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 138 - DNA, 125 bp, synthetic construct
  source 1..125: mol_type = other DNA
gtaagtatcaaggttacaagacaggtttaaggagaccaatagaaactgggcttgtcgagacagagaagactcttgcgtttctgataggcacctattggtcttactgacatccactttgcctttct

SEQ ID NO: 139 - DNA, 1602 bp, synthetic construct
  source 1..1602: mol_type = other DNA
atagtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaattgcccttgttaatgtctacccagtgggggaagattactacgcttgcacagagaccaactttattacaaagattaatccagagaccttggagacaattaagcaggttgatctttgcaactaagtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcagctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 140 - DNA, 596 bp, synthetic construct
  source 1..596: mol_type = other DNA
cgagtttaattggtttatagaactcttcaaacaaattaaaccaaaaatttcaatgccaagaaagggtctttaaaacgaaattacagaaggaccaaatgataaggaagaaaaatgcagagataaaagtaatatcaattaggatcataagctacttattatcaatgaaaagtaacagaaacatagatgctgcagaaatcttctgaggagtagcttcaacgcctcagggtgtggacaatgtattcagcatagaggtccctgtaatggggatatcagaatccagagttgctttaatgttacaaactaaaaaagatgtaagagagtttggttcttgataaagaaacagaggcttacattgagtactggatagcttcaaccgcagactcagatggcagaaaatcattcactgcaacttccttgttctcgtttttcttgtctgtaagatattagagttaaagggaaaaactaatacttgttgagagatcaatagagatgaataaggaggaacactgaagaaaaaggatacagtcttcgaagaaacgacggatttcagagagacggtgaggaggaagttctttgatgtcagtgtagtgcttata

SEQ ID NO: 141 - DNA, 1096 bp, synthetic construct
  source 1..1096: mol_type = other DNA
cgagtttaattggtttatagaactcttcaaacaaattaaaccaaaaatttcaatgccaagaaagggtctttaaaacgaaattacagaaggaccaaatgataaggaagaaaaatgcagagataaaagtaatatcaattaggatcataagctacttattatcaatgaaaagtaacagaaacatagatgctgcagaaatcttctgaggagtagcttcaacgcctcagggtgtggacaatgtattcagcatagaggtccctgtaatggggatatcagaatccagagttgctttaatgttacaaactaaaaaagatgtaagagagtttggttcttgataaagaaacagaggcttacattgagtactggatagcttcaaccgcagactcagatggcagaaaatcattcactgcaacttccttgttctcgtttttcttgtctgtaagatattagagttaaagggaaaaactaatacttgttgagagatcaatagagatgaataaggaggaacactgaagaaaaaggatacagtcttcgaagaaacgacggatttcagagagacggtgaggaggaagttctttgatgtcagtgtagtgcttatattcaggatcatcaacacacactgcaatgatcttgtcatctttttcaccctaaaattacagcgccaaaaatacaagattggagtacaagaccatttaaactgacctaaaggattagagtaagagaaaaaaaaaacagagtcttttcattgatcaagtttaggttttacctggtcaatcataggcattaatccaatggctctggcacgcagaaaacaacccggaagcacaggttcctacacaaagataataatatatatttgaaatacaaaaaattggtgcaaatagtatagggataatatgagaaagaaagaaagagtaatacctgcatgatgactaagacatcaatggggtcattgtcttcacacaatgtgcgaggaacaaaaccatagttgtgagggtacacaactgatgagtagagaatacgatcaacctgaatgagagatatcaaacttgttgagattgattttgctataagaaaaccattcatataaaaaataaaa

SEQ ID NO: 142 - DNA, 1596 bp, synthetic construct
  source 1..1596: mol_type = other DNA
cgagtttaattggtttatagaactcttcaaacaaattaaaccaaaaatttcaatgccaagaaagggtctttaaaacgaaattacagaaggaccaaatgataaggaagaaaaatgcagagataaaagtaatatcaattaggatcataagctacttattatcaatgaaaagtaacagaaacatagatgctgcagaaatcttctgaggagtagcttcaacgcctcagggtgtggacaatgtattcagcatagaggtccctgtaatggggatatcagaatccagagttgctttaatgttacaaactaaaaaagatgtaagagagtttggttcttgataaagaaacagaggcttacattgagtactggatagcttcaaccgcagactcagatggcagaaaatcattcactgcaacttccttgttctcgtttttcttgtctgtaagatattagagttaaagggaaaaactaatacttgttgagagatcaatagagatgaataaggaggaacactgaagaaaaaggatacagtcttcgaagaaacgacggatttcagagagacggtgaggaggaagttctttgatgtcagtgtagtgcttatattcaggatcatcaacacacactgcaatgatcttgtcatctttttcaccctaaaattacagcgccaaaaatacaagattggagtacaagaccatttaaactgacctaaaggattagagtaagagaaaaaaaaaacagagtcttttcattgatcaagtttaggttttacctggtcaatcataggcattaatccaatggctctggcacgcagaaaacaacccggaagcacaggttcctacacaaagataataatatatatttgaaatacaaaaaattggtgcaaatagtatagggataatatgagaaagaaagaaagagtaatacctgcatgatgactaagacatcaatggggtcattgtcttcacacaatgtgcgaggaacaaaaccatagttgtgagggtacacaactgatgagtagagaatacgatcaacctgaatgagagatatcaaacttgttgagattgattttgctataagaaaaccattcatataaaaaataaactttgttctcatctaaccttgatgagtcctgtctttttgtcaagctcgtatttgaccttgcttcctttagtgatctcaacaacctaataatcatccaaagataaaatgattagagaatctaataacaacatactctgtttagaacaaagagtaggaaaaaacttaccacattgaaaatctgtggagctccaggtcctagatagattatccagacttatgatttagtaacagaatacaaaagtatgaaatcaaaaagtagcatgtttagaatgatttatataccaatctcaagatcatgccatggatgagcagctacggatcttcttgacaaggatgagagaatcctctcgttaagacgaggagctggtcgctgcagcctctggttatctttagtttcttcactcatctgtcaaaatcagaacgtttcatcactcattgatattgactgaatctaacatcataaccctaattggcagagagagaatcaatcgaatcaagaga

SEQ ID NO: 143 - DNA, 2002 bp, synthetic construct
  source 1..2002: mol_type = other DNA
cgagtttaattggtttatagaactcttcaaacaaattaaaccaaaaatttcaatgccaagaaagggtctttaaaacgaaattacagaaggaccaaatgataaggaagaaaaatgcagagataaaagtaatatcaattaggatcataagctacttattatcaatgaaaagtaacagaaacatagatgctgcagaaatcttctgaggagtagcttcaacgcctcagggtgtggacaatgtattcagcatagaggtccctgtaatggggatatcagaatccagagttgctttaatgttacaaactaaaaaagatgtaagagagtttggttcttgataaagaaacagaggcttacattgagtactggatagcttcaaccgcagactcagatggcagaaaatcattcactgcaacttccttgttctcgtttttcttgtctgtaagatattagagttaaagggaaaaactaatacttgttgagagatcaatagagatgaataaggaggaacactgaagaaaaaggatacagtcttcgaagaaacgacggatttcagagagacggtgaggaggaagttctttgatgtcagtgtagtgcttatattcaggatcatcaacacacactgcaatgatcttgtcatctttttcaccctaaaattacagcgccaaaaatacaagattggagtacaagaccatttaaactgacctaaaggattagagtaagagaaaaaaaaaacagagtcttttcattgatcaagtttaggttttacctggtcaatcataggcattaatccaatggctctggcacgcagaaaacaacccggaagcacaggttcctacacaaagataataatatatatttgaaatacaaaaaattggtgcaaatagtatagggataatatgagaaagaaagaaagagtaatacctgcatgatgactaagacatcaatggggtcattgtcttcacacaatgtgcgaggaacaaaaccatagttgtgagggtacacaactgatgagtagagaatacgatcaacctgaatgagagatatcaaacttgttgagattgattttgctataagaaaaccattcatataaaaaataaactttgttctcatctaaccttgatgagtcctgtctttttgtcaagctcgtatttgaccttgcttcctttagtgatctcaacaacctaataatcatccaaagataaaatgattagagaatctaataacaacatactctgtttagaacaaagagtaggaaaaaacttaccacattgaaaatctgtggagctccaggtcctagatagattatccagacttatgatttagtaacagaatacaaaagtatgaaatcaaaaagtagcatgtttagaatgatttatataccaatctcaagatcatgccatggatgagcagctacggatcttcttgacaaggatgagagaatcctctcgttaagacgaggagctggtcgctgcagcctctggttatctttagtttcttcactcatctgtcaaaatcagaacgtttcatcactcattgatattgactgaatctaacatcataaccctaattggcagagagagaatcaatcgaatcaagagtattaaatggaaaaagcgaatcaagaccccacaagggaaaacaatccttaaagcagacttgagatcgatcatacccaaattatggattcatatattgttaacgtatcgattactgaaaagatgtataccaaatctgttcactttttctctatagactcgatggatgattgagatttgaagcaacaaaatacccagaagattaaacatggaaaagcatcaaactttgatgatcttagaacgatgacaaaagaaaaaaaaacgtacctttggatcgaaacgaaacagccgattgttgttttctttatcgcaaggatgatgaagaaactttgggagagaaacaagtgaagcccgttggtctagcaagtgattgtaaaatgtatatatgagtcaccaccgagatatacgga

(19) United States
(12) Patent Application Publication
(10) Pub. No.: US 2024/0409955 A1
(43) Pub. Date: Dec. 12, 2024

(54) AUF1 COMBINATION THERAPIES FOR TREATMENT OF MUSCLE DEGENERATIVE DISEASE

(71) Applicant: NEW YORK UNIVERSITY, New York, NY (US)

(72) Inventors: Dounia ABBADI, New York, NY (US); Robert J. SCHNEIDER, New York, NY (US); Subha KARUMUTHIL-MELETHIL, Germantown, MD (US); Chunping QIAO, Chapel Hill, NC (US); Kirk ELLIOTT, Rockville, MD (US); Ye LIU, Clarksville, MD (US); Olivier DANOS, Princeton, NJ (US); Steven FOLTZ, Hyattsville, MD (US)

(21) Appl. No.: 18/580,093

(22) PCT Filed: Jul. 19, 2022
(86) PCT No.: PCT/US2022/073908

§ 371 Date: Jan. 17, 2024

Related U.S. Application Data
(60) Provisional application No. 63/223,494, filed on Jul. 19, 2021.

(51) Int. Cl. C12N 15/86; A61K 38/17; A61P 21/00

(52) U.S. Cl. CPC C12N 15/86; A61K 38/1709; A61K 38/1719; A61P 21/00; C12N 2750/14143; C12N 2830/008

(57) ABSTRACT

Provided are methods of treating or ameliorating the symptoms of dystrophinopathies, such as Duchenne muscular dystrophy and Becker muscular dystrophy by administration of therapeutically effective doses of recombinant adeno-associated viruses (rAAV) containing a transgene encoding AUF1 and a second rAAV encoding a microdystrophin or other therapeutic effective to treat the dystrophinopathy. Also provided are rAAV vectors encoding AUF1 proteins.

### 1. FIELD OF THE INVENTION

The present invention relates to treatment of muscle degenerative disease, such as dystrophinopathies, by administration of doses of gene therapy vectors, such as AAV gene therapy vectors in which the transgene encodes AUF1 in combination with a second therapeutic, including a gene therapy vector encoding a microdystrophin for treating dystrophinopathies. Also provided are rAAV gene therapy vectors encoding an AUF1 protein and methods of treatment using same.

### 2. BACKGROUND

A group of neuromuscular diseases called dystrophinopathies are caused by mutations in the DMD gene. Each dystrophinopathy has a distinct phenotype, with all patients suffering from muscle weakness and ultimately cardiomyopathy with ranging severity. Duchenne muscular dystrophy (DMD) is a severe, X-linked, progressive neuromuscular disease affecting approximately one in 3,600 to 9,200 live male births. The disorder is caused by frameshift mutations in the dystrophin gene abolishing the expression of the dystrophin protein. Due to the lack of the dystrophin protein, skeletal muscle, and ultimately heart and respiratory muscles (e.g., intercostal muscles and diaphragm), degenerate causing premature death. Progressive weakness and muscle atrophy begin in childhood. Affected individuals experience breathing difficulties, respiratory infections, and swallowing problems. Almost all DMD patients will develop cardiomyopathy. Pneumonia compounded by cardiac involvement is the most frequent cause of death, which frequently occurs before the third decade.

Becker muscular dystrophy (BMD) has less severe symptoms than DMD, but still leads to premature death. Compared to DMD, BMD is characterized by later-onset skeletal muscle weakness. Whereas DMD patients are wheelchair dependent before age 13, those with BMD lose ambulation and require a wheelchair after age 16. BMD patients also exhibit preservation of neck flexor muscle strength, unlike their counterparts with DMD. Despite milder skeletal muscle involvement, heart failure from DMD-associated dilated cardiomyopathy (DCM) is a common cause of morbidity and the most common cause of death in BMD, which occurs on average in the mid-40s.

Dystrophin is a cytoplasmic protein encoded by the DMD gene, and functions to link cytoskeletal actin filaments to membrane proteins. Normally, the dystrophin protein, located primarily in skeletal and cardiac muscles, with smaller amounts expressed in the brain, acts as a shock absorber during muscle fiber contraction by linking the actin of the contractile apparatus to the layer of connective tissue that surrounds each muscle fiber. In muscle, dystrophin is localized at the cytoplasmic face of the sarcolemma membrane.

The DMD gene is the largest known human gene. The most common mutations that cause DMD or BMD are large deletion mutations of one or more exons (60-70%), but duplication mutations (5-10%), and single nucleotide variants (including small deletions or insertions, single-base changes, and splice site changes accounting for approximately 25-35% of pathogenic variants in males with DMD and about 10-20% of males with BMD), can also cause pathogenic dystrophin variants. In DMD, mutations often lead to a frame shift resulting in a premature stop codon and a truncated, non-functional or unstable protein. Nonsense point mutations can also result in premature termination codons with the same result. While mutations causing DMD can affect any exon, exons 2-20 and 45-55 are common hotspots for large deletion and duplication mutations. In-frame deletions result in the less severe Becker muscular dystrophy (BMD), in which patients express a truncated, partially functional dystrophin.

Muscle wasting diseases represent a major source of human disease. They can be genetic in origin (primarily muscular dystrophies), related to aging (sarcopenia), or the result of traumatic muscle injury, among others. There are few treatment options available for individuals with myopathies, or those who have suffered severe muscle trauma, or the loss of muscle mass with aging (known as sarcopenia). The physiology of myopathies is well understood and founded on a common pathogenesis of relentless cycles of muscle degeneration and regeneration, typically leading to functional exhaustion of muscle stem (satellite) cells and their progenitor cells that fail to reactivate, and at times their loss as well (Carlson & Conboy, “Loss of Stem Cell Regenerative Capacity Within Aged Niches,” Aging Cell 6(3):371-82 (2007); Shefer et al., “Satellite-cell Pool Size Does Matter: Defining the Myogenic Potency of Aging Skeletal Muscle,” Dev. Biol. 294(1):50-66 (2006); Bernet et al., “p38 MAPK Signaling Underlies a Cell-autonomous Loss of Stem Cell Self-renewal in Skeletal Muscle of Aged Mice,” Nat. Med. 20(3):265-71 (2014); and Dumont et al., “Intrinsic and Extrinsic Mechanisms Regulating Satellite Cell Function,” Development 142(9):1572-1581 (2015)).

Age-related skeletal muscle loss and atrophy is characterized by the progressive loss of muscle mass, strength, and endurance with age. It can be a significant source of frailty, increased fractures, and mortality in the elderly population (Vermeiren et al., “Frailty and the Prediction of Negative Health Outcomes: A Meta-Analysis,” J. Am. Med. Dir. Assoc. 17(12):1163.e1-1163.e17 (2016) and Buford, T. W., “Sarcopenia: Relocating the Forest among the Trees,” Toxicol. Pathol. 45(7):957-960 (2017)). Although different strategies have been investigated to counter muscle loss and atrophy, regular resistance exercise is the most effective in slowing muscle loss and atrophy, but compliance and physical limitations are significant barriers (Wilkinson et al., “The Age-Related loss of Skeletal Muscle Mass and Function: Measurement and Physiology of Muscle Fibre Atrophy and Muscle Fibre Loss in Humans,” Ageing Res. Rev. 47:123-132 (2018)). Consequently, with an aging global population, therapeutic strategies need to be developed to reverse age-related muscle decline.

Muscle regeneration is initiated by skeletal muscle stem (satellite) cells that reside between striated muscle fibers (myofibers), which are the contractile cellular bundles, and the basal lamina that surrounds them (Carlson & Conboy, “Loss of Stem Cell Regenerative Capacity within Aged Niches,” Aging Cell 6(3):371-382 (2007) and Schiaffino & Reggiani, “Fiber Types in Mammalian Skeletal Muscles,” Physiol. Rev. 91(4):1447-1531 (2011)). Upon physical injury to muscle, the anatomical niche is disrupted, normally quiescent satellite cells become activated and proliferate asymmetrically. Some satellite cells reconstitute the stem cell population while most others differentiate and fuse to form new myofibers (Hindi et al., “Signaling Mechanisms in Mammalian Myoblast Fusion,” Sci. Signal. 6(272):re2 (2013)). Studies have demonstrated the singular importance of the satellite cell/myoblast population in muscle regeneration (Shefer et al., “Satellite-cell Pool Size Does Matter: Defining the Myogenic Potency of Aging Skeletal Muscle,” Dev. Biol. 294(1):50-66 (2006); Dumont et al., “Intrinsic and Extrinsic Mechanisms Regulating Satellite Cell Function,” Development 142(9):1572-1581 (2015); Briggs & Morgan, “Recent Progress in Satellite Cell/Myoblast Engraftment—Relevance for Therapy, FEBS J. 280(17):4281-93 (2013); Morgan & Zammit, “Direct Effects of the Pathogenic Mutation on Satellite Cell Function in Muscular Dystrophy,” Exp. Cell Res. 316(18):3100-8 (2010); and Relaix & Zammit, “Satellite Cells are Essential for Skeletal Muscle Regeneration: The Cell on the Edge Returns Centre Stage,” Development 139(16):2845-56 (2012)).

Myofibers are divided into two types that display different contractile and metabolic properties: slow-twitch (Type I) and fast-twitch (Type II). Slow- and fast-twitch myofibers are defined according to their contraction speed, metabolism, and type of myosin gene expressed (Schiaffino & Reggiani, “Fiber Types in Mammalian Skeletal Muscles,” Physiol. Rev. 91(4):1447-1531 (2011) and Bassel-Duby & Olson, “Signaling Pathways in Skeletal Muscle Remodeling,” Annu. Rev. Biochem. 75:19-37 (2006)). Slow-twitch myofibers are rich in mitochondria, preferentially utilize oxidative metabolism, and provide resistance to fatigue at the expense of speed of contraction. Fast-twitch myofibers more readily atrophy in response to nutrient deprivation, traumatic damage, advanced age-related loss (sarcopenia), and cancer-mediated cachexia, whereas slow-twitch myofibers are more resilient (Wang & Pessin, “Mechanisms for Fiber-Type Specificity of Skeletal Muscle Atrophy,” Curr. Opin. Clin. Nutr. Metab. Care 16(3):243-250 (2013); Tonkin et al., “SIRT1 Signaling as Potential Modulator of Skeletal Muscle Diseases,” Curr. Opin. Pharmacol. 12(3):372-376 (2012); and Arany, Z, “PGC-1 Coactivators and Skeletal Muscle Adaptations in Health and Disease,” Curr. Opin. Genet. Dev. 18(5):426-434 (2008)). Peroxisome proliferator-activated receptor gamma co-activator 1-alpha (PGC1α or Ppargc1) is a major physiological regulator of mitochondrial biogenesis and Type I myofiber specification (Lin et al., “Transcriptional Co-Activator PGC-1 Alpha Drives the Formation of Slow-Twitch Muscle Fibres,” Nature 418 (6899):797-801 (2002)). PGC1α stimulates mitochondrial biogenesis and oxidative metabolism through increased expression of nuclear respiratory factors (NRFs) such as NRF1 and 2 that stimulate mitochondrial biosynthesis, mitochondria transcription factor A (Tfam), and in addition to mitochondrial biosynthesis, also promote slow myofiber formation through increased expression of Mef2 proteins (Lin et al., “Transcriptional Co-Activator PGC-1 Alpha Drives the Formation of Slow-Twitch Muscle Fibres,” Nature 418 (6899):797-801 (2002); Lai et al., “Effect of Chronic Contractile Activity on mRNA Stability in Skeletal Muscle,” Am. J. Physiol. Cell. Physiol. 299(1):C155-163 (2010); Ekstrand et al., “Mitochondrial Transcription Factor A Regulates mtDNA Copy Number in Mammals,” Hum. Mol. Genet. 13(9):935-944 (2004); and Scarpulla, RC, “Transcriptional Paradigms in Mammalian Mitochondrial Biogenesis and Function,” Physiol. Rev. 88(2): 611-638 (2008)). Importantly, PGClu protects muscle from atrophy due to disuse, certain myopathies, starvation, sarcopenia, cachexia, and other causes (Wiggs, M. P., “Can Endurance Exercise Preconditioning Prevention Disuse Muscle Atrophy?,” Front. Physiol. 6:63 (2015); Wing et al., “Proteolysis in Illness-Associated Skeletal Muscle Atrophy: From Pathways to Networks,” Crit. Rev. Clin. Lab. Sci. 48(2):49-70 (2011); Bost & Kaminski, “The Metabolic Modulator PGC-1alpha in Cancer,” Am. J. Cancer Res. 9(2):198-211 (2019); and Dos Santos et al., “The Effect of Exercise on Skeletal Muscle Glucose Uptake in type 2 Diabetes: An Epigenetic Perspective,” Metabolism 64(12):1619-1628 (2015)).

Skeletal muscle can remodel between slow- and fast-twitch myofibers in response to physiological stimuli, load bearing, atrophy, disease, and injury (Bassel-Duby & Olson, “Signaling Pathways in Skeletal Muscle Remodeling,” Annu. Rev. Biochem. 75:19-37 (2006)), involving transcriptional, metabolic, and post-transcriptional control mechanisms (Schiaffino & Reggiani, “Fiber Types in Mammalian Skeletal Muscles,” Physiol. Rev. 91(4):1447-1531 (2011) and Robinson & Dilworth, “Epigenetic Regulation of Adult Myogenesis,” Curr. Top Dev. Biol. 126:235-284 (2018)). The ability to selectively promote slow-twitch muscle has been a long-standing goal, because endurance slow-twitch Type I myofibers provide greater resistance to muscle atrophy (Talbot & Maves, “Skeletal Muscle Fiber Type: Using Insights from Muscle Developmental Biology to Dissect Targets for Susceptibility and Resistance to Muscle Disease,” Wiley Interdiscip. Rev. Dev. Biol. 5(4):518-534 (2016)), and could be an effective therapy for sarcopenia, Duchenne Muscular Dystrophy, cachexia, and other muscle wasting diseases (Selsby et al., “Rescue of Dystrophic Skeletal Muscle By PGC-1alpha Involves A Fast To Slow Fiber Type Shift In The Mdx Mouse,” PLoS One 7(1):e30063 (2012); von Maltzahn et al., “Wnt7a Treatment Ameliorates Muscular Dystrophy,” Proc. Natl. Acad. Sci. USA 109(50):20614-20619 (2012); and Ljubicic et al., “The Therapeutic Potential Of Skeletal Muscle Plasticity In Duchenne Muscular Dystrophy: Phenotypic Modifiers As Pharmacologic Targets,” FASEB J. 28(2):548-568 (2014)).

The myogenesis program is controlled by genes that encode myogenic regulatory factors (MRFs) (Mok & Sweetman, “Many Routes to the Same Destination: Lessons From Skeletal Muscle Development,” Reproduction 141(3):301-12 (2011)), which orchestrate differentiation of the activated satellite cell to become myoblasts, arrest their proliferation, cause them to differentiate, and fuse with multi-nucleated myofibers (Mok & Sweetman, “Many Routes to the Same Destination: Lessons From Skeletal Muscle Development,” Reproduction 141(3):301-12 (2011)). Unique expression markers identify and stage skeletal muscle regeneration. PAX7 is a transcription factor expressed by quiescent and early activated satellite cells (Brack, A. S., “Pax7 is Back,” Skelet. Muscle 4(1):24 (2014) and Gunther, S., et al., “Myf5-positive Satellite Cells Contribute to Pax7-dependent Long-term Maintenance of Adult Muscle Stem Cells,” Cell Stem Cell 13(5):590-601 (2013)).

As satellite cells age, they lose their ability to maintain a quiescent population (Dumont et al., “Intrinsic and Extrinsic Mechanisms Regulating Satellite Cell Function,” Development 142(9):1572-1581 (2015)), and become depleted or functionally exhausted, a primary cause of sarcopenia (muscle loss) with aging and in myopathic diseases (Bernet et al., “p38 MAPK Signaling Underlies a Cell-autonomous Loss of Stem Cell Self-renewal in Skeletal Muscle of Aged Mice,” Nat. Med. 20(3):265-71 (2014); Dumont et al., “Intrinsic and Extrinsic Mechanisms Regulating Satellite Cell Function,” Development 142(9):1572-1581 (2015); Kudryashova et al., “Satellite Cell Senescence Underlies Myopathy in a Mouse Model of Limb-girdle Muscular Dystrophy 2H,” J. Clin. Invest. 122(5):1764-76 (2012); and Silva et al., “Inhibition of Stat3 Activation Suppresses Caspase-3 and the Ubiquitin-proteasome System, Leading to Preservation of Muscle Mass in Cancer Cachexia,” J. Biol. Chem. 290(17):11177-87 (2015)).

Thus, there remains an urgent need for effective therapeutic options that address the primary underlying cause of myopathic diseases (e.g., sarcopenia, Duchenne muscular dystrophy, traumatic muscle injury), which include, e.g., loss of muscle fiber strength, loss of muscle stem cells, loss of muscle regenerative capacity, and attenuation of the exacerbating destructive effects of the pathological immune response on muscle health and integrity.

With advances in use of adeno-associated virus (AAV) mediated gene therapy to potentially treat a variety of rare diseases, there has been hope and interest that AAV could be used to treat DMD, BMD and less severe dystrophinopathies.

Thus, there exists a need in the art for methods of administering AAV vectors encoding microdystrophins in combination with other therapeutics for treatment or amelioration of symptoms of dystrophinopathies, including DMD or BMD, and minimizing immune responses to the therapeutic protein.

### 3. SUMMARY OF THE INVENTION

Increased AU-rich mRNA binding factor 1 (AUF1) expression in muscle cells promotes muscle regeneration, restores or increases muscle mass, function or performance, and/or reduces or reverses muscle atrophy. Further, AUF1 expression in muscle cells increases expression of components of the dystrophin glycoprotein complex (DGC), also referred to herein as the dystrophin associated protein complex or DAPC, and increases participation of components in the DGC, which can stabilize the sarcolemma. AUF1 has further shown activity in enhancing muscle mass and endurance in mdx mice, supporting activity in treatment of dystrophinopathies. Accordingly, provided are combination therapies for treatment and amelioration of symptoms of dystrophinopathies comprising AUF1 therapeutics, including AUF1 gene therapy constructs, with microdystrophin therapeutics, including rAAV gene therapy vectors expressing a microdystrophin, and/or optionally other therapies for dystrophinopathies. Also provided are rAAV gene therapy vectors for delivery of AUF1, and methods of treatment, including for dystrophinopathies, diseases associated with muscle wasting and muscle injury, using those gene therapy vectors.

In embodiments, provided are methods of treating or ameliorating the symptoms of (or pharmaceutical compositions for use in treating or ameliorating the symptoms of) a dystrophinopathy, including Duchenne muscular dystrophy (DMD), Becker muscular dystrophy (BMD), X-linked dilated cardiomyopathy or limb-girdle muscular dystrophy, in a subject (which may be a human subject) in need thereof, comprising administering to the subject a first therapeutic and a second therapeutic which is different from said first therapeutic, wherein the first therapeutic is a first rAAV particle comprising a nucleic acid molecule encoding an AU-rich mRNA binding factor 1 (AUF1) protein, or functional fragment thereof, operatively coupled to a muscle cell-specific promoter and flanked by inverted terminal repeat (ITR) sequences. In embodiments, the second therapeutic is an rAAV gene therapy vector that encodes a microdystrophin. The first and second therapeutics may be administered concurrently or may be administered separately (for example, the doses may be separated by 1 hour, 2 hours, 3 hours, 4 hours, 12 hours, 1 day, 2 day, 3, days, 4 days, 5 days, 6 days, 7 days, or 2 weeks). In certain embodiments, the AUF1 gene therapy vector (the first therapeutic) is administered prior to the microdystrophin gene therapy vector (the second therapeutic). In certain embodiments, the AUF1 gene therapy vector (the first therapeutic) is administered subsequent to the administration of the microdystrophin gene therapy vector (the second therapeutic).

In embodiments, the AUF1 is a human AUF1 p37^{AUF1}, p4^{AUF1}, p42^{AUF1}, orp45^{AUF1} isoform, including, for example, the p40^{AUF1} isoform, and may be encoded by a codon optimized, CpG deleted nucleotide sequence, for example, the nucleotide sequence of SEQ ID NO: 17. In additional embodiments, in the AUF1 gene therapy vector, including rAAV gene therapy vector, the muscle cell-specific promoter is a muscle creatine kinase (MCK) promoter, a syn100 promoter, a CK6 promoter, a CK7 promoter, a CK8 promoter, a CK9 promoter, a dMCK promoter, a tMCK promoter, a smooth muscle 22 (SM22) promoter, a myo-3 promoter, a Spc5-12 promoter (including modified Spc5-12 promoters SpcV1 (SEQ ID NO: 127) or SpcV2 (SEQ ID NO: 128), a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter (see also Table 10).

In particular embodiments, the first therapeutic is a first rAAV particle comprises a recombinant genome having the nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1). The rAAV particle is, in embodiments, an AAV8 or AAV9 serotype and has a capsid that is at least 95% identical to SEQ ID NO: 114 (AAV8 capsid) or SEQ ID NO: 115 (AAV9 capsid). In particular embodiments, the first therapeutic is administered systemically, including intravenously at a dose of 1E13 to 1E14 vg/kg or a dose of 2E13 vg/kg (vector genomes/kg (vg/kg) and genome copies/kg (gc/kg) are used interchangeably herein as are EX and X10^{X}).

In further embodiments, the methods and compositions provided include treatment of (and pharmaceutical compositions for use in treatment of) a dystrophinopathy in a subject (including a human subject) in need thereof with the first therapeutic, AUF1 gene therapy, in combination with the second therapeutic which is a microdystrophin pharmaceutical composition. In specific embodiments, the microdystrophin protein consists of dystrophin domains arranged from amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site, and, in certain embodiments, has the amino acid sequence of SEQ ID NO: 96 or SEQ ID NO: 94. In other embodiments, the microdystrophin has an amino acid sequence of SEQ ID NO: 133-137 (Table 5). In embodiments, the microdystrophin is administered by delivery of a viral vector, including an rAAV particle, that comprises a transgene the microdystrophin protein operatively coupled to a regulatory sequence that promotes expression in muscle cells, which transgene is flanked by ITRs. In embodiments, the transcriptional regulatory element comprises a muscle-specific promoter. Specific artificial genomes include the nucleotide sequence of SEQ ID NO: 94 or 96 or alternatively SEQ ID Nos: 129 to 131 having modified Spc5-12 promoters. In embodiments, the rAAV encoding the microdystrophin is an AAV8, AAV9 or AAVhu.32 serotype and has a capsid that is at least 95% identical to SEQ ID NO: 114 (AAV8 capsid), SEQ ID NO: 115 (AAV9 capsid), or SEQ ID NO: 118 (AAVhu.32 capsid). In embodiments, the therapeutically effective amount of the second rAAV particle is administered intravenously or intramuscularly at dose of 2×10¹³ to 1×10¹⁵ genome copies/kg. In addition, in specific embodiments, the ratio of the vector genomes of the first rAAV particle (the AUF1 gene therapy vector) in the first therapeutic to the vector genomes of the second rAAV particle (the microdystrophin gene therapy vector) in the second therapeutic is 0.5 to 1; 0.25 to 1; 0.2 to 1; 0.1 to 1; 1 to 1; 1 to 2; 1 to 5; 1 to 10; 1 to 20; 1 to 100; or 1 to 1000.

Alternatively, in embodiments, the second therapeutic may be a microdystrophin pharmaceutical composition which comprises a therapeutically effective amount of SGT-001, GNT 004, rAAVrh74.MHCK7, micro-dystrophin (SRP-9001) or PF-06939926.

In embodiments, either the second therapeutic is a therapy which is not an AUF1 or microdystrophin therapy and may be a mutation suppression therapy, an exon skipping therapy, a steroid therapy, an immunosuppressive/anti-inflammatory therapy, or a therapy that treats one or more symptoms of the dystrophinopathy. In further embodiments, in addition to administration of the combination of the first and second therapeutics, where the second therapeutic is a microdystrophin therapy, a third or even additional therapeutics are administered, which may be a mutation suppression therapy, an exon skipping therapy, a steroid therapy, an immunosuppressive/anti-inflammatory therapy, or a therapy that treats one or more symptoms of the dystrophinopathy.

In other embodiments, provided is a nucleic acid comprising a nucleotide sequence of SEQ ID NO: 17 encoding human AUF1 p40, which is a codon optimized, reduced CpG sequence. Provided are vectors comprising this sequence (SEQ ID NO: 17) operably linked to a muscle cell-specific promoter, which may a muscle creatine kinase (MCK) promoter, a Syn promoter, a syn100 promoter, a CK6 promoter, a CK7 promoter, a CK8 promoter, a CK9 promoter, a dMCK promoter, a tMCK promoter, a smooth muscle 22 (SM22) promoter, a myo-3 promoter, a Spc5-12 promoter (including variant Spc5-12 promoters Spc5v1 (SEQ ID NO:127) and Spc5v2 (SEQ ID NO: 128), a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter (see, for example, Table 10). In embodiments, the nucleotide sequence of SEQ ID NO: 17, in addition to being operably linked to the muscle specific promoter sequence is further operably linked to an intron sequence, such as a VH4 intron sequence, a polyadenylation signal sequence, such as a rabbit beta globin polyadenylation signal sequence, and/or a WPRE sequence (as disclosed herein). The vector may be a cis plasmid for packaging rAAV or an rAAV genome, which is flanked by ITR sequences. The genome in the rAAV particle may be single stranded or may be self complementary. In addition, in view of the size of the human AUF1p40 sequence, the rAAV vector sequence may also comprise 5′ and/or 3′ stuffer sequences (see Table 12) and/or a SV40 polyadenylation signal sequence.

In specific embodiments, the vector comprises a nucleotide sequence of SEQ ID NO: 17, encoding human AUF1 p40, operably linked to regulatory sequence that promotes expression in muscle, including muscle specific promoters (or constitutive promoters) as disclosed herein (see, for example, Table 10, and may have, in embodiments, a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) and rAAV particles, pharmaceutical compositions and methods of using same comprising these nucleotides sequences are further provided. The rAAV particle is, in embodiments an AAV8, AAV9 or AAVhu.32 serotype, or capsid in Table 13, including having a capsid that is at least 95% identical to SEQ ID NO: 114 (AAV8 capsid), SEQ ID NO: 115 (AAV9 capsid), or SEQ ID NO: 118 (AAVhu.32 capsid).

In embodiments, the AUF1 rAAV vectors disclosed herein, including vectors comprising a human AUF1 p40 coding sequence of SEQ ID NO: 17 operably linked to a regulatory sequence that promotes expression in muscle, including muscle specific promoters (or constitutive promoters) as disclosed herein (see, for example, Table 10), and includes vectors comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1)), and is, in embodiments, an AAV8, AAV9, AAVhu.32 serotype, are in compositions for use in methods or treatment or are administered in therapeutically effective amounts for methods of treatment including, methods of stabilizing sarcolemma, including methods where one or more of α-dystroglycan, β-dystroglycan, α-sarcoglycan, β-sarcoglycan, δ-sarcoglycan, γ-sarcoglycan, ε-Sarcoglycan, ζ-sarcoglycan, sarcospan, α-syntrophin, β-syntrophin, α-dystrobrevin, β-dystrobrevin, caveolin-3, or nNOS is increased in expression and/or in the DGC.

Also provided are compositions for use in and methods of increasing muscle mass in a subject having age-related muscle loss or treating sarcopenia in a subject (in embodiments, a human subject) in need thereof comprising administering to the subject a pharmaceutical composition comprising therapeutically effective amount of an rAAV particle comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype); and a pharmaceutically acceptable carrier. In embodiments, the human subject is elderly and may be over 65 years old, over 75 years old, over 85 years old or over 90 years old.

In embodiments, provided are compositions for use in and methods of treating or ameliorating the symptoms of a dystrophinopathy in a subject (including a human subject) in need thereof comprising administering to the subject a pharmaceutical composition comprising a therapeutically effective amount the rAAV particle comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), and a pharmaceutically acceptable carrier. The dystrophinopathy may be Duchenne muscular dystrophy (DMD), Becker muscular dystrophy (BMD), X-linked dilated cardiomyopathy or limb-girdle muscular dystrophy.

Also provided are embodiments encompassing a composition for use in or a method of increasing utrophin in a dystrophin glycoprotein complex (DGC) in a subject (including a human subject) in need thereof, comprising administering to the subject a pharmaceutical composition comprising a therapeutically effective amount of an rAAV particle comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), and a pharmaceutically acceptable carrier. The subject may have a mutated dystrophin and, further, the composition or method may promote the replacement of the mutated dystrophin with utrophin in the DGC of the subject.

In embodiments, provided are compositions for use in and methods of increasing healing of traumatic muscle injury in a subject (including a human subject) in need thereof, said method comprising administering to the subject, either systemically or locally, a pharmaceutical composition comprising a therapeutically effective amount the rAAV particle comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), and a pharmaceutically acceptable carrier.

In the compositions for and methods of treatment with an rAAV particle comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), the administration increases muscle mass, increase muscle strength, reduce expression of biomarkers of muscle atrophy, enhance muscle performance, increase muscle stamina, increase muscle resistance to fatigue and/or increase proportion of slow twitch fibers to fast twitch fibers.

In these methods of administering the AUF1 gene therapy rAAV particles disclosed herein, comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), the rAAV particle is, in embodiments, administered intravenously or intramuscularly and, in embodiments at a dose of 1E13 to 1E14 vg/kg.

Also provided are host cells for producing rAAV particles comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1) (and is, in embodiments, an AAV8 or AAV9 serotype), where the host cell contains an artificial genome comprising a nucleotide sequence of SEQ ID NO: 31 (spc-hu-opti-AUF1-CpG(−)), SEQ ID NO: 32 (tMCK-huAUF1), SEQ ID NO: 33 (spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1); a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; and sufficient adenovirus helper functions to permit replication and packaging of the artificial genome by the AAV capsid proteins. The capsid protein may be an AAV8, AAV9 or AAVhu.32 capsid protein and, including where the capsid protein is at least 95% identical to SEQ ID NO: 114 (AAV8 capsid), SEQ ID NO: 115 (AAV9 capsid) or SEQ ID NO: 118 (AAVhu.32). Provided are methods of producing the rAAV particles by culturing the host cells and recovering recombinant AAV encapsidating the artificial genome from the cell culture.

### 3.1 Embodiments

1. A method of treating a dystrophinopathy in a subject in need thereof, comprising administering to the subject a first therapeutic and a second therapeutic which is different from said first therapeutic,
wherein the first therapeutic is a first rAAV particle comprising a nucleic acid molecule encoding an AU-rich mRNA binding factor 1 (AUF1) protein, or functional fragment thereof, operatively coupled to a muscle cell-specific promoter and flanked by inverted terminal repeat (ITR) sequences.
2. A pharmaceutical composition for use in treating a dystrophinopathy in a subject in need thereof, said pharmaceutical composition comprising a first therapeutic administered in combination with a second therapeutic which is different from said first therapeutic,
wherein the first therapeutic is a first rAAV particle comprising a nucleic acid molecule encoding an AU-rich mRNA binding factor 1 (AUF1) protein, or functional fragment thereof, operatively coupled to a muscle cell-specific promoter and flanked by inverted terminal repeat (ITR) sequences.
3. The method of embodiment 1 or the composition of embodiment 2, wherein the muscle cell-specific promoter is a muscle creatine kinase (MCK) promoter, a syn100 promoter, a CK6 promoter, a CK7 promoter, a CK8 promoter, or a CK9 promoter, a dMCK promoter, a tMCK promoter, a smooth muscle 22 (SM22) promoter, a myo-3 promoter, a Spc5-12 promoter, an Spc5V1 promoter, an Spc5V2 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter.
4. The method or composition of embodiment 3, wherein the muscle cell-specific promoter is a tMCK promoter, a Spc5-12 promoter, or a CK7 promoter.
5. The method or composition of any of the preceding claims, wherein the nucleic acid molecule encodes one or more of human p37^{AUF1}, p4^{AUF1}, p42^{AUF1}, or p45^{AUF1}.
6. The method or composition of any one of the preceding embodiments, wherein the nucleotide sequence encoding the p40^{AUF1} protein is the nucleotide sequence of SEQ ID NO: 17.
7. The method or composition of any one of the preceding embodiments, wherein the nucleotide sequence encoding the AUF1 protein further comprises a polyadenylation signal, optionally with a nucleotide sequence of SEQ ID NO: 23 or 25.
8. The method or composition of any one of the preceding embodiments, wherein the nucleotide sequence further comprises an intron sequence 5′ of the nucleotide sequence encoding the AUF1 protein, optionally, comprising a nucleotide sequence of SEQ ID NO: 111, 112, 113 or 138.
9. The method or composition of any one of the preceding embodiments, wherein the nucleotide sequence further comprises a 5′ and/or a 3′ stuffer sequence, optionally having a nucleotide sequence of one or more of SEQ ID Nos: 139-143 and/or a WPRE (SEQ ID NO: 24).
10. The method or composition of any one of the preceding embodiments, wherein the first rAAV particle comprises a recombinant genome having the nucleotide sequence of SEQ ID NO: 31 (spc-hu-opti-AUF1-CpG(−)), SEQ ID NO: 32 (tMCK-huAUF1), SEQ ID NO: 33 (Spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1).
11. The method or composition of any one of the preceding embodiments wherein the nucleic acid encoding the AUF 1 protein is a single stranded or self-complementary recombinant artificial genome.
12. The method or composition of any one of the preceding embodiments wherein the AAV has a capsid that is at least 95%, 99% or 100% identical to SEQ ID NO: 114 (AAV8 capsid), SEQ ID NO: 115 (AAV9 capsid), or SEQ ID NO: 118 (AAVhu.32).
13. The method or composition of any one of the preceding embodiments, wherein the rAAV is administered at a dose of 1E13 to 1E14 vg/kg or a dose of 2E13 vg/kg.
14. The method or composition of any one of the preceding embodiments, wherein the second therapeutic is a microdystrophin pharmaceutical composition.
15. The method or composition of embodiment 14, wherein the microdystrophin protein consists of dystrophin domains arranged from amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site.
16. The method or composition of embodiment 15, wherein the microdystrophin pharmaceutical composition encodes for a protein having the amino acid sequence of SEQ ID NO: 52 or SEQ ID NO: 54.
17. The method or composition of embodiment 14, wherein the microdystrophin protein has an amino acid sequence of one of SEQ ID NO: 133 to 137.
18. The method or composition of any one of embodiments 14-17, wherein the microdystrophin pharmaceutical composition comprises a therapeutically effective amount of a second rAAV particle comprising an artificial genome comprising a nucleic acid that encodes the microdystrophin protein operatively coupled to a regulatory sequence that promotes expression in muscle cells, which transgene is flanked by ITRs; and a pharmaceutically acceptable carrier.
19. The method or composition of embodiment 18, wherein the regulatory sequence comprises a muscle-specific promoter.
20. The method or composition of embodiment 19, wherein the muscle-specific promoter is a muscle creatine kinase (MCK) promoter, a syn100 promoter, a CK6 promoter, a CK7 promoter, a CK8 promoter, or a CK9 promoter, a dMCK promoter, a tMCK promoter, a smooth muscle 22 (SM22) promoter, a myo-3 promoter, a Spc5-12 promoter, an Spc5V1 promoter, an Spc5V2 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter
21. The method or composition of embodiment 20, wherein the muscle specific promoter is Spc5-12, Spc5V1 or Spc5V2.
22. The method or composition of any one of embodiments 18-21, wherein the artificial genome comprises the nucleotide sequence of SEQ ID NO:94, 96, 130 or 132.
23. The method or composition of any one of embodiments 18-22, wherein the AAV has a capsid that is at least 95%, 99% or 100% identical to SEQ ID NO: 114 (AAV8 capsid), SEQ ID NO: 115 (AAV9 capsid) or SEQ ID NO: 118 (AAVhu.32 capsid).
24. The method or composition of any one of embodiments 18-23, wherein the therapeutically effective amount of the second rAAV particle is administered intravenously or intramuscularly at dose of 2×10¹³ to 1×10¹⁵ genome copies/kg.
25. The method or composition of any one of embodiments 18-24 wherein the first therapeutic and the second therapeutic are administered concurrently or within 1 week or within 2 weeks of each other.
26. The method or composition of any one of embodiments 18-25 wherein the ratio of the vector genomes of the first rAAV particle in the first therapeutic to the vector genomes of the second rAAV particle in the second therapeutic is 0.5 to 1; 0.25 to 1; 0.2 to 1; 0.1 to 1; 1 to 1; 1 to 2; 1 to 5; 1 to 10; 1 to 20; 1 to 100; or 1 to 1000.
27. The method or composition of embodiment 14 wherein the microdystrophin pharmaceutical composition comprises a therapeutically effective amount of SGT-001, GNT 004, rAAVrh74.MHCK7, micro-dystrophin (SRP-9001) or PF-06939926.
28. The method or composition of any one of embodiments 1-13, wherein the second therapeutic is a mutation suppression therapy, an exon skipping therapy, a steroid therapy, an immunosuppressive/anti-inflammatory therapy, or a therapy that treats one or more symptoms of the dystrophinopathy.
29. The method or composition of any one of the preceding embodiments, wherein the first therapeutic is administered intravenously.
30. The method or composition of any one of the preceding embodiments, wherein the second therapeutic is administered intravenously.
31. The method or composition of any one of the preceding embodiments, wherein the dystrophinopathy is Duchenne muscular dystrophy (DMD), Becker muscular dystrophy (BMD), X-linked dilated cardiomyopathy or limb-girdle muscular dystrophy.
32. A nucleic acid comprising a nucleotide sequence of SEQ ID NO: 17 encoding AUF1 p40.
33. A vector comprising the nucleic acid of embodiment 29 operably linked to a muscle cell-specific promoter.
34. The vector of embodiment 33, wherein the muscle cell-specific promoter is a muscle creatine kinase (MCK) promoter, a syn100 promoter, a CK6 promoter, a CK7 promoter, a CK8 promoter, or a CK9 promoter, a dMCK promoter, a tMCK promoter, a smooth muscle 22 (SM22) promoter, a myo-3 promoter, a Spc5-12 promoter, an SpcV1 promoter, an SpcV2 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter.
35. The vector of embodiment 34, wherein the muscle cell-specific promoter is a tMCK promoter, an Spc5-12 promoter, or a CK7 promoter.
36. The vector of any one of embodiments 33-35 further comprising a polyadenylation signal, optionally with a nucleotide sequence of SEQ ID NO: 23 or 25.
37. The vector of any one of embodiments 33 to 36 which further comprises an intron sequence 5′ of the nucleotide sequence encoding the AUF1 protein, optionally, comprising a nucleotide sequence of SEQ ID NO: 111, 112, 113 or 138.
38. The vector of any one of embodiments 33-37 further comprising a 5′ and/or a 3′ stuffer sequence, optionally having a nucleotide sequence of one or more of SEQ ID Nos: 139-143 and/or a WPRE (SEQ ID NO: 24).
39. The vector of any of embodiments 33-38 wherein the nucleic acid encoding AUF1 and regulatory elements is flanked by ITR sequences.
40. The vector of any of embodiments 33-39 which comprises a nucleotide sequence of SEQ ID NO: 31 (spc-hu-opti-AUF1-CpG(−)), SEQ ID NO: 32 (tMCK-huAUF1), SEQ ID NO: 33 (Spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1).
41. An rAAV particle comprising the vector of any one of embodiments 33-40.
42. The rAAV particle of embodiment 41 which has a capsid that is at least 95%, 99% or 100% identical to SEQ ID NO: 114 (AAV8 capsid), SEQ ID NO: 115 (AAV9 capsid) or SEQ ID NO: 118 (AAVhu.32).
43. A pharmaceutical composition comprising the rAAV particle of embodiments 41 or 42; and a pharmaceutically acceptable carrier.
44. A method of stabilizing sarcolemma in a subject comprising administering to the subject a pharmaceutical composition comprising a therapeutically effective amount the rAAV particle of embodiment 38 or 39 and a pharmaceutically acceptable carrier.
45. A pharmaceutical composition for use in stabilizing sarcolemma in a subject, said pharmaceutical composition comprising a therapeutically effective amount the rAAV particle of embodiment 38 or 39 and a pharmaceutically acceptable carrier.
46. The method or composition of embodiment 44 or 45, wherein one or more of α-dystroglycan, β-dystroglycan, α-sarcoglycan, β-sarcoglycan, δ-sarcoglycan, γ-sarcoglycan, ε-Sarcoglycan, ζ-sarcoglycan, α-dystroglycan, β-dystroglycan, sarcospan, α-syntrophin, β-syntrophin, α-dystrobrevin, β-dystrobrevin, caveolin-3, or nNOS is increased in a DGC.
47 A method of increasing muscle mass in a subject having age-related muscle loss comprising administering to the subject a pharmaceutical composition comprising a therapeutically effective amount the rAAV particle of embodiment 44 or 45 and a pharmaceutically acceptable carrier.
48. A pharmaceutical composition for use in increasing muscle mass in a subject having age-related muscle loss, said pharmaceutical composition comprising a therapeutically effective amount the rAAV particle of embodiment 44 or 45 and a pharmaceutically acceptable carrier.
49. The method of embodiment 47 or composition of embodiment 48, wherein the subject is over 65 years old, over 75 years old, over 85 years old or over 90 years old.
50. A method of treating sarcopenia in a subject in need thereof, said method comprising administering to the subject a pharmaceutical composition comprising a therapeutically effective amount the rAAV particle of embodiment 44 or 45 and a pharmaceutically acceptable carrier.
51. A pharmaceutical composition for use increasing muscle mass in a subject having age-related muscle loss, said pharmaceutical composition comprising a therapeutically effective amount the rAAV particle of embodiment 38 or 39 and a pharmaceutically acceptable carrier.
52. The method of embodiment 50 or the composition of embodiment 51, wherein the subject is over 65 years old, over 75 years old, over 85 years old or over 90 years old.
53. A method of treating a dystrophinopathy in a subject in need thereof comprising administering to the subject a pharmaceutical composition comprising a therapeutically effective amount the rAAV particle of embodiment 44 or 45 and a pharmaceutically acceptable carrier.
54. A pharmaceutical composition for use in treating a dystrophinopathy in a subject in need thereof, said pharmaceutical composition comprising a therapeutically effective amount the rAAV particle of embodiment 44 or 45 and a pharmaceutically acceptable carrier.
55. The method of embodiment 53 or composition of embodiment 54, wherein the dystrophinopathy is Duchenne muscular dystrophy (DMD), Becker muscular dystrophy (BMD), X-linked dilated cardiomyopathy or limb-girdle muscular dystrophy.
56. A method of increasing utrophin in a dystrophin glycoprotein complex (DGC) in a subject comprising administering to the subject a pharmaceutical composition comprising a therapeutically effective amount the rAAV particle of embodiment 44 or 45 and a pharmaceutically acceptable carrier.
57. A pharmaceutical composition for use in increasing utrophin in a dystrophin glycoprotein complex (DGC) in a subject, said pharmaceutical composition comprising a therapeutically effective amount the rAAV particle of embodiment 44 or 45 and a pharmaceutically acceptable carrier.
58. The method of embodiment 56 or the composition of embodiment 57, wherein the subject has a mutated dystrophin.
59. The method or composition of embodiment 58, wherein the method promotes replacement of the mutated dystrophin with utrophin in the DGC.
60. A method of increasing healing of traumatic muscle injury in a subject in need thereof, said method comprising administering to the subject a pharmaceutical composition comprising a therapeutically effective amount the rAAV particle of embodiment 44 or 45 and a pharmaceutically acceptable carrier.
61. The method or composition of any of embodiments 44 to 60, wherein said administration increases muscle mass, increase muscle strength, reduce expression of biomarkers of muscle atrophy, enhance muscle performance, increase muscle stamina, increase muscle resistance to fatigue and/or increase proportion of slow twitch fibers to fast twitch fibers.
62. The method or composition of any one of embodiments 44 to 61, wherein the therapeutically effective amount of the rAAV particle is administered at dose of 1E13 to 1E14 vg/kg.
63. The method or composition of any of embodiments 44 to 62, wherein the pharmaceutical composition is administered intravenously or intramuscularly.
64. A method of producing recombinant AAVs comprising:
culturing a host cell containing:
an artificial genome comprising the vector of any of embodiments 33-40;
a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans;
sufficient adenovirus helper functions to permit replication and packaging of the artificial genome by the AAV capsid proteins; and
recovering recombinant AAV encapsidating the artificial genome from the cell culture.
65 A host cell comprising the nucleic acid of embodiment 29.

### 4. BRIEF DESCRIPTION OF THE FIGURES

FIG. 1 illustrates vector gene expression cassettes and AUF1 constructs for use in a cis plasmid for production of AAV gene therapy vectors. DNA length for each construct is provided. Hu-AUF1-CpG(−): CpG depleted human AUF1 p40 coding sequence; Stuffer: non-coding stuffer or filler sequence; Spc5-12: synthetic muscle-specific promoter; vh-4 in: VH4: human immunoglobulin heavy chain variable region intron; tMCK: truncated muscle creatine kinase promoter; CK7: creatine kinase 7 promoter; RBG-PA: rabbit beta-globin polyA signal sequence; SV40 pA: SV40 polyA signal sequence; and WPRE: woodchuck hepatitis virus post-transcriptional regulatory element.

FIGS. 2A-2E depict the characterization of AUF1-p40 expression in differentiated C2C12 cells transfected by AUF1 cis plasmids containing different promoters and regulatory elements flanking the p40 coding sequence. A. Western blot analysis of protein detected by anti-AUF1 antibody. Lane 1=spc-hu-opti-AUF1-CpG(−); Lane 2=tMCK-huAUF1; Lane 3=spc5-12-hu-opti-AUF1-WPRE; Lane 4=spc-hu-AUF1-No-Intron; Lane 5=GFP control. Arrow indicated the transfected AUF1-p40 expression, whereas other bands represent endogenous AUF1 isoform protein in these cells. B. Quantification of the ratio of AUF1-p40 expression band to α-actinin endogenous control expression band in the Western blot. C-E. Quantification of RNA expression and DNA copy numbers in differentiated C2C12 myotubes by digital PCR after transfection of cis plasmids. 1=spc-hu-opti-AUF1-CpG(−); 2=tMCK-huAUF1; 3=spc5-12-hu-opti-AUF1-WPRE; 4=spc-hu-AUF1-No-Intron (see Table 3 for construct nucleotide sequences). C. AUF1 RNA expression generated by different plasmids in differentiated C2C12 cells by digital PCR. D. AUF1 DNA copy numbers in transfected cells by digital PCR. E. AUF1 RNA expression normalized by DNA copy numbers.

FIG. 3 depicts serum creatine kinase (CK) activity (mU/mL) in wild-type (WT) (C57/B16) mice and mdx mice 1 month after administration of AAV8-mAUF1, AAV8-huAUF1 (AAV8-tMCK-huAUF1), AAV8-RGX-DYS5 or a combination of AAV8-RGX-DYS5 and AAV8-hAUF1.

FIGS. 4 A-B show Hematoxylin and Eosin (H&E) staining of the diaphragm muscle in WT mice and mdx mice administered AAV8-mAUF1, AAV-hAUF1 (AAV8-tMCK-huAUF1), AAV8-RGX-DYS5 or a combination of AAV8-RGX-DYS5 and AAV8-huAUF1 at low magnification (scale bar 1000 μm) (A) and high magnification (scale bar 400 m) (B). C. Percent of degenerative region of the diaphragm in WT mice and mdx mice administered AAV8-mAUF1, AAV8-hAUF1, AAV8-RGX-DYS5 or a combination of AAV8-RGX-DYS5 and AAV8-hAUF1.

FIGS. 5A-B show immunoblot analysis of WT mice and mdx mice administered AAV8-mAUF1, AAV-hAUF1 (AAV8-tMCK-huAUF1), AAV8-RGX-DYS5 or a combination of AAV8-RGX-DYS5 and AAV8-hAUF1 showing DAPC proteins (nNOS, γ-sarcoglycan and β-dystroglycan) are increased by AAV8-hAUF1, AAV8-RGX-DYS5 and combination therapy in the gastrocnemius muscle. B. Quantification of protein levels (Utrophin/GAPDH) from immunoblot results from 3 independent studies as shown in FIG. 5A.

FIGS. 6 A-B show H&E staining of diaphragm muscle three months following treatment in WT mice and mdx mice administered AAV8-mAUF1, AAV8-hAUF1 (AAV8-tMCK-huAUF1), AAV8-RGX-DYS5 or a combination of AAV8-RGX-DYS5 and AAV8-hAUF1 in unblinded studies (A) and blinded studies (B).

FIGS. 7 A-D. A-C show quantification by image J of the percentage of eMHC positive myofibers (A), the percentage of central nuclei myofibers (B) and the area of central nuclei CSA (μm²) (C). FIG. 7D shows the percentage of central nuclei myofibers CSA as a function of their cross-sectional areas from multiple diaphragm muscles.

FIGS. 8 A-D depict muscle function studies on mdx mice three months post administration of AAV8-RGX-DYS5, AAV8-hAUF1 (AAV8-tMCK-huAUF1) and AAV8-RGX-DYS5+AAV8-hAUF1. A. Time to exhaustion (secs) B. Distance to exhaustion (m) C. Maximum speed (cm/s) D. grid hanging time (seconds; absolute value).

FIG. 9 depicts muscle exercise function tests in mdx mice three months post administration of a higher dose of AAV8-hAUF1 (AAV8-tMCK-huAUF1) (6E13 vg/kg), AAV8-RGX-DYS5 (1E14 vg/kg) or in combination. A. Time to exhaustion (secs) B. Distance to exhaustion (m) C. Maximum speed (cm/x).

FIG. 10 shows H&E staining of diaphragm muscle in mdx mice administered AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIGS. 11A and B show immunofluorescence images (A) and Evans blue staining (B) of diaphragm muscle in mdx mice administered AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIG. 12 shows Evans blue staining of muscles from mdx mice six months after administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIG. 13 shows SDH activity staining in mdx mice three months after administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIGS. 14 A-D show the central nuclei CSA area (μm²) (A, C) and central nuclei myofiber csa percentage (B, D) in WT and mdx mice treated with lower dose AAV8-hAUF1 (AAV8-tMCK-huAUF1) (2E13 vg/kg) (A, B) and higher dose AAV8-hAUF1 (6E13 vg/kg) (C, D).

FIGS. 15 A-C depict muscle exercise function tests in mdx mice six months after administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg). A. Time to exhaustion (secs) B. Distance to exhaustion (m) C. Maximum speed (cm/s).

FIGS. 16 A and B depict muscle grip strength function tests (N/g) (ANOVA analysis (A) or Multiple T test analysis (B)) in mdx mice 6 months after administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIGS. 17 A-I depict the percentage of live myeloid cells (A), the number of myeloid cells per g tissue (B), the percentage of live macrophages (C), the number of macrophages per g tissue (D), the percentage of live M1 macrophages (E), the number of M1 macrophages per g tissue (F), the percentage of live M2 macrophages (G), the number of M2 macrophages per g tissue (H) and the ratio of M1 to M2 macrophages (I) in WT and mdx mice after administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIG. 18 shows the percent atrophy after injection of 1.2% of BaCl₂ in the tibialis anterior muscle of mdx mice 3 months post-administration of AAV8-hAUF1 (AAV8-tMCK-huAUF1) (6E13 vg/kg), AAV8-RGX-DYS5 (1E14 vg/kg) or AAV8-hAUF1 (6E13 vg/kg)+AAV8-RGX-DYS5 (1E14 vg/kg).

FIGS. 19A-19D depict quantitation of DNA copies (genome copies) and RNA expression of transgene in liver resulting from administration of a combination of microdystrophin (μDys) and human AUF1 vectors, μDys vector alone, human AUF1 vector alone, mouse AUF1 vector and eGFP vector, or eGFP vector alone to mdx mouse groups. A control wild-type mouse group receiving no vector was tested for background. The μDys vector is driven by an Spc5-12 promoter and the human AUF1 is driven by a truncated MCK promoter.

FIGS. 20A-20D depict quantitation of DNA copies (genome copies) and RNA expression of transgene in muscle (EDL) (20A and 20B) or heart (20C and 20D) resulting from administration of a combination microdystrophin (μDys) and human AUF1 vectors, μDys vector alone, human AUF1 vector alone, mouse AUF1 vector and eGFP vector, or eGFP vector alone to mdx mouse groups. A control wild-type mouse group receiving no vector was tested for background. The μDys vector is driven by an Spc5-12 promoter and the human AUF1 is driven by a truncated MCK promoter.

FIGS. 21A-21B depict quantitation of DNA and RNA copy numbers in spleen (2E13 vg/kg of AUF1 dosage) resulting from administration of a combination microdystrophin (μDys) and human AUF1 vectors, μDys vector alone, human AUF1 vector alone, eGFP vector alone to mdx mouse groups. The μDys vector is driven by an Spc5-12 promoter and the human AUF1 is driven by a truncated MCK promoter.

FIGS. 22A-22B illustrate RNA expression levels of tMCK-hAUF1 or Spc5-12-μDys vectors in EDL, heart and liver compared to a control transcript (TBP). The transgene RNA expression in AAV vectors was assessed by analyzing the RNA copies of the transgene microdystrophin/μDys (driven by the spc5-12 promoter) or AUF1 (driven by the tMCK promoter) to an endogenous control TBP (TATA box binding protein) ratio in different tissues. The RNA total per TBP was then expressed as a ratio compared to the DNA copies of each transgene to understand the promoter activity to express the transgene per diploid genome of each cell. For reference, FIG. 21B provides the total endogenous TBP RNA copies per μg of total RNA in each tissue (extensor digitorum longus (EDL) muscle, heart, liver or spleen). This indicates the EDL muscle, heart, and liver have similar levels of endogenous control TBP mRNA expression. Therefore, the difference in transgene mRNA expression/TBP/vector copies reflects how much mRNA produced per AAV vector genome, indicating promoter activity.

### 5. DETAILED DESCRIPTION

Provided are methods of treating (and pharmaceutical compositions for use in treating) dystrophinopathies, including, Duchenne muscular dystrophy (DMD), Becker muscular dystrophy (BMD), X-linked dilated cardiomyopathy, and limb-girdle muscular dystrophy by administering to a subject in need thereof a combination of gene therapy vectors, particularly, rAAV vectors, in which a first gene therapy vector comprises a genome with a transgene encoding an AUF1 protein operably linked to a regulatory element that promotes expression in muscle cells in a therapeutically effective amount and a second gene therapy vector comprising a genome with a transgene encoding a microdystrophin or other protein (other than AUF1) effective to treat the dystrophinopathy operably linked to a regulatory element that promotes expression in muscle cells in a therapeutically effective amount. The first and second gene therapy vectors may be administered concurrently (either in the same or in separate pharmaceutical compositions) or may be administered sequentially, with either the first gene therapy vector being administered before the second gene therapy vector or, vice versa, the first gene therapy vector being administered after the second gene therapy vector (for example within 1 day, 2 days, 3 days, 4 days, 5 days, 6 days, 1 week, 2 weeks or more). In other embodiments, AUF1 protein or nucleic acid encoding AUF1 is administered in combination with another therapeutic for use in treating a dystrophinopathy.

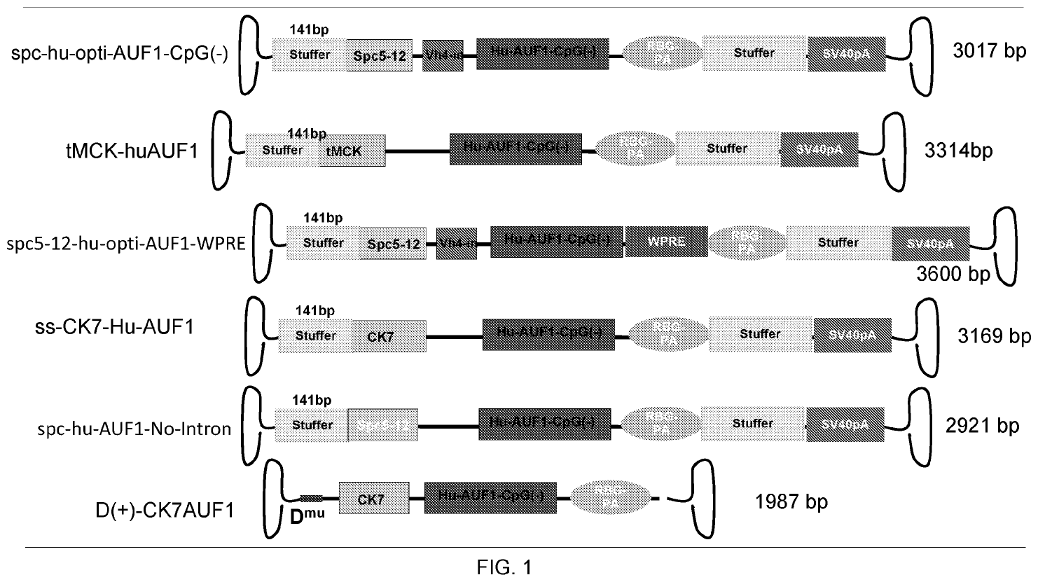

Also provided are AUF1 AAV gene therapy constructs. The constructs have a codon optimized, CpG depleted coding sequence for human p40 AUF1 (SEQ ID NO: 17) operably linked to a regulatory element that promotes expression in muscle cells (see, e.g., Table 10) and optionally other regulatory elements such as polyadenylation sequences, intron sequences, WPRE or other element, and/or stuffer sequences, including, for example, as disclosed herein. Exemplary constructs are depicted, for example, in FIG. 1 (see also Table 3). The constructs, including flanking ITR sequences, may have nucleotide sequences of SEQ ID NOs: 31 to 36. The gene therapy vectors may be, e.g., AAV8 serotype vectors, AAV9 serotype vectors, AAVhu.32 serotype vectors (see, for example, capsids in Table 13) or other appropriate AAV serotype capsids. Accordingly, provided are compositions comprising, and methods of administering, the AUF1 AAV gene therapy vectors described herein (for example, as depicted in FIG. 1 and Table 3) for restoring or increasing muscle mass, muscle function or performance, and/or reducing or reversing muscle atrophy. Such methods include stabilizing the sarcolemma of the muscle cell by reducing leakiness (for example, as measured by creatine kinase levels), increasing expression of β-sarcoglycan or utrophin and/or its presence in the dystrophin-glycoprotein complex of muscle cells by providing AUF1 protein. Other methods provided include administering the AUF1 AAV gene therapy constructs disclosed herein for treatment, prevention or amelioration of the symptoms of muscle wasting including sarcopenia, including in the elderly, traumatic injury, and diseases or disorders associated with a lack or loss of muscle mass, function or performance, such as, but not limited to dystrophinopathies and other related muscle diseases or disorders. Such methods include promoting an increase in muscle cell mass, number of muscle fibers, size of muscle fibers, muscle cell regeneration, reduction in or reverse of muscle cell atrophy, satellite cell activation and differentiation, improvement in muscle cell function (for example, by increasing mitochondrial oxidative capacity), and increasing proportion of slow twitch fiber in muscle (including by conversion of fast to slow twitch muscle fibers).

Also provided are pharmaceutical compositions formulated for peripheral, including, intravenous, administration of the AUF1-encoding rAAV described herein.

### 5.1. Definitions

The term “vector” is used interchangeably with “expression vector.” The term “vector” may refer to viral or non-viral, prokaryotic or eukaryotic, DNA or RNA sequences that are capable of being transfected into a cell, referred to as “host cell,” so that all or a part of the sequences are transcribed. It is not necessary for the transcript to be expressed. It is also not necessary for a vector to comprise a transgene having a coding sequence. Vectors are frequently assembled as composites of elements derived from different viral, bacterial, or mammalian genes. Vectors contain various coding and non-coding sequences, such as sequences coding for selectable markers, sequences that facilitate their propagation in bacteria, or one or more transcription units that are expressed only in certain cell types. For example, mammalian expression vectors often contain both prokaryotic sequences that facilitate the propagation of the vector in bacteria and one or more eukaryotic transcription units that are expressed only in eukaryotic cells. It will be appreciated by those skilled in the art that the design of the expression vector can depend on such factors as the choice of the host cell to be transformed, the level of expression of protein desired, etc.

The term “promoter” is used interchangeably with “promoter element” and “promoter sequence.” Likewise, the term “enhancer” is used interchangeably with “enhancer element” and “enhancer sequence.” The term “promoter” refers to a minimal sequence of a transgene that is sufficient to initiate transcription of a coding sequence of the transgene. Promoters may be constitutive or inducible. A constitutive promoter is considered to be a strong promoter if it drives expression of a transgene at a level comparable to that of the cytomegalovirus promoter (CMV) (Boshart et al., “A Very Strong Enhancer is Located Upstream of an Immediate Early Gene of Human Cytomegalovirus,” Cell 41:521 (1985), which is hereby incorporated by reference in its entirety). Promoters may be synthetic, modified, or hybrid promoters. Promoters may be coupled with other regulatory sequences/elements which, when bound to appropriate intracellular regulatory factors, enhance (“enhancers”) or repress (“repressors”) promoter-dependent transcription. A promoter, enhancer, or repressor, is said to be “operably linked” to a transgene when such element(s) control(s) or affect(s) transgene transcription rate or efficiency. For example, a promoter sequence located proximally to the 5′ end of a transgene coding sequence is usually operably linked with the transgene. As used herein, the term “regulatory elements” is used interchangeably with “regulatory sequences” and refers to promoters, enhancers, and other expression control elements, or any combination of such elements.

Promoters are positioned 5′ (upstream) to the genes that they control. Many eukaryotic promoters contain two types of recognition sequences: TATA box and the upstream promoter elements. The TATA box, located 25-30 bp upstream of the transcription initiation site, is thought to be involved in directing RNA polymerase II to begin RNA synthesis at the correct site. In contrast, the upstream promoter elements determine the rate at which transcription is initiated. These elements can act regardless of their orientation, but they must be located within 100 to 200 bp upstream of the TATA box.

Enhancer elements can stimulate transcription up to 1000-fold from linked homologous or heterologous promoters. Enhancer elements often remain active even if their orientation is reversed (Li et al., “High Level Desmin Expression Depends on a Muscle-Specific Enhancer,” J. Bio. Chem. 266(10):6562-6570 (1991), which is hereby incorporated by reference in its entirety). Furthermore, unlike promoter elements, enhancers can be active when placed downstream from the transcription initiation site, e.g., within an intron, or even at a considerable distance from the promoter (Yutzey et al., “An Internal Regulatory Element Controls Troponin I Gene Expression,” Mol. Cell. Bio. 9(4):1397-1405 (1989), which is hereby incorporated by reference in its entirety).

The term “muscle cell-specific” refers to the capability of regulatory elements, such as promoters and enhancers, to drive expression of an operatively linked nucleic acid molecule (e.g., a nucleic acid molecule encoding an AU-rich mRNA binding factor 1 (AUF1) protein or a functional fragment thereof) exclusively or preferentially in muscle cells or muscle tissue.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein having a modified sequence and/or a peptide insertion into the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap helper plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural protein needed for replication and production of virus.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

Amino acid residues as disclosed herein can be modified by conservative substitutions to maintain, or substantially maintain, overall polypeptide structure and/or function. As used herein, “conservative amino acid substitution” indicates that: hydrophobic amino acids (i.e., Ala, Cys, Gly, Pro, Met, Val, lie, and Leu) can be substituted with other hydrophobic amino acids; hydrophobic amino acids with bulky side chains (i.e., Phe, Tyr, and Trp) can be substituted with other hydrophobic amino acids with bulky side chains; amino acids with positively charged side chains (i.e., Arg, His, and Lys) can be substituted with other amino acids with positively charged side chains; amino acids with negatively charged side chains (i.e., Asp and Glu) can be substituted with other amino acids with negatively charged side chains; and amino acids with polar uncharged side chains (i.e., Ser, Thr, Asn, and Gln) can be substituted with other amino acids with polar uncharged side chains.

The terms “subject”, “host”, and “patient” are used interchangeably. A subject may be a mammal such as a non-primate (e.g., cows, pigs, horses, cats, dogs, rats etc.) or a primate (e.g., monkey and human), and includes a human.

The terms “therapeutic agent” refers to any agent which can be used in treating, managing, or ameliorating symptoms associated with a disease or disorder, where the disease or disorder is associated with a function to be provided by a transgene. A “therapeutically effective amount” refers to the amount of agent, (e.g., an amount of product expressed by the transgene) that provides at least one therapeutic benefit in the treatment or management of the target disease or disorder, when administered to a subject suffering therefrom. Further, a therapeutically effective amount with respect to an agent of the invention means that amount of agent alone, or when in combination with other therapies, that provides at least one therapeutic benefit in the treatment or management of the disease or disorder.

The term “prophylactic agent” refers to any agent which can be used in the prevention, reducing the likelihood of, delay, or slowing down of the progression of a disease or disorder, where the disease or disorder is associated with a function to be provided by a transgene. A “prophylactically effective amount” refers to the amount of the prophylactic agent (e.g., an amount of product expressed by the transgene) that provides at least one prophylactic benefit in the prevention or delay of the target disease or disorder, when administered to a subject predisposed thereto. A prophylactically effective amount also may refer to the amount of agent sufficient to prevent, reduce the likelihood of, or delay the occurrence of the target disease or disorder; or slow the progression of the target disease or disorder; the amount sufficient to delay or minimize the onset of the target disease or disorder; or the amount sufficient to prevent or delay the recurrence or spread thereof. A prophylactically effective amount also may refer to the amount of agent sufficient to prevent or delay the exacerbation of symptoms of a target disease or disorder. Further, a prophylactically effective amount with respect to a prophylactic agent of the invention means that amount of prophylactic agent alone, or when in combination with other agents, that provides at least one prophylactic benefit in the prevention or delay of the disease or disorder.

A prophylactic agent of the invention can be administered to a subject “pre-disposed” to a target disease or disorder. A subject that is “pre-disposed” to a disease or disorder is one that shows symptoms associated with the development of the disease or disorder, or that has a genetic makeup, environmental exposure, or other risk factor for such a disease or disorder, but where the symptoms are not yet at the level to be diagnosed as the disease or disorder. For example, a patient with a family history of a disease associated with a missing gene (to be provided by a transgene) may qualify as one predisposed thereto. Further, a patient with a dormant tumor that persists after removal of a primary tumor may qualify as one predisposed to recurrence of a tumor.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients, or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the nucleic acid molecule described herein.

The term “CpG islands” means those distinctive regions of the genome that contain the dinucleotide CpG (e.g., C (cytosine) base followed immediately by a G (guanine) base (a CpG)) at high frequency, thus the G+C content of CpG islands is significantly higher than that of non-island DNA. CpG islands can be identified by analysis of nucleotide length, nucleotide composition, and frequency of CpG dinucleotides. CpG island content in any particular nucleotide sequence or genome may be measured using the following criteria: island size greater than 100, GC Percent greater than 50.0%, and ratio greater than 0.6 of observed number of CG dinucleotides to the expected number on the basis of the number of Gs and Cs in the segment (Obs/Exp greater than 0.6).

Obs/Exp CpG=Number of CpG*N/(Number of C*Number of G)

where N=length of sequence.

Various software tools are available for such calculations, such as world-wide-web.urogene.org/cgi-bin/methprimer/methprimer.cgi, world-wide-web.cpgislands.usc.edu/, world-wide-web.ebi.ac.uk/Tools/emboss/cpgplot/index.html and world-wide-web.bioinformatics.org/sms2/cpg_islands.html. (See also Gardiner-Garden and Frommer, J Mol Biol. 1987 Jul. 20; 196(2):261-82; Li L C and Dahiya R. MethPrimer: designing primers for methylation PCRs. Bioinformatics. 2002 November; 18(11):1427-31.). In one embodiment the algorithm to identify CpG islands is found at www.urogene.org/cgi-bin/methprimer/methprimer.cgi.

### 5.2. AU-Rich mRNA Binding Factor 1 Vectors

5.2.1 AU-Rich mRNA Binding Factor 1 Transgenes

Provided are nucleic acids, including transgenes, encoding AUF1s, including the p37, p40, p42 and p45 isoforms of human and mouse AUF1, or therapeutically functional fragments thereof, and vectors and viral particles, including rAAVs, containing same and methods of using same in methods of treatment, prevention or amelioration of symptoms of conditions associated with loss of muscle mass or performance or where an increase in muscle mass or performance is desired or useful. The AUF1 gene therapy vectors are used in methods of treating or ameliorating the symptoms of dystrophinopathy by administering the AUF1 gene therapy vectors in combination with gene therapy vectors encoding microdystrophins.

Genes involved in rapid response to cell stimuli are highly regulated and typically encode mRNAs that are selectively and rapidly degraded to quickly terminate protein expression and reprogram the cell (Moore et al., “Physiological Networks and Disease Functions of RNA-binding Protein AUF1,” Wiley Interdiscip. Rev. RNA 5(4):549-64 (2014), which is hereby incorporated by reference in its entirety). These include growth factors, inflammatory cytokines (Moore et al., “Physiological Networks and Disease Functions of RNA-binding Protein AUF1,” Wiley Interdiscip Rev RNA 5(4):549-64 (2014) and Zhang et al., “Purification, Characterization, and cDNA Cloning of an AU-rich Element RNA-binding Protein, AUF1,” Mol. Cell. Biol. 13(12):7652-65 (1993), which are hereby incorporated by reference in their entirety), and tissue stem cell fate-determining mRNAs (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety) that have very short half-lives of 5-30 minutes.

Short-lived mRNAs typically contain an AU-rich element (“ARE”) in the 3′ untranslated region (“3′UTR”) of the mRNA, having the repeated sequence AUUUA (Moore et al., “Physiological Networks and Disease Functions of RNA-binding Protein AUF1,” Wiley Interdiscip Rev. RNA 5(4):549-64 (2014), which is hereby incorporated by reference in its entirety), which confers rapid decay or in some cases stabilization. The ARE serves as a binding site for regulatory proteins known as AU-rich binding proteins (AUBPs) that control the stability and in some cases the translation of the mRNA (Moore et al., “Physiological Networks and Disease Functions of RNA-binding Protein AUF1,” Wiley Interdiscip. Rev. RNA 5(4):549-64 (2014); Zhang et al., “Purification, Characterization, and cDNA Cloning of an AU-rich Element RNA-binding Protein, AUF1,” Mol. Cell. Biol. 13(12):7652-65 (1993); and Halees et al., “ARED Organism: Expansion of ARED Reveals AU-rich Element Cluster Variations Between Human And Mouse,” Nucleic Acids Res 36(Database issue):D137-40 (2008), which are hereby incorporated by reference in their entirety).

AU-rich mRNA binding factor 1 (AUF1; also known as Heterogeneous Nuclear Ribonucleoprotein D0, hnRNP D0; HNRNPD gene) binds with high affinity to repeated AU-rich elements (“AREs”) located in the 3′ untranslated region (“3′ UTR”) found in approximately 5% of mRNAs. Although AUF1 typically targets ARE-mRNAs for rapid degradation, while not as well understood, it can oppositely stabilize and increase the translation of some ARE-mRNAs (Moore et al., “Physiological Networks and Disease Functions of RNA-Binding Protein AUF1,” Wiley Interdiscip. Rev. RNA 5(4):549-564 (2014), which is hereby incorporated by reference in its entirety). It was previously reported that mice with AUF1 deficiency undergo an accelerated loss of muscle mass due to an inability to carry out the myogenesis program (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-90 (2016), which is hereby incorporated by reference in its entirety). It was also found that AUF1 expression is severely reduced with age in skeletal muscle, and this significantly contributes to loss and atrophy of muscle, loss of muscle mass, and reduced strength (Abbadi et al., “Muscle Development and Regeneration Controlled by AUF1-mediated Stage-specific Degradation of Fate-determining Checkpoint mRNAs,” Proc. Natl. Acad. Sci. USA 116(23):11285-11290 (2019), and Abbadi et al. “AUF1 Gene Transfer Increases Exercise Performance and Improves Skeletal Muscle Deficit in Adult Mice” Molecular Therapy 22:222-236 (2021), which are hereby incorporated by reference in their entireties). It was also found that AUF1 controls all major stages of skeletal muscle development, starting with satellite cell activation and lineage commitment, by selectively targeting for rapid degradation the major differentiation checkpoint mRNAs that block entry into each next step of muscle development.

AUF1 has four related protein isoforms identified by their molecular weight (p37^{AUF1}, p40^{AUF1}, p42^{AUF1}, p45^{AUF1}) derived by differential splicing of a single pre-mRNA (Moore et al., “Physiological Networks and Disease Functions of RNA-Binding Protein AUF1,” Wiley Interdiscip. Rev. RNA 5(4):549-564 (2014); Chen & Shyu, “AU-Rich Elements: Characterization and Importance in mRNA Degradation,” Trends Biochem. Sci. 20(11):465-470 (1995); and Kim et al., “Emerging Roles of RNA and RNA-Binding Protein Network in Cancer Cells,” BMB Rep. 42(3):125-130 (2009), which are hereby incorporated by reference in their entirety). Each of these four isoforms include two centrally-positioned, tandemly arranged RNA recognition motifs (“RRMs”) which mediate RNA binding (DeMaria et al., “Structural Determinants in AUF 1 Required for High Affinity Binding to A+U-rich Elements,” J. Biol. Chem. 272:27635-27643 (1997), which is hereby incorporated by reference in its entirety).

The general organization of an RRM is a β-α-β-β-α-β RNA binding platform of anti-parallel O-sheets backed by the α-helices (Zucconi & Wilson, “Modulation of Neoplastic Gene Regulatory Pathways by the RNA-binding Factor AUF1,” Front. Biosci. 16:2307-2325 (2013); Nagai et al., “The RNP Domain: A Sequence-specific RNA-binding Domain Involved in Processing and Transport of RNA,” Trends Biochem. Sci. 20:235-240 (1995), which are hereby incorporated by reference in their entirety). Structures of individual AUF1 RRM domains resolved by NMR are largely consistent with this overall tertiary fold (Zucconi & Wilson, “Modulation of Neoplastic Gene Regulatory Pathways by the RNA-binding Factor AUF1,” Front. Biosci. 16:2307-2325 (2013); Nagata et al., “Structure and Interactions with RNA of the N-terminal UUAG-specific RNA-binding Domain of hnRNP D0,” J. Mol. Biol. 287:221-237 (1999); and Katahira et al., “Structure of the C-terminal RNA-binding Domain of hnRNP D0 (AUF1), its Interactions with RNA and DNA, and Change in Backbone Dynamics Upon Complex Formation with DNA,” J. Mol. Biol. 311:973-988 (2001), which are hereby incorporated by reference in their entirety).

Mutations and/or polymorphisms in AUF1 are linked to human limb girdle muscular dystrophy (LGMD) type 1G (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety), suggesting a critical requirement for AUF1 in post-natal skeletal muscle regeneration and maintenance.

The term “fragment” or “portion” when used herein with respect to a given polypeptide sequence (e.g., AUF1), refers to a contiguous stretch of amino acids of the given polypeptide's sequence that is shorter than the given polypeptide's full-length sequence. A fragment of a polypeptide may be defined by its first position and its final position, in which the first and final positions each correspond to a position in the sequence of the given full-length polypeptide. The sequence position corresponding to the first position is situated N-terminal to the sequence position corresponding to the final position. The sequence of the fragment or portion is the contiguous amino acid sequence or stretch of amino acids in the given polypeptide that begins at the sequence position corresponding to the first position and ends at the sequence position corresponding to the final position. Functional or active fragments are fragments that retain functional characteristics, e.g., of the native sequence or other reference sequence. Typically, active fragments are fragments that retain substantially the same activity as the wild-type protein. A fragment may, for example, contain a functionally important domain, such as a domain that is important for receptor or ligand binding. Functional fragments are at least 10, 15, 20, 50, 75, 100, 150, 200, 250 or 300 contiguous amino acids of a full length AUF1 (including the p37, p40, p42 or p45 isoforms thereof) and retain one or more AUF1 functions.

Accordingly, in certain embodiments, functional fragments of AUF1 as described herein include at least one RNA recognition domain (“RRM”) domain. In certain embodiments, functional fragments of AUF1 as described herein include two RRM domains.

AUF1 or functional fragments thereof as described herein may be derived from a mammalian AUF1. In one embodiment, the AUF1 or functional fragment thereof is a human AUF1 or functional fragment thereof. In another embodiment, the AUF1 or functional fragment thereof is a murine AUF1 or a functional fragment thereof. The AUF1 protein according to embodiments described herein may include one or more of the AUF1 isoforms p37^{AUF1}, p40^{AUF1}, p42^{AUF1}, and p45^{AUF1}. The GenBank accession numbers corresponding to the nucleotide and amino acid sequences of each human and mouse isoform is found in Table 1 below, each of which is hereby incorporated by reference in its entirety.

TABLE 1
Summary of GenBank Accession Numbers of AUF1 Sequences

| | Human | | Mouse | |
|---|---|---|---|---|
| Isoform | Nucleotide | Amino Acid | Nucleotide | Amino Acid |
| p37^{AUF1} | NM_001003810.2 | NP_001003810.1 | NM_001077267.2 | NP_001070735.1 |
| | (SEQ ID NO: 1) | (SEQ ID NO: 2) | (SEQ ID NO: 3) | (SEQ ID NO: 4) |
| p40^{AUF1} | NM_002138.3 | NP_002129.2 | NM_007516.3 | NP_031542.2 |
| | (SEQ ID NO: 5) | (SEQ ID NO: 6) | (SEQ ID NO: 7) | (SEQ ID NO: 8) |
| p42^{AUF1} | NM_031369.2 | NP_112737.1 | NM_001077266.2 | NP_001070734.1 |
| | (SEQ ID NO: 9) | (SEQ ID NO: 10) | (SEQ ID NO: 11) | (SEQ ID NO: 12) |
| p45^{AUF1} | NM_031370.2 | NP_112738.1 | NM_001077265.2 | NP_001070733.1 |
| | (SEQ ID NO: 13) | (SEQ ID NO: 14) | (SEQ ID NO: 15) | (SEQ ID NO: 16) |

The sequences referred to in Table 1 are reproduced below.

The human p37^{AUF1} nucleotide sequence of GenBank Accession No. NM_001003810.1 (SEQ ID NO: 1) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GGAAAATGTT TATAGGAGGC CTTAGCTGGG ACACTACAAA GAAAGATCTG | 600 |
| AAGGACTACT TTTCCAAATT TGGTGAAGTT GTAGACTGCA CTCTGAAGTT AGATCCTATC | 660 |
| ACAGGGCGAT CAAGGGGTTT TGGCTTTGTG CTATTTAAAG AATCGGAGAG TGTAGATAAG | 720 |
| GTCATGGATC AAAAAGAACA TAAATTGAAT GGGAAGGTGA TTGATCCTAA AAGGGCCAAA | 780 |
| GCCATGAAAA CAAAAGAGCC GGTTAAAAAA ATTTTTGTTG GTGGCCTTTC TCCAGATACA | 840 |
| CCTGAAGAGA AAATAAGGGA GTACTTTGGT GGTTTTGGTG AGGTGGAATC CATAGAGCTC | 900 |
| CCCATGGACA ACAAGACCAA TAAGAGGCGT GGGTTCTGCT TTATTACCTT TAAGGAAGAA | 960 |
| GAACCAGTGA AGAAGATAAT GGAAAAGAAA TACCACAATG TTGGTCTTAG TAAATGTGAA | 1020 |
| ATAAAAGTAG CCATGTCGAA GGAACAATAT CAGCAACAGC AACAGTGGGG ATCTAGAGGA | 1080 |
| GGATTTGCAG GAAGAGCTCG TGGAAGAGGT GGTGACCAGC AGAGTGGTTA TGGGAAGGTA | 1140 |
| TCCAGGCGAG GTGGTCATCA AAATAGCTAC AAACCATACT AAATTATTCC ATTTGCAACT | 1200 |
| TATCCCCAAC AGGTGGTGAA GCAGTATTTT CCAATTTGAA GATTCATTTG AAGGTGGCTC | 1260 |
| CTGCCACCTG CTAATAGCAG TTCAAACTAA ATTTTTTGTA TCAAGTCCCT GAATGGAAGT | 1320 |
| ATGACGTTGG GTCCCTCTGA AGTTTAATTC TGAGTTCTCA TTAAAAGAAA TTTGCTTTCA | 1380 |
| TTGTTTTATT TCTTAATTGC TATGCTTCAG AATCAATTTG TGTTTTATGC CCTTTCCCCC | 1440 |
| AGTATTGTAG AGCAAGTCTT GTGTTAAAAG CCCAGTGTGA CAGTGTCATG ATGTAGTAGT | 1500 |
| GTCTTACTGG TTTTTTAATA AATCCTTTTG TATAAAAATG TATTGGCTCT TTTATCATCA | 1560 |
| GAATAGGAAA AATTGTCATG GATTCAAGTT ATTAAAAGCA TAAGTTTGGA AGACAGGCTT | 1620 |
| GCCGAAATTG AGGACATGAT TAAAATTGCA GTGAAGTTTG AAATGTTTTT AGCAAAATCT | 1680 |
| AATTTTTGCC ATAATGTGTC CTCCCTGTCC AAATTGGGAA TGACTTAATG TCAATTTGTT | 1740 |
| TGTTGGTTGT TTTAATAATA CTTCCTTATG TAGCCATTAA GATTTATATG AATATTTTCC | 1800 |
| CAAATGCCCA GTTTTTGCTT AATATGTATT GTGCTTTTTA GAACAAATCT GGATAAATGT | 1860 |
| GCAAAAGTAC CCCTTTGCAC AGATAGTTAA TGTTTTATGC TTCCATTAAA TAAAAAGGAC | 1920 |
| TTAAAATCTG TTAATTATAA TAGAAATGCG GCTAGTTCAG AGAGATTTTT AGAGCTGTGG | 1980 |
| TGGACTTCAT AGATGAATTC AAGTGTTGAG GGAGGATTAA AGAAATATAT ACCGTGTTTA | 2040 |
| TGTGTGTGTG CTT | |

The human p37^{AUF1} amino acid sequence of GenBank Accession No. NP_001003810.1 (SEQ ID NO: 2) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGDQQSG YGKVSRRGGH QNSYKPY | |

The human p40^{AUF1} nucleotide sequence of GenBank Accession No. NM_002138.3 (SEQ ID NO: 5) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GCCATTCAAA CTCCTCCCCA CGACACTCTG AAGCAGCGAC GGCACAGCGG | 600 |
| GAAGAATGGA AAATGTTTAT AGGAGGCCTT AGCTGGGACA CTACAAAGAA AGATCTGAAG | 660 |
| GACTACTTTT CCAAATTTGG TGAAGTTGTA GACTGCACTC TGAAGTTAGA TCCTATCACA | 720 |
| GGGCGATCAA GGGGTTTTGG CTTTGTGCTA TTTAAAGAAT CGGAGAGTGT AGATAAGGTC | 780 |
| ATGGATCAAA AAGAACATAA ATTGAATGGG AAGGTGATTG ATCCTAAAAG GGCCAAAGCC | 840 |
| ATGAAAACAA AAGAGCCGGT TAAAAAAATT TTTGTTGGTG GCCTTTCTCC AGATACACCT | 900 |
| GAAGAGAAAA TAAGGGAGTA CTTTGGTGGT TTTGGTGAGG TGGAATCCAT AGAGCTCCCC | 960 |
| ATGGACAACA AGACCAATAA GAGGCGTGGG TTCTGCTTTA TTACCTTTAA GGAAGAAGAA | 1020 |
| CCAGTGAAGA AGATAATGGA AAAGAAATAC CACAATGTTG GTCTTAGTAA ATGTGAAATA | 1080 |
| AAAGTAGCCA TGTCGAAGGA ACAATATCAG CAACAGCAAC AGTGGGGATC TAGAGGAGGA | 1140 |
| TTTGCAGGAA GAGCTCGTGG AAGAGGTGGT GACCAGCAGA GTGGTTATGG GAAGGTATCC | 1200 |
| AGGCGAGGTG GTCATCAAAA TAGCTACAAA CCATACTAAA TTATTCCATT TGCAACTTAT | 1260 |
| CCCCAACAGG TGGTGAAGCA GTATTTTCCA ATTTGAAGAT TCATTTGAAG GTGGCTCCTG | 1320 |
| CCACCTGCTA ATAGCAGTTC AAACTAAATT TTTTGTATCA AGTCCCTGAA TGGAAGTATG | 1380 |
| ACGTTGGGTC CCTCTGAAGT TTAATTCTGA GTTCTCATTA AAAGAAATTT GCTTTCATTG | 1440 |
| TTTTATTTCT TAATTGCTAT GCTTCAGAAT CAATTTGTGT TTTATGCCCT TTCCCCCAGT | 1500 |
| ATTGTAGAGC AAGTCTTGTG TTAAAAGCCC AGTGTGACAG TGTCATGATG TAGTAGTGTC | 1560 |
| TTACTGGTTT TTTAATAAAT CCTTTTGTAT AAAAATGTAT TGGCTCTTTT ATCATCAGAA | 1620 |
| TAGGAAAAAT TGTCATGGAT TCAAGTTATT AAAAGCATAA GTTTGGAAGA CAGGCTTGCC | 1680 |
| GAAATTGAGG ACATGATTAA AATTGCAGTG AAGTTTGAAA TGTTTTTAGC AAAATCTAAT | 1740 |
| TTTTGCCATA ATGTGTCCTC CCTGTCCAAA TTGGGAATGA CTTAATGTCA ATTTGTTTGT | 1800 |
| TGGTTGTTTT AATAATACTT CCTTATGTAG CCATTAAGAT TTATATGAAT ATTTTCCCAA | 1860 |
| ATGCCCAGTT TTTGCTTAAT ATGTATTGTG CTTTTTAGAA CAAATCTGGA TAAATGTGCA | 1920 |
| AAAGTACCCC TTTGCACAGA TAGTTAATGT TTTATGCTTC CATTAAATAA AAAGGACTTA | 1980 |
| AAATCTGTTA ATTATAATAG AAATGCGGCT AGTTCAGAGA GATTTTTAGA GCTGTGGTGG | 2040 |
| ACTTCATAGA TGAATTCAAG TGTTGAGGGA GGATTAAAGA AATATATACC GTGTTTATGT | 2100 |
| GTGTGTGCTT | |

The human p40^{AUF1} amino acid sequence of GenBank Accession No. NP_002129.2 (SEQ ID NO: 6) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGHS NSSPRHSEAA TAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGDQQSGY GKVSRRGGHQ | 300 |
| NSYKPY | |

The human p42^{AUF1} nucleotide sequence of GenBank Accession No. NM_031369.2 (SEQ ID NO: 9) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GGAAAATGTT TATAGGAGGC CTTAGCTGGG ACACTACAAA GAAAGATCTG | 600 |
| AAGGACTACT TTTCCAAATT TGGTGAAGTT GTAGACTGCA CTCTGAAGTT AGATCCTATC | 660 |
| ACAGGGCGAT CAAGGGGTTT TGGCTTTGTG CTATTTAAAG AATCGGAGAG TGTAGATAAG | 720 |
| GTCATGGATC AAAAAGAACA TAAATTGAAT GGGAAGGTGA TTGATCCTAA AAGGGCCAAA | 780 |
| GCCATGAAAA CAAAAGAGCC GGTTAAAAAA ATTTTTGTTG GTGGCCTTTC TCCAGATACA | 840 |
| CCTGAAGAGA AAATAAGGGA GTACTTTGGT GGTTTTGGTG AGGTGGAATC CATAGAGCTC | 900 |
| CCCATGGACA ACAAGACCAA TAAGAGGCGT GGGTTCTGCT TTATTACCTT TAAGGAAGAA | 960 |
| GAACCAGTGA AGAAGATAAT GGAAAAGAAA TACCACAATG TTGGTCTTAG TAAATGTGAA | 1020 |
| ATAAAAGTAG CCATGTCGAA GGAACAATAT CAGCAACAGC AACAGTGGGG ATCTAGAGGA | 1080 |
| GGATTTGCAG GAAGAGCTCG TGGAAGAGGT GGTGGCCCCA GTCAAAACTG GAACCAGGGA | 1140 |
| TATAGTAACT ATTGGAATCA AGGCTATGGC AACTATGGAT ATAACAGCCA AGGTTACGGT | 1200 |
| GGTTATGGAG GATATGACTA CACTGGTTAC AACAACTACT ATGGATATGG TGATTATAGC | 1260 |
| AACCAGCAGA GTGGTTATGG GAAGGTATCC AGGCGAGGTG GTCATCAAAA TAGCTACAAA | 1320 |
| CCATACTAAA TTATTCCATT TGCAACTTAT CCCCAACAGG TGGTGAAGCA GTATTTTCCA | 1380 |
| ATTTGAAGAT TCATTTGAAG GTGGCTCCTG CCACCTGCTA ATAGCAGTTC AAACTAAATT | 1440 |
| TTTTGTATCA AGTCCCTGAA TGGAAGTATG ACGTTGGGTC CCTCTGAAGT TTAATTCTGA | 1500 |
| GTTCTCATTA AAAGAAATTT GCTTTCATTG TTTTATTTCT TAATTGCTAT GCTTCAGAAT | 1560 |
| CAATTTGTGT TTTATGCCCT TTCCCCCAGT ATTGTAGAGC AAGTCTTGTG TTAAAAGCCC | 1620 |
| AGTGTGACAG TGTCATGATG TAGTAGTGTC TTACTGGTTT TTTAATAAAT CCTTTTGTAT | 1680 |
| AAAAATGTAT TGGCTCTTTT ATCATCAGAA TAGGAAAAAT TGTCATGGAT TCAAGTTATT | 1740 |
| AAAAGCATAA GTTTGGAAGA CAGGCTTGCC GAAATTGAGG ACATGATTAA AATTGCAGTG | 1800 |
| AAGTTTGAAA TGTTTTTAGC AAAATCTAAT TTTTGCCATA ATGTGTCCTC CCTGTCCAAA | 1860 |
| TTGGGAATGA CTTAATGTCA ATTTGTTTGT TGGTTGTTTT AATAATACTT CCTTATGTAG | 1920 |
| CCATTAAGAT TTATATGAAT ATTTTCCCAA ATGCCCAGTT TTTGCTTAAT ATGTATTGTG | 1980 |
| CTTTTTAGAA CAAATCTGGA TAAATGTGCA AAAGTACCCC TTTGCACAGA TAGTTAATGT | 2040 |
| TTTATGCTTC CATTAAATAA AAAGGACTTA AAATCTGTTA ATTATAATAG AAATGCGGCT | 2100 |
| AGTTCAGAGA GATTTTTAGA GCTGTGGTGG ACTTCATAGA TGAATTCAAG TGTTGAGGGA | 2160 |
| GGATTAAAGA AATATATACC GTGTTTATGT GTGTGTGCTT | |

The human p42^{AUF1} amino acid sequence of GenBank Accession No. NP_112737.1 (SEQ ID NO: 10) is as follows:

| | |
|---|---|
| MSEEQFGGDG AAAAATAAVG GSAGEQEGAM VAATQGAAAA AGSGAGTGGG TASGGTEGGS | 61 |
| AESEGAKIDA SKNEEDEGKM FIGGLSWDTT KKDLKDYFSK FGEVVDCTLK LDPITGRSRG | 121 |
| FGFVLFKESE SVDKVMDQKE HKLNGKVIDP KRAKAMKTKE PVKKIFVGGL SPDTPEEKIR | 181 |
| EYFGGFGEVE SIELPMDNKT NKRRGFCFIT FKEEEPVKKI MEKKYHNVGL SKCEIKVAMS | 241 |
| KEQYQQQQQW GSRGGFAGRA RGRGGGPSQN WNQGYSNYWN QGYGNYGYNS QGYGGYGGYD | 301 |
| YTGYNNYYGY GDYSNQQSGY GKVSRRGGHQ NSYKPY | |

The human p45^{AUF1} nucleotide sequence of GenBank Accession No. NM_031370.2 (SEQ ID NO: 13) is as follows:

| | |
|---|---|
| CTTCCGTCGG CCATTTTAGG TGGTCCGCGG CGGCGCCATT AAAGCGAGGA GGAGGCGAGA | 60 |
| GCGGCCGCCG CTGGTGCTTA TTCTTTTTTA GTGCAGCGGG AGAGAGCGGG AGTGTGCGCC | 120 |
| GCGCGAGAGT GGGAGGCGAA GGGGGCAGGC CAGGGAGAGG CGCAGGAGCC TTTGCAGCCA | 180 |
| CGCGCGCGCC TTCCCTGTCT TGTGTGCTTC GCGAGGTAGA GCGGGCGCGC GGCAGCGGCG | 240 |
| GGGATTACTT TGCTGCTAGT TTCGGTTCGC GGCAGCGGCG GGTGTAGTCT CGGCGGCAGC | 300 |
| GGCGGAGACA CTAGCACTAT GTCGGAGGAG CAGTTCGGCG GGGACGGGGC GGCGGCAGCG | 360 |
| GCAACGGCGG CGGTAGGCGG CTCGGCGGGC GAGCAGGAGG GAGCCATGGT GGCGGCGACA | 420 |
| CAGGGGGCAG CGGCGGCGGC GGGAAGCGGA GCCGGGACCG GGGGCGGAAC CGCGTCTGGA | 480 |
| GGCACCGAAG GGGGCAGCGC CGAGTCGGAG GGGGCGAAGA TTGACGCCAG TAAGAACGAG | 540 |
| GAGGATGAAG GCCATTCAAA CTCCTCCCCA CGACACTCTG AAGCAGCGAC GGCACAGCGG | 600 |
| GAAGAATGGA AAATGTTTAT AGGAGGCCTT AGCTGGGACA CTACAAAGAA AGATCTGAAG | 660 |
| GACTACTTTT CCAAATTTGG TGAAGTTGTA GACTGCACTC TGAAGTTAGA TCCTATCACA | 720 |
| GGGCGATCAA GGGGTTTTGG CTTTGTGCTA TTTAAAGAAT CGGAGAGTGT AGATAAGGTC | 780 |
| ATGGATCAAA AAGAACATAA ATTGAATGGG AAGGTGATTG ATCCTAAAAG GGCCAAAGCC | 840 |
| ATGAAAACAA AAGAGCCGGT TAAAAAAATT TTTGTTGGTG GCCTTTCTCC AGATACACCT | 900 |
| GAAGAGAAAA TAAGGGAGTA CTTTGGTGGT TTTGGTGAGG TGGAATCCAT AGAGCTCCCC | 960 |
| ATGGACAACA AGACCAATAA GAGGCGTGGG TTCTGCTTTA TTACCTTTAA GGAAGAAGAA | 1020 |
| CCAGTGAAGA AGATAATGGA AAAGAAATAC CACAATGTTG GTCTTAGTAA ATGTGAAATA | 1080 |
| AAAGTAGCCA TGTCGAAGGA ACAATATCAG CAACAGCAAC AGTGGGGATC TAGAGGAGGA | 1140 |
| TTTGCAGGAA GAGCTCGTGG AAGAGGTGGT GGCCCCAGTC AAAACTGGAA CCAGGGATAT | 1200 |
| AGTAACTATT GGAATCAAGG CTATGGCAAC TATGGATATA ACAGCCAAGG TTACGGTGGT | 1260 |
| TATGGAGGAT ATGACTACAC TGGTTACAAC AACTACTATG GATATGGTGA TTATAGCAAC | 1320 |
| CAGCAGAGTG GTTATGGGAA GGTATCCAGG CGAGGTGGTC ATCAAAATAG CTACAAACCA | 1380 |
| TACTAAATTA TTCCATTTGC AACTTATCCC CAACAGGTGG TGAAGCAGTA TTTTCCAATT | 1440 |
| TGAAGATTCA TTTGAAGGTG GCTCCTGCCA CCTGCTAATA GCAGTTCAAA CTAAATTTTT | 1500 |
| TGTATCAAGT CCCTGAATGG AAGTATGACG TTGGGTCCCT CTGAAGTTTA ATTCTGAGTT | 1560 |
| CTCATTAAAA GAAATTTGCT TTCATTGTTT TATTTCTTAA TTGCTATGCT TCAGAATCAA | 1620 |
| TTTGTGTTTT ATGCCCTTTC CCCCAGTATT GTAGAGCAAG TCTTGTGTTA AAAGCCCAGT | 1680 |
| GTGACAGTGT CATGATGTAG TAGTGTCTTA CTGGTTTTTT AATAAATCCT TTTGTATAAA | 1740 |
| AATGTATTGG CTCTTTTATC ATCAGAATAG GAAAAATTGT CATGGATTCA AGTTATTAAA | 1800 |
| AGCATAAGTT TGGAAGACAG GCTTGCCGAA ATTGAGGACA TGATTAAAAT TGCAGTGAAG | 1860 |
| TTTGAAATGT TTTTAGCAAA ATCTAATTTT TGCCATAATG TGTCCTCCCT GTCCAAATTG | 1920 |
| GGAATGACTT AATGTCAATT TGTTTGTTGG TTGTTTTAAT AATACTTCCT TATGTAGCCA | 1980 |
| TTAAGATTTA TATGAATATT TTCCCAAATG CCCAGTTTTT GCTTAATATG TATTGTGCTT | 2040 |
| TTTAGAACAA ATCTGGATAA ATGTGCAAAA GTACCCCTTT GCACAGATAG TTAATGTTTT | 2100 |
| ATGCTTCCAT TAAATAAAAA GGACTTAAAA TCTGTTAATT ATAATAGAAA TGCGGCTAGT | 2160 |
| TCAGAGAGAT TTTTAGAGCT GTGGTGGACT TCATAGATGA ATTCAAGTGT TGAGGGAGGA | 2220 |
| TTAAAGAAAT ATATACCGTG TTTATGTGTG TGTGCTT | |

The human p45^{AUF1} amino acid sequence of GenBank Accession No. NP_112738.1 (SEQ ID NO: 14) is as follows:

| | |
|---|---|
| MSEEQFGGDG AAAAATAAVG GSAGEQEGAM VAATQGAAAA AGSGAGTGGG TASGGTEGGS | 60 |
| AESEGAKIDA SKNEEDEGHS NSSPRHSEAA TAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGGPSQNW NQGYSNYWNQ | 300 |
| GYGNYGYNSQ GYGGYGGYDY TGYNNYYGYG DYSNQQSGYG KVSRRGGHON SYKPY | |

The mouse p37^{AUF1} nucleotide sequence of GenBank Accession No. NM_001077267.2 (SEQ ID NO: 3) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGGAA AATGTTTATA GGAGGCCTTA GCTGGGACAC CACAAAGAAA GATCTGAAGG | 600 |
| ACTACTTTTC CAAATTTGGT GAAGTTGTAG ACTGCACTCT GAAGTTAGAT CCTATCACAG | 660 |
| GGCGATCAAG GGGTTTTGGC TTTGTGCTAT TTAAAGAGTC GGAGAGTGTA GATAAGGTCA | 720 |
| TGGATCAGAA AGAACATAAA TTGAATGGGA AAGTCATTGA TCCTAAAAGG GCCAAAGCCA | 780 |
| TGAAAACAAA AGAGCCTGTC AAAAAAATTT TTGTTGGTGG CCTTTCTCCA GACACACCTG | 840 |
| AAGAAAAAAT AAGAGAGTAC TTTGGTGGTT TTGGTGAGGT TGAATCCATA GAGCTCCCTA | 900 |
| TGGACAACAA GACCAATAAG AGGCGTGGGT TCTGTTTTAT TACCTTTAAG GAAGAGGAGC | 960 |
| CAGTGAAGAA GATAATGGAA AAGAAATACC ACAATGTTGG TCTTAGTAAA TGTGAAATAA | 1020 |
| AAGTAGCCAT GTCAAAGGAA CAGTATCAGC AGCAGCAGCA GTGGGGATCT AGAGGAGGGT | 1080 |
| TTGCAGGCAG AGCTCGCGGA AGAGGTGGAG ATCAGCAGAG TGGTTATGGG AAAGTATCCA | 1140 |
| GGCGAGGTGG ACATCAAAAT AGCTACAAAC CATACTAAAT TATTCCATTT GCAACTTATC | 1200 |
| CCCAACAGGT GGTGAAGCAG TATTTTCCAA TTTGAAGATT CATTTGAAGG TGGCTCCTGC | 1260 |
| CACCTGCTAA TAGCAGTTCA AACTAAATTT TTTCTATCAA GTTCCTGAAT GGAAGTATGA | 1320 |
| CGTTGGGTCC CTCTGAAGTT TAATTCTGAG TTCTCATTAA AAGAATTTGC TTTCATTGTT | 1380 |
| TTATTTCTTA ATTGCTATGC TTCAGTATCA ATTTGTGTTT TATGCCCCCC CTCCCCCCCA | 1440 |
| GTATTGTAGA GCAAGTCTTG TGTTAAAAAA AGCCCAGTGT GACAGTGTCA TGATGTAGTA | 1500 |
| GTGTCTTACT GGTTTTTTAA TAAATCCTTT TGTATAAAAA TGTATTGGCT CTTTTATCAT | 1560 |
| CAGAATAGGA GGAAGTGAAA TACTACAAAT GTTTGTCTTG GATTCAAGTC ACTAGAAGCA | 1620 |
| TAAATTTGAG GGGATAAAAA CAACGGTAAA CTTTGTCTGA AAGAGGGCAT GGTTAAAAAT | 1680 |
| GTAGTGAATT TTAAATGTTT TTAGCAAAAT TTGATTTTGC CCAAGAATCC CTGTCTGAAT | 1740 |
| TGGAAATGAC TTAATGTAGT CAATGTGCTT GTTGGTTGTC TTAATATTAC TTCTGTAGCC | 1800 |
| ATTAAGTTTT ATGAGTAACT TCCCAAATAC CCACGTTTTT CTTTATATGT ATTGTGCTTT | 1860 |
| TTAAAAACAA ATCTGGAAAA ATGGGCAAGA ACATTTGCAG ACAATTGTTT TTAAGCTTCC | 1920 |
| ATTAAATAAA AAAAATGTGG ACTTAAGGAA ATCTATTAAT TTAAATAGAA CTGCAGCTAG | 1980 |
| TTTAGAGAGT ATTTTTTTCT TAAAGCTTTG GTGTAATTAG GGAAGATTTT AAAAAATGCA | 2040 |
| TAGTGTTTAT TTGTATGTGT GCTCTTTTTT TAAGTCAATT TTTGGGGGGT TGGTCTGTTA | 2100 |
| ACTGAGTCTA GGATTTAAAG GTAAGATGTT CCTAGAAATC TTGTCATCCC AAAGGGGCGG | 2160 |
| GCGCTAAGGT GAAACTTCAG GGTTCAGTCA GGGTCACTGC TTTATGTGTG AAATCACTCA | 2220 |
| AATTGGTAAG TCTCTTATGT TAGCATTCAG GACATTGATT TCAACTTGGA TGGACAATTT | 2280 |
| ATAGTTACTA CTGAATTGTG TGTTAATGTG TTCAGTCCTG GTAAGTTTTC AGTTTGATCA | 2340 |
| GTTAGTTGGA AGCAGACTTG AAGAGCTGTT AGTCACGTGA GCCATGGGTG CAGTCGATCT | 2400 |
| GTGGTCAGAT GCCTGAGTCT GTGATAGTGA ATTGTGTCTA AAGACATTTT AATGATAAAA | 2460 |
| GTCAGTGCTG TAAAGTTGAA AGTTCATGAG AGACATACAA TGAGGGCTGC AGCCCATTTT | 2520 |
| TAAAAACATT ATAATACAAA AGTATGCACA TTTGTTTACA TATCCCTGCC TTTGTATTAC | 2580 |
| AGTGGCAGGT TTGTGTACTT AAACTGGGAA AGCCTCAGAT CTATGATTAC CTGGCCTATC | 2640 |
| ATAGAAAGTG TCTAAATAAA TCACTCTGTC AATTGAATAC ATTAGTATTA GCTAGCATAC | 2700 |
| TTCATTATGC CTGTTTTCCA TAAATACCAC ACCAAAAACT TGCTTGGGGC AGTTTGAGCC | 2760 |
| TAGTTCATGA GCTGCTATCA GATTGGTCTT GATCCTATAT AATAGGCCAA ATGTCTGTAA | 2820 |
| ACAGCTGTGC TGGTGGAATG TAGAAAGTCA CTGCACTCAG ATTCAACTTC CTGATTGGAA | 2880 |
| GTCATCACAG TGTGATTAAA CATTTTCACA AAGAATAGTA GATAAATAAC TTGGTTTTTA | 2940 |
| ATGTTAACTT TGTTTCCATT AAGTCACATT TAAAAACTTA TCCTCACGCC TACCTGAGTT | 3000 |
| AATTATCTGT TGACCTAGAT ATCTTTCTGG CCACTCACTG ACTTATTTCT TGAACTTTTG | 3060 |
| CCATTTGCAT AAATCTTGTC AGCTTTGTTC TTGATTATGC ATTGTCCAGG CTGAGCTAGT | 3120 |
| TGTCTTTCCA GGAATCCCTT TGTCTCTGAA TTAGGTCCTT TGTTTCCTAA ATCATCCTGC | 3180 |
| TTGTTTGGCA CAAGTCTTCC CAGGCCAGTG AGACCTCCGT GTCCTCTCAG CACCATAGGG | 3240 |
| GTAGGTAACC CTGGTTAGGC TGGACAGGGG TTTGCTGAGG GAGTTTGTTC ATTTGAATCT | 3300 |
| AGGTCTTACA TGACGTCTTT CAAATAGGGT TTTTACCTTG ACACTAAACT GTCCAGTCTA | 3360 |
| AGCAGTTCTG CAAAATGTGA GGGAATTATG AACTTCTTCC TGCAGTGGGT TTTTATGGTT | 3420 |
| TTGGTTTGTT TTTTGTTGTT TTGGTTCTTT GTTGAGCCCT GGACAAAAAC TTCCCTAGTT | 3480 |
| CTGGTTTCTA CAATTTAAAT TAAAAACAGA ATTCATCTTA GAATTTTTCA CCCTCTTCCC | 3540 |
| CAACTATTCT AATCAATCTT AAGTATGCCC TTCATCTTTT TTCCTTCCTA AGGCTTTTAC | 3600 |
| TGATAGTGTA ATTCCGTACT CTTCAACCCT GGGAAGGCTG AAGTGGATTC TTGAGCTCAT | 3660 |
| TTCAAGGCTG ACCTGGGTGT TGGCAAGAAC CCAGCTTAGA ACAAACACAT GCAAGGCCAT | 3720 |
| CTTACCTTAC ATCCTGTTGC TTGGACTTCT TCCTGCTCAA AGTTTTTAGT GGATGCTAAG | 3780 |
| TGATCTTTGC TTCCACTGAG GAGTGGAACA CTTTAGAATG AACCTCTAGA TAGATATTTT | 3840 |
| TATTGTCTGG TGAGGGTTAC TGGAGTTTCC CACCCTGCCT GAAGGGTGAA TCTGGCTTAC | 3900 |
| AGTGTTCTCA TCTCAAAGGG AAGAAGGCAG ATGGCTGTGT CCAGAGAGAG CCATCACAGT | 3960 |
| TTGCTTCAGA GACACTAGAA TGGGCTGGAA GATCTAGTGG TCTTAATCAG ACTTGAAACC | 4020 |
| TGGCCTTTCT TCATTACCCA TATGTCTACC AGTACTTGGG CTAACACTTA AGCCATTAGG | 4080 |
| GCCTTTGTAG GGGTGTTTTG AGACCCCCTC CATGCTAACA AATATACAGG TTTCTTAACA | 4140 |
| TTTGCTCATA AACTTGTAAA GCTTACTTTC TCTTAATCCA CCCCACATTT AACAAGCCCT | 4200 |
| GGTACTTAGA ATTTCAGAAG AGTAATGGCA GGTAGGTGTG TGTGTGTGTG TGTGTGTGTG | 4260 |
| TGTGTGTGTG TGTGTGTGAG AGAGAGAGAG AGAGAGAGAG AGAGAGAGAG AGAGAGAGAG | 4320 |
| AGAGAGAGAG AGAAGTTTGT GGAAAATCAG GTAATGACAG CTCATCCTTT TAGAATTGTA | 4380 |
| CTTCAGAATA GAAACATTTG GTGGGCTGTT AGGTAGCTTT GATTACTTGT GGGTAGACCT | 4440 |
| GCTAGTATTG CCAGTCCTCA AGCAATGAGC TTTCTGTATC TTGTTTACTA GATATATACT | 4500 |
| ACCAGGTGAG TCATTTCCTG GGGTTCTGTT TTCTTTTAAA ATCTTTCCCT AAACTTAATA | 4560 |
| TGTATTAAAA AGTCTGGCTT TTCAGTCCAT TCTTTGTGCA CTGGGATGGC AATTGCTTCA | 4620 |
| TTATATGACA ATTGCTGTTC CCAAGTCAGA ATTCAGTGTG CTGATTTGAC ATCAGTTCGT | 4680 |
| CCCGAATAAG TTCCTGTTAC CAGGATTTAC ATTCAGCACA TTAGAAACTT GTTGGTGTGC | 4740 |
| TTTTATTCTT GGAGCATTTT CCTTAGACTA CCTTCCACTT TGAGTGCTCT GTTTAGGATG | 4800 |
| TTGAGGTGTT AGGATTCTTG ACAGCCAGAA AGACTGAACC CACTATCTGG GCACAGTGTT | 4860 |
| CGTGTTGCTC TATAAATGTA TGCTTTTTTT GATTTGGGGT TGTTTTACCT ACATTGTCAA | 4920 |
| ACTAGATCCA TGCTTAACAG TGATAATGAA GGCTTTTTGT TTGTTTTGTT TGTGGGTCCT | 4980 |
| CCCCCCCCCC CCAAGACAGG GTTTCTCTGT AGGCTGTCCT AGAACTTGTT CTTTTTTAAC | 5040 |
| CAAAATTTGG CAAGGCTGAA AATGGAATCC TATAATCAAT GCTGGCCACA TTAAAGTTAA | 5100 |
| TAGTTGAGAA GTCTTGTCTG AATTTCCTTG GGCAAAAAGA TTCTAGCCAG TTCAATACCC | 5160 |
| TGTTGTGCAA ATTCAATTTG CTGTTATAAT TTGCTCTCAG TTATCAGTTG GAAGGAGGIT | 5220 |
| AATTCTAATG TACTTGGAAG AGGCCTGTAG ACCATCTATA ACTGCATCAG TTGTACAGCG | 5280 |
| TTGTTGCCTG GGATTCTCTA GTTCACATAA ACTCCCAAGT CTTAGCCGTG GTGATGGCTA | 5340 |
| CAGTGTGGAA GATGGTGAGC ATTCTAGTGA GTATCGCGAT GACGGCAGTA AAGAGCAGCA | 5400 |
| GGCAGCCGTG GCTGGGCTCA CTGACCGTGG CTGTAAGTTA CGGAGGCAGC ACACACTTCT | 5460 |
| GTACACACCT CTCATCAGTT ACCGGAGTCA TTGCATTGCG GACTAACTGG CTGACTCAAG | 5520 |
| TTGTCTTGCT ACTGAAGTCT TGAGTTGGTC TCATGCATTT ACCCTGTTGA CTTGAGCACC | 5580 |
| TTAAAGTCGA AAGGATGTCT GGTTGTGGCT TTATTGTAAA CAGCCTTAGG TAAAGAGGGG | 5640 |
| AGTATATCGG TTAGGAAGGT GAAAAATGAT ACTTCCAAGT TCAGTGGGAA ACCCTGGGTT | 5700 |
| TATCCCCCAG CTTAAGAAAG AATGCCTAAC AATGTTTCAG AATTAGATTC TGTGGAAGGT | 5760 |
| GAGGGTGTTA GAACAGTCCA AATTTGTTAT TGTAGACTTG CAGTGGGAGG AATTTTTAAA | 5820 |
| TATACAGATC AGTCGACACT CATTAACTTC ACTGATAAAG GTGGAAACGG ATGTGGCAAC | 5880 |
| ACTTCTAAGT TCATTTGTAT ATGTTTGTAA TTTGATTGGT TGTATTCTGT TGCACTCTAG | 5940 |
| AATTTGAAGG CAAGGTTACC TCTGCTTTTT AATTTTTTTT TTTTTAAAGA AAGAAAAAAC | 6000 |
| ACTGAAAGAA ACTTCAAAAG ATCTGTTAAT GCTAATACCT GAATGTGGCA TTTAACATGT | 6060 |
| CATGGAAACT GCTTTGAATA AATACTTGAG AAAAGGAATG AAATAATTGC CGTTTTTGTT | 6120 |
| GTTGAGTGAA TGGGTGTGGT TTAATGAGCG TAATCATTTT TATAAAACAG CTGTGAGACT | 6180 |
| GAAGTGGAAT CCTTATTAAA TGTGGAAAAT GGCCTTTGAG GATTACAGTA GAGATTCAAC | 6240 |
| TAAGAGAGTA AATAAAGCTT GAAACTAATT CGTTGTAAAT TGCTTCTACA ATCATTGCTC | 6300 |
| TATATAGCAT GCTATTGCCA ATCAGTTTTA TGTATTAAGA CCTATCAGCA TGTCTTTTTT | 6360 |
| AGGTTGACCT CATTTTAAAT TATAAGATGC TCTCTGTACC GTTTTAACAT TTCCAGGATT | 6420 |
| TATTCTTTCT AGGCAAATTC CACTGGACTG TTTCCATTGT AGAAGCTTCC TTATAGATTC | 6480 |
| TTCAAATGAA GCTTACAGTG TGCTTTCTTG GGGTTTTGAT TTGCACTAAA TTTTATTTTC | 6540 |
| TGAAAGATCA CTTATGTTTA TAATGTAGTG CTTTGTCTTA ACAATTAAAC TTTCCAGCAC | 6600 |
| TCATGCA | |

The mouse p37^{AUF1} amino acid sequence of GenBank Accession No. NP_001070735.1 (SEQ ID NO: 4) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGDQQSG YGKVSRRGGH QNSYKPY | |

The mouse p40^{AUF1} nucleotide sequence of GenBank Accession No. NM_007516.3 (SEQ ID NO: 7) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGCCA TTCAAACTCC TCCCCACGAC ACACTGAAGC AGCGGCGGCA CAGCGGGAAG | 600 |
| AATGGAAAAT GTTTATAGGA GGCCTTAGCT GGGACACCAC AAAGAAAGAT CTGAAGGACT | 660 |
| ACTTTTCCAA ATTTGGTGAA GTTGTAGACT GCACTCTGAA GTTAGATCCT ATCACAGGGC | 720 |
| GATCAAGGGG TTTTGGCTTT GTGCTATTTA AAGAGTCGGA GAGTGTAGAT AAGGTCATGG | 780 |
| ATCAGAAAGA ACATAAATTG AATGGGAAAG TCATTGATCC TAAAAGGGCC AAAGCCATGA | 840 |
| AAACAAAAGA GCCTGTCAAA AAAATTTTTG TTGGTGGCCT TTCTCCAGAC ACACCTGAAG | 900 |
| AAAAAATAAG AGAGTACTTT GGTGGTTTTG GTGAGGTTGA ATCCATAGAG CTCCCTATGG | 960 |
| ACAACAAGAC CAATAAGAGG CGTGGGTTCT GTTTTATTAC CTTTAAGGAA GAGGAGCCAG | 1020 |
| TGAAGAAGAT AATGGAAAAG AAATACCACA ATGTTGGTCT TAGTAAATGT GAAATAAAAG | 1080 |
| TAGCCATGTC AAAGGAACAG TATCAGCAGC AGCAGCAGTG GGGATCTAGA GGAGGGTTTG | 1140 |
| CAGGCAGAGC TCGCGGAAGA GGTGGAGATC AGCAGAGTGG TTATGGGAAA GTATCCAGGC | 1200 |
| GAGGTGGACA TCAAAATAGC TACAAACCAT ACTAAATTAT TCCATTTGCA ACTTATCCCC | 1260 |
| AACAGGTGGT GAAGCAGTAT TTTCCAATTT GAAGATTCAT TTGAAGGTGG CTCCTGCCAC | 1320 |
| CTGCTAATAG CAGTTCAAAC TAAATTTTTT CTATCAAGTT CCTGAATGGA AGTATGACGT | 1380 |
| TGGGTCCCTC TGAAGTTTAA TTCTGAGTTC TCATTAAAAG AATTTGCTTT CATTGTTTTA | 1440 |
| TTTCTTAATT GCTATGCTTC AGTATCAATT TGTGTTTTAT GCCCCCCCTC CCCCCCAGTA | 1500 |
| TTGTAGAGCA AGTCTTGTGT TAAAAAAAGC CCAGTGTGAC AGTGTCATGA TGTAGTAGTG | 1560 |
| TCTTACTGGT TTTTTAATAA ATCCTTTTGT ATAAAAATGT ATTGGCTCTT TTATCATCAG | 1620 |
| AATAGGAGGA AGTGAAATAC TACAAATGTT TGTCTTGGAT TCAAGTCACT AGAAGCATAA | 1680 |
| ATTTGAGGGG ATAAAAACAA CGGTAAACTT TGTCTGAAAG AGGGCATGGT TAAAAATGTA | 1740 |
| GTGAATTTTA AATGTTTTTA GCAAAATTTG ATTTTGCCCA AGAATCCCTG TCTGAATTGG | 1800 |
| AAATGACTTA ATGTAGTCAA TGTGCTTGTT GGTTGTCTTA ATATTACTTC TGTAGCCATT | 1860 |
| AAGTTTTATG AGTAACTTCC CAAATACCCA CGTTTTTCTT TATATGTATT GTGCTTTTTA | 1920 |
| AAAACAAATC TGGAAAAATG GGCAAGAACA TTTGCAGACA ATTGTTTTTA AGCTTCCATT | 1980 |
| AAATAAAAAA AATGTGGACT TAAGGAAATC TATTAATTTA AATAGAACTG CAGCTAGTTT | 2040 |
| AGAGAGTATT TTTTTCTTAA AGCTTTGGTG TAATTAGGGA AGATTTTAAA AAATGCATAG | 2100 |
| TGTTTATTTG TATGTGTGCT CTTTTTTTAA GTCAATTTTT GGGGGGTTGG TCTGTTAACT | 2160 |
| GAGTCTAGGA TTTAAAGGTA AGATGTTCCT AGAAATCTTG TCATCCCAAA GGGGGGGGCG | 2220 |
| CTAAGGTGAA ACTTCAGGGT TCAGTCAGGG TCACTGCTTT ATGTGTGAAA TCACTCAAAT | 2280 |
| TGGTAAGTCT CTTATGTTAG CATTCAGGAC ATTGATTTCA ACTTGGATGG ACAATTTATA | 2340 |
| GTTACTACTG AATTGTGTGT TAATGTGTTC AGTCCTGGTA AGTTTTCAGT TTGATCAGTT | 2400 |
| AGTTGGAAGC AGACTTGAAG AGCTGTTAGT CACGTGAGCC ATGGGTGCAG TCGATCTGTG | 2460 |
| GTCAGATGCC TGAGTCTGTG ATAGTGAATT GTGTCTAAAG ACATTTTAAT GATAAAAGTC | 2520 |
| AGTGCTGTAA AGTTGAAAGT TCATGAGAGA CATACAATGA GGGCTGCAGC CCATTTTTAA | 2580 |
| AAACATTATA ATACAAAAGT ATGCACATTT GTTTACATAT CCCTGCCTTT GTATTACAGT | 2640 |
| GGCAGGTTTG TGTACTTAAA CTGGGAAAGC CTCAGATCTA TGATTACCTG GCCTATCATA | 2700 |
| GAAAGTGTCT AAATAAATCA CTCTGTCAAT TGAATACATT AGTATTAGCT AGCATACTTC | 2760 |
| ATTATGCCTG TTTTCCATAA ATACCACACC AAAAACTTGC TTGGGGCAGT TTGAGCCTAG | 2820 |
| TTCATGAGCT GCTATCAGAT TGGTCTTGAT CCTATATAAT AGGCCAAATG TCTGTAAACA | 2880 |
| GCTGTGCTGG TGGAATGTAG AAAGTCACTG CACTCAGATT CAACTTCCTG ATTGGAAGTC | 2940 |
| ATCACAGTGT GATTAAACAT TTTCACAAAG AATAGTAGAT AAATAACTTG GTTTTTAATG | 3000 |
| TTAACTTTGT TTCCATTAAG TCACATTTAA AAACTTATCC TCACGCCTAC CTGAGTTAAT | 3060 |
| TATCTGTTGA CCTAGATATC TTTCTGGCCA CTCACTGACT TATTTCTTGA ACTTTTGCCA | 3120 |
| TTTGCATAAA TCTTGTCAGC TTTGTTCTTG ATTATGCATT GTCCAGGCTG AGCTAGTTGT | 3180 |
| CTTTCCAGGA ATCCCTTTGT CTCTGAATTA GGTCCTTTGT TTCCTAAATC ATCCTGCTTG | 3240 |
| TTTGGCACAA GTCTTCCCAG GCCAGTGAGA CCTCCGTGTC CTCTCAGCAC CATAGGGGTA | 3300 |
| GGTAACCCTG GTTAGGCTGG ACAGGGGTTT GCTGAGGGAG TTTGTTCATT TGAATCTAGG | 3360 |
| TCTTACATGA CGTCTTTCAA ATAGGGTTTT TACCTTGACA CTAAACTGTC CAGTCTAAGC | 3420 |
| AGTTCTGCAA AATGTGAGGG AATTATGAAC TTCTTCCTGC AGTGGGTTTT TATGGTTTTG | 3480 |
| GTTTGTTTTT TGTTGTTTTG GTTCTTTGTT GAGCCCTGGA CAAAAACTTC CCTAGTTCTG | 3540 |
| GTTTCTACAA TTTAAATTAA AAACAGAATT CATCTTAGAA TTTTTCACCC TCTTCCCCAA | 3600 |
| CTATTCTAAT CAATCTTAAG TATGCCCTTC ATCTTTTTTC CTTCCTAAGG CTTTTACTGA | 3660 |
| TAGTGTAATT CCGTACTCTT CAACCCTGGG AAGGCTGAAG TGGATTCTTG AGCTCATTTC | 3720 |
| AAGGCTGACC TGGGTGTTGG CAAGAACCCA GCTTAGAACA AACACATGCA AGGCCATCTT | 3780 |
| ACCTTACATC CTGTTGCTTG GACTTCTTCC TGCTCAAAGT TTTTAGTGGA TGCTAAGTGA | 3840 |
| TCTTTGCTTC CACTGAGGAG TGGAACACTT TAGAATGAAC CTCTAGATAG ATATTTTTAT | 3900 |
| TGTCTGGTGA GGGTTACTGG AGTTTCCCAC CCTGCCTGAA GGGTGAATCT GGCTTACAGT | 3960 |
| GTTCTCATCT CAAAGGGAAG AAGGCAGATG GCTGTGTCCA GAGAGAGCCA TCACAGTTTG | 4020 |
| CTTCAGAGAC ACTAGAATGG GCTGGAAGAT CTAGTGGTCT TAATCAGACT TGAAACCTGG | 4080 |
| CCTTTCTTCA TTACCCATAT GTCTACCAGT ACTTGGGCTA ACACTTAAGC CATTAGGGCC | 4140 |
| TTTGTAGGGG TGTTTTGAGA CCCCCTCCAT GCTAACAAAT ATACAGGTTT CTTAACATTT | 4200 |
| GCTCATAAAC TTGTAAAGCT TACTTTCTCT TAATCCACCC CACATTTAAC AAGCCCTGGT | 4260 |
| ACTTAGAATT TCAGAAGAGT AATGGCAGGT AGGTGTGTGT GTGTGTGTGT GTGTGTGTGT | 4320 |
| GTGTGTGTGT GTGTGAGAGA GAGAGAGAGA GAGAGAGAGA GAGAGAGAGA GAGAGAGAGA | 4380 |
| GAGAGAGAGA AGTTTGTGGA AAATCAGGTA ATGACAGCTC ATCCTTTTAG AATTGTACTT | 4440 |
| CAGAATAGAA ACATTTGGTG GGCTGTTAGG TAGCTTTGAT TACTTGTGGG TAGACCTGCT | 4500 |
| AGTATTGCCA GTCCTCAAGC AATGAGCTTT CTGTATCTTG TTTACTAGAT ATATACTACC | 4560 |
| AGGTGAGTCA TTTCCTGGGG TTCTGTTTTC TTTTAAAATC TTTCCCTAAA CTTAATATGT | 4620 |
| ATTAAAAAGT CTGGCTTTTC AGTCCATTCT TIGTGCACTG GGATGGCAAT TGCTTCATTA | 4680 |
| TATGACAATT GCTGTTCCCA AGTCAGAATT CAGTGTGCTG ATTTGACATC AGTTCGTCCC | 4740 |
| GAATAAGTTC CTGTTACCAG GATTTACATT CAGCACATTA GAAACTTGTT GGTGTGCTTT | 4800 |
| TATTCTTGGA GCATTTTCCT TAGACTACCT TCCACTTTGA GTGCTCTGTT TAGGATGTTG | 4860 |
| AGGTGTTAGG ATTCTTGACA GCCAGAAAGA CTGAACCCAC TATCTGGGCA CAGTGTTCGT | 4920 |
| GTTGCTCTAT AAATGTATGC TTTTTTTGAT TTGGGGTTGT TTTACCTACA TTGTCAAACT | 4980 |
| AGATCCATGC TTAACAGTGA TAATGAAGGC TTTTTGTTTG TTTTGTTTGT GGGTCCTCCC | 5040 |
| CCCCCCCCCA AGACAGGGTT TCTCTGTAGG CTGTCCTAGA ACTTGTTCTT TTTTAACCAA | 5100 |
| AATTTGGCAA GGCTGAAAAT GGAATCCTAT AATCAATGCT GGCCACATTA AAGTTAATAG | 5160 |
| TTGAGAAGTC TTGTCTGAAT TTCCTTGGGC AAAAAGATTC TAGCCAGTTC AATACCCTGT | 5220 |
| TGTGCAAATT CAATTTGCTG TTATAATTTG CTCTCAGTTA TCAGTTGGAA GGAGGTTAAT | 5280 |
| TCTAATGTAC TTGGAAGAGG CCTGTAGACC ATCTATAACT GCATCAGTTG TACAGCGTTG | 5340 |
| TTGCCTGGGA TTCTCTAGTT CACATAAACT CCCAAGTCTT AGCCGTGGTG ATGGCTACAG | 5400 |
| TGTGGAAGAT GGTGAGCATT CTAGTGAGTA TCGCGATGAC GGCAGTAAAG AGCAGCAGGC | 5460 |
| AGCCGTGGCT GGGCTCACTG ACCGTGGCTG TAAGTTACGG AGGCAGCACA CACTTCTGTA | 5520 |
| CACACCTCTC ATCAGTTACC GGAGTCATTG CATTGCGGAC TAACTGGCTG ACTCAAGTTG | 5580 |
| TCTTGCTACT GAAGTCTTGA GTTGGTCTCA TGCATTTACC CTGTTGACTT GAGCACCTTA | 5640 |
| AAGTCGAAAG GATGTCTGGT TGTGGCTTTA TTGTAAACAG CCTTAGGTAA AGAGGGGAGT | 5700 |
| ATATCGGTTA GGAAGGTGAA AAATGATACT TCCAAGTTCA GTGGGAAACC CTGGGTTTAT | 5760 |
| CCCCCAGCTT AAGAAAGAAT GCCTAACAAT GTTTCAGAAT TAGATTCTGT GGAAGGTGAG | 5820 |
| GGTGTTAGAA CAGTCCAAAT TTGTTATTGT AGACTTGCAG TGGGAGGAAT TTTTAAATAT | 5880 |
| ACAGATCAGT CGACACTCAT TAACTTCACT GATAAAGGTG GAAACGGATG TGGCAACACT | 5940 |
| TCTAAGTTCA TTTGTATATG TTTGTAATTT GATTGGTTGT ATTCTGTTGC ACTCTAGAAT | 6000 |
| TTGAAGGCAA GGTTACCTCT GCTTTTTAAT TTTTTTTTTT TTAAAGAAAG AAAAAACACT | 6060 |
| GAAAGAAACT TCAAAAGATC TGTTAATGCT AATACCTGAA TGTGGCATTT AACATGTCAT | 6120 |
| GGAAACTGCT TTGAATAAAT ACTTGAGAAA AGGAATGAAA TAATTGCCGT TTTTGTTGTT | 6180 |
| GAGTGAATGG GTGTGGTTTA ATGAGCGTAA TCATTTTTAT AAAACAGCTG TGAGACTGAA | 6240 |
| GTGGAATCCT TATTAAATGT GGAAAATGGC CTTTGAGGAT TACAGTAGAG ATTCAACTAA | 6300 |
| GAGAGTAAAT AAAGCTTGAA ACTAATTCGT TGTAAATTGC TTCTACAATC ATTGCTCTAT | 6360 |
| ATAGCATGCT ATTGCCAATC AGTTTTATGT ATTAAGACCT ATCAGCATGT CTTTTTTAGG | 6420 |
| TTGACCTCAT TTTAAATTAT AAGATGCTCT CTGTACCGTT TTAACATTTC CAGGATTTAT | 6480 |
| TCTTTCTAGG CAAATTCCAC TGGACTGTTT CCATTGTAGA AGCTTCCTTA TAGATTCTTC | 6540 |
| AAATGAAGCT TACAGTGTGC TTTCTTGGGG TTTTGATTTG CACTAAATTT TATTTTCTGA | 6600 |
| AAGATCACTT ATGTTTATAA TGTAGTGCTT TGTCTTAACA ATTAAACTTT CCAGCACTCA | 6660 |
| TGCA | |

The mouse p40^{AUF1} amino acid sequence of GenBank Accession No. NP_031542.2 (SEQ ID NO: 8) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGHS NSSPRHTEAA AAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGDQQSGY GKVSRRGGHQ | 300 |
| NSYKPY | |

The mouse p42^{AUF1} nucleotide sequence of GenBank Accession No. NM_001077266.2 (SEQ ID NO: 11) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGGAA AATGTTTATA GGAGGCCTTA GCTGGGACAC CACAAAGAAA GATCTGAAGG | 600 |
| ACTACTTTTC CAAATTTGGT GAAGTTGTAG ACTGCACTCT GAAGTTAGAT CCTATCACAG | 660 |
| GGCGATCAAG GGGTTTTGGC TTTGTGCTAT TTAAAGAGTC GGAGAGTGTA GATAAGGTCA | 720 |
| TGGATCAGAA AGAACATAAA TTGAATGGGA AAGTCATTGA TCCTAAAAGG GCCAAAGCCA | 780 |
| TGAAAACAAA AGAGCCTGTC AAAAAAATTT TTGTTGGTGG CCTTTCTCCA GACACACCTG | 840 |
| AAGAAAAAAT AAGAGAGTAC TTTGGTGGTT TTGGTGAGGT TGAATCCATA GAGCTCCCTA | 900 |
| TGGACAACAA GACCAATAAG AGGCGTGGGT TCTGTTTTAT TACCTTTAAG GAAGAGGAGC | 960 |
| CAGTGAAGAA GATAATGGAA AAGAAATACC ACAATGTTGG TCTTAGTAAA TGTGAAATAA | 1020 |
| AAGTAGCCAT GTCAAAGGAA CAGTATCAGC AGCAGCAGCA GTGGGGATCT AGAGGAGGGT | 1080 |
| TTGCAGGCAG AGCTCGCGGA AGAGGTGGAG GCCCCAGTCA AAACTGGAAC CAGGGATATA | 1140 |
| GTAACTATTG GAATCAAGGC TATGGCAACT ATGGATATAA CAGCCAAGGT TACGGAGGTT | 1200 |
| ATGGAGGATA TGACTACACT GGTTACAACA ACTACTATGG ATATGGTGAT TATAGCAATC | 1260 |
| AGCAGAGTGG TTATGGGAAA GTATCCAGGC GAGGTGGACA TCAAAATAGC TACAAACCAT | 1320 |
| ACTAAATTAT TCCATTTGCA ACTTATCCCC AACAGGTGGT GAAGCAGTAT TTTCCAATTT | 1380 |
| GAAGATTCAT TTGAAGGTGG CTCCTGCCAC CTGCTAATAG CAGTTCAAAC TAAATTTTTT | 1440 |
| CTATCAAGTT CCTGAATGGA AGTATGACGT TGGGTCCCTC TGAAGTTTAA TTCTGAGTTC | 1500 |
| TCATTAAAAG AATTTGCTTT CATTGTTTTA TTTCTTAATT GCTATGCTTC AGTATCAATT | 1560 |
| TGTGTTTTAT GCCCCCCCTC CCCCCCAGTA TTGTAGAGCA AGTCTTGTGT TAAAAAAAGC | 1620 |
| CCAGTGTGAC AGTGTCATGA TGTAGTAGTG TCTTACTGGT TTTTTAATAA ATCCTTTTGT | 1680 |
| ATAAAAATGT ATTGGCTCTT TTATCATCAG AATAGGAGGA AGTGAAATAC TACAAATGTT | 1740 |
| TGTCTTGGAT TCAAGTCACT AGAAGCATAA ATTTGAGGGG ATAAAAACAA CGGTAAACTT | 1800 |
| TGTCTGAAAG AGGGCATGGT TAAAAATGTA GTGAATTTTA AATGTTTTTA GCAAAATTTG | 1860 |
| ATTTTGCCCA AGAATCCCTG TCTGAATTGG AAATGACTTA ATGTAGTCAA TGTGCTTGTT | 1920 |
| GGTTGTCTTA ATATTACTTC TGTAGCCATT AAGTTTTATG AGTAACTTCC CAAATACCCA | 1980 |
| CGTTTTTCTT TATATGTATT GTGCTTTTTA AAAACAAATC TGGAAAAATG GGCAAGAACA | 2040 |
| TTTGCAGACA ATTGTTTTTA AGCTTCCATT AAATAAAAAA AATGTGGACT TAAGGAAATC | 2100 |
| TATTAATTTA AATAGAACTG CAGCTAGTTT AGAGAGTATT TTTTTCTTAA AGCTTTGGTG | 2160 |
| TAATTAGGGA AGATTTTAAA AAATGCATAG TGTTTATTTG TATGTGTGCT CTTTTTTTAA | 2220 |
| GTCAATTTTT GGGGGGTTGG TCTGTTAACT GAGTCTAGGA TTTAAAGGTA AGATGTTCCT | 2280 |
| AGAAATCTTG TCATCCCAAA GGGGGGGGCG CTAAGGTGAA ACTTCAGGGT TCAGTCAGGG | 2340 |
| TCACTGCTTT ATGTGTGAAA TCACTCAAAT TGGTAAGTCT CTTATGTTAG CATTCAGGAC | 2400 |
| ATTGATTTCA ACTTGGATGG ACAATTTATA GTTACTACTG AATTGTGTGT TAATGTGTTC | 2460 |
| AGTCCTGGTA AGTTTTCAGT TTGATCAGTT AGTTGGAAGC AGACTTGAAG AGCTGTTAGT | 2520 |
| CACGTGAGCC ATGGGTGCAG TCGATCTGTG GTCAGATGCC TGAGTCTGTG ATAGTGAATT | 2580 |
| GTGTCTAAAG ACATTTTAAT GATAAAAGTC AGTGCTGTAA AGTTGAAAGT TCATGAGAGA | 2640 |
| CATACAATGA GGGCTGCAGC CCATTTTTAA AAACATTATA ATACAAAAGT ATGCACATTT | 2700 |
| GTTTACATAT CCCTGCCTTT GTATTACAGT GGCAGGTTTG TGTACTTAAA CTGGGAAAGC | 2760 |
| CTCAGATCTA TGATTACCTG GCCTATCATA GAAAGTGTCT AAATAAATCA CTCTGTCAAT | 2820 |
| TGAATACATT AGTATTAGCT AGCATACTTC ATTATGCCTG TTTTCCATAA ATACCACACC | 2880 |
| AAAAACTTGC TTGGGGCAGT TTGAGCCTAG TTCATGAGCT GCTATCAGAT TGGTCTTGAT | 2940 |
| CCTATATAAT AGGCCAAATG TCTGTAAACA GCTGTGCTGG TGGAATGTAG AAAGTCACTG | 3000 |
| CACTCAGATT CAACTTCCTG ATTGGAAGTC ATCACAGTGT GATTAAACAT TTTCACAAAG | 3060 |
| AATAGTAGAT AAATAACTTG GTTTTTAATG TTAACTTTGT TTCCATTAAG TCACATTTAA | 3120 |
| AAACTTATCC TCACGCCTAC CTGAGTTAAT TATCTGTTGA CCTAGATATC TTTCTGGCCA | 3180 |
| CTCACTGACT TATTTCTTGA ACTTTTGCCA TTTGCATAAA TCTTGTCAGC TTTGTTCTTG | 3240 |
| ATTATGCATT GTCCAGGCTG AGCTAGTTGT CTTTCCAGGA ATCCCTTTGT CTCTGAATTA | 3300 |
| GGTCCTTTGT TTCCTAAATC ATCCTGCTTG TTTGGCACAA GTCTTCCCAG GCCAGTGAGA | 3360 |
| CCTCCGTGTC CTCTCAGCAC CATAGGGGTA GGTAACCCTG GTTAGGCTGG ACAGGGGTTT | 3420 |
| GCTGAGGGAG TTTGTTCATT TGAATCTAGG TCTTACATGA CGTCTTTCAA ATAGGGTTTT | 3480 |
| TACCTTGACA CTAAACTGTC CAGTCTAAGC AGTTCTGCAA AATGTGAGGG AATTATGAAC | 3540 |
| TTCTTCCTGC AGTGGGTTTT TATGGTTTTG GTTTGTTTTT TGTTGTTTTG GTTCTTTGTT | 3600 |
| GAGCCCTGGA CAAAAACTTC CCTAGTTCTG GTTTCTACAA TTTAAATTAA AAACAGAATT | 3660 |
| CATCTTAGAA TTTTTCACCC TCTTCCCCAA CTATTCTAAT CAATCTTAAG TATGCCCTTC | 3720 |
| ATCTTTTTTC CTTCCTAAGG CTTTTACTGA TAGTGTAATT CCGTACTCTT CAACCCTGGG | 3780 |
| AAGGCTGAAG TGGATTCTTG AGCTCATTTC AAGGCTGACC TGGGTGTTGG CAAGAACCCA | 3840 |
| GCTTAGAACA AACACATGCA AGGCCATCTT ACCTTACATC CTGTTGCTTG GACTTCTTCC | 3900 |
| TGCTCAAAGT TTTTAGTGGA TGCTAAGTGA TCTTTGCTTC CACTGAGGAG TGGAACACTT | 3960 |
| TAGAATGAAC CTCTAGATAG ATATTTTTAT TGTCTGGTGA GGGTTACTGG AGTTTCCCAC | 4020 |
| CCTGCCTGAA GGGTGAATCT GGCTTACAGT GTTCTCATCT CAAAGGGAAG AAGGCAGATG | 4080 |
| GCTGTGTCCA GAGAGAGCCA TCACAGTTTG CTTCAGAGAC ACTAGAATGG GCTGGAAGAT | 4140 |
| CTAGTGGTCT TAATCAGACT TGAAACCTGG CCTTTCTTCA TTACCCATAT GTCTACCAGT | 4200 |
| ACTTGGGCTA ACACTTAAGC CATTAGGGCC TTTGTAGGGG TGTTTTGAGA CCCCCTCCAT | 4260 |
| GCTAACAAAT ATACAGGTTT CTTAACATTT GCTCATAAAC TTGTAAAGCT TACTTTCTCT | 4320 |
| TAATCCACCC CACATTTAAC AAGCCCTGGT ACTTAGAATT TCAGAAGAGT AATGGCAGGT | 4380 |
| AGGTGTGTGT GTGTGTGTGT GTGTGTGTGT GTGTGTGTGT GTGTGAGAGA GAGAGAGAGA | 4440 |
| GAGAGAGAGA GAGAGAGAGA GAGAGAGAGA GAGAGAGAGA AGTTTGTGGA AAATCAGGTA | 4500 |
| ATGACAGCTC ATCCTTTTAG AATTGTACTT CAGAATAGAA ACATTTGGTG GGCTGTTAGG | 4560 |
| TAGCTTTGAT TACTTGTGGG TAGACCTGCT AGTATTGCCA GTCCTCAAGC AATGAGCITT | 4620 |
| CTGTATCTTG TTTACTAGAT ATATACTACC AGGTGAGTCA TTTCCTGGGG TTCTGTTTTC | 4680 |
| TTTTAAAATC TTTCCCTAAA CTTAATATGT ATTAAAAAGT CTGGCTTTTC AGTCCATTCT | 4740 |
| TTGTGCACTG GGATGGCAAT TGCTTCATTA TATGACAATT GCTGTTCCCA AGTCAGAATT | 4800 |
| CAGTGTGCTG ATTTGACATC AGTTCGTCCC GAATAAGTTC CTGTTACCAG GATTTACATT | 4860 |
| CAGCACATTA GAAACTTGTT GGTGTGCTTT TATTCTTGGA GCATTTTCCT TAGACTACCT | 4920 |
| TCCACTTTGA GTGCTCTGTT TAGGATGTTG AGGTGTTAGG ATTCTTGACA GCCAGAAAGA | 4980 |
| CTGAACCCAC TATCTGGGCA CAGTGTTCGT GTTGCTCTAT AAATGTATGC TTTTTTTGAT | 5040 |
| TTGGGGTTGT TTTACCTACA TTGTCAAACT AGATCCATGC TTAACAGTGA TAATGAAGGC | 5100 |
| TTTTTGTTTG TTTTGTTTGT GGGTCCTCCC CCCCCCCCCA AGACAGGGTT TCTCTGTAGG | 5160 |
| CTGTCCTAGA ACTTGTTCTT TTTTAACCAA AATTTGGCAA GGCTGAAAAT GGAATCCTAT | 5220 |
| AATCAATGCT GGCCACATTA AAGTTAATAG TTGAGAAGTC TTGTCTGAAT TTCCTTGGGC | 5280 |
| AAAAAGATTC TAGCCAGTTC AATACCCTGT TGTGCAAATT CAATTTGCTG TTATAATTTG | 5340 |
| CTCTCAGTTA TCAGTTGGAA GGAGGTTAAT TCTAATGTAC TTGGAAGAGG CCTGTAGACC | 5400 |
| ATCTATAACT GCATCAGTTG TACAGCGTTG TTGCCTGGGA TTCTCTAGTT CACATAAACT | 5460 |
| CCCAAGTCTT AGCCGTGGTG ATGGCTACAG TGTGGAAGAT GGTGAGCATT CTAGTGAGTA | 5520 |
| TCGCGATGAC GGCAGTAAAG AGCAGCAGGC AGCCGTGGCT GGGCTCACTG ACCGTGGCTG | 5580 |
| TAAGTTACGG AGGCAGCACA CACTTCTGTA CACACCTCTC ATCAGTTACC GGAGTCATTG | 5640 |
| CATTGCGGAC TAACTGGCTG ACTCAAGTTG TCTTGCTACT GAAGTCTTGA GTTGGTCTCA | 5700 |
| TGCATTTACC CTGTTGACTT GAGCACCTTA AAGTCGAAAG GATGTCTGGT TGTGGCTTTA | 5760 |
| TTGTAAACAG CCTTAGGTAA AGAGGGGAGT ATATCGGTTA GGAAGGTGAA AAATGATACT | 5820 |
| TCCAAGTTCA GTGGGAAACC CTGGGTTTAT CCCCCAGCTT AAGAAAGAAT GCCTAACAAT | 5880 |
| GTTTCAGAAT TAGATTCTGT GGAAGGTGAG GGTGTTAGAA CAGTCCAAAT TTGTTATTGT | 5940 |
| AGACTTGCAG TGGGAGGAAT TTTTAAATAT ACAGATCAGT CGACACTCAT TAACTTCACT | 6000 |
| GATAAAGGTG GAAACGGATG TGGCAACACT TCTAAGTTCA TTTGTATATG TTTGTAATTT | 6060 |
| GATTGGTTGT ATTCTGTTGC ACTCTAGAAT TTGAAGGCAA GGTTACCTCT GCTTTTTAAT | 6120 |
| TTTTTTTTTT TTAAAGAAAG AAAAAACACT GAAAGAAACT TCAAAAGATC TGTTAATGCT | 6180 |
| AATACCTGAA TGTGGCATTT AACATGTCAT GGAAACTGCT TTGAATAAAT ACTTGAGAAA | 6240 |
| AGGAATGAAA TAATTGCCGT TTTTGTTGTT GAGTGAATGG GTGTGGTTTA ATGAGCGTAA | 6300 |
| TCATTTTTAT AAAACAGCTG TGAGACTGAA GTGGAATCCT TATTAAATGT GGAAAATGGC | 6360 |
| CTTTGAGGAT TACAGTAGAG ATTCAACTAA GAGAGTAAAT AAAGCTTGAA ACTAATTCGT | 6420 |
| TGTAAATTGC TTCTACAATC ATTGCTCTAT ATAGCATGCT ATTGCCAATC AGTTTTATGT | 6480 |
| ATTAAGACCT ATCAGCATGT CTTTTTTAGG TTGACCTCAT TTTAAATTAT AAGATGCTCT | 6540 |
| CTGTACCGTT TTAACATTTC CAGGATTTAT TCTTTCTAGG CAAATTCCAC TGGACTGTTT | 6600 |
| CCATTGTAGA AGCTTCCTTA TAGATTCTTC AAATGAAGCT TACAGTGTGC TTTCTTGGGG | 6660 |
| TTTTGATTTG CACTAAATTT TATTTTCTGA AAGATCACTT ATGTTTATAA TGTAGTGCTT | 6720 |
| TGTCTTAACA ATTAAACTTT CCAGCACTCA TGCA | |

The mouse p42^{AUF1} amino acid sequence of GenBank Accession No. NP_001070734.1 (SEQ ID NO: 12) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGKM FIGGLSWDTT KKDLKDYFSK FGEVVDCTLK LDPITGRSRG | 120 |
| FGFVLFKESE SVDKVMDQKE HKLNGKVIDP KRAKAMKTKE PVKKIFVGGL SPDTPEEKIR | 180 |
| EYFGGFGEVE SIELPMDNKT NKRRGFCFIT FKEEEPVKKI MEKKYHNVGL SKCEIKVAMS | 240 |
| KEQYQQQQQW GSRGGFAGRA RGRGGGPSQN WNQGYSNYWN QGYGNYGYNS QGYGGYGGYD | 300 |
| YTGYNNYYGY GDYSNQQSGY GKVSRRGGHQ NSYKPY | |

The mouse p45^{AUF1} nucleotide sequence of GenBank Accession No. NM_001077265.2 (SEQ ID NO: 15) is as follows:

| | |
|---|---|
| CCATTTTAGG TGGTCCGCGG CGGCGCCATT AAAGCGAGGA GGAGGCGAGA GTGGCCGCCG | 60 |
| CTGCTACTTC ATTCTTTTTT TTTTCAGTGC AGCCGGGGAG AGCGAGAGAG CGCGCTGCGC | 120 |
| GAGAGTGGGA GGCGAGGGGG GCAGGCCGGG GAGAGGCGCA GGAGCCCTTG CAGCCACGCG | 180 |
| CGCGCCTTGT CTAGGGTGCC TCGCGAGGTA GAGCGGGCAT CGCGCGGCGG CGGCGGGGAT | 240 |
| TACTTTGCTG CTAGTTTCGG TTCGCGGCGG CGGCGGCGTC GGCGGGTGTC GTCTTCGGCG | 300 |
| GCGGCAGTAG CACTATGTCG GAGGAGCAGT TCGGAGGGGA CGGGGCGGCG GCGGCGGCAA | 360 |
| CGGCGGCGGT AGGCGGCTCG GCGGGCGAGC AGGAGGGAGC CATGGTGGCG GCGGCGGCGC | 420 |
| AGGGGCCGGC GGCGGCGGCG GGAAGCGGGA GCGGCGGCGG CGGCTCTGCG GCCGGAGGCA | 480 |
| CCGAAGGAGG CAGCGCCGAG GCAGAGGGAG CCAAGATCGA CGCCAGTAAG AACGAGGAGG | 540 |
| ATGAAGGCCA TTCAAACTCC TCCCCACGAC ACACTGAAGC AGCGGCGGCA CAGCGGGAAG | 600 |
| AATGGAAAAT GTTTATAGGA GGCCTTAGCT GGGACACCAC AAAGAAAGAT CTGAAGGACT | 660 |
| ACTTTTCCAA ATTTGGTGAA GTTGTAGACT GCACTCTGAA GTTAGATCCT ATCACAGGGC | 720 |
| GATCAAGGGG TTTTGGCTTT GTGCTATTTA AAGAGTCGGA GAGTGTAGAT AAGGTCATGG | 780 |
| ATCAGAAAGA ACATAAATTG AATGGGAAAG TCATTGATCC TAAAAGGGCC AAAGCCATGA | 840 |
| AAACAAAAGA GCCTGTCAAA AAAATTTTTG TTGGTGGCCT TTCTCCAGAC ACACCTGAAG | 900 |
| AAAAAATAAG AGAGTACTTT GGTGGTTTTG GTGAGGTTGA ATCCATAGAG CTCCCTATGG | 960 |
| ACAACAAGAC CAATAAGAGG CGTGGGTTCT GTTTTATTAC CTTTAAGGAA GAGGAGCCAG | 1020 |
| TGAAGAAGAT AATGGAAAAG AAATACCACA ATGTTGGTCT TAGTAAATGT GAAATAAAAG | 1080 |
| TAGCCATGTC AAAGGAACAG TATCAGCAGC AGCAGCAGTG GGGATCTAGA GGAGGGTTTG | 1140 |
| CAGGCAGAGC TCGCGGAAGA GGTGGAGGCC CCAGTCAAAA CTGGAACCAG GGATATAGTA | 1200 |
| ACTATTGGAA TCAAGGCTAT GGCAACTATG GATATAACAG CCAAGGTTAC GGAGGTTATG | 1260 |
| GAGGATATGA CTACACTGGT TACAACAACT ACTATGGATA TGGTGATTAT AGCAATCAGC | 1320 |
| AGAGTGGTTA TGGGAAAGTA TCCAGGCGAG GTGGACATCA AAATAGCTAC AAACCATACT | 1380 |
| AAATTATTCC ATTTGCAACT TATCCCCAAC AGGIGGTGAA GCAGTATTTT CCAATTTGAA | 1440 |
| GATTCATTTG AAGGTGGCTC CTGCCACCTG CTAATAGCAG TTCAAACTAA ATTTTTTCTA | 1500 |
| TCAAGTTCCT GAATGGAAGT ATGACGTTGG GTCCCTCTGA AGTTTAATTC TGAGTTCTCA | 1560 |
| TTAAAAGAAT TTGCTTTCAT TGTTTTATTT CTTAATTGCT ATGCTTCAGT ATCAATTTGT | 1620 |
| GTTTTATGCC CCCCCTCCCC CCCAGTATTG TAGAGCAAGT CTTGTGTTAA AAAAAGCCCA | 1680 |
| GTGTGACAGT GTCATGATGT AGTAGTGTCT TACTGGTTTT TTAATAAATC CTTTTGTATA | 1740 |
| AAAATGTATT GGCTCTTTTA TCATCAGAAT AGGAGGAAGT GAAATACTAC AAATGTTTGT | 1800 |
| CTTGGATTCA AGTCACTAGA AGCATAAATT TGAGGGGATA AAAACAACGG TAAACTTTGT | 1860 |
| CTGAAAGAGG GCATGGTTAA AAATGTAGTG AATTTTAAAT GTTTTTAGCA AAATTTGATT | 1920 |
| TTGCCCAAGA ATCCCTGTCT GAATTGGAAA TGACTTAATG TAGTCAATGT GCTTGTTGGT | 1980 |
| TGTCTTAATA TTACTTCTGT AGCCATTAAG TTTTATGAGT AACTTCCCAA ATACCCACGT | 2040 |
| TTTTCTTTAT ATGTATTGTG CTTTTTAAAA ACAAATCTGG AAAAATGGGC AAGAACATTT | 2100 |
| GCAGACAATT GTTTTTAAGC TTCCATTAAA TAAAAAAAAT GTGGACTTAA GGAAATCTAT | 2160 |
| TAATTTAAAT AGAACTGCAG CTAGTTTAGA GAGTATTTTT TTCTTAAAGC TTTGGTGTAA | 2220 |
| TTAGGGAAGA TTTTAAAAAA TGCATAGTGT TTATTTGTAT GTGTGCTCTT TTTTTAAGTC | 2280 |
| AATTTTTGGG GGGTTGGTCT GTTAACTGAG TCTAGGATTT AAAGGTAAGA TGTTCCTAGA | 2340 |
| AATCTTGTCA TCCCAAAGGG GCGGGCGCTA AGGTGAAACT TCAGGGTTCA GTCAGGGTCA | 2400 |
| CTGCTTTATG TGTGAAATCA CTCAAATTGG TAAGTCTCTT ATGTTAGCAT TCAGGACATT | 2460 |
| GATTTCAACT TGGATGGACA ATTTATAGTT ACTACTGAAT TGTGTGTTAA TGTGTTCAGT | 2520 |
| CCTGGTAAGT TTTCAGTTTG ATCAGTTAGT TGGAAGCAGA CTTGAAGAGC TGTTAGTCAC | 2580 |
| GTGAGCCATG GGTGCAGTCG ATCTGTGGTC AGATGCCTGA GTCTGTGATA GTGAATTGTG | 2640 |
| TCTAAAGACA TTTTAATGAT AAAAGTCAGT GCTGTAAAGT TGAAAGTTCA TGAGAGACAT | 2700 |
| ACAATGAGGG CTGCAGCCCA TTTTTAAAAA CATTATAATA CAAAAGTATG CACATTTGTT | 2760 |
| TACATATCCC TGCCTTTGTA TTACAGTGGC AGGTTTGTGT ACTTAAACTG GGAAAGCCTC | 2820 |
| AGATCTATGA TTACCTGGCC TATCATAGAA AGTGTCTAAA TAAATCACTC TGTCAATTGA | 2880 |
| ATACATTAGT ATTAGCTAGC ATACTTCATT ATGCCTGTTT TCCATAAATA CCACACCAAA | 2940 |
| AACTTGCTTG GGGCAGTTTG AGCCTAGTTC ATGAGCTGCT ATCAGATTGG TCTTGATCCT | 3000 |
| ATATAATAGG CCAAATGTCT GTAAACAGCT GTGCTGGTGG AATGTAGAAA GTCACTGCAC | 3060 |
| TCAGATTCAA CTTCCTGATT GGAAGTCATC ACAGTGTGAT TAAACATTTT CACAAAGAAT | 3120 |
| AGTAGATAAA TAACTTGGTT TTTAATGTTA ACTTTGTTTC CATTAAGTCA CATTTAAAAA | 3180 |
| CTTATCCTCA CGCCTACCTG AGTTAATTAT CTGTTGACCT AGATATCTTT CTGGCCACTC | 3240 |
| ACTGACTTAT TTCTTGAACT TTTGCCATTT GCATAAATCT TGTCAGCTTT GTTCTTGATT | 3300 |
| ATGCATTGTC CAGGCTGAGC TAGTTGTCTT TCCAGGAATC CCTTTGTCTC TGAATTAGGT | 3360 |
| CCTTTGTTTC CTAAATCATC CTGCTTGTTT GGCACAAGTC TTCCCAGGCC AGTGAGACCT | 3420 |
| CCGTGTCCTC TCAGCACCAT AGGGGTAGGT AACCCTGGTT AGGCTGGACA GGGGTTTGCT | 3480 |
| GAGGGAGTTT GTTCATTTGA ATCTAGGTCT TACATGACGT CTTTCAAATA GGGTTTTTAC | 3540 |
| CTTGACACTA AACTGTCCAG TCTAAGCAGT TCTGCAAAAT GTGAGGGAAT TATGAACTTC | 3600 |
| TTCCTGCAGT GGGTTTTTAT GGTTTTGGTT TGTTTTTTGT TGTTTTGGTT CTTTGTTGAG | 3660 |
| CCCTGGACAA AAACTTCCCT AGTTCTGGTT TCTACAATTT AAATTAAAAA CAGAATTCAT | 3720 |
| CTTAGAATTT TTCACCCTCT TCCCCAACTA TTCTAATCAA TCTTAAGTAT GCCCTTCATC | 3780 |
| TTTTTTCCTT CCTAAGGCTT TTACTGATAG TGTAATTCCG TACTCTTCAA CCCTGGGAAG | 3840 |
| GCTGAAGTGG ATTCTTGAGC TCATTTCAAG GCTGACCTGG GTGTTGGCAA GAACCCAGCT | 3900 |
| TAGAACAAAC ACATGCAAGG CCATCTTACC TTACATCCTG TTGCTTGGAC TTCTTCCTGC | 3960 |
| TCAAAGTTTT TAGTGGATGC TAAGTGATCT TTGCTTCCAC TGAGGAGTGG AACACTTTAG | 4020 |
| AATGAACCTC TAGATAGATA TTTTTATTGT CTGGTGAGGG TTACTGGAGT TTCCCACCCT | 4080 |
| GCCTGAAGGG TGAATCTGGC TTACAGTGTT CTCATCTCAA AGGGAAGAAG GCAGATGGCT | 4140 |
| GTGTCCAGAG AGAGCCATCA CAGTTTGCTT CAGAGACACT AGAATGGGCT GGAAGATCTA | 4200 |
| GTGGTCTTAA TCAGACTTGA AACCTGGCCT TTCTTCATTA CCCATATGTC TACCAGTACT | 4260 |
| TGGGCTAACA CTTAAGCCAT TAGGGCCTTT GTAGGGGTGT TTTGAGACCC CCTCCATGCT | 4320 |
| AACAAATATA CAGGTTTCTT AACATTTGCT CATAAACTTG TAAAGCTTAC TTTCTCTTAA | 4380 |
| TCCACCCCAC ATTTAACAAG CCCTGGTACT TAGAATTTCA GAAGAGTAAT GGCAGGTAGG | 4440 |
| TGTGTGTGTG TGTGTGTGTG TGTGTGTGTG TGTGTGTGTG TGAGAGAGAG AGAGAGAGAG | 4500 |
| AGAGAGAGAG AGAGAGAGAG AGAGAGAGAG AGAGAGAAGT TTGTGGAAAA TCAGGTAATG | 4560 |
| ACAGCTCATC CTTTTAGAAT TGTACTTCAG AATAGAAACA TTTGGTGGGC TGTTAGGTAG | 4620 |
| CTTTGATTAC TTGTGGGTAG ACCTGCTAGT ATTGCCAGTC CTCAAGCAAT GAGCTTTCTG | 4680 |
| TATCTTGTTT ACTAGATATA TACTACCAGG TGAGTCATTT CCTGGGGTTC TGTTTTCTTT | 4740 |
| TAAAATCTTT CCCTAAACTT AATATGTATT AAAAAGTCTG GCTTTTCAGT CCATTCTTTG | 4800 |
| TGCACTGGGA TGGCAATTGC TTCATTATAT GACAATTGCT GTTCCCAAGT CAGAATTCAG | 4860 |
| TGTGCTGATT TGACATCAGT TCGTCCCGAA TAAGTTCCTG TTACCAGGAT TTACATTCAG | 4920 |
| CACATTAGAA ACTTGTTGGT GTGCTTTTAT TCTTGGAGCA TTTTCCTTAG ACTACCTTCC | 4980 |
| ACTTTGAGTG CTCTGTTTAG GATGTTGAGG TGTTAGGATT CTTGACAGCC AGAAAGACTG | 5040 |
| AACCCACTAT CTGGGCACAG TGTTCGTGTT GCTCTATAAA TGTATGCTTT TTTTGATTTG | 5100 |
| GGGTTGTTTT ACCTACATTG TCAAACTAGA TCCATGCTTA ACAGTGATAA TGAAGGCTTT | 5160 |
| TTGTTTGTTT TGTTTGTGGG TCCTCCCCCC CCCCCCAAGA CAGGGTTTCT CTGTAGGCTG | 5220 |
| TCCTAGAACT TGTTCTTTTT TAACCAAAAT TTGGCAAGGC TGAAAATGGA ATCCTATAAT | 5280 |
| CAATGCTGGC CACATTAAAG TTAATAGTTG AGAAGTCTTG TCTGAATTTC CTTGGGCAAA | 5340 |
| AAGATTCTAG CCAGTTCAAT ACCCTGTTGT GCAAATTCAA TTTGCTGTTA TAATTTGCTC | 5400 |
| TCAGTTATCA GTTGGAAGGA GGTTAATTCT AATGTACTTG GAAGAGGCCT GTAGACCATC | 5460 |
| TATAACTGCA TCAGTIGTAC AGCGTTGTTG CCTGGGATTC TCTAGTTCAC ATAAACTCCC | 5520 |
| AAGTCTTAGC CGTGGTGATG GCTACAGTGT GGAAGATGGT GAGCATTCTA GTGAGTATCG | 5580 |
| CGATGACGGC AGTAAAGAGC AGCAGGCAGC CGTGGCTGGG CTCACTGACC GTGGCTGTAA | 5640 |
| GTTACGGAGG CAGCACACAC TTCTGTACAC ACCTCTCATC AGTTACCGGA GTCATTGCAT | 5700 |
| TGCGGACTAA CTGGCTGACT CAAGTIGTCT TGCTACTGAA GTCTTGAGTT GGTCTCATGC | 5760 |
| ATTTACCCTG TTGACTTGAG CACCTTAAAG TCGAAAGGAT GTCTGGTTGT GGCTTTATTG | 5820 |
| TAAACAGCCT TAGGTAAAGA GGGGAGTATA TCGGTTAGGA AGGTGAAAAA TGATACTTCC | 5880 |
| AAGTTCAGTG GGAAACCCTG GGTTTATCCC CCAGCTTAAG AAAGAATGCC TAACAATGTT | 5940 |
| TCAGAATTAG ATTCTGTGGA AGGTGAGGGT GTTAGAACAG TCCAAATTTG TTATTGTAGA | 6000 |
| CTTGCAGTGG GAGGAATTTT TAAATATACA GATCAGTCGA CACTCATTAA CTTCACTGAT | 6060 |
| AAAGGTGGAA ACGGATGTGG CAACACTTCT AAGTTCATTT GTATATGTTT GTAATTTGAT | 6120 |
| TGGTTGTATT CTGTTGCACT CTAGAATTTG AAGGCAAGGT TACCTCTGCT TTTTAATTTT | 6180 |
| TTTTTTTTTA AAGAAAGAAA AAACACTGAA AGAAACTTCA AAAGATCTGT TAATGCTAAT | 6240 |
| ACCTGAATGT GGCATTTAAC ATGTCATGGA AACTGCTTTG AATAAATACT TGAGAAAAGG | 6300 |
| AATGAAATAA TTGCCGTTTT TGTTGTTGAG TGAATGGGTG TGGTTTAATG AGCGTAATCA | 6360 |
| TTTTTATAAA ACAGCTGTGA GACTGAAGTG GAATCCTTAT TAAATGTGGA AAATGGCCTT | 6420 |
| TGAGGATTAC AGTAGAGATT CAACTAAGAG AGTAAATAAA GCTTGAAACT AATTCGTTGT | 6480 |
| AAATTGCTTC TACAATCATT GCTCTATATA GCATGCTATT GCCAATCAGT TTTATGTATT | 6540 |
| AAGACCTATC AGCATGTCTT TTTTAGGTTG ACCTCATTTT AAATTATAAG ATGCTCTCTG | 6600 |
| TACCGTTTTA ACATTTCCAG GATTTATTCT TTCTAGGCAA ATTCCACTGG ACTGTTTCCA | 6660 |
| TTGTAGAAGC TTCCTTATAG ATTCTTCAAA TGAAGCTTAC AGTGTGCTTT CTTGGGGTTT | 6720 |
| TGATTTGCAC TAAATTTTAT TTTCTGAAAG ATCACTTATG TTTATAATGT AGTGCTTTGT | 6780 |
| CTTAACAATT AAACTTTCCA GCACTCATGC A | |

The mouse p45^{AUF1} amino acid sequence of GenBank Accession No. NP_001070733.1 (SEQ ID NO: 16) is as follows:

| | |
|---|---|
| MSEEQFGGDG AAAAATAAVG GSAGEQEGAM VAAAAQGPAA AAGSGSGGGG SAAGGTEGGS | 60 |
| AEAEGAKIDA SKNEEDEGHS NSSPRHTEAA AAQREEWKMF IGGLSWDTTK KDLKDYFSKF | 120 |
| GEVVDCTLKL DPITGRSRGF GFVLFKESES VDKVMDQKEH KLNGKVIDPK RAKAMKTKEP | 180 |
| VKKIFVGGLS PDTPEEKIRE YFGGFGEVES IELPMDNKTN KRRGFCFITF KEEEPVKKIM | 240 |
| EKKYHNVGLS KCEIKVAMSK EQYQQQQQWG SRGGFAGRAR GRGGGPSQNW NQGYSNYWNQ | 300 |
| GYGNYGYNSQ GYGGYGGYDY TGYNNYYGYG DYSNQQSGYG KVSRRGGHON SYKPY | |

It is noted that the sequences described herein may be described with reference to accession numbers, for example, as provided in Table 1, that include, e.g., a coding sequence or protein sequence with or without additional sequence elements or portions (e.g., leader sequences, tags, immature portions, regulatory regions, etc.). Thus, reference to such sequence accession numbers or corresponding sequence identification numbers refers to either the sequence fully described therein or some portion thereof (e.g., that portion encoding a protein or polypeptide of interest to the technology described herein (e.g., AUF1 or a functional fragment thereof); the mature protein sequence that is described within a longer amino acid sequence; a regulatory region of interest (e.g., promoter sequence or regulatory element) disclosed within a longer sequence described herein; etc.). Likewise, variants and isoforms of accession numbers and corresponding sequence identification numbers described herein are also contemplated.

Accordingly, in certain embodiments, the AUF1 protein referred to herein has an amino acid sequence as set forth in Table 1 and the sequences disclosed herein, or is a functional fragment thereof. In certain embodiments, the AUF1 is a p37, p40, p42 or p45 form of human AUF1 and has an amino acid sequence of SEQ ID NO: 2, 6, 10, or 14, respectively. In other embodiments, the AUF1 is a p37, p40, p42 or p45 form of mouse AUF1 and has an amino acid sequence of SEQ ID NO: 4, 8, 12, or 16, respectively. In certain embodiments, the AUF1 has 90%, 95% or 99% sequence identity to the amino acid sequence of SEQ ID NO: 2, 6, 10, or 14 and has AUF1 functional activity. In certain embodiments, the AUF1 has 90%, 95% or 99% sequence identity to the amino acid sequence of SEQ ID NO: 4, 8, 12, or 16 and has AUF1 functional activity. In one embodiment, the functional fragment as referred to herein includes an amino acid sequence that has at least 80%, at least 85%, at least 90%, at least 95%, at least 97%, or at least 99% amino acid sequence identity to amino acid sequence of SEQ ID NO: 2, 6, 10, or 14 for human AUF1 or in other embodiments to the amino acid sequence of SEQ ID NO: 4, 8, 12, or 16 for mouse AUF1.

Also provided are nucleic acids comprising nucleotide sequences encoding a human AUF1 protein, or functional fragment thereof, for example, the nucleotide sequences of SEQ ID NO: 1, 5, 9, or 13. Also provided are nucleic acids comprising nucleotide sequences having 80%, 85%, 90%, 95%, or 99% sequence identity to one of the nucleotide sequences of SEQ ID NO: 1, 5, 9, or 13 and encoding a human AUF1 protein having an amino acid sequence of SEQ ID NO: 2, 6, 10, or 14, or a functional fragment thereof. Provided are codon optimized sequences encoding an AUF1 protein, including, a codon optimized version of the human p40 AUF1 coding sequence is the nucleotide sequence of SEQ ID NO: 17. Also provided are nucleic acids comprising nucleotide sequences having 80%, 85%, 90%, 95%, or 99% sequence identity to one of the nucleotide sequences of SEQ ID NO: 3, 7, 11, or 15 and encoding a mouse AUF1 protein having an amino acid sequence of SEQ ID NO: 4, 8, 12, or 16, or a functional fragment thereof.

In some embodiments, the AAV vectors and viral particles described herein comprise a nucleic acid molecule comprising a nucleotide sequence set forth in Table 1 (or described herein), or portions thereof that encode a functional fragment of an AUF1 protein as described supra, particularly in an expression cassette as described herein for expression in the cells of a subject, particularly, muscle cells of a subject.

### 5.2.2 AUF1 Gene Cassettes

Another aspect provided herein relates to nucleic acid expression cassettes comprising a nucleic acid encoding an AUF1 (including human p37, p40, p42 or p45 AUF1, including a combination thereof) or a functional fragment thereof operably linked to regulatory elements, including promoter elements, and optionally enhancer elements and/or introns, to enhance or facilitate expression of the nucleic acid encoding the AUF1 or functional fragment thereof, including, for example, in muscle cells. The expression cassettes or transgenes provided herein may comprise nucleotide sequences encoding a human AUF1 protein having an amino acid sequence of SEQ ID NO: 2, 6, 10, or 14, or a functional fragment thereof (or, alternatively, for example, for mouse model studies, the expression cassette comprises a nucleotide sequence encoding a mouse AUF1 protein having an amino acid sequence of SEQ ID NO: 4, 8, 12, or 16, or a functional fragment thereof). In embodiments, the nucleotide sequence encoding the human AUF1 is SEQ ID NO: 1, 5, 9, or 13 (or the nucleotide sequence encoding mouse AUF1 is SEQ ID NO: 3, 7, 11, or 15). In certain embodiments, the nucleotide sequence is SEQ ID NO: 17, which encodes human p40 AUF1 and codon and CpG optimized. In certain embodiments, the AUF1 protein has no more than 1, 2, 3, 4, 5, 10, 15 amino acid substitutions, including conservative substitutions, with respect to the amino acid sequence of SEQ ID NO: 2, 6, 10, or 14, or a functional fragment thereof (or, alternatively, for example, for mouse model studies, with respect to the amino acid sequence of SEQ ID NO: 12, 16, 20 or 24), where the AUF1 protein has one or more AUF1 functions. In embodiments, the regulatory control elements include promoters and may be either constitutive or may be tissue-specific, that is, active (or substantially more active or significantly more active) only in the target cell/tissue. In particular, provided are promoter and other regulatory elements that promote muscle specific expression, such as those in Table 10 infra. In embodiments, including for use as a transgene in a recombinant AAV particle, the expression cassette or transgene is flanked by inverted terminal repeats (ITRs) (for example AAV2 ITR, including forms of ITRs for single-stranded AAV genomes or self-complementary AAV genomes. For example, the 5′ and 3′ ITR sequences are SEQ ID NO: 28 and 29, respectively. In an embodiment, the 5′ ITR is mutated for a self-complementary vector and may have, for example, the nucleotide sequence of SEQ ID NO: 30.

### 5.2.2.1 Codon Optimization and CpG Depletion

In one aspect the nucleotide sequence encoding the AUF1 is modified by codon optimization and CpG dinucleotide and CpG island depletion. Immune response against a transgene is a concern for human clinical application. AAV-directed immune responses can be inhibited by reducing the number of CpG di-nucleotides in the AAV genome [Faust, S. M., et al., CpG-depleted adeno-associated virus vectors evade immune detection. J Clin Invest, 2013. 123(7): p. 2994-3001]. Depleting the transgene sequence of CpG motifs may diminish the role of TLR9 in activation of innate immunity upon recognition of the transgene as non-self, and thus provide stable and prolonged transgene expression. [See also Wang, D., P. W. L. Tai, and G. Gao, Adeno-associated virus vector as a platform for gene therapy delivery. Nat Rev Drug Discov, 2019. 18(5): p. 358-378.; and Rabinowitz, J., Y. K. Chan, and R. J. Samulski, Adeno-associated Virus (AAV) versus Immune Response. Viruses, 2019. 11(2)]. In embodiments, the AUF1 nucleotide sequence and the expression cassette is human codon-optimized with CpG depletion. Codon-optimized and CpG depleted nucleotide sequences may be designed by any method known in the art, including for example, by Thermo Fisher Scientific GeneArt Gene Synthesis tools utilizing GeneOptimizer (Waltham, MA USA)). Nucleotide sequence SEQ ID NO: 17 described herein represents codon-optimized and CpG depleted sequence.

5.2.3 AUF1 rAAV Genome Constructs

Provided are constructs that are useful as cis plasmids for rAAV construction that comprise a nucleotide sequence that encodes AUF1, including the p37, p40, p42 or p45 (including mouse and human) isoform thereof, operably linked to regulatory sequences that promote AUF1 expression in muscle cells.

rAAV genome constructs comprising an AUF1 transgene, including the codon optimized, CpG deleted human AUF1 p40 coding sequence of SEQ ID NO: 17, operably linked to regulatory sequences that promote expression in muscle cells, are provided herein. In certain embodiments, the constructs have a muscle specific promoter, which may be Spc5-12 (including modified Spc5-12 promoters Spc5v1 or Spc5v2 (SEQ ID Nos: 127 and 128, respectively, disclosed herein), tMCK or CK7 (see also Table 10 herein for promoters), optionally with an intron sequence between the promoter and the AUF1 coding sequence, such as a VH4 intron (see Table 11 for intron sequences), polyA signal sequences, such as rabbit beta globin poly A signal sequence (SEQ ID NO: 23), and optionally an WPRE sequence (SEQ ID NO: 24). The constructs may also include 5′ and/or 3′ stuffer sequences (SEQ ID Nos: 26 and 27 in Table 2, or any stuffer sequence known in the art, including, for example, stuffer sequences disclosed in Table 12, infra), and a SV40 polyadenylation signal sequence reversed with respect to the coding sequence and adjacent to the 3′ ITR sequence. In certain embodiments, the constructs have one or more components from Table 2.

TABLE 2
Components of AUF1 Constructs

| | Sequence (5′ to 3′ sequence of cassette top strand for |
|---|---|
| Description | nucleotides is provided) |
| Human AUF1 | RefSeq NM_002138.3 |
| isoform 3 also | See Table 1 |
| known as p40 (wild | |
| type coding | |
| sequence) | |
| SEQ ID NO: 5 | |
| Human AUF1 | RefSeq NM_031370.2 |
| isoform 1 also | See Table 1 |
| known as p45 | |
| (wild-type) | |
| SEQ ID NO: 13 | |
| Human AUF1 | RefSeq NM_031369.2 |
| isoform 2 also | See Table 1 |
| known as p42 | |
| (wild-type) | |
| SEQ ID NO: 9 | |
| Human AUF1 | RefSeq NM_001003810.2 |
| isoform 4 also | See Table 1 |
| known as p37 | |
| (wild-type) | |
| SEQ ID NO: 1 | |
| Human Codon | ATGTCTGAGGAACAGTTTGGTGGTGATGGGGCTGCTGCTGCAGCTACA |
| optimized, CpG | GCTGCTGTTGGAGGATCTGCTGGGGAACAAGAGGGTGCCATGGTTGCT |
| depleted AUF1 p40 | GCTACACAAGGTGCTGCAGCTGCTGCTGGTAGTGGTGCTGGAACAGGT |
| sequence | GGTGGAACAGCCAGTGGTGGCACAGAAGGAGGCTCTGCTGAATCTGAA |
| (921 bp) | GGGGCCAAGATTGATGCCAGCAAGAATGAGGAAGATGAGGGCCACAGC |
| SEQ ID NO: 17 | AACAGCTCCCCAAGACACTCTGAAGCTGCCACAGCTCAGAGGGAAGAG |
| | TGGAAGATGTTCATTGGAGGCCTGAGCTGGGACACCACCAAGAAGGAC |
| | CTGAAGGACTACTTCAGCAAGTTTGGAGAAGTGGTGGACTGCACCCTG |
| | AAGCTGGACCCTATCACAGGCAGAAGCAGAGGCTTTGGCTTTGTGCTG |
| | TTCAAAGAATCTGAGTCTGTGGACAAAGTGATGGACCAGAAAGAACAC |
| | AAGCTGAATGGGAAAGTGATTGACCCCAAGAGGGCCAAAGCCATGAAG |
| | ACCAAAGAGCCTGTCAAGAAGATCTTTGTTGGAGGGCTGTCCCCTGAC |
| | ACACCTGAGGAAAAGATCAGAGAGTACTTTGGAGGATTTGGAGAGGTG |
| | GAATCCATTGAGCTGCCCATGGACAACAAGACCAACAAGAGAAGAGGC |
| | TTCTGCTTCATCACCTTCAAAGAGGAAGAACCAGTCAAGAAAATCATG |
| | GAAAAGAAATACCACAATGTGGGCCTGAGCAAGTGTGAAATCAAGGTG |
| | GCCATGAGCAAAGAGCAGTACCAGCAACAACAGCAGTGGGGCTCCAGA |
| | GGAGGTTTTGCTGGCAGAGCTAGAGGCAGAGGTGGTGACCAGCAGTCT |
| | GGCTATGGCAAGGTGTCCAGAAGAGGTGGACATCAGAACAGCTACAAG |
| | CCCTACTGA |
| Human AUF1 | MSEEQFGGDGAAAAATAAVGGSAGEQEGAMVAATQGAAAAAGSGAGTG |
| isoform 3 protein, | GGTASGGTEGGSAESEGAKIDASKNEEDEGHSNSSPRHSEAATAQREE |
| p40 | WKMFIGGLSWDTTKKDLKDYFSKFGEVVDCTLKLDPITGRSRGFGFVL |
| (306 aa) | FKESESVDKVMDQKEHKLNGKVIDPKRAKAMKTKEPVKKIFVGGLSPD |
| SEQ ID NO: 6 | TPEEKIREYFGGFGEVESIELPMDNKTNKRRGFCFITFKEEEPVKKIM |
| | EKKYHNVGLSKCEIKVAMSKEQYQQQQQWGSRGGFAGRARGRGGDQQS |
| | GYGKVSRRGGHQNSYKPY |
| | (UNIPROTKB Q14103-3, ALSO REFSEQ NP_002129.2) |
| Spc5-12 promoter | GGCCGTCCGCCCTCGGCACCATCCTCACGACACCCAAATATGGCGACG |
| SEQ ID NO: 18 | GGTGAGGAATGGTGGGGAGTTATTTTTAGAGCGGTGAGGAAGGTGGGC |
| | AGGCAGCAGGTGTTGGCGCTCTAAAAATAACTCCCGGGAGTTATTTTT |
| | AGAGCGGAGGAATGGTGGACACCCAAATATGGCGACGGTTCCTCACCC |
| | GTCGCCATATTTGGGTGTCCGCCCTCGGCCGGGGCCGCATTCCTGGGG |
| | GCCGGGCGGTGCTCCCGCCCGCCTCGATAAAAGGCTCCGGGGCCGGCG |
| | GCGGCCCACGAGCTACCCGGAGGAGCGGGAGGCGCCAAGC |
| VH4 intron | GTGAGTATCTCAGGGATCCAGACATGGGGATATGGGAGGTGCCTCTGA |
| SEQ ID NO: 19 | TCCCAGGGCTCACTGTGGGTCTCTCTGTTCACAG |
| Spc5-12 promoter + | GGCCGTCCGCCCTCGGCACCATCCTCACGACACCCAAATATGGCGACG |
| VH4 intron | GGTGAGGAATGGTGGGGAGTTATTTTTAGAGCGGTGAGGAAGGTGGGC |
| (includes splice | AGGCAGCAGGTGTTGGCGCTCTAAAAATAACTCCCGGGAGTTATTTTT |
| sites (SS)) | AGAGCGGAGGAATGGTGGACACCCAAATATGGCGACGGTTCCTCACCC |
| SEQ ID NO: 20 | GTCGCCATATTTGGGTGTCCGCCCTCGGCCGGGGCCGCATTCCTGGGG |
| | GCCGGGCGGTGCTCCCGCCCGCCTCGATAAAAGGCTCCGGGGCCGGCG |
| | GCGGCCCACGAGCTACCCGGAGGAGCGGGAGGCGCCAAGCCCGCGGAA |
| | CAGGTGAGTATCTCAGGGATCCAGACATGGGGATATGGGAGGTGCCTC |
| | TGATCCCAGGGCTCACTGTGGGTCTCTCTGTTCACAGGTT |
| tMCK promoter | GCCACTACGGGTCTAGGCTGCCCATGTAAGGAGGCAAGGCCTGGGGAC |
| SEQ ID NO: 21 | ACCCGAGATGCCTGGTTATAATTAACCCCAACACCTGCTGCCCCCCCC |
| | CCCCAACACCTGCTGCCTGAGCCTGAGCGGTTACCCCACCCCGGTGCC |
| | TGGGTCTTAGGCTCTGTACACCATGGAGGAGAAGCTCGCTCTAAAAAT |
| | AACCCTGTCCCTGGTGGATCGCCACTACGGGTCTAGGCTGCCCATGTA |
| | AGGAGGCAAGGCCTGGGGACACCCGAGATGCCTGGTTATAATTAACCC |
| | CAACACCTGCTGCCCCCCCCCCCCAACACCTGCTGCCTGAGCCTGAGC |
| | GGTTACCCCACCCCGGTGCCTGGGTCTTAGGCTCTGTACACCATGGAG |
| | GAGAAGCTCGCTCTAAAAATAACCCTGTCCCTGGTGGATCGCCACTAC |
| | GGGTCTAGGCTGCCCATGTAAGGAGGCAAGGCCTGGGGACACCCGAGA |
| | TGCCTGGTTATAATTAACCCCAACACCTGCTGCCCCCCCCCCCCAACA |
| | CCTGCTGCCTGAGCCTGAGCGGTTACCCCACCCCGGTGCCTGGGTCTT |
| | AGGCTCTGTACACCATGGAGGAGAAGCTCGCTCTAAAAATAACCCTGT |
| | CCCTGGTGGATCCCTCCCTGGGGACAGCCCCTCCTGGCTAGTCACACC |
| | CTGTAGGCTCCTCTATATAACCCAGGGGCACAGGGGCTGCCCCCGGGT |
| | CACC |
| CK7 promoter | CCACTACGGGTTTAGGCTGCCCATGTAAGGAGGCAAGGCCTGGGGACA |
| SEQ ID NO: 22 | CCCGAGATGCCTGGTTATAATTAACCCAGACATGTGGCTGCCCCCCCC |
| | CCCCCCAACACCTGCTGCCTCTAAAAATAACCCTGTCCCTGGTGGATC |
| | CCCTGCATGCGAAGATCTTCGAACAAGGCTGTGGGGGACTGAGGGCAG |
| | GCTGTAACAGGCTTGGGGGCCAGGGCTTATACGTGCCTGGGACTCCCA |
| | AAGTATTACTGTTCCATGTTCCCGGCGAAGGGCCAGCTGTCCCCCGCC |
| | AGCTAGACTCAGCACTTAGTTTAGGAACCAGTGAGCAAGTCAGCCCTT |
| | GGGGCAGCCCATACAAGGCCATGGGGCTGGGCAAGCTGCACGCCTGGG |
| | TCCGGGGTGGGCACGGTGCCCGGGCAACGAGCTGAAAGCTCATCTGCT |
| | CTCAGGGGCCCCTCCCTGGGGACAGCCCCTCCTGGCTAGTCACACCCT |
| | GTAGGCTCCTCTATATAACCCAGGGGCACAGGGGCTGCCCTCATTCTA |
| | CCACCACCTCCACAGCACAGACAGACACTCAGGAGCCAGCCAGCGTCG |
| | A |
| Rabbit globin poly | GATCTTTTTCCCTCTGCCAAAAATTATGGGGACATCATGAAGCCCCTT |
| A signal sequence | GAGCATCTGACTTCTGGCTAATAAAGGAAATTTATTTTCATTGCAATA |
| SEQ ID NO: 23 | GTGTGTTGGAATTTTTTGTGTCTCTCACTCG |
| WPRE | AATCAACCTCTGGATTACAAAATTTGTGAAAGATTGACTGGTATTCTT |
| SEQ ID NO: 24 | AACTATGTTGCTCCTTTTACGCTATGTGGATACGCTGCTTTAATGCCT |
| | TTGTATCATGCTATTGCTTCCCGTATGGCTTTCATTTTCTCCTCCTTG |
| | TATAAATCCTGGTTGCTGTCTCTTTATGAGGAGTTGTGGCCCGTTGTC |
| | AGGCAACGTGGCGTGGTGTGCACTGTGTTTGCTGACGCAACCCCCACT |
| | GGTTGGGGCATTGCCACCACCTGTCAGCTCCTTTCCGGGACTTTCGCT |
| | TTCCCCCTCCCTATTGCCACGGCGGAACTCATCGCCGCCTGCCTTGCC |
| | CGCTGCTGGACAGGGGCTCGGCTGTTGGGCACTGACAATTCCGTGGTG |
| | TTGTCGGGGAAATCATCGTCCTTTCCTTGGCTGCTCGCCTGTGTTGCC |
| | ACCTGGATTCTGCGCGGGACGTCCTTCTGCTACGTCCCTTCGGCCCTC |
| | AATCCAGCGGACCTTCCTTCCCGCGGCCTGCTGCCGGCTCTGCGGCCT |
| | CTTCCGCGTCTTCGCCTTCGCCCTCAGACGAGTCGGATCTCCCTTTGG |
| | GCCGCCTCCCCGC |
| SV40 polyA signal | GATCCAGACATGATAAGATACATTGATGAGTTTGGACAAACCACAACT |
| sequence | AGAATGCAGTGAAAAAAATGCTTTATTTGTGAAATTTGTGATGCTATT |
| SEQ ID NO: 25 | GCTTTATTTGTAACCATTATAAGCTGCAATAAACAAGTT |
| Stuffer (141 bp) | AAAAAGTACCTCAATAATAAATACAGAACTTCTCCTTTCAACCTCTTC |
| SEQ ID NO: 26 | CATCACATCAACACCTATGAAGACAATGGGTTTCTGATTGTGGATCTC |
| | TGCTGCTGGAAAGGATTTGAGTTTGTTTATAATTACTTATATTTA |
| Stuffer (893 bp) | GCTTGAGCATCCTGCTGGTGGTTACAAGAAACTGTTTGAAACTGTGGA |
| SEQ ID NO: 27 | GGAACTGTCCTCGCCGCTCACAGCTCATGTAACAGGCAGGATCCCCCT |
| | CTGGCTCACCGGCAGTCTCCTTCGATGTGGGCCAGGACTCTTTGAAGT |
| | TGGATCTGAGCCATTTTACCACCTGTTTGATGGGCAAGCCCTCCTGCA |
| | CAAGTTTGACTTTAAAGAAGGACATGTCACATACCACAGAAGGTTCAT |
| | CCGCACTGATGCTTACGTACGGGCAATGACTGAGAAAAGGATCGTCAT |
| | AACAGAATTTGGCACCTGTGCTTTCCCAGATCCCTGCAAGAATATATT |
| | TTCCAGGTTTTTTTCTTACTTTCGAGGAGTAGAGGTTACTGACAATTG |
| | CCCTTGTTAATGTCTACCCAGTGGGGGAAGATTACTACGCTTGCACAG |
| | AGACCAACTTTATTACAAAGATTAATCCAGAGACCTTGGAGACAATTA |
| | AGCAGGTTGATCTTTGCAACTAAGTCTCTGTCAATGGGGCCACTGCTC |
| | ACCCCCACATTGAAAATGATGGAACCGTTTACAATATTGGTAATTGCT |
| | TTGGAAAAAATTTTTCAATTGCCTACAACATTGTAAAGATCCCACCAC |
| | TGCAAGCAGACAAGGAAGATCCAATAAGCAAGTCAGAGATCGTTGTAC |
| | AATTCCCCTGCAGTGACCGATTCAAGCCATCTTACGTTCATAGTTTTG |
| | GTCTGACTCCCAACTATATCGTTTTTGTGGAGACACCAGTCAAAATTA |
| | ACCTGTTCAAGTTCCTTTCTTCATGGAGTCTTTGGGGAGCCAACTACA |
| | TGGATTGTTTTGAGTCCAATGAAACCATGGGGTTTGGCTTCATATTGC |
| | TGACAAAAAAAGGAAAAAGTACCTCAATA |
| 5′ ITR | CTGCGCGCTCGCTCGCTCACTGAGGCCGCCCGGGCAAAGCCCGGGCGT |
| SEQ ID NO: 28 | CGGGCGACCTTTGGTCGCCCGGCCTCAGTGAGCGAGCGAGCGCGCAGA |
| | GAGGGAGTGGCCAACTCCATCACTAGGGGTTCCT |
| 3′ ITR | AGGAACCCCTAGTGATGGAGTTGGCCACTCCCTCTCTGCGCGCTCGCT |
| SEQ ID NO: 29 | CGCTCACTGAGGCCGGGCGACCAAAGGTCGCCCGACGCCCGGGCTTTG |
| | CCCGGGCGGCCTCAGTGAGCGAGCGAGCGCGCAG |
| mITR (5′) [mutant | CTGCGCGCTCGCTCGCTCACTGAGGCCGCCCGGGCAAAGCCCGGGCGT |
| 5′ ITR for scAAV] | CGGGCGACCTTTGGTCGCCCGGCCTCAGTGAGCGAGCGAGCGCGCAGA |
| SEQ ID NO: 30 | GAGGGAGTGG |

In some embodiments, the rAAV genome comprises the following components: (1) AAV inverted terminal repeats that flank an expression cassette; (2) regulatory control elements, such as a) promoter/enhancers, b) a poly A signal, and c) optionally an intron; and (3) nucleic acid sequences coding for AUF1. In a specific embodiment, the constructs described herein comprise the following components: (1) AAV2 or AAV8 inverted terminal repeats (ITRs) that flank the expression cassette; (2) control elements, which include a muscle-specific Spc5-12 promoter, tMCK promoter or CK7 promoter and a poly A signal, including a rabbit beta globin poly A signal; and (3) transgene providing (e.g., coding for) a nucleic acid encoding AUF1 as described herein, including the codon optimized, CpG depleted AUF1 p40 coding sequence. In a specific embodiment, provided are rAAV AUF1 constructs comprising the following components: (1) AAV2 or AAV8 ITRs that flank the expression cassette; (2) control elements, which include a) the muscle-specific Spc5-12 promoter, the tMCK promoter or the CK7 promoter; b) an intron (e.g., a VH4) and c) a poly A signal sequence, such as a rabbit beta globin poly A signal sequence; and (3) a nucleotide sequence encoding AUF1 as described herein, including the codon optimized, CpG depleted AUF1 p40 coding sequence (SEQ ID NO: 17). Optionally, the construct includes a WPRE element 3′ of the coding sequence and 5′ of the polyA signal sequence. The construct may also include 5′ and 3′ “stuffer sequences” between the ITR sequences and the expression cassette comprising the coding sequence and the regulatory operably linked thereto and an SV40 polyA signal sequence adjacent to and 5′ of the 3′ ITR sequence. In certain embodiments, the vectors are single stranded and have a 5′ITR and a 3′ ITR, for example, as provided in Table 2 as SEQ ID NO: 28 and SEQ ID NO: 29, respectively. In certain other embodiments, the vectors are self-complementary vectors and have an altered 5′ ITR, an mITR, for example, that of SEQ ID NO: 30 and a 3′ ITR, as provided in Table 2, such as SEQ ID NO: 29.

Exemplary rAAV genomes and sequences contained within cis plasmids are depicted in FIG. 1 and Table 3, and include:

spc-hu-opti-AUF1-CpG(−):Codon optimized, CpG depleted Human AUF1 sequence driven by Spc5-12 promoter+VH4 intron, including 5′ (141 bp) stuffer and 3′ (893 bp) stuffer with a downstream SV40 polyA signal (reverse); having a nucleotide sequence of SEQ ID NO: 31 (including the ITR sequences).

tMCK-huAUF1: Codon optimized, CpG depleted Human AUF1 sequence driven by tMCK promoter (no intron), including 5′ (141 bp) stuffer and 3′ (893 bp) stuffer-downstream SV40 polyA signal (reverse); having a nucleotide sequence of SEQ ID NO: 32 (including the ITR sequences)

spc5-12-hu-opti-AUF1-WPRE: Codon optimized, CpG depleted Human AUF1 sequence driven by Spc5-12 promoter+VH4 intron, including 3′ WPRE upstream of polyA (including 5′ (141 bp) stuffer and 3′ (893 bp) stuffer)—downstream SV40 polyA signal (reverse); SEQ ID NO: 33 (including the ITR sequences).

ss-CK7-Hu-AUF1: Codon optimized, CpG depleted Human AUF1 sequence driven by CK7 promoter (no intron), including 5′ (141 bp) stuffer and 3′ (893 bp) stuffer)—downstream SV40 polyA signal (reverse); SEQ ID NO: 34 (including the ITR sequences).

spc-hu-AUF1-No-Intron: Codon optimized, CpG depleted Human AUF1 sequence driven by Spc5-12 promoter (no intron) (including 5′ (141 bp) stuffer and 3′ (893 bp) stuffer)—downstream SV40 polyA signal (reverse); SEQ ID NO: 35 (including ITR sequences).

D(+)-CK7AUF1: Self-complementary vector, Codon optimized, CpG depleted Human AUF1 sequence driven by CK7 promoter (no stuffers); SEQ ID NO:36 (including ITR sequences).

Nucleotide sequences of these AUF1 constructs are presented in Table 3.

TABLE 3

| | Full genome sequence |
|---|---|
| Short description | (ITR to ITR) |
| spc-hu-opti- | CTGCGCGCTCGCTCGCTCACTGAGGCCGCCCGGGCAAAGCCCGGGCGTCG |
| AUF1-CpG(-) | GGCGACCTTTGGTCGCCCGGCCTCAGTGAGCGAGCGAGCGCGCAGAGAGG |
| 3017 bp | GAGTGGCCAACTCCATCACTAGGGGTTCCTCATATGCAGGGTAATGGGGA |
| SEQ ID NO: 31 | TCCTCTAGATATAGCTAGTCGACAAAAAGTACCTCAATAATAAATACAGA |
| | ACTTCTCCTTTCAACCTCTTCCATCACATCAACACCTATGAAGACAATGG |
| | GTTTCTGATTGTGGATCTCTGCTGCTGGAAAGGATTTGAGTTTGTTTATA |
| | ATTACTTATATTTAGTTACCGGTCGGCCGTCCGCCCTCGGCACCATCCTC |
| | ACGACACCCAAATATGGCGACGGGTGAGGAATGGTGGGGAGTTATTTTTA |
| | GAGCGGTGAGGAAGGTGGGCAGGCAGCAGGTGTTGGCGCTCTAAAAATAA |
| | CTCCCGGGAGTTATTTTTAGAGCGGAGGAATGGTGGACACCCAAATATGG |
| | CGACGGTTCCTCACCCGTCGCCATATTTGGGTGTCCGCCCTCGGCCGGGG |
| | CCGCATTCCTGGGGGCCGGGCGGTGCTCCCGCCCGCCTCGATAAAAGGCT |
| | CCGGGGCCGGCGGCGGCCCACGAGCTACCCGGAGGAGCGGGAGGCGCCAA |
| | GCCCGCGGAACAGGTGAGTATCTCAGGGATCCAGACATGGGGATATGGGA |
| | GGTGCCTCTGATCCCAGGGCTCACTGTGGGTCTCTCTGTTCACAGGTTCC |
| | TTAAGGGCCGTGCCACCATGTCTGAGGAACAGTTTGGTGGTGATGGGGCT |
| | GCTGCTGCAGCTACAGCTGCTGTTGGAGGATCTGCTGGGGAACAAGAGGG |
| | TGCCATGGTTGCTGCTACACAAGGTGCTGCAGCTGCTGCTGGTAGTGGTG |
| | CTGGAACAGGTGGTGGAACAGCCAGTGGTGGCACAGAAGGAGGCTCTGCT |
| | GAATCTGAAGGGGCCAAGATTGATGCCAGCAAGAATGAGGAAGATGAGGG |
| | CCACAGCAACAGCTCCCCAAGACACTCTGAAGCTGCCACAGCTCAGAGGG |
| | AAGAGTGGAAGATGTTCATTGGAGGCCTGAGCTGGGACACCACCAAGAAG |
| | GACCTGAAGGACTACTTCAGCAAGTTTGGAGAAGTGGTGGACTGCACCCT |
| | GAAGCTGGACCCTATCACAGGCAGAAGCAGAGGCTTTGGCTTTGTGCTGT |
| | TCAAAGAATCTGAGTCTGTGGACAAAGTGATGGACCAGAAAGAACACAAG |
| | CTGAATGGGAAAGTGATTGACCCCAAGAGGGCCAAAGCCATGAAGACCAA |
| | AGAGCCTGTCAAGAAGATCTTTGTTGGAGGGCTGTCCCCTGACACACCTG |
| | AGGAAAAGATCAGAGAGTACTTTGGAGGATTTGGAGAGGTGGAATCCATT |
| | GAGCTGCCCATGGACAACAAGACCAACAAGAGAAGAGGCTTCTGCTTCAT |
| | CACCTTCAAAGAGGAAGAACCAGTCAAGAAAATCATGGAAAAGAAATACC |
| | ACAATGTGGGCCTGAGCAAGTGTGAAATCAAGGTGGCCATGAGCAAAGAG |
| | CAGTACCAGCAACAACAGCAGTGGGGCTCCAGAGGAGGTTTTGCTGGCAG |
| | AGCTAGAGGCAGAGGTGGTGACCAGCAGTCTGGCTATGGCAAGGTGTCCA |
| | GAAGAGGTGGACATCAGAACAGCTACAAGCCCTACTGATGACGCGTTAAT |
| | GAGGTACCTCGAGGATCTTTTTCCCTCTGCCAAAAATTATGGGGACATCA |
| | TGAAGCCCCTTGAGCATCTGACTTCTGGCTAATAAAGGAAATTTATTTTC |
| | ATTGCAATAGTGTGTTGGAATTTTTTGTGTCTCTCACTCGCATGCTTGAG |
| | CATCCTGCTGGTGGTTACAAGAAACTGTTTGAAACTGTGGAGGAACTGTC |
| | CTCGCCGCTCACAGCTCATGTAACAGGCAGGATCCCCCTCTGGCTCACCG |
| | GCAGTCTCCTTCGATGTGGGCCAGGACTCTTTGAAGTTGGATCTGAGCCA |
| | TTTTACCACCTGTTTGATGGGCAAGCCCTCCTGCACAAGTTTGACTTTAA |
| | AGAAGGACATGTCACATACCACAGAAGGTTCATCCGCACTGATGCTTACG |
| | TACGGGCAATGACTGAGAAAAGGATCGTCATAACAGAATTTGGCACCTGT |
| | GCTTTCCCAGATCCCTGCAAGAATATATTTTCCAGGTTTTTTTCTTACTT |
| | TCGAGGAGTAGAGGTTACTGACAATTGCCCTTGTTAATGTCTACCCAGTG |
| | GGGGAAGATTACTACGCTTGCACAGAGACCAACTTTATTACAAAGATTAA |
| | TCCAGAGACCTTGGAGACAATTAAGCAGGTTGATCTTTGCAACTAAGTCT |
| | CTGTCAATGGGGCCACTGCTCACCCCCACATTGAAAATGATGGAACCGTT |
| | TACAATATTGGTAATTGCTTTGGAAAAAATTTTTCAATTGCCTACAACAT |
| | TGTAAAGATCCCACCACTGCAAGCAGACAAGGAAGATCCAATAAGCAAGT |
| | CAGAGATCGTTGTACAATTCCCCTGCAGTGACCGATTCAAGCCATCTTAC |
| | GTTCATAGTTTTGGTCTGACTCCCAACTATATCGTTTTTGTGGAGACACC |
| | AGTCAAAATTAACCTGTTCAAGTTCCTTTCTTCATGGAGTCTTTGGGGAG |
| | CCAACTACATGGATTGTTTTGAGTCCAATGAAACCATGGGGTTTGGCTTC |
| | ATATTGCTGACAAAAAAAGGAAAAAGTACCTCAATAGACTAGTCGATCCA |
| | GACATGATAAGATACATTGATGAGTTTGGACAAACCACAACTAGAATGCA |
| | GTGAAAAAAATGCTTTATTTGTGAAATTTGTGATGCTATTGCTTTATTTG |
| | TAACCATTATAAGCTGCAATAAACAAGTTGCGGCCGCAGGAACCCCTAGT |
| | GATGGAGTTGGCCACTCCCTCTCTGCGCGCTCGCTCGCTCACTGAGGCCG |
| | GGCGACCAAAGGTCGCCCGACGCCCGGGCTTTGCCCGGGCGGCCTCAGTG |
| | AGCGAGCGAGCGCGCAG |
| tMCK-huAUF1 | CTGCGCGCTCGCTCGCTCACTGAGGCCGCCCGGGCAAAGCCCGGGCGTCG |
| 3314 bp | GGCGACCTTTGGTCGCCCGGCCTCAGTGAGCGAGCGAGCGCGCAGAGAGG |
| SEQ ID NO: 32 | GAGTGGCCAACTCCATCACTAGGGGTTCCTCATATGCAGGGTAATGGGGA |
| | TCCTCTAGATATAGCTAGTCGACAAAAAGTACCTCAATAATAAATACAGA |
| | ACTTCTCCTTTCAACCTCTTCCATCACATCAACACCTATGAAGACAATGG |
| | GTTTCTGATTGTGGATCTCTGCTGCTGGAAAGGATTTGAGTTTGTTTATA |
| | ATTACTTATATTTAGTTACCGGTGCCACTACGGGTCTAGGCTGCCCATGT |
| | AAGGAGGCAAGGCCTGGGGACACCCGAGATGCCTGGTTATAATTAACCCC |
| | AACACCTGCTGCCCCCCCCCCCCAACACCTGCTGCCTGAGCCTGAGCGGT |
| | TACCCCACCCCGGTGCCTGGGTCTTAGGCTCTGTACACCATGGAGGAGAA |
| | GCTCGCTCTAAAAATAACCCTGTCCCTGGTGGATCGCCACTACGGGTCTA |
| | GGCTGCCCATGTAAGGAGGCAAGGCCTGGGGACACCCGAGATGCCTGGTT |
| | ATAATTAACCCCAACACCTGCTGCCCCCCCCCCCCAACACCTGCTGCCTG |
| | AGCCTGAGCGGTTACCCCACCCCGGTGCCTGGGTCTTAGGCTCTGTACAC |
| | CATGGAGGAGAAGCTCGCTCTAAAAATAACCCTGTCCCTGGTGGATCGCC |
| | ACTACGGGTCTAGGCTGCCCATGTAAGGAGGCAAGGCCTGGGGACACCCG |
| | AGATGCCTGGTTATAATTAACCCCAACACCTGCTGCCCCCCCCCCCCAAC |
| | ACCTGCTGCCTGAGCCTGAGCGGTTACCCCACCCCGGTGCCTGGGTCTTA |
| | GGCTCTGTACACCATGGAGGAGAAGCTCGCTCTAAAAATAACCCTGTCCC |
| | TGGTGGATCCCTCCCTGGGGACAGCCCCTCCTGGCTAGTCACACCCTGTA |
| | GGCTCCTCTATATAACCCAGGGGCACAGGGGCTGCCCCCGGGTCACCTTA |
| | AGGGCCGTGCCACCATGTCTGAGGAACAGTTTGGTGGTGATGGGGCTGCT |
| | GCTGCAGCTACAGCTGCTGTTGGAGGATCTGCTGGGGAACAAGAGGGTGC |
| | CATGGTTGCTGCTACACAAGGTGCTGCAGCTGCTGCTGGTAGTGGTGCTG |
| | GAACAGGTGGTGGAACAGCCAGTGGTGGCACAGAAGGAGGCTCTGCTGAA |
| | TCTGAAGGGGCCAAGATTGATGCCAGCAAGAATGAGGAAGATGAGGGCCA |
| | CAGCAACAGCTCCCCAAGACACTCTGAAGCTGCCACAGCTCAGAGGGAAG |
| | AGTGGAAGATGTTCATTGGAGGCCTGAGCTGGGACACCACCAAGAAGGAC |
| | CTGAAGGACTACTTCAGCAAGTTTGGAGAAGTGGTGGACTGCACCCTGAA |
| | GCTGGACCCTATCACAGGCAGAAGCAGAGGCTTTGGCTTTGTGCTGTTCA |
| | AAGAATCTGAGTCTGTGGACAAAGTGATGGACCAGAAAGAACACAAGCTG |
| | AATGGGAAAGTGATTGACCCCAAGAGGGCCAAAGCCATGAAGACCAAAGA |
| | GCCTGTCAAGAAGATCTTTGTTGGAGGGCTGTCCCCTGACACACCTGAGG |
| | AAAAGATCAGAGAGTACTTTGGAGGATTTGGAGAGGTGGAATCCATTGAG |
| | CTGCCCATGGACAACAAGACCAACAAGAGAAGAGGCTTCTGCTTCATCAC |
| | CTTCAAAGAGGAAGAACCAGTCAAGAAAATCATGGAAAAGAAATACCACA |
| | ATGTGGGCCTGAGCAAGTGTGAAATCAAGGTGGCCATGAGCAAAGAGCAG |
| | TACCAGCAACAACAGCAGTGGGGCTCCAGAGGAGGTTTTGCTGGCAGAGC |
| | TAGAGGCAGAGGTGGTGACCAGCAGTCTGGCTATGGCAAGGTGTCCAGAA |
| | GAGGTGGACATCAGAACAGCTACAAGCCCTACTGATGACGCGTTAATGAG |
| | GTACCTCGAGGATCTTTTTCCCTCTGCCAAAAATTATGGGGACATCATGA |
| | AGCCCCTTGAGCATCTGACTTCTGGCTAATAAAGGAAATTTATTTTCATT |
| | GCAATAGTGTGTTGGAATTTTTTGTGTCTCTCACTCGCATGCTTGAGCAT |
| | CCTGCTGGTGGTTACAAGAAACTGTTTGAAACTGTGGAGGAACTGTCCTC |
| | GCCGCTCACAGCTCATGTAACAGGCAGGATCCCCCTCTGGCTCACCGGCA |
| | GTCTCCTTCGATGTGGGCCAGGACTCTTTGAAGTTGGATCTGAGCCATTT |
| | TACCACCTGTTTGATGGGCAAGCCCTCCTGCACAAGTTTGACTTTAAAGA |
| | AGGACATGTCACATACCACAGAAGGTTCATCCGCACTGATGCTTACGTAC |
| | GGGCAATGACTGAGAAAAGGATCGTCATAACAGAATTTGGCACCTGTGCT |
| | TTCCCAGATCCCTGCAAGAATATATTTTCCAGGTTTTTTTCTTACTTTCG |
| | AGGAGTAGAGGTTACTGACAATTGCCCTTGTTAATGTCTACCCAGTGGGG |
| | GAAGATTACTACGCTTGCACAGAGACCAACTTTATTACAAAGATTAATCC |
| | AGAGACCTTGGAGACAATTAAGCAGGTTGATCTTTGCAACTAAGTCTCTG |
| | TCAATGGGGCCACTGCTCACCCCCACATTGAAAATGATGGAACCGTTTAC |
| | AATATTGGTAATTGCTTTGGAAAAAATTTTTCAATTGCCTACAACATTGT |
| | AAAGATCCCACCACTGCAAGCAGACAAGGAAGATCCAATAAGCAAGTCAG |
| | AGATCGTTGTACAATTCCCCTGCAGTGACCGATTCAAGCCATCTTACGTT |
| | CATAGTTTTGGTCTGACTCCCAACTATATCGTTTTTGTGGAGACACCAGT |
| | CAAAATTAACCTGTTCAAGTTCCTTTCTTCATGGAGTCTTTGGGGAGCCA |
| | ACTACATGGATTGTTTTGAGTCCAATGAAACCATGGGGTTTGGCTTCATA |
| | TTGCTGACAAAAAAAGGAAAAAGTACCTCAATAGACTAGTCGATCCAGAC |
| | ATGATAAGATACATTGATGAGTTTGGACAAACCACAACTAGAATGCAGTG |
| | AAAAAAATGCTTTATTTGTGAAATTTGTGATGCTATTGCTTTATTTGTAA |
| | CCATTATAAGCTGCAATAAACAAGTTGCGGCCGCAGGAACCCCTAGTGAT |
| | GGAGTTGGCCACTCCCTCTCTGCGCGCTCGCTCGCTCACTGAGGCCGGGC |
| | GACCAAAGGTCGCCCGACGCCCGGGCTTTGCCCGGGCGGCCTCAGTGAGC |
| | GAGCGAGCGCGCAG |
| spc5-12-hu-opti- | CTGCGCGCTCGCTCGCTCACTGAGGCCGCCCGGGCAAAGCCCGGGCGTCG |
| AUF1-WPRE | GGCGACCTTTGGTCGCCCGGCCTCAGTGAGCGAGCGAGCGCGCAGAGAGG |
| 3600 bp | GAGTGGCCAACTCCATCACTAGGGGTTCCTCATATGCAGGGTAATGGGGA |
| SEQ ID NO: 33 | TCCTCTAGATATAGCTAGTCGACAAAAAGTACCTCAATAATAAATACAGA |
| | ACTTCTCCTTTCAACCTCTTCCATCACATCAACACCTATGAAGACAATGG |
| | GTTTCTGATTGTGGATCTCTGCTGCTGGAAAGGATTTGAGTTTGTTTATA |
| | ATTACTTATATTTAGTTACCGGTCGGCCGTCCGCCCTCGGCACCATCCTC |
| | ACGACACCCAAATATGGCGACGGGTGAGGAATGGTGGGGAGTTATTTTTA |
| | GAGCGGTGAGGAAGGTGGGCAGGCAGCAGGTGTTGGCGCTCTAAAAATAA |
| | CTCCCGGGAGTTATTTTTAGAGCGGAGGAATGGTGGACACCCAAATATGG |
| | CGACGGTTCCTCACCCGTCGCCATATTTGGGTGTCCGCCCTCGGCCGGGG |
| | CCGCATTCCTGGGGGCCGGGCGGTGCTCCCGCCCGCCTCGATAAAAGGCT |
| | CCGGGGCCGGCGGCGGCCCACGAGCTACCCGGAGGAGCGGGAGGCGCCAA |
| | GCCCGCGGAACAGGTGAGTATCTCAGGGATCCAGACATGGGGATATGGGA |
| | GGTGCCTCTGATCCCAGGGCTCACTGTGGGTCTCTCTGTTCACAGGTTCC |
| | TTAAGGGCCGTGCCACCATGTCTGAGGAACAGTTTGGTGGTGATGGGGCT |
| | GCTGCTGCAGCTACAGCTGCTGTTGGAGGATCTGCTGGGGAACAAGAGGG |
| | TGCCATGGTTGCTGCTACACAAGGTGCTGCAGCTGCTGCTGGTAGTGGTG |
| | CTGGAACAGGTGGTGGAACAGCCAGTGGTGGCACAGAAGGAGGCTCTGCT |
| | GAATCTGAAGGGGCCAAGATTGATGCCAGCAAGAATGAGGAAGATGAGGG |
| | CCACAGCAACAGCTCCCCAAGACACTCTGAAGCTGCCACAGCTCAGAGGG |
| | AAGAGTGGAAGATGTTCATTGGAGGCCTGAGCTGGGACACCACCAAGAAG |
| | GACCTGAAGGACTACTTCAGCAAGTTTGGAGAAGTGGTGGACTGCACCCT |
| | GAAGCTGGACCCTATCACAGGCAGAAGCAGAGGCTTTGGCTTTGTGCTGT |
| | TCAAAGAATCTGAGTCTGTGGACAAAGTGATGGACCAGAAAGAACACAAG |
| | CTGAATGGGAAAGTGATTGACCCCAAGAGGGCCAAAGCCATGAAGACCAA |
| | AGAGCCTGTCAAGAAGATCTTTGTTGGAGGGCTGTCCCCTGACACACCTG |
| | AGGAAAAGATCAGAGAGTACTTTGGAGGATTTGGAGAGGTGGAATCCATT |
| | GAGCTGCCCATGGACAACAAGACCAACAAGAGAAGAGGCTTCTGCTTCAT |
| | CACCTTCAAAGAGGAAGAACCAGTCAAGAAAATCATGGAAAAGAAATACC |
| | ACAATGTGGGCCTGAGCAAGTGTGAAATCAAGGTGGCCATGAGCAAAGAG |
| | CAGTACCAGCAACAACAGCAGTGGGGCTCCAGAGGAGGTTTTGCTGGCAG |
| | AGCTAGAGGCAGAGGTGGTGACCAGCAGTCTGGCTATGGCAAGGTGTCCA |
| | GAAGAGGTGGACATCAGAACAGCTACAAGCCCTACTGATGACGCGTAATC |
| | AACCTCTGGATTACAAAATTTGTGAAAGATTGACTGGTATTCTTAACTAT |
| | GTTGCTCCTTTTACGCTATGTGGATACGCTGCTTTAATGCCTTTGTATCA |
| | TGCTATTGCTTCCCGTATGGCTTTCATTTTCTCCTCCTTGTATAAATCCT |
| | GGTTGCTGTCTCTTTATGAGGAGTTGTGGCCCGTTGTCAGGCAACGTGGC |
| | GTGGTGTGCACTGTGTTTGCTGACGCAACCCCCACTGGTTGGGGCATTGC |
| | CACCACCTGTCAGCTCCTTTCCGGGACTTTCGCTTTCCCCCTCCCTATTG |
| | CCACGGCGGAACTCATCGCCGCCTGCCTTGCCCGCTGCTGGACAGGGGCT |
| | CGGCTGTTGGGCACTGACAATTCCGTGGTGTTGTCGGGGAAATCATCGTC |
| | CTTTCCTTGGCTGCTCGCCTGTGTTGCCACCTGGATTCTGCGCGGGACGT |
| | CCTTCTGCTACGTCCCTTCGGCCCTCAATCCAGCGGACCTTCCTTCCCGC |
| | GGCCTGCTGCCGGCTCTGCGGCCTCTTCCGCGTCTTCGCCTTCGCCCTCA |
| | GACGAGTCGGATCTCCCTTTGGGCCGCCTCCCCGCGGTACCTCGAGGATC |
| | TTTTTCCCTCTGCCAAAAATTATGGGGACATCATGAAGCCCCTTGAGCAT |
| | CTGACTTCTGGCTAATAAAGGAAATTTATTTTCATTGCAATAGTGTGTTG |
| | GAATTTTTTGTGTCTCTCACTCGCATGCTTGAGCATCCTGCTGGTGGTTA |
| | CAAGAAACTGTTTGAAACTGTGGAGGAACTGTCCTCGCCGCTCACAGCTC |
| | ATGTAACAGGCAGGATCCCCCTCTGGCTCACCGGCAGTCTCCTTCGATGT |
| | GGGCCAGGACTCTTTGAAGTTGGATCTGAGCCATTTTACCACCTGTTTGA |
| | TGGGCAAGCCCTCCTGCACAAGTTTGACTTTAAAGAAGGACATGTCACAT |
| | ACCACAGAAGGTTCATCCGCACTGATGCTTACGTACGGGCAATGACTGAG |
| | AAAAGGATCGTCATAACAGAATTTGGCACCTGTGCTTTCCCAGATCCCTG |
| | CAAGAATATATTTTCCAGGTTTTTTTCTTACTTTCGAGGAGTAGAGGTTA |
| | CTGACAATTGCCCTTGTTAATGTCTACCCAGTGGGGGAAGATTACTACGC |
| | TTGCACAGAGACCAACTTTATTACAAAGATTAATCCAGAGACCTTGGAGA |
| | CAATTAAGCAGGTTGATCTTTGCAACTAAGTCTCTGTCAATGGGGCCACT |
| | GCTCACCCCCACATTGAAAATGATGGAACCGTTTACAATATTGGTAATTG |
| | CTTTGGAAAAAATTTTTCAATTGCCTACAACATTGTAAAGATCCCACCAC |
| | TGCAAGCAGACAAGGAAGATCCAATAAGCAAGTCAGAGATCGTTGTACAA |
| | TTCCCCTGCAGTGACCGATTCAAGCCATCTTACGTTCATAGTTTTGGTCT |
| | GACTCCCAACTATATCGTTTTTGTGGAGACACCAGTCAAAATTAACCTGT |
| | TCAAGTTCCTTTCTTCATGGAGTCTTTGGGGAGCCAACTACATGGATTGT |
| | TTTGAGTCCAATGAAACCATGGGGTTTGGCTTCATATTGCTGACAAAAAA |
| | AGGAAAAAGTACCTCAATAGACTAGTCGATCCAGACATGATAAGATACAT |
| | TGATGAGTTTGGACAAACCACAACTAGAATGCAGTGAAAAAAATGCTTTA |
| | TTTGTGAAATTTGTGATGCTATTGCTTTATTTGTAACCATTATAAGCTGC |
| | AATAAACAAGTTGCGGCCGCAGGAACCCCTAGTGATGGAGTTGGCCACTC |
| | CCTCTCTGCGCGCTCGCTCGCTCACTGAGGCCGGGCGACCAAAGGTCGCC |
| | CGACGCCCGGGCTTTGCCCGGGCGGCCTCAGTGAGCGAGCGAGCGCGCAG |
| ss-CK7-Hu- | CTGCGCGCTCGCTCGCTCACTGAGGCCGCCCGGGCAAAGCCCGGGCGTCG |
| AUF1 | GGCGACCTTTGGTCGCCCGGCCTCAGTGAGCGAGCGAGCGCGCAGAGAGG |
| 3169 bp | GAGTGGCCAACTCCATCACTAGGGGTTCCTCATATGCAGGGTAATGGGGA |
| SEQ ID NO: 34 | TCCTCTAGATATAGCTAGTCGACAAAAAGTACCTCAATAATAAATACAGA |
| | ACTTCTCCTTTCAACCTCTTCCATCACATCAACACCTATGAAGACAATGG |
| | GTTTCTGATTGTGGATCTCTGCTGCTGGAAAGGATTTGAGTTTGTTTATA |
| | ATTACTTATATTTAGTTACCGGTCCACTACGGGTTTAGGCTGCCCATGTA |
| | AGGAGGCAAGGCCTGGGGACACCCGAGATGCCTGGTTATAATTAACCCAG |
| | ACATGTGGCTGCCCCCCCCCCCCCCAACACCTGCTGCCTCTAAAAATAAC |
| | CCTGTCCCTGGTGGATCCCCTGCATGCGAAGATCTTCGAACAAGGCTGTG |
| | GGGGACTGAGGGCAGGCTGTAACAGGCTTGGGGGCCAGGGCTTATACGTG |
| | CCTGGGACTCCCAAAGTATTACTGTTCCATGTTCCCGGCGAAGGGCCAGC |
| | TGTCCCCCGCCAGCTAGACTCAGCACTTAGITTAGGAACCAGTGAGCAAG |
| | TCAGCCCTTGGGGCAGCCCATACAAGGCCATGGGGCTGGGCAAGCTGCAC |
| | GCCTGGGTCCGGGGTGGGCACGGTGCCCGGGCAACGAGCTGAAAGCTCAT |
| | CTGCTCTCAGGGGCCCCTCCCTGGGGACAGCCCCTCCTGGCTAGTCACAC |
| | CCTGTAGGCTCCTCTATATAACCCAGGGGCACAGGGGCTGCCCTCATTCT |
| | ACCACCACCTCCACAGCACAGACAGACACTCAGGAGCCAGCCAGCGTCGA |
| | CCTTAAGGGCCGTGCCACCATGTCTGAGGAACAGTTTGGTGGTGATGGGG |
| | CTGCTGCTGCAGCTACAGCTGCTGTTGGAGGATCTGCTGGGGAACAAGAG |
| | GGTGCCATGGTTGCTGCTACACAAGGTGCTGCAGCTGCTGCTGGTAGTGG |
| | TGCTGGAACAGGTGGTGGAACAGCCAGTGGTGGCACAGAAGGAGGCTCTG |
| | CTGAATCTGAAGGGGCCAAGATTGATGCCAGCAAGAATGAGGAAGATGAG |
| | GGCCACAGCAACAGCTCCCCAAGACACTCTGAAGCTGCCACAGCTCAGAG |
| | GGAAGAGTGGAAGATGTTCATTGGAGGCCTGAGCTGGGACACCACCAAGA |
| | AGGACCTGAAGGACTACTTCAGCAAGTTTGGAGAAGTGGTGGACTGCACC |
| | CTGAAGCTGGACCCTATCACAGGCAGAAGCAGAGGCTTTGGCTTTGTGCT |
| | GTTCAAAGAATCTGAGTCTGTGGACAAAGTGATGGACCAGAAAGAACACA |
| | AGCTGAATGGGAAAGTGATTGACCCCAAGAGGGCCAAAGCCATGAAGACC |
| | AAAGAGCCTGTCAAGAAGATCTTTGTTGGAGGGCTGTCCCCTGACACACC |
| | TGAGGAAAAGATCAGAGAGTACTTTGGAGGATTTGGAGAGGTGGAATCCA |
| | TTGAGCTGCCCATGGACAACAAGACCAACAAGAGAAGAGGCTTCTGCTTC |
| | ATCACCTTCAAAGAGGAAGAACCAGTCAAGAAAATCATGGAAAAGAAATA |
| | CCACAATGTGGGCCTGAGCAAGTGTGAAATCAAGGTGGCCATGAGCAAAG |
| | AGCAGTACCAGCAACAACAGCAGTGGGGCTCCAGAGGAGGTTTTGCTGGC |
| | AGAGCTAGAGGCAGAGGTGGTGACCAGCAGTCTGGCTATGGCAAGGTGTC |
| | CAGAAGAGGTGGACATCAGAACAGCTACAAGCCCTACTGATGACGCGTTA |
| | ATGAGGTACCTCGAGGATCTTTTTCCCTCTGCCAAAAATTATGGGGACAT |
| | CATGAAGCCCCTTGAGCATCTGACTTCTGGCTAATAAAGGAAATTTATTT |
| | TCATTGCAATAGTGTGTTGGAATTTTTTGTGTCTCTCACTCGCATGCTTG |
| | AGCATCCTGCTGGTGGTTACAAGAAACTGTTTGAAACTGTGGAGGAACTG |
| | TCCTCGCCGCTCACAGCTCATGTAACAGGCAGGATCCCCCTCTGGCTCAC |
| | CGGCAGTCTCCTTCGATGTGGGCCAGGACTCTTTGAAGTTGGATCTGAGC |
| | CATTTTACCACCTGTTTGATGGGCAAGCCCTCCTGCACAAGTTTGACTTT |
| | AAAGAAGGACATGTCACATACCACAGAAGGTTCATCCGCACTGATGCTTA |
| | CGTACGGGCAATGACTGAGAAAAGGATCGTCATAACAGAATTTGGCACCT |
| | GTGCTTTCCCAGATCCCTGCAAGAATATATTTTCCAGGTTTTTTTCTTAC |
| | TTTCGAGGAGTAGAGGTTACTGACAATTGCCCTTGTTAATGTCTACCCAG |
| | TGGGGGAAGATTACTACGCTTGCACAGAGACCAACTTTATTACAAAGATT |
| | AATCCAGAGACCTTGGAGACAATTAAGCAGGTTGATCTTTGCAACTAAGT |
| | CTCTGTCAATGGGGCCACTGCTCACCCCCACATTGAAAATGATGGAACCG |
| | TTTACAATATTGGTAATTGCTTTGGAAAAAATTTTTCAATTGCCTACAAC |
| | ATTGTAAAGATCCCACCACTGCAAGCAGACAAGGAAGATCCAATAAGCAA |
| | GTCAGAGATCGTTGTACAATTCCCCTGCAGTGACCGATTCAAGCCATCTT |
| | ACGTTCATAGTTTTGGTCTGACTCCCAACTATATCGTTTTTGTGGAGACA |
| | CCAGTCAAAATTAACCTGTTCAAGTTCCTTTCTTCATGGAGTCTTTGGGG |
| | AGCCAACTACATGGATTGTTTTGAGTCCAATGAAACCATGGGGTTTGGCT |
| | TCATATTGCTGACAAAAAAAGGAAAAAGTACCTCAATAGACTAGTCGATC |
| | CAGACATGATAAGATACATTGATGAGTTTGGACAAACCACAACTAGAATG |
| | CAGTGAAAAAAATGCTTTATTTGTGAAATTTGTGATGCTATTGCTTTATT |
| | TGTAACCATTATAAGCTGCAATAAACAAGTTGCGGCCGCAGGAACCCCTA |
| | GTGATGGAGTTGGCCACTCCCTCTCTGCGCGCTCGCTCGCTCACTGAGGC |
| | CGGGCGACCAAAGGTCGCCCGACGCCCGGGCTTTGCCCGGGCGGCCTCAG |
| | TGAGCGAGCGAGCGCGCAG |
| spc-hu-AUF1- | CTGCGCGCTCGCTCGCTCACTGAGGCCGCCCGGGCAAAGCCCGGGCGTCG |
| No-Intron | GGCGACCTTTGGTCGCCCGGCCTCAGTGAGCGAGCGAGCGCGCAGAGAGG |
| 2921 bp | GAGTGGCCAACTCCATCACTAGGGGTTCCTCATATGCAGGGTAATGGGGA |
| SEQ ID NO: 35 | TCCTCTAGATATAGCTAGTCGACAAAAAGTACCTCAATAATAAATACAGA |
| | ACTTCTCCTTTCAACCTCTTCCATCACATCAACACCTATGAAGACAATGG |
| | GTTTCTGATTGTGGATCTCTGCTGCTGGAAAGGATTTGAGTTTGTTTATA |
| | ATTACTTATATTTAGTTACCGGTCGGCCGTCCGCCCTCGGCACCATCCTC |
| | ACGACACCCAAATATGGCGACGGGTGAGGAATGGTGGGGAGTTATTTTTA |
| | GAGCGGTGAGGAAGGTGGGCAGGCAGCAGGTGTTGGCGCTCTAAAAATAA |
| | CTCCCGGGAGTTATTTTTAGAGCGGAGGAATGGTGGACACCCAAATATGG |
| | CGACGGTTCCTCACCCGTCGCCATATTTGGGTGTCCGCCCTCGGCCGGGG |
| | CCGCATTCCTGGGGGCCGGGCGGTGCTCCCGCCCGCCTCGATAAAAGGCT |
| | CCGGGGCCGGCGGCGGCCCACGAGCTACCCGGAGGAGCGGGAGGCGCCAA |
| | GCCCTTAAGGGCCGTGCCACCATGTCTGAGGAACAGTTTGGTGGTGATGG |
| | GGCTGCTGCTGCAGCTACAGCTGCTGTTGGAGGATCTGCTGGGGAACAAG |
| | AGGGTGCCATGGTTGCTGCTACACAAGGTGCTGCAGCTGCTGCTGGTAGT |
| | GGTGCTGGAACAGGTGGTGGAACAGCCAGTGGTGGCACAGAAGGAGGCTC |
| | TGCTGAATCTGAAGGGGCCAAGATTGATGCCAGCAAGAATGAGGAAGATG |
| | AGGGCCACAGCAACAGCTCCCCAAGACACTCTGAAGCTGCCACAGCTCAG |
| | AGGGAAGAGTGGAAGATGTTCATTGGAGGCCTGAGCTGGGACACCACCAA |
| | GAAGGACCTGAAGGACTACTTCAGCAAGTTTGGAGAAGTGGTGGACTGCA |
| | CCCTGAAGCTGGACCCTATCACAGGCAGAAGCAGAGGCTTTGGCTTTGTG |
| | CTGTTCAAAGAATCTGAGTCTGTGGACAAAGTGATGGACCAGAAAGAACA |
| | CAAGCTGAATGGGAAAGTGATTGACCCCAAGAGGGCCAAAGCCATGAAGA |
| | CCAAAGAGCCTGTCAAGAAGATCTTTGTTGGAGGGCTGTCCCCTGACACA |
| | CCTGAGGAAAAGATCAGAGAGTACTTTGGAGGATTTGGAGAGGTGGAATC |
| | CATTGAGCTGCCCATGGACAACAAGACCAACAAGAGAAGAGGCTTCTGCT |
| | TCATCACCTTCAAAGAGGAAGAACCAGTCAAGAAAATCATGGAAAAGAAA |
| | TACCACAATGTGGGCCTGAGCAAGTGTGAAATCAAGGTGGCCATGAGCAA |
| | AGAGCAGTACCAGCAACAACAGCAGTGGGGCTCCAGAGGAGGTTTTGCTG |
| | GCAGAGCTAGAGGCAGAGGTGGTGACCAGCAGTCTGGCTATGGCAAGGTG |
| | TCCAGAAGAGGTGGACATCAGAACAGCTACAAGCCCTACTGATGACGCGT |
| | TAATGAGGTACCTCGAGGATCTTTTTCCCTCTGCCAAAAATTATGGGGAC |
| | ATCATGAAGCCCCTTGAGCATCTGACTTCTGGCTAATAAAGGAAATTTAT |
| | TTTCATTGCAATAGTGTGTTGGAATTTTTTGTGTCTCTCACTCGCATGCT |
| | TGAGCATCCTGCTGGTGGTTACAAGAAACTGTTTGAAACTGTGGAGGAAC |
| | TGTCCTCGCCGCTCACAGCTCATGTAACAGGCAGGATCCCCCTCTGGCTC |
| | ACCGGCAGTCTCCTTCGATGTGGGCCAGGACTCTTTGAAGTTGGATCTGA |
| | GCCATTTTACCACCTGTTTGATGGGCAAGCCCTCCTGCACAAGTTTGACT |
| | TTAAAGAAGGACATGTCACATACCACAGAAGGTTCATCCGCACTGATGCT |
| | TACGTACGGGCAATGACTGAGAAAAGGATCGTCATAACAGAATTTGGCAC |
| | CTGTGCTTTCCCAGATCCCTGCAAGAATATATTTTCCAGGTTTTTTTCTT |
| | ACTTTCGAGGAGTAGAGGTTACTGACAATTGCCCTTGTTAATGTCTACCC |
| | AGTGGGGGAAGATTACTACGCTTGCACAGAGACCAACTTTATTACAAAGA |
| | TTAATCCAGAGACCTTGGAGACAATTAAGCAGGTTGATCTTTGCAACTAA |
| | GTCTCTGTCAATGGGGCCACTGCTCACCCCCACATTGAAAATGATGGAAC |
| | CGTTTACAATATTGGTAATTGCTTTGGAAAAAATTTTTCAATTGCCTACA |
| | ACATTGTAAAGATCCCACCACTGCAAGCAGACAAGGAAGATCCAATAAGC |
| | AAGTCAGAGATCGTTGTACAATTCCCCTGCAGTGACCGATTCAAGCCATC |
| | TTACGTTCATAGTTTTGGTCTGACTCCCAACTATATCGTTTTTGTGGAGA |
| | CACCAGTCAAAATTAACCTGTTCAAGTTCCTTTCTTCATGGAGTCTTTGG |
| | GGAGCCAACTACATGGATTGTTTTGAGTCCAATGAAACCATGGGGTTTGG |
| | CTTCATATTGCTGACAAAAAAAGGAAAAAGTACCTCAATAGACTAGTCGA |
| | TCCAGACATGATAAGATACATTGATGAGTTTGGACAAACCACAACTAGAA |
| | TGCAGTGAAAAAAATGCTTTATTTGTGAAATTTGTGATGCTATTGCTTTA |
| | TTTGTAACCATTATAAGCTGCAATAAACAAGTTGCGGCCGCAGGAACCCC |
| | TAGTGATGGAGTTGGCCACTCCCTCTCTGCGCGCTCGCTCGCTCACTGAG |
| | GCCGGGCGACCAAAGGTCGCCCGACGCCCGGGCTTTGCCCGGGCGGCCTC |
| | AGTGAGCGAGCGAGCGCGCAG |
| D(+)-CK7AUF1 | CTGCGCGCTCGCTCGCTCACTGAGGCCGCCCGGGCAAAGCCCGGGCGTCG |
| 1987 bp | GGCGACCTTTGGTCGCCCGGCCTCAGTGAGCGAGCGAGCGCGCAGAGAGG |
| SEQ ID NO: 36 | GAGTGGAATTCACGCGTACCTAGACCACTACGGGTTTAGGCTGCCCATGT |
| | AAGGAGGCAAGGCCTGGGGACACCCGAGATGCCTGGTTATAATTAACCCA |
| | GACATGTGGCTGCCCCCCCCCCCCCCAACACCTGCTGCCTCTAAAAATAA |
| | CCCTGTCCCTGGTGGATCCCCTGCATGCGAAGATCTTCGAACAAGGCTGT |
| | GGGGGACTGAGGGCAGGCTGTAACAGGCTTGGGGGCCAGGGCTTATACGT |
| | GCCTGGGACTCCCAAAGTATTACTGTTCCATGTTCCCGGCGAAGGGCCAG |
| | CTGTCCCCCGCCAGCTAGACTCAGCACTTAGTTTAGGAACCAGTGAGCAA |
| | GTCAGCCCTTGGGGCAGCCCATACAAGGCCATGGGGCTGGGCAAGCTGCA |
| | CGCCTGGGTCCGGGGTGGGCACGGTGCCCGGGCAACGAGCTGAAAGCTCA |
| | TCTGCTCTCAGGGGCCCCTCCCTGGGGACAGCCCCTCCTGGCTAGTCACA |
| | CCCTGTAGGCTCCTCTATATAACCCAGGGGCACAGGGGCTGCCCTCATTC |
| | TACCACCACCTCCACAGCACAGACAGACACTCAGGAGCCAGCCAGCGTCG |
| | AGCCGCGGAACGGCCGTGCCACCATGTCTGAGGAACAGTTTGGTGGTGAT |
| | GGGGCTGCTGCTGCAGCTACAGCTGCTGTTGGAGGATCTGCTGGGGAACA |
| | AGAGGGTGCCATGGTTGCTGCTACACAAGGTGCTGCAGCTGCTGCTGGTA |
| | GTGGTGCTGGAACAGGTGGTGGAACAGCCAGTGGTGGCACAGAAGGAGGC |
| | TCTGCTGAATCTGAAGGGGCCAAGATTGATGCCAGCAAGAATGAGGAAGA |
| | TGAGGGCCACAGCAACAGCTCCCCAAGACACTCTGAAGCTGCCACAGCTC |
| | AGAGGGAAGAGTGGAAGATGTTCATTGGAGGCCTGAGCTGGGACACCACC |
| | AAGAAGGACCTGAAGGACTACTTCAGCAAGTTTGGAGAAGTGGTGGACTG |
| | CACCCTGAAGCTGGACCCTATCACAGGCAGAAGCAGAGGCTTTGGCTTTG |
| | TGCTGTTCAAAGAATCTGAGTCTGTGGACAAAGTGATGGACCAGAAAGAA |
| | CACAAGCTGAATGGGAAAGTGATTGACCCCAAGAGGGCCAAAGCCATGAA |
| | GACCAAAGAGCCTGTCAAGAAGATCTTTGTTGGAGGGCTGTCCCCTGACA |
| | CACCTGAGGAAAAGATCAGAGAGTACTTTGGAGGATTTGGAGAGGTGGAA |
| | TCCATTGAGCTGCCCATGGACAACAAGACCAACAAGAGAAGAGGCTTCTG |
| | CTTCATCACCTTCAAAGAGGAAGAACCAGTCAAGAAAATCATGGAAAAGA |
| | AATACCACAATGTGGGCCTGAGCAAGTGTGAAATCAAGGTGGCCATGAGC |
| | AAAGAGCAGTACCAGCAACAACAGCAGTGGGGCTCCAGAGGAGGTTTTGC |
| | TGGCAGAGCTAGAGGCAGAGGTGGTGACCAGCAGTCTGGCTATGGCAAGG |
| | TGTCCAGAAGAGGTGGACATCAGAACAGCTACAAGCCCTACTGATGAAGC |
| | GGCCATCCTCGAGGGTACCGATCTTTTTCCCTCTGCCAAAAATTATGGGG |
| | ACATCATGAAGCCCCTTGAGCATCTGACTTCTGGCTAATAAAGGAAATTT |
| | ATTTTCATTGCAATAGTGTGTTGGAATTTTTTGTGTCTCTCACTCGGCTA |
| | GCGAAGCAATTCTAGCAGGCATGCTGGGGAGAGATCGATCTGAGGAACCC |
| | CTAGTGATGGAGTTGGCCACTCCCTCTCTGCGCGCTCGCTCGCTCACTGA |
| | GGCCGCCCGGGCAAAGCCCGGGCGTCGGGCGACCTTTGGTCGCCCGGCCT |
| | CAGTGAGCGAGCGAGCGCGCAGAGAGGGAGTGGCCAA |

Provided are rAAV particles comprising these recombinant genomes encoding AUF1 and cis plasmid vectors comprising these sequences used to produce rAAV particles, including AAV8 serotype, AAV9 serotype or AAVhu.32 serotype particles as described herein, which may be useful in the methods for treating, preventing or ameliorating diseases or disorders in subjects, including human subjects, in need thereof by promoting or increasing muscle mass, muscle function or performance, and/or reducing or reversing muscle atrophy as described further herein. In further embodiments, these rAAV genomes and rAAV particles produced from cis plasmids comprising these sequences described herein, including those in Table 3, are administered in combination with an rAAV comprising a transgene encoding a microdystrophin for treatment of dystrophinopathies in subjects, including human subjects, in need thereof, including Duchenne muscular dystrophy (DMD), Becker muscular dystrophy (BMD), X-linked dilated cardiomyopathy, or limb-girdle muscular dystrophy. The microdystrophin rAAV particles for use herein, include those comprising transgenes encoding microdystrophins having an amino acid sequence of SEQ ID NO: 52, 53 or 54, encoded by a nucleotide sequence of SEQ ID NO: 91, 92, or 93, and those rAAV particles having a genome having the sequence of SEQ ID NO: 94, 95, or 96, which may be an AAV8, AAV9, or AAVhu.32 serotype. In other embodiments, provided are methods of treating dystrophinopathies in subjects, including human subjects, in need thereof by administering an rAAV gene therapy vector comprising a transgene encoding AUF1, including the rAAV genomes in Table 3, in combination with another therapy effective to treat dystrophinopathies, including those described herein.

### 5.3. Microdystrophin Vectors

### 5.3.1 Microdystrophins Encoded by the Transgenes

In some embodiments, encoded by the one of transgenes provided herein for the methods of the invention are microdystrophins that consist of dystrophin domains arranged amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is a hinge 4 region of dystrophin, CR is a cysteine-rich region of dystrophin and CT is the C terminal domain (and comprises at least the portion of the CT domain containing the α1-syntrophin binding site, including SEQ ID NO:50). Table 4 below has the amino acid sequences for these components, in particular from the full length human DMD protein (UniProtDB-11532, which is incorporated by reference herein) and they are encoded by the nucleotide sequences in Tables 6 and 7 (including the wild type and codon optimized sequences).

To overcome the packaging limitation that is typical of AAV vectors, many of the microdystrophin genes developed for clinical use are lacking the CT domain. Several researchers have indicated that the Dystrophin Associated Protein Complex (DAPC) does not even require the C-terminal domain in order to assemble or that the C-terminus is non-essential [Crawford, et al., J Cell Biol, 2000, 150(6):1399-1409; and Ramos, J. N, et al. Molecular Therapy 2019, 27(3):1-13]. However, overexpression of a microdystrophin gene containing helix 1 of the coiled-coil motif of the CT domain in skeletal muscle of mdx mice increased the recruitment α1-syntrophin and α-dystrobrevin, which are members of the DAP complex, serving as modular adaptors for signaling proteins recruited to the sarcolemma membrane [Koo, T., et al., Delivery of AAV2/9-microdystrophin genes incorporating helix 1 of the coiled-coil motif in the C-terminal domain of dystrophin improves muscle pathology and restores the level of α1-syntrophin and α-dystrobrevin in skeletal muscles of mdx mice. Hum Gene Ther, 2011. 22(11): p. 1379-88]. Overexpression of the longer version of microdystrophin also improved the muscle resistance to lengthening contraction-induced muscle damage in the mdx mice as compared with the shorter version [Koo, T., et al. 2011, supra]. The CT domain does play a role in the formation of the DAPC (see FIG. 1B).

The CT domain of dystrophin contains two polypeptide stretches that are predicted to form α-helical coiled coils similar to those in the rod domain (see H1 indicated by single underlining and H2 indicated by double underlining in SEQ ID 48 in Table 4 below). Each coiled coil has a conserved repeating heptad (a,b,c,d,e,f,g), similar to those found in leucine zippers where leucine predominates at the “d” position. This domain has been named the CC (coiled coil) domain. The CC region of dystrophin forms the binding site for dystrobrevin and may modulate the interaction between α1-syntrophin and other dystrophin-associated proteins.

Both syntrophin isoforms, α1-syntrophin and β1-syntrophin are thought to interact directly with dystrophin through more than one binding site in dystrophin exons 73 and 74 (Yang et al, JBC 270(10):4975-8 (1995)). α1- and β1-syntrophin bind separately to the dystrophin C-terminal domain, and the binding site for α1-syntrophin reportedly resides at least within the amino acid residues 3447 to 3481, while that for β1-syntrophin has been reported to reside within the amino acid residues 3495 to 3535 (as numbered in the DMD protein of UniProtDB-11532 (SEQ ID NO:51), see also Table 4, SEQ ID NO: 48, italic). Alpha1- (α1-) syntrophin and alpha-syntrophin are used interchangeably throughout.

In certain embodiments, the microdystrophin protein has a C-terminal domain that “increases binding” to α1-syntrophin, β-syntrophin and/or dystrobrevin compared to a comparable microdystrophin that does not contain the C-terminal domain (but has the same amino acid sequence otherwise, that is a “reference microdystrophin protein”), meaning that the DAPC is stabilized or anchored to the sarcolemma, to a greater extent than a reference microdystrophin that does not have the C-terminal domain (but has the same amino acid sequence otherwise as the microdystrophin), as determined by greater levels of one or more DAPC components in the muscle membrane by immunostaining of muscle sections or western blot analysis of muscle tissue lysates or muscle membrane preparations for one of more DAPC components, including α1-syntrophin, β-syntrophin, α-dystrobrevin, β-dystroglycan or nNOS in mdx mouse muscle treated with the microdystrophin having the C-terminal domain, as compared to the mdx mouse muscle treated with the reference microdystrophin protein (having the same sequence and dystrophin components except not having the C-terminal domain).

In some embodiments, the microdystrophin construct including a C-terminal domain of dystrophin comprises an α1-syntrophin binding site and/or a dystrobrevin binding site in the C-terminal domain. In some embodiments, the C-terminal domain comprising an α1-syntrophin binding site is a truncated C-terminal domain. The α1-syntrophin binding site functions in part to recruit and anchor nNOS to the sarcolemma through α1-syntrophin.

The embodiments described herein can comprise all or a portion of the CT domain comprising the Helix 1 of the coiled-coil motif. The C Terminal sequence may be defined by the coding sequence of the exons of the DMD gene, in particular exons 70 to 74, and a portion of exon 75 (in particular, the nucleotide sequence encoding the first 36 amino acids of the amino acid sequence encoded by exon 75, or by the sequence of the human DMD protein, for example, the sequence of UniProtKB-P11532 (SEQ ID NO: 51) (the CT is amino acids 3361 to 3554 of the UniProtKB-P11532 sequence), or comprising or consisting of binding sites for dystrobrevin and/or α1-syntrophin (indicated in Table 4, SEQ ID NO: 48). In certain embodiments, the CT domain consists or comprises the 194 C-terminal amino acids of the DMD protein, for example, residues 3361 to 3554 of the amino acid sequence of UniProtKB-P11532 (SEQ ID NO: 51), the amino acids encoded by exons 70 to 74, and the nucleotide sequence encoding the first 36 nucleotides of the nucleotide sequence of exon 75 of the DMD gene, or the amino acid sequence of SEQ ID NO: 48 (see Table 4). For example, RGX-DYS1 has the 194 amino acid CT sequence of SEQ ID NO: 48. In other embodiments, the amino acid sequence of the C-terminal domain is truncated and comprises at least the binding sites for dystrobrevin and/or α1-syntrophin. In certain embodiments, the truncated C-terminal domain comprises the amino acid sequence MENSNGSYLNDSISPNESIDDEHLLIQHYCQSLNQ (α1-syntrophin binding site) (SEQ ID NO: 50). In particular embodiments, the CT domain sequence has the amino acid sequence of SEQ ID NO: 49 or amino acids 3361 to 3500 of the UniProtKB-P11532 human DMD sequence. For example, RGX-DYS5 has a CT domain having the amino acid sequence of SEQ ID NO: 49. In alternative embodiments, the microdystrophin lacks a CT domain, and may have the domains arranged as follows: ABD1-L1-H1-L2-R1-R2-L3-R3-H3-L4-R24-H4-CR, for example RGX-DYS3 (SEQ ID NO: 53).

The NH₂ terminus and a region in the rod domain of dystrophin bind directly to but do not cross-link cytoskeletal actin. The rod domain of wild type dystrophin is composed of 24 repeating units that are similar to the triple helical repeats of spectrin. This repeating unit accounts for the majority of the dystrophin protein and is thought to give the molecule a flexible rod-like structure similar to β-spectrin. These α-helical coiled-coil repeats are interrupted by four proline-rich hinge regions. At the end of the 24th repeat is the fourth hinge region that is immediately followed by the WW domain [Blake, D. et al, Function and Genetics of Dystrophin and Dystrophin-Related Proteins in Muscle. Physiol. Rev. 82: 291-329, 2002]. Microdystrophins disclosed herein do not include R4 to R23, and only include 3 of the 4 hinge regions or portions thereof. In some embodiments, no new amino acid residues or linkers are introduced into the microdystrophin.

In some embodiments, microdystrophin comprises an H3 domain. In embodiments, H3 can be a full endogenous H3 domain from N-terminus to C-terminus. Stated another way, some microdystrophin embodiments do not contain a fragment of the H3 domain but contain the entire H3 domain. In some embodiments, the C-terminal amino acid of the R3 domain is coupled directly (or covalently bonded to) the N-terminal amino acid of the H3 domain. In some embodiments, the C-terminal amino acid of the R3 domain coupled to the N-terminal amino acid of the H3 domain is Q. In some embodiments, the 5′ amino acid of the H3 domain coupled to the R3 domain is Q.

Without being bound by any one theory, a full hinge domain may be appropriate in any microdystrophin construct in order to convey full activity upon the derived microdystrophin protein. Hinge segments of dystrophin have been recognized as being proline-rich in nature and may therefore confer flexibility to the protein product (Koenig and Kunkel, 265(6):4560-4566, 1990). Any deletion of a portion of the hinge, especially removal of one or more proline residues, may reduce its flexibility and therefore reduce its efficacy by hindering its interaction with other proteins in the DAP complex.

Microdystrophins disclosed herein comprise the wild-type dystrophin H4 sequence (which contains the WW domain) to and including the CR domain (which contains the ZZ domain, represented by a single underline (UniProtKB-P11532 aa 3307-3354) in SEQ ID NO: 47). The WW domain is a protein-binding module found in several signaling and regulatory molecules. The WW domain binds to proline-rich substrates in an analogous manner to the src homology-3 (SH3) domain. This region mediates the interaction between β-dystroglycan and dystrophin, since the cytoplasmic domain of 0-dystroglycan is proline rich. The WW domain is in the Hinge 4 (H4 region). The CR domain contains two EF-hand motifs that are similar to those in α-actinin and that could bind intracellular Ca²⁺. The ZZ domain contains a number of conserved cysteine residues that are predicted to form the coordination sites for divalent metal cations such as Zn²⁺. The ZZ domain is similar to many types of zinc finger and is found both in nuclear and cytoplasmic proteins. The ZZ domain of dystrophin binds to calmodulin in a Ca²⁺-dependent manner. Thus, the ZZ domain may represent a functional calmodulin-binding site and may have implications for calmodulin binding to other dystrophin-related proteins.

Microdystrophin embodiments can further comprise linkers (L1, L2, L3, L4, L4.1 and/or L4.2) or portions thereof connected the domains as shown as follows: ABD1-L1-H1-L2-R1-R2-L3-R3-H3-L4-R24-H4-CR-CT (e.g., SEQ ID NO: 91 or 93) or ABD1-L1-H1-L2-R1-R2-L3-R3-H3-L4-R24-H4-CR (e.g., SEQ ID NO: 92) L1 can be an endogenous linker L1 (e.g., SEQ ID NO: 38) that can couple ABD1 to H1. L2 can be an endogenous linker L2 (e.g., SEQ ID NO: 40) that can couple H1 to R1. L3 can be an endogenous linker L3 that can couple R2 to R3.

L4 can also be an endogenous linker that can couple H3 and R24. In some embodiments, L4 is 3 amino acids, e.g. TLE that precede R24 in the native dystrophin sequence. In other embodiments, L4 can be the 4 amino acids that precede R24 in the native dystrophin sequence (SEQ ID NO: 51) or the 2 amino acids that precede R24. In other embodiments, there is no linker, L4 or otherwise, in between H3 and R24. On the 5′ end of H3, as mentioned above, no linker is present, but rather R3 is directly coupled to H3, or alternatively H2.

The above described components of microdystrophin other domains not specifically described can have the amino acid sequences as provided in Table 4 below. The amino acid sequences for the domains provided herein correspond to the dystrophin isoform of UniProtKB-P11532 (DMD_HUMAN) (SEQ ID NO: 51), which is herein incorporated by reference. Other embodiments can comprise the domains from naturally-occurring functional dystrophin isoforms known in the art, such as UniProtKB-A0A075B6G3 (A0A075B6G3_HUMAN), (incorporated by reference herein) wherein, for example, R24 has an R substituted for the Q at amino acid 3 of SEQ ID NO: 51.

Additional embodiments are disclosed in International Application PCT/US2020/062484, filed Nov. 27, 2020, which is hereby incorporated by reference in its entirety.

TABLE 4
segment amino acid sequences

| Structure | SEQ ID | Sequence |
|---|---|---|
| ABD1 | 37 | MLWWEEVEDCYEREDVQKKTFTKWVNAQFSKFGKQHIENLFSDLQD |
| | | GRRLLDLLEGLTGQKLPKEKGSTRVHALNNVNKALRVLQNNNVDLV |
| | | NIGSTDIVDGNHKLTLGLIWNIILHWQVKNVMKNIMAGLQQTNSEK |
| | | ILLSWVRQSTRNYPQVNVINFTTSWSDGLALNALIHSHRPDLFDWN |
| | | SVVCQQSATQRLEHAFNIARYQLGIEKLLDPEDVDTTYPDKKSILM |
| | | YITSLFQVLP |
| L1 | 38 | QQVSIEAIQEVE |
| H1 | 39 | MLPRPPKVTKEEHFQLHHQMHYSQQITVSLAQGYERTSSPKPRFKS |
| | | YAYTQAAYVTTSDPTRSPFPSQHLEAPED |
| L2 | 40 | KSFGSSLME |
| R1 | 41 | SEVNLDRYQTALEEVLSWLLSAEDTLQAQGEISNDVEVVKDQFHTH |
| | | EGYMMDLTAHQGRVGNILQLGSKLIGTGKLSEDEETEVQEQMNLLN |
| | | SRWECLRVASMEKQSNLHR |
| R2 | 42 | VLMDLQNQKLKELNDWLTKTEERTRKMEEEPLGPDLEDLKRQVQQH |
| | | KVLQEDLEQEQVRVNSLTHMVVVVDESSGDHATAALEEQLKVLGDR |
| | | WANICRWTEDRWVLLQD |
| L3 | | IL |
| R3 | 43 | LKWQRLTEEQCLFSAWLSEKEDAVNKIHTTGFKDQNEMLSSLQKLA |
| | | VLKADLEKKKQSMGKLYSLKQDLLSTLKNKSVTQKTEAWLDNFARC |
| | | WDNLVQKLEKSTAQISQ |
| H3 | 44 | QPDLAPGLTTIGASPTQTVTLVTQPVVTKETAISKLEMPSSLMLEV |
| | | P |
| L4 | | TLE |
| R24 | 45 | RLQELQEATDELDLKLRQAEVIKGSWQPVGDLLIDSLQDHLEKVKA |
| | | LRGEIAPLKENVSHVNDLARQLTTLGIQLSPYNLSTLEDLNTRWKL |
| | | LQVAVEDRVRQLHE |
| H4 | 46 | AHRDFGPASQHELSTSVQGPWERAISPNKVPYYINHETQTTCWDHP |
| | | KMTELYQSLADLNNVRFSAYRTAMKL |
| | | WW domain is represented by a single underline (UniProtKB- |
| | | P11532 aa 3055-3088) |
| Cysteine- | 47 | RRLQKALCLDLLSLSAACDALDQHNLKQNDQPMDILQIINCLTTIY |
| rich | | DRLEQEHNNLVNVPLCVDMCLNWLLNVYDTGRTGRIRVLSFKTGII |
| domain | | SLCKAHLEDKYRYLFKQVASSTGFCDQRRLGLLLHDSIQIPRQLGE |
| (CR) | | VASFGGSNIEPSVRSCFQFANNKPEIEAALFLDWMRLEPQSMVWLP |
| | | VLHRVAAAETAKHQAKCNICKECPIIGFRYRSLKHFNYDICQSCFF |
| | | SGRVAKGHKMHYPMVEYC |
| | | ZZ domain is represented by a single underline (UniProtKB- |
| | | P11532 aa 3307-3354) |
| C-terminal | 48 | TPTTSGEDVRDFAKVLKNKFRTKRYFAKHPRMGYLPVQTVLEGDNM |
| Domain | | ETPVTLINFWPVDSAPASSPQLSHDDTHSRIEHYASRLAEMENSNG |
| (CT) | | SYLNDSISPNESIDDEHLLIQHYCQSLNQDSPLSQPRSPAQILISL |
| | | ESEERGELERILADLEEENRNLQAEYDRLKQQHEHKGLSPLPSPPE |
| | | MMPTSPQSPR |
| | | Coiled-coil motif H1 is represented by a single underline; |
| | | motif H2 is represented by a double underline; dystrobrevin- |
| | | binding side is in italics. |
| Minimal/ | 49 | TPTTSGEDVRDFAKVLKNKFRTKRYFAKHPRMGYLPVQTVLEGDNM |
| truncated | | ETPVTLINFWPVDSAPASSPQLSHDDTHSRIEHYASRLAEMENSNG |
| C-terminal | | SYLNDSISPNESIDDEHLLIQHYCQSLNQDSPLSQPRSPAQILISL |
| Domain | | ES |
| (CT1.5) | | α1-syntrophin-binding site is in italics. |
| L4 | | ETLE |
| L4 | | LE |
| Minimal | 50 | MENSNGSYLNDSISPNESIDDEHLLIQHYCQSLNQ |
| alpha- | | |
| syntrophin | | |
| binding site | | |
| Human | 51 | MLWWEEVEDC YEREDVQKKT FTKWVNAQFS KFGKQHIENL |
| dystrophin | | FSDLQDGRRL LDLLEGLTGQ KLPKEKGSTR VHALNNVNKA |
| (UniProtK | | LRVLQNNNVD LVNIGSTDIV DGNHKLTLGL IWNIILHWQV |
| B- | | KNVMKNIMAG LQQTNSEKIL LSWVRQSTRN YPQVNVINFT |
| P11532) | | TSWSDGLALN ALIHSHRPDL FDWNSVVCQQ SATQRLEHAF |
| | | NIARYQLGIE KLLDPEDVDT TYPDKKSILM YITSLFQVLP |
| | | QQVSIEAIQE VEMLPRPPKV TKEEHFQLHH QMHYSQQITV |
| | | SLAQGYERTS SPKPRFKSYA YTQAAYVITS DPTRSPFPSQ |
| | | HLEAPEDKSF GSSLMESEVN LDRYQTALEE VLSWLLSAED |
| | | TLQAQGEISN DVEVVKDQFH THEGYMMDLT AHQGRVGNIL |
| | | QLGSKLIGTG KLSEDEETEV QEQMNLLNSR WECLRVASME |
| | | KQSNLHRVLM DLQNQKLKEL NDWLTKTEER TRKMEEEPLG |
| | | PDLEDLKRQV QQHKVLQEDL EQEQVRVNSL THMVVVVDES |
| | | SGDHATAALE EQLKVLGDRW ANICRWTEDR WVLLQDILLK |
| | | WQRLTEEQCL FSAWLSEKED AVNKIHTTGF KDQNEMLSSL |
| | | QKLAVLKADL EKKKQSMGKL YSLKQDLLST LKNKSVTQKT |
| | | EAWLDNFARC WDNLVQKLEK STAQISQAVT TTQPSLTQTT |
| | | VMETVTTVTT REQILVKHAQ EELPPPPPQK KRQITVDSEI |
| | | RKRLDVDITE LHSWITRSEA VLQSPEFAIF RKEGNFSDLK |
| | | EKVNAIEREK AEKFRKLQDA SRSAQALVEQ MVNEGVNADS |
| | | IKQASEQLNS RWIEFCQLLS ERLNWLEYQN NIIAFYNQLQ |
| | | QLEQMTTTAE NWLKIQPTTP SEPTAIKSQL KICKDEVNRL |
| | | SDLQPQIERL KIQSIALKEK GQGPMFLDAD FVAFTNHFKQ |
| | | VFSDVQAREK ELQTIFDTLP PMRYQETMSA IRTWVQQSET |
| | | KLSIPQLSVT DYEIMEQRLG ELQALQSSLQ EQQSGLYYLS |
| | | TTVKEMSKKA PSEISRKYQS EFEEIEGRWK KLSSQLVEHC |
| | | QKLEEQMNKL RKIQNHIQTL KKWMAEVDVF LKEEWPALGD |
| | | SEILKKQLKQ CRLLVSDIQT IQPSLNSVNE GGQKIKNEAE |
| | | PEFASRLETE LKELNTQWDH MCQQVYARKE ALKGGLEKTV |
| | | SLQKDLSEMH EWMTQAEEEY LERDFEYKTP DELQKAVEEM |
| | | KRAKEEAQQK EAKVKLLTES VNSVIAQAPP VAQEALKKEL |
| | | ETLTTNYQWL CTRLNGKCKT LEEVWACWHE LLSYLEKANK |
| | | WLNEVEFKLK TTENIPGGAE EISEVLDSLE NLMRHSEDNP |
| | | NQIRILAQTL TDGGVMDELI NEELETENSR WRELHEEAVR |
| | | RQKLLEQSIQ SAQETEKSLH LIQESLTFID KQLAAYIADK |
| | | VDAAQMPQEA QKIQSDLTSH EISLEEMKKH NQGKEAAQRV |
| | | LSQIDVAQKK LQDVSMKFRL FQKPANFEQR LQESKMILDE |
| | | VKMHLPALET KSVEQEVVQS QLNHCVNLYK SLSEVKSEVE |
| | | MVIKTGRQIV QKKQTENPKE LDERVTALKL HYNELGAKVT |
| | | ERKQQLEKCL KLSRKMRKEM NVLTEWLAAT DMELTKRSAV |
| | | EGMPSNLDSE VAWGKATQKE IEKQKVHLKS ITEVGEALKT |
| | | VLGKKETLVE DKLSLLNSNW IAVTSRAEEW LNLLLEYQKH |
| | | METFDQNVDH ITKWIIQADT LLDESEKKKP QQKEDVLKRL |
| | | KAELNDIRPK VDSTRDQAAN LMANRGDHCR KLVEPQISEL |
| | | NHRFAAISHR IKTGKASIPL KELEQFNSDI QKLLEPLEAE |
| | | IQQGVNLKEE DENKDMNEDN EGTVKELLQR GDNLQQRITD |
| | | ERKREEIKIK QQLLQTKHNA LKDLRSQRRK KALEISHQWY |
| | | QYKRQADDLL KCLDDIEKKL ASLPEPRDER KIKEIDRELQ |
| | | KKKEELNAVR RQAEGLSEDG AAMAVEPTQI QLSKRWREIE |
| | | SKFAQFRRLN FAQIHTVREE TMMVMTEDMP LEISYVPSTY |
| | | LTEITHVSQA LLEVEQLLNA PDLCAKDFED LFKQEESLKN |
| | | IKDSLQQSSG RIDIIHSKKT AALQSATPVE RVKLQEALSQ |
| | | LDFQWEKVNK MYKDRQGRED RSVEKWRRFH YDIKIFNQWL |
| | | TEAEQFLRKT QIPENWEHAK YKWYLKELQD GIGQRQTVVR |
| | | ILNATGEEII QQSSKTDASI LQEKLGSLNL RWQEVCKQLS |
| | | DRKKRLEEQK NILSEFQRDL NEFVLWLEEA DNIASIPLEP |
| | | GKEQQLKEKL EQVKLLVEEL PLRQGILKQL NETGGPVLVS |
| | | APISPEEQDK LENKLKQTNL QWIKVSRALP EKQGEIEAQI |
| | | KDLGQLEKKL EDLEEQLNHL LLWLSPIRNQ LEIYNQPNQE |
| | | GPFDVKETEI AVQAKQPDVE EILSKGQHLY KEKPATQPVK |
| | | RKLEDLSSEW KAVNRLLQEL RAKQPDLAPG LTTIGASPTQ |
| | | TVTLVTQPVV TKETAISKLE MPSSLMLEVP ALADENRAWT |
| | | ELTDWLSLLD QVIKSQRVMV GDLEDINEMI IKQKATMQDL |
| | | EQRRPQLEEL ITAAQNLKNK TSNQEARTII TDRIERIQNQ |
| | | WDEVQEHLQN RRQQLNEMLK DSTQWLEAKE EAEQVLGQAR |
| | | AKLESWKEGP YTVDAIQKKI TETKQLAKDL RQWQTNVDVA |
| | | NDLALKLLRD YSADDTRKVH MITENINASW RSIHKRVSER |
| | | EAALEETHRL LQQFPLDLEK FLAWLTEAET TANVLQDATR |
| | | KERLLEDSKG VKELMKQWQD LQGEIEAHTD VYHNLDENSQ |
| | | KILRSLEGSD DAVLLQRRLD NMNFKWSELR KKSLNIRSHL |
| | | EASSDQWKRL HLSLQELLVW LQLKDDELSR QAPIGGDEPA |
| | | VQKQNDVHRA FKRELKTKEP VIMSTLETVR IFLTEQPLEG |
| | | LEKLYQEPRE LPPEERAQNV TRLLRKQAEE VNTEWEKLNL |
| | | HSADWQRKID ETLERLQELQ EATDELDLKL RQAEVIKGSW |
| | | QPVGDLLIDS LQDHLEKVKA LRGEIAPLKE NVSHVNDLAR |
| | | QLTTLGIQLS PYNLSTLEDL NTRWKLLQVA VEDRVRQLHE |
| | | AHRDFGPASQ HELSTSVQGP WERAISPNKV PYYINHETQT |
| | | TCWDHPKMTE LYQSLADLNN VRESAYRTAM KLRRLQKALC |
| | | LDLLSLSAAC DALDQHNLKQ NDQPMDILQI INCLTTIYDR |
| | | LEQEHNNLVN VPLCVDMCLN WLLNVYDTGR TGRIRVLSFK |
| | | TGIISLCKAH LEDKYRYLFK QVASSTGFCD QRRLGLLLHD |
| | | SIQIPRQLGE VASEGGSNIE PSVRSCFQFA NNKPEIEAAL |
| | | FLDWMRLEPQ SMVWLPVLHR VAAAETAKHQ AKCNICKECP |
| | | IIGFRYRSLK HENYDICQSC FFSGRVAKGH KMHYPMVEYC |
| | | TPTTSGEDVR DFAKVLKNKF RTKRYFAKHP RMGYLPVQTV |
| | | LEGDNMETPV TLINFWPVDS APASSPQLSH DDTHSRIEHY |
| | | ASRLAEMENS NGSYLNDSIS PNESIDDEHL LIQHYCQSLN |
| | | QDSPLSQPRS PAQILISLES EERGELERIL ADLEEENRNL |
| | | QAEYDRLKQQ HEHKGLSPLP SPPEMMPTSP QSPRDAELIA |
| | | EAKLLRQHKG RLEARMQILE DHNKQLESQL HRLRQLLEQP |
| | | QAEAKVNGTT VSSPSTSLQR SDSSQPMLLR VVGSQTSDSM |
| | | GEEDLLSPPQ DTSTGLEEVM EQLNNSFPSS RGRNTPGKPM |
| | | REDTM |

The present disclosure also contemplates variants of these sequences so long as the function of each domain and linker is substantially maintained and/or the therapeutic efficacy of microdystrophin comprising such variants is substantially maintained. Functional activity includes (1) binding to one of, a combination of, or all of actin, β-dystroglycan, α1-syntrophin, α-dystrobrevin, and nNOS; (2) improved muscle function in an animal model (for example, in the mdx mouse model described herein) or in human subjects; and/or (3) cardioprotective or improvement in cardiac muscle function in animal models or human patients.

Table 5 provides the amino acid sequences of the microdystrophin embodiments in accordance with the present disclosure. In certain embodiments, the microdystrophin has an amino acid sequence of SEQ ID NOs: 52 (DYS1), 53 (DYS3), or 54 (DYS5). In other embodiments, the microdystrophin has an amino acid sequence of SEQ ID NO: 133 (human MD1 (R4-R23/ACT), SEQ ID NO: 134 (microdystrophin), SEQ ID NO: 135 (Dys3978), SEQ ID NO: 136 (MD3) or SEQ ID NO: 137 (MD4). It is also contemplated that other embodiments are substituted variants of microdystrophins as defined by SEQ ID NOs: 52 (DYS1), 53 (DYS3), or 54 (DYS5). For example, conservative substitutions can be made to SEQ ID NOs: 52, 53, or 54 (or alternatively SEQ ID NO; 133-137) and substantially maintain its functional activity. In embodiments, microdystrophin may have at least 60%, at least 70%, at least 80%, at least 85%, at least 90%, at least 95%, at least 96%, at least 97%, at least 98%, or at least 99% sequence identity to the amino acid sequence of SEQ ID NOs: 52, 53, or 54 (or alternatively SEQ ID NO: 137) and maintain functional microdystrophin activity, as determined, for example, by one or more of the in vitro assays or in vivo assays in animal models disclosed in Section 5.7 infra.

TABLE 5
Amino acid sequences of RGX-DYS and Microdystrophin proteins

| | SEQ | |
|---|---|---|
| | ID | |
| Structure | NO: | Amino Acid Sequence |
| DYS1 | 52 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQQPDLAPGLTTIGASPTQTVTLVTQPVVTKETAI |
| | | SKLEMPSSLMLEVPTLERLQELQEATDELDLKLRQAEVIKGSWQPVGDLL |
| | | IDSLQDHLEKVKALRGEIAPLKENVSHVNDLARQLTTLGIQLSPYNLSTL |
| | | EDLNTRWKLLQVAVEDRVRQLHEAHRDFGPASQHELSTSVQGPWERAISP |
| | | NKVPYYINHETQTTCWDHPKMTELYQSLADLNNVRFSAYRTAMKLRRLQK |
| | | ALCLDLLSLSAACDALDQHNLKQNDQPMDILQIINCLTTIYDRLEQEHNN |
| | | LVNVPLCVDMCLNWLLNVYDTGRTGRIRVLSFKTGIISLCKAHLEDKYRY |
| | | LFKQVASSTGFCDQRRLGLLLHDSIQIPRQLGEVASEGGSNIEPSVRSCF |
| | | QFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGE |
| | | DVRDFAKVLKNKFRTKRYFAKHPRMGYLPVQTVLEGDNMETPVTLINFWP |
| | | VDSAPASSPQLSHDDTHSRIEHYASRLAEMENSNGSYLNDSISPNESIDD |
| | | EHLLIQHYCQSLNQDSPLSQPRSPAQILISLESEERGELERILADLEEEN |
| | | RNLQAEYDRLKQQHEHKGLSPLPSPPEMMPTSPQSPR |
| DYS3 | 53 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQQPDLAPGLITIGASPTQTVTLVTQPVVTKETAI |
| | | SKLEMPSSLMLEVPTLERLQELQEATDELDLKLRQAEVIKGSWQPVGDLL |
| | | IDSLQDHLEKVKALRGEIAPLKENVSHVNDLARQLTTLGIQLSPYNLSTL |
| | | EDLNTRWKLLQVAVEDRVRQLHEAHRDFGPASQHELSTSVQGPWERAISP |
| | | NKVPYYINHETQTTCWDHPKMTELYQSLADLNNVRFSAYRTAMKLRRLQK |
| | | ALCLDLLSLSAACDALDQHNLKQNDQPMDILQIINCLTTIYDRLEQEHNN |
| | | LVNVPLCVDMCLNWLLNVYDTGRTGRIRVLSFKTGIISLCKAHLEDKYRY |
| | | LFKQVASSTGFCDQRRLGLLLHDSIQIPRQLGEVASFGGSNIEPSVRSCF |
| | | QFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGE |
| | | DVRDFAKVLKNKFRTKRYFAKHPRMGYLPVQTVLEGDNMET |
| DYS5 | 54 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQQPDLAPGLTTIGASPTQTVTLVTQPVVTKETAI |
| | | SKLEMPSSLMLEVPTLERLQELQEATDELDLKLRQAEVIKGSWQPVGDLL |
| | | IDSLQDHLEKVKALRGEIAPLKENVSHVNDLARQLTTLGIQLSPYNLSTL |
| | | EDLNTRWKLLQVAVEDRVRQLHEAHRDFGPASQHELSTSVQGPWERAISP |
| | | NKVPYYINHETQTTCWDHPKMTELYQSLADLNNVRESAYRTAMKLRRLQK |
| | | ALCLDLLSLSAACDALDQHNLKQNDQPMDILQIINCLTTIYDRLEQEHNN |
| | | LVNVPLCVDMCLNWLLNVYDTGRTGRIRVLSFKTGIISLCKAHLEDKYRY |
| | | LFKQVASSTGFCDQRRLGLLLHDSIQIPRQLGEVASEGGSNIEPSVRSCF |
| | | QFANNKPEIEAALFLDWMRLEPQSMVWLPVLHRVAAAETAKHQAKCNICK |
| | | ECPIIGFRYRSLKHFNYDICQSCFFSGRVAKGHKMHYPMVEYCTPTTSGE |
| | | DVRDFAKVLKNKFRTKRYFAKHPRMGYLPVQTVLEGDNMETPVTLINFWP |
| | | VDSAPASSPQLSHDDTHSRIEHYASRLAEMENSNGSYLNDSISPNESIDD |
| | | EHLLIQHYCQSLNQDSPLSQPRSPAQILISLES |
| human MD1 | 133 | MLWWEEVEDCYEREDVQKKTFTKWVNAQFSKFGKQHIENLFSDLQDGRRL |
| (R4-R23/ACT) | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQAVTTTQPSLTQTTVMETVTTVTTREQILVKHAQ |
| | | EELPPPPPQKKRQITVDTLERLQELQEATDELDLKLRQAEVIKGSWQPVG |
| | | DLLIDSLQDHLEKVKALRGEIAPLKENVSHVNDLARQLTTLGIQLSPYNL |
| | | STLEDLNTRWKLLQVAVEDRVRQLHEAHRDFGPASQHELSTSVQGPWERA |
| | | ISPNKVPYYINHETQTTCWDHPKMTELYQSLADLNNVRESAYRTAMKLRR |
| | | LQKALCLDLLSLSAACDALDQHNLKQNDQPMDILQIINCLTTIYDRLEQE |
| | | HNNLVNVPLCVDMCLNWLLNVYDTGRTGRIRVLSFKTGIISLCKAHLEDK |
| | | YRYLFKQVASSTGFCDQRRLGLLLHDSIQIPRQLGEVASEGGSNIEPSVR |
| | | SCFQFANNKPEIEAALFLDWMRLEPQSMVWLPVLHRVAAAETAKHQAKCN |
| | | ICKECPIIGFRYRSLKHFNYDICQSCFFSGRVAKGHKMHYPMVEYCTPTT |
| | | SGEDVRDFAKVLKNKFRTKRYFAKHPRMGYLPVQTVLEGDNMETDTM |
| Human | 134 | MLWWEEVEDCYEREDVQKKTFTKWVNAQFSKFGKQHIENLFSDLQDGRRL |
| micro- | | LDLLEGLTGQKLPKEKGSTRVHALNNVNKALRVLQNNNVDLVNIGSTDIV |
| dystrophin | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQAVTTTQPSLTQTTVMETVTTVTTREQILVKHAQ |
| | | EELPPPPPQKKRTLERLQELQEATDELDLKLRQAEVIKGSWQPVGDLLID |
| | | SLQDHLEKVKALRGEIAPLKENVSHVNDLARQLTTLGIQLSPYNLSTLED |
| | | LNTRWKLLQVAVEDRVRQLHEAHRDFGPASQHELSTSVQGPWERAISPNK |
| | | VPYYINHETQTTCWDHPKMTELYQSLADLNNVRFSAYRTAMKLRRLQKAL |
| | | CLDLLSLSAACDALDQHNLKQNDQPMDILQIINCLTTIYDRLEQEHNNLV |
| | | NVPLCVDMCLNWLLNVYDTGRTGRIRVLSFKTGIISLCKAHLEDKYRYLF |
| | | KQVASSTGFCDQRRLGLLLHDSIQIPRQLGEVASFGGSNIEPSVRSCFQF |
| | | ANNKPEIEAALFLDWMRLEPQSMVWLPVLHRVAAAETAKHQAKCNICKEC |
| | | PIIGFRYRSLKHFNYDICQSCFFSGRVAKGHKMHYPMVEYCTPTTSGEDV |
| | | RDFAKVLKNKFRTKRYFAKHPRMGYLPVQTVLEGDNMETDTM |
| Dys3978 | 135 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDQPDLAPGLTTIGASPTQTVTLVTQPVVTKETAISKLEMPSSLML |
| | | EVPTHRLLQQFPLDLEKFLAWLTEAETTANVLQDATRKERLLEDSKGVKE |
| | | LMKQWQDLQGEIEAHTDVYHNLDENSQKILRSLEGSDDAVLLQRRLDNMN |
| | | FKWSELRKKSLNIRSHLEASSDQWKRLHLSLQELLVWLQLKDDELSRQAP |
| | | IGGDFPAVQKQNDVHRAFKRELKTKEPVIMSTLETVRIFLTEQPLEGLEK |
| | | LYQEPRELPPEERAQNVTRLLRKQAEEVNTEWEKLNLHSADWQRKIDETL |
| | | ERLQELQEATDELDLKLRQAEVIKGSWQPVGDLLIDSLQDHLEKVKALRG |
| | | EIAPLKENVSHVNDLARQLTTLGIQLSPYNLSTLEDLNTRWKLLQVAVED |
| | | RVRQLHEAHRDFGPASQHELSTSVQGPWERAISPNKVPYYINHETQTTCW |
| | | DHPKMTELYQSLADLNNVRFSAYRTAMKLRRLQKALCLDLLSLSAACDAL |
| | | DQHNLKQNDQPMDILQIINCLTTIYDRLEQEHNNLVNVPLCVDMCLNWLL |
| | | NVYDTGRTGRIRVLSFKTGIISLCKAHLEDKYRYLFKQVASSTGFCDQRR |
| | | LGLLLHDSIQIPRQLGEVASFGGSNIEPSVRSCFQFANNKPEIEAALFLD |
| | | WMRLEPQSMVWLPVLHRVAAAETAKHQAKCNICKECPIIGFRYRSLKHEN |
| | | YDICQSCFFSGRVAKGHKMHYPMVEYCTPTTSGEDVRDFAKVLKNKFRTK |
| | | RYFAKHPRMGYLPVQTVLEGDNMET |
| Human MD3 | 136 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQAVTTTQPSLTQTTVMETVTTVTTREQILVKHAQ |
| | | EELPPPPPQKKRQITVDTLERLQELQEATDELDLKLRQAEVIKGSWQPVG |
| | | DLLIDSLQDHLEKVKALRGEIAPLKENVSHVNDLARQLTTLGIQLSPYNL |
| | | STLEDLNTRWKLLQVAVEDRVRQLHEAHRDFGPASQHFLSTSVQGPWERA |
| | | ISPNKVPYYINHETQTTCWDHPKMTELYQSLADLNNVRESAYRTAMKLRR |
| | | LQKALCLDLLSLSAACDALDQHNLKQNDQPMDILQIINCLTTIYDRLEQE |
| | | HNNLVNVPLCVDMCLNWLLNVYDTGRTGRIRVLSFKTGIISLCKAHLEDK |
| | | YRYLFKQVASSTGFCDQRRLGLLLHDSIQIPRQLGEVASFGGSNIEPSVR |
| | | SCFQFANNKPEIEAALFLDWMRLEPQSMVWLPVLHRVAAAETAKHQAKCN |
| | | ICKECPIIGFRYRSLKHFNYDICQSCFFSGRVAKGHKMHYPMVEYCTPTT |
| | | SGEDVRDFAKVLKNKFRTKRYFAKHPRMGYLPVQTVLEGDNMETPVTLIN |
| | | FWPVDSAPASSPQLSHDDTHSRIEHYASRLAEMENSNGSYLNDSISPNES |
| | | IDDEHLLIQHYCQSLNQDSPLSQPRSPAQILISLESEERGELERILADLE |
| | | EENRNLQAEYDRLKQQHEHKGLSPLPSPPEMMPTSPQSPRDAELIAEAKL |
| | | LRQHKGRLEARMQILEDHNKQLESQLHRLRQLLEQPQAEDTM |
| Human MD4 | 137 | MLWWEEVEDCYEREDVQKKTFTKWVNAQFSKFGKQHIENLFSDLQDGRRL |
| | | LDLLEGLTGQKLPKEKGSTRVHALNNVNKALRVLQNNNVDLVNIGSTDIV |
| | | DGNHKLTLGLIWNIILHWQVKNVMKNIMAGLQQTNSEKILLSWVRQSTRN |
| | | YPQVNVINFTTSWSDGLALNALIHSHRPDLFDWNSVVCQQSATQRLEHAF |
| | | NIARYQLGIEKLLDPEDVDTTYPDKKSILMYITSLFQVLPQQVSIEAIQE |
| | | VEMLPRPPKVTKEEHFQLHHQMHYSQQITVSLAQGYERTSSPKPRFKSYA |
| | | YTQAAYVTTSDPTRSPFPSQHLEAPEDKSFGSSLMESEVNLDRYQTALEE |
| | | VLSWLLSAEDTLQAQGEISNDVEVVKDQFHTHEGYMMDLTAHQGRVGNIL |
| | | QLGSKLIGTGKLSEDEETEVQEQMNLLNSRWECLRVASMEKQSNLHRVLM |
| | | DLQNQKLKELNDWLTKTEERTRKMEEEPLGPDLEDLKRQVQQHKVLQEDL |
| | | EQEQVRVNSLTHMVVVVDESSGDHATAALEEQLKVLGDRWANICRWTEDR |
| | | WVLLQDILLKWQRLTEEQCLFSAWLSEKEDAVNKIHTTGFKDQNEMLSSL |
| | | QKLAVLKADLEKKKQSMGKLYSLKQDLLSTLKNKSVTQKTEAWLDNFARC |
| | | WDNLVQKLEKSTAQISQAVTTTQPSLTQTTVMETVTTVTTREQILVKHAQ |
| | | EELPPPPPQKKRQITVDTLERLQELQEATDELDLKLRQAEVIKGSWQPVG |
| | | DLLIDSLQDHLEKVKALRGEIAPLKENVSHVNDLARQLTTLGIQLSPYNL |
| | | STLEDLNTRWKLLQVAVEDRVRQLHEAHRDFGPASQHELSTSVQGPWERA |
| | | ISPNKVPYYINHETQTTCWDHPKMTELYQSLADLNNVRESAYRTAMKLRR |
| | | LQKALCLDLLSLSAACDALDQHNLKQNDQPMDILQIINCLTTIYDRLEQE |
| | | HNNLVNVPLCVDMCLNWLLNVYDTGRTGRIRVLSFKTGIISLCKAHLEDK |
| | | YRYLFKQVASSTGFCDQRRLGLLLHDSIQIPRQLGEVASEGGSNIEPSVR |
| | | SCFQFANNKPEIEAALFLDWMRLEPQSMVWLPVLHRVAAAETAKHQAKCN |
| | | ICKECPIIGFRYRSLKHFNYDICQSCFFSGRVAKGHKMHYPMVEYCTPTT |
| | | SGEDVRDFAKVLKNKFRTKRYFAKHPRMGYLPVQTVLEGDNMETPVTLIN |
| | | FWPVDSAPASSPQLSHDDTHSRIEHYASRLAEMENSNGSYLNDSISPNES |
| | | IDDEHLLIQHYCQSLNQDSPLSQPRSPAQILISLESEERGELERILADLE |
| | | EENRNLQAEYDRLKQQHEHKGLSPLPSPPEMMPTSPQSPRDAELIAEAKL |
| | | LRQHKGRLEARMQILEDHNKQLESQLHRLRQLLEQPQAEAKVNGTTVSSP |
| | | STSLQRSDSSQPMLLRVVGSQTSDSMGEEDLLSPPQDTSTGLEEVMEQLN |
| | | NSFPSSRGRNTPGKPMREDTM |

### 5.3.2 Nucleic Acid Compositions Encoding Microdystrophin

Another aspect of the present disclosure are nucleic acids comprising a nucleotide sequence encoding a microdystrophin as described herein. Such nucleic acids comprise nucleotide sequences that encode the microdystrophin that has the domains arranged N-terminal to C-terminal as follows: ABD1-H1-R1-R2-R3-H3-R24-H4-CR-CT as detailed, supra. The nucleotide sequence can be any nucleotide sequence that encodes the domains. The nucleotide sequence may be codon optimized and/or depleted of CpG islands for expression in the appropriate context. In particular embodiments, the nucleotide sequences encode a microdystrophin having an amino acid sequence of SEQ ID NO: 52, 53, or 54. The nucleotide sequence can be any sequence that encodes the microdystrophin, including the microdystrophin of SEQ ID NO: 52, SEQ ID NO: 53, or SEQ ID NO: 54, which nucleotide sequence may vary due to the degeneracy of the code. Tables 6 and 7 provide exemplary nucleotide sequences that encode the DMD domains. Table 6 provides the wild type DMD nucleotide sequence for the component and Table 7 provides the nucleotide sequence for the DMD component used in the constructs herein, including sequences that have been codon optimized and/or CpG depleted of CpG islands as follows:

TABLE 6
Dystrophin segment nucleotide sequences

| | SEQ | |
|---|---|---|
| Structure | ID | Nucleic Acid Sequence |
| ABD1 | 55 | ATGCTTTGGTGGGAAGAAGTAGAGGACTGTTATGAAAGAGA |
| | | AGATGTTCAAAAGAAAACATTCACAAAATGGGTAAATGCAC |
| | | AATTTTCTAAGTTTGGGAAGCAGCATATTGAGAACCTCTTC |
| | | AGTGACCTACAGGATGGGAGGCGCCTCCTAGACCTCCTCGA |
| | | AGGCCTGACAGGGCAAAAACTGCCAAAAGAAAAAGGATCCA |
| | | CAAGAGTTCATGCCCTGAACAATGTCAACAAGGCACTGCGG |
| | | GTTTTGCAGAACAATAATGTTGATTTAGTGAATATTGGAAG |
| | | TACTGACATCGTAGATGGAAATCATAAACTGACTCTTGGTT |
| | | TGATTTGGAATATAATCCTCCACTGGCAGGTCAAAAATGTA |
| | | ATGAAAAATATCATGGCTGGATTGCAACAAACCAACAGTGA |
| | | AAAGATTCTCCTGAGCTGGGTCCGACAATCAACTCGTAATT |
| | | ATCCACAGGTTAATGTAATCAACTTCACCACCAGCTGGTCT |
| | | GATGGCCTGGCTTTGAATGCTCTCATCCATAGTCATAGGCC |
| | | AGACCTATTTGACTGGAATAGTGTGGTTTGCCAGCAGTCAG |
| | | CCACACAACGACTGGAACATGCATTCAACATCGCCAGATAT |
| | | CAATTAGGCATAGAGAAACTACTCGATCCTGAAGATGTTGA |
| | | TACCACCTATCCAGATAAGAAGTCCATCTTAATGTACATCA |
| | | CATCACTCTTCCAAGTTTTGCCT |
| L1 | 56 | CAACAAGTGAGCATTGAAGCCATCCAGGAAGTGGAA |
| H1 | 57 | ATGTTGCCAAGGCCACCTAAAGTGACTAAAGAAGAACATTT |
| | | TCAGTTACATCATCAAATGCACTATTCTCAACAGATCACGG |
| | | TCAGTCTAGCACAGGGATATGAGAGAACTTCTTCCCCTAAG |
| | | CCTCGATTCAAGAGCTATGCCTACACACAGGCTGCTTATGT |
| | | CACCACCTCTGACCCTACACGGAGCCCATTTCCTTCACAGC |
| | | ATTTGGAAGCTCCTGAAGAC |
| L2 | 58 | AAGTCATTTGGCAGTTCATTGATGGAG |
| R1 | 59 | AGTGAAGTAAACCTGGACCGTTATCAAACAGCTTTAGAAGA |
| | | AGTATTATCGTGGCTTCTTTCTGCTGAGGACACATTGCAAG |
| | | CACAAGGAGAGATTTCTAATGATGTGGAAGTGGTGAAAGAC |
| | | CAGTTTCATACTCATGAGGGGTACATGATGGATTTGACAGC |
| | | CCATCAGGGCCGGGTTGGTAATATTCTACAATTGGGAAGTA |
| | | AGCTGATTGGAACAGGAAAATTATCAGAAGATGAAGAAACT |
| | | GAAGTACAAGAGCAGATGAATCTCCTAAATTCAAGATGGGA |
| | | ATGCCTCAGGGTAGCTAGCATGGAAAAACAAAGCAATTTAC |
| | | ATAGA |
| R2 | 60 | GTTTTAATGGATCTCCAGAATCAGAAACTGAAAGAGTTGAA |
| | | TGACTGGCTAACAAAAACAGAAGAAAGAACAAGGAAAATGG |
| | | AGGAAGAGCCTCTTGGACCTGATCTTGAAGACCTAAAACGC |
| | | CAAGTACAACAACATAAGGTGCTTCAAGAAGATCTAGAACA |
| | | AGAACAAGTCAGGGTCAATTCTCTCACTCACATGGTGGTGG |
| | | TAGTTGATGAATCTAGTGGAGATCACGCAACTGCTGCTTTG |
| | | GAAGAACAACTTAAGGTATTGGGAGATCGATGGGCAAACAT |
| | | CTGTAGATGGACAGAAGACCGCTGGGTTCTTTTACAAGAC |
| L3 | 61 | ATCCTT |
| R3 | 62 | CTCAAATGGCAACGTCTTACTGAAGAACAGTGCCTTTTTAG |
| | | TGCATGGCTTTCAGAAAAAGAAGATGCAGTGAACAAGATTC |
| | | ACACAACTGGCTTTAAAGATCAAAATGAAATGTTATCAAGT |
| | | CTTCAAAAACTGGCCGTTTTAAAAGCGGATCTAGAAAAGAA |
| | | AAAGCAATCCATGGGCAAACTGTATTCACTCAAACAAGATC |
| | | TTCTTTCAACACTGAAGAATAAGTCAGTGACCCAGAAGACG |
| | | GAAGCATGGCTGGATAACTTTGCCCGGTGTTGGGATAATTT |
| | | AGTCCAAAAACTTGAAAAGAGTACAGCACAGATTTCACAG |
| L4.2 | 63 | CAAACCCTTGAA |
| H3 | 64 | CAGCCTGACCTAGCTCCTGGACTGACCACTATTGGAGCCTC |
| | | TCCTACTCAGACTGTTACTCTGGTGACACAACCTGTGGTTA |
| | | CTAAGGAAACTGCCATCTCCAAACTAGAAATGCCATCTTCC |
| | | TTGATGTTGGAGGTACCT |
| L4 | 65 | ACCCTTGAA |
| R24 | 66 | AGACTCCAACTTCAAGAGGCCACGGATGAGCTGGACCTCAA |
| | | GCTGCGCCAAGCTGAGGTGATCAAGGGATCCTGGCAGCCCG |
| | | TGGGCGATCTCCTCATTGACTCTCTCCAAGATCACCTCGAG |
| | | AAAGTCAAGGCACTTCGAGGAGAAATTGCGCCTCTGAAAGA |
| | | GAACGTGAGCCACGTCAATGACCTTGCTCGCCAGCTTACCA |
| | | CTTTGGGCATTCAGCTCTCACCGTATAACCTCAGCACTCTG |
| | | GAAGACCTGAACACCAGATGGAAGCTTCTGCAGGTGGCCGT |
| | | CGAGGACCGAGTCAGGCAGCTGCATGAA |
| H4 | 67 | GCCCACAGGGACTTTGGTCCAGCATCTCAGCACTTTCTTTC |
| | | CACGTCTGTCCAGGGTCCCTGGGAGAGAGCCATCTCGCCAA |
| | | ACAAAGTGCCCTACTATATCAACCACGAGACTCAAACAACT |
| | | TGCTGGGACCATCCCAAAATGACAGAGCTCTACCAGTCTTT |
| | | AGCTGACCTGAATAATGTCAGATTCTCAGCTTATAGGACTG |
| | | CCATGAAACTC |
| Cysteine- | 68 | CGAAGACTGCAGAAGGCCCTTTGCTTGGATCTCTTGAGCCT |
| rich domain | | GTCAGCTGCATGTGATGCCTTGGACCAGCACAACCTCAAGC |
| (CR) | | AAAATGACCAGCCCATGGATATCCTGCAGATTATTAATTGT |
| | | TTGACCACTATTTATGACCGCCTGGAGCAAGAGCACAACAA |
| | | TTTGGTCAACGTCCCTCTCTGCGTGGATATGTGTCTGAACT |
| | | GGCTGCTGAATGTTTATGATACGGGACGAACAGGGAGGATC |
| | | CGTGTCCTGTCTTTTAAAACTGGCATCATTTCCCTGTGTAA |
| | | AGCACATTTGGAAGACAAGTACAGATACCTTTTCAAGCAAG |
| | | TGGCAAGTTCAACAGGATTTTGTGACCAGCGCAGGCTGGGC |
| | | CTCCTTCTGCATGATTCTATCCAAATTCCAAGACAGTTGGG |
| | | TGAAGTTGCATCCTTTGGGGGCAGTAACATTGAGCCAAGTG |
| | | TCCGGAGCTGCTTCCAATTTGCTAATAATAAGCCAGAGATC |
| | | GAAGCGGCCCTCTTCCTAGACTGGATGAGACTGGAACCCCA |
| | | GTCCATGGTGTGGCTGCCCGTCCTGCACAGAGTGGCTGCTG |
| | | CAGAAACTGCCAAGCATCAGGCCAAATGTAACATCTGCAAA |
| | | GAGTGTCCAATCATTGGATTCAGGTACAGGAGTCTAAAGCA |
| | | CTTTAATTATGACATCTGCCAAAGCTGCTTTTTTTCTGGTC |
| | | GAGTTGCAAAAGGCCATAAAATGCACTATCCCATGGTGGAA |
| | | TATTGC |
| C-terminal | 69 | ACTCCGACTACATCAGGAGAAGATGTTCGAGACTTTGCCAA |
| (CT) | | GGTACTAAAAAACAAATTTCGAACCAAAAGGTATTTTGCGA |
| Domain | | AGCATCCCCGAATGGGCTACCTGCCAGTGCAGACTGTCTTA |
| | | GAGGGGGACAACATGGAAACTCCCGTTACTCTGATCAACTT |
| | | CTGGCCAGTAGATTCTGCGCCTGCCTCGTCCCCTCAGCTTT |
| | | CACACGATGATACTCATTCACGCATTGAACATTATGCTAGC |
| | | AGGCTAGCAGAAATGGAAAACAGCAATGGATCTTATCTAAA |
| | | TGATAGCATCTCTCCTAATGAGAGCATAGATGATGAACATT |
| | | TGTTAATCCAGCATTACTGCCAAAGTTTGAACCAGGACTCC |
| | | CCCCTGAGCCAGCCTCGTAGTCCTGCCCAGATCTTGATTTC |
| | | CTTAGAGAGTGAGGAAAGAGGGGAGCTAGAGAGAATCCTAG |
| | | CAGATCTTGAGGAAGAAAACAGGAATCTGCAAGCAGAATAT |
| | | GACCGTCTAAAGCAGCAGCACGAACATAAAGGCCTGTCCCC |
| | | ACTGCCGTCCCCTCCTGAAATGATGCCCACCTCTCCCCAGA |
| | | GTCCCCGG |
| L4 | 70 | GAGACCCTTGAA |
| L4 | 71 | CTTGAA |

TABLE 7
RGX-DYS segment nucleotide sequences (codon optimized and CpG
depleted

| | SEQ | |
|---|---|---|
| Structure | ID | Nucleic Acid Sequence |
| ABD | 72 | ATGCTTTGGTGGGAAGAGGTGGAAGATTGCTATGAGAGGGAAGATGTG |
| | | CAGAAGAAAACCTTCACCAAATGGGTCAATGCCCAGTTCAGCAAGTTT |
| | | GGCAAGCAGCACATTGAGAACCTGTTCAGTGACCTGCAGGATGGCAGA |
| | | AGGCTGCTGGATCTGCTGGAAGGCCTGACAGGCCAGAAGCTGCCTAAA |
| | | GAGAAGGGCAGCACAAGAGTGCATGCCCTGAACAATGTGAACAAGGCC |
| | | CTGAGAGTGCTGCAGAACAACAATGTGGACCTGGTCAATATTGGCAGC |
| | | ACAGACATTGTGGATGGCAACCACAAGCTGACCCTGGGCCTGATCTGG |
| | | AACATCATCCTGCACTGGCAAGTGAAGAATGTGATGAAGAACATCATG |
| | | GCTGGCCTGCAGCAGACCAACTCTGAGAAGATCCTGCTGAGCTGGGTC |
| | | AGACAGAGCACCAGAAACTACCCTCAAGTGAATGTGATCAACTTCACC |
| | | ACCTCTTGGAGTGATGGACTGGCCCTGAATGCCCTGATCCACAGCCAC |
| | | AGACCTGACCTGTTTGACTGGAACTCTGTTGTGTGCCAGCAGTCTGCC |
| | | ACACAGAGACTGGAACATGCCTTCAACATTGCCAGATACCAGCTGGGA |
| | | ATTGAGAAACTGCTGGACCCTGAGGATGTGGACACCACCTATCCTGAC |
| | | AAGAAATCCATCCTCATGTACATCACCAGCCTGTTCCAGGTGCTGCCC |
| L1 | 73 | CAGCAAGTGTCCATTGAGGCCATTCAAGAGGTTGAG |
| H1 | 74 | ATGCTGCCCAGACCTCCTAAAGTGACCAAAGAGGAACACTTCCAGCTG |
| | | CACCACCAGATGCACTACTCTCAGCAGATCACAGTGTCTCTGGCCCAG |
| | | GGATATGAGAGAACAAGCAGCCCCAAGCCTAGGTTCAAGAGCTATGCC |
| | | TACACACAGGCTGCCTATGTGACCACATCTGACCCCACAAGAAGCCCA |
| | | TTTCCAAGCCAGCATCTGGAAGCCCCTGAGGAC |
| L2 | 75 | AAGAGCTTTGGCAGCAGCCTGATGGAA |
| R1 | 76 | TCTGAAGTGAACCTGGATAGATACCAGACAGCCCTGGAAGAAGTGCTG |
| | | TCCTGGCTGCTGTCTGCTGAGGATACACTGCAGGCTCAGGGTGAAATC |
| | | AGCAATGATGTGGAAGTGGTCAAGGACCAGTTTCACACCCATGAGGGC |
| | | TACATGATGGACCTGACAGCCCACCAGGGCAGAGTGGGAAATATCCTG |
| | | CAGCTGGGCTCCAAGCTGATTGGCACAGGCAAGCTGTCTGAGGATGAA |
| | | GAGACAGAGGTGCAAGAGCAGATGAACCTGCTGAACAGCAGATGGGAG |
| | | TGTCTGAGAGTGGCCAGCATGGAAAAGCAGAGCAACCTGCACAGA |
| R2 | 77 | GTGCTCATGGACCTGCAGAATCAGAAACTGAAAGAACTGAATGACTGG |
| | | CTGACCAAGACAGAAGAAAGGACTAGGAAGATGGAAGAGGAACCTCTG |
| | | GGACCAGACCTGGAAGATCTGAAAAGACAGGTGCAGCAGCATAAGGTG |
| | | CTGCAAGAGGACCTTGAGCAAGAGCAAGTCAGAGTGAACAGCCTGACA |
| | | CACATGGTGGTGGTTGTGGATGAGTCCTCTGGGGATCATGCCACAGCT |
| | | GCTCTGGAAGAACAGCTGAAGGTGCTGGGAGACAGATGGGCCAACATC |
| | | TGTAGGTGGACAGAGGATAGATGGGTGCTGCTCCAGGAC |
| L3 | 78 | ATTCTG |
| R3 | 79 | CTGAAGTGGCAGAGACTGACAGAGGAACAGTGCCTGTTTTCTGCCTGG |
| | | CTCTCTGAGAAAGAGGATGCTGTCAACAAGATCCATACCACAGGCTTC |
| | | AAGGATCAGAATGAGATGCTCAGCTCCCTGCAGAAACTGGCTGTGCTG |
| | | AAGGCTGACCTGGAAAAGAAAAAGCAGTCCATGGGCAAGCTCTACAGC |
| | | CTGAAGCAGGACCTGCTGTCTACCCTGAAGAACAAGTCTGTGACCCAG |
| | | AAAACTGAGGCCTGGCTGGACAACTTTGCTAGATGCTGGGACAACCTG |
| | | GTGCAGAAGCTGGAAAAGTCTACAGCCCAGATCAGCCAG |
| H3 | 80 | CAACCTGATCTTGCCCCTGGCCTGACCACAATTGGAGCCTCTCCAACA |
| | | CAGACTGTGACCCTGGTTACCCAGCCAGTGGTCACCAAAGAGACAGCC |
| | | ATCAGCAAACTGGAAATGCCCAGCTCTCTGATGCTGGAAGTCCCC |
| L4 | 81 | ACACTGGAA |
| L4.1 | 82 | AGTGTG |
| L4.2 | 83 | CAGACACTGGAA |
| R24 | 84 | AGGCTGCAAGAACTTCAAGAGGCCACAGATGAGCTGGACCTGAAGCTG |
| | | AGACAGGCTGAAGTGATCAAAGGCAGCTGGCAGCCAGTTGGGGACCTG |
| | | CTCATTGATAGCCTGCAGGACCATCTGGAAAAAGTGAAAGCCCTGAGG |
| | | GGAGAGATTGCCCCTCTGAAAGAAAATGTGTCCCATGTGAATGACCTG |
| | | GCCAGACAGCTGACCACACTGGGAATCCAGCTGAGCCCCTACAACCTG |
| | | AGCACCCTTGAGGACCTGAACACCAGGTGGAAGCTCCTCCAGGTGGCA |
| | | GTGGAAGATAGAGTCAGGCAGCTGCATGAG |
| H4 | 85 | GCCCACAGAGATTTTGGACCAGCCAGCCAGCACTTTCTGTCTACCTCT |
| | | GTGCAAGGCCCCTGGGAGAGAGCTATCTCTCCTAACAAGGTGCCCTAC |
| | | TACATCAACCATGAGACACAGACCACCTGTTGGGATCACCCCAAGATG |
| | | ACAGAGCTGTACCAGAGTCTGGCAGACCTCAACAATGTCAGATTCAGT |
| | | GCCTACAGGACTGCCATGAAGCTC |
| Cysteine- | 86 | AGAAGGCTCCAGAAAGCTCTGTGCCTGGACCTGCTTTCCCTGAGTGCA |
| rich | | GCTTGTGATGCCCTGGACCAGCACAATCTGAAGCAGAATGACCAGCCT |
| domain | | ATGGACATCCTCCAGATCATCAACTGCCTCACCACCATCTATGATAGG |
| (CR) | | CTGGAACAAGAGCACAACAATCTGGTCAATGTGCCCCTGTGTGTGGAC |
| | | ATGTGCCTGAATTGGCTGCTGAATGTGTATGACACAGGCAGAACAGGC |
| | | AGGATCAGAGTCCTGTCCTTCAAGACAGGCATCATCTCCCTGTGCAAA |
| | | GCCCACTTGGAGGACAAGTACAGATACCTGTTCAAGCAAGTGGCCTCC |
| | | AGCACAGGCTTTTGTGACCAGAGAAGGCTGGGCCTGCTCCTGCATGAC |
| | | AGCATTCAGATCCCTAGACAGCTGGGAGAAGTGGCTTCCTTTGGAGGC |
| | | AGCAATATTGAGCCATCAGTCAGGTCCTGTTTTCAGTTTGCCAACAAC |
| | | AAGCCTGAGATTGAGGCTGCCCTGTTCCTGGACTGGATGAGACTTGAG |
| | | CCTCAGAGCATGGTCTGGCTGCCTGTGCTTCATAGAGTGGCTGCTGCT |
| | | GAGACTGCCAAGCACCAGGCCAAGTGCAACATCTGCAAAGAGTGCCCC |
| | | ATCATTGGCTTCAGATACAGATCCCTGAAGCACTTCAACTATGATATC |
| | | TGCCAGAGCTGCTTCTTTAGTGGCAGGGTTGCCAAGGGCCACAAAATG |
| | | CACTACCCCATGGTGGAATACTGC |
| C-terminal | 87 | ACCCCAACAACCTCTGGGGAAGATGTTAGAGACTTTGCCAAGGTGCTG |
| (CT) | | AAAAACAAGTTCAGGACCAAGAGATACTTTGCTAAGCACCCCAGAATG |
| Domain | | GGCTACCTGCCTGTCCAGACAGTGCTTGAGGGTGACAACATGGAAACC |
| (DYS1) | | CCTGTGACACTGATCAATTTCTGGCCAGTGGACTCTGCCCCTGCCTCA |
| | | AGTCCACAGCTGTCCCATGATGACACCCACAGCAGAATTGAGCACTAT |
| | | GCCTCCAGACTGGCAGAGATGGAAAACAGCAATGGCAGCTACCTGAAT |
| | | GATAGCATCAGCCCCAATGAGAGCATTGATGATGAGCATCTGCTGATC |
| | | CAGCACTACTGTCAGTCCCTGAACCAGGACTCTCCACTGAGCCAGCCT |
| | | AGAAGCCCTGCTCAGATCCTGATCAGCCTTGAGTCTGAGGAAAGGGGA |
| | | GAGCTGGAAAGAATCCTGGCAGATCTTGAGGAAGAGAACAGAAACCTG |
| | | CAGGCAGAGTATGACAGGCTCAAACAGCAGCATGAGCACAAGGGACTG |
| | | AGCCCTCTGCCTTCTCCTCCTGAAATGATGCCCACCTCTCCACAGTCT |
| | | CCAAGGTGATGA (stop codons underlined) |
| Minimal | 88 | ACCCCAACAACCTCTGGGGAAGATGTTAGAGACTTTGCCAAGGTGCTG |
| C-terminal | | AAAAACAAGTTCAGGACCAAGAGATACTTTGCTAAGCACCCCAGAATG |
| (CT1.5) | | GGCTACCTGCCTGTCCAGACAGTGCTTGAGGGTGACAACATGGAAACC |
| Domain | | CCTGTGACACTGATCAATTTCTGGCCAGTGGACTCTGCCCCTGCCTCA |
| (DYS5,) | | AGTCCACAGCTGTCCCATGATGACACCCACAGCAGAATTGAGCACTAT |
| | | GCCTCCAGACTGGCAGAGATGGAAAACAGCAATGGCAGCTACCTGAAT |
| | | GATAGCATCAGCCCCAATGAGAGCATTGATGATGAGCATCTGCTGATC |
| | | CAGCACTACTGTCAGTCCCTGAACCAGGACTCTCCACTGAGCCAGCCT |
| | | AGAAGCCCTGCTCAGATCCTGATCAGCCTTGAGTCTTGATGA (stop |
| | | codons underlined) |
| L4 | 89 | GA(A/G)ACACTGGAA or GAGACACTGGAA |
| L4 | 90 | CTGGAA |

In various embodiments, the nucleic acid comprises a nucleotide sequence encoding the microdystrophin having the amino acid sequence of SEQ ID NO: 52, SEQ ID NO: 53, or SEQ ID NO: 54. In embodiments, the nucleic acid comprises a nucleotide sequence which is encompassed by SEQ ID NO: 91, SEQ ID NO: 92, or SEQ ID NO: 93 (encoding the microdystrophins of SEQ ID NO: 52, SEQ ID NO: 53, or SEQ ID NO: 54, respectively). In various embodiments, the nucleotide sequence encoding a microdystrophin may have at least 50%, at least 60%, at least 70%, at least 75%, at least 80%, at least 85%, at least 90%, at least 95%, at least 98% or at least 99% sequence identity to the nucleotide sequence of SEQ ID NO: 91, 92, or 93 (Table 8) or the reverse complement thereof and encode a therapeutically effective microdystrophin.

TABLE 8
RGX-DYS Encoding nucleotide sequences

| Structure | SEQ ID | Nucleic Acid Sequence |
|---|---|---|
| DYS1 | 91 | ATGCTTTGGTGGGAAGAGGTGGAAGATTGCTATGAGAGGGAAGATGTGCA |
| | | GAAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCTG |
| | | CTGGATCTGCTGGAAGGCCTGACAGGCCAGAAGCTGCCTAAAGAGAAGGG |
| | | CAGCACAAGAGTGCATGCCCTGAACAATGTGAACAAGGCCCTGAGAGTGC |
| | | TGCAGAACAACAATGTGGACCTGGTCAATATTGGCAGCACAGACATTGTG |
| | | GATGGCAACCACAAGCTGACCCTGGGCCTGATCTGGAACATCATCCTGCA |
| | | CTGGCAAGTGAAGAATGTGATGAAGAACATCATGGCTGGCCTGCAGCAGA |
| | | CCAACTCTGAGAAGATCCTGCTGAGCTGGGTCAGACAGAGCACCAGAAAC |
| | | TACCCTCAAGTGAATGTGATCAACTTCACCACCTCTTGGAGTGATGGACT |
| | | GGCCCTGAATGCCCTGATCCACAGCCACAGACCTGACCTGTTTGACTGGA |
| | | ACTCTGTTGTGTGCCAGCAGTCTGCCACACAGAGACTGGAACATGCCTTC |
| | | AACATTGCCAGATACCAGCTGGGAATTGAGAAACTGCTGGACCCTGAGGA |
| | | TGTGGACACCACCTATCCTGACAAGAAATCCATCCTCATGTACATCACCA |
| | | GCCTGTTCCAGGTGCTGCCCCAGCAAGTGTCCATTGAGGCCATTCAAGAG |
| | | GTTGAGATGCTGCCCAGACCTCCTAAAGTGACCAAAGAGGAACACTTCCA |
| | | GCTGCACCACCAGATGCACTACTCTCAGCAGATCACAGTGTCTCTGGCCC |
| | | AGGGATATGAGAGAACAAGCAGCCCCAAGCCTAGGTTCAAGAGCTATGCC |
| | | TACACACAGGCTGCCTATGTGACCACATCTGACCCCACAAGAAGCCCATT |
| | | TCCAAGCCAGCATCTGGAAGCCCCTGAGGACAAGAGCTTTGGCAGCAGCC |
| | | TGATGGAATCTGAAGTGAACCTGGATAGATACCAGACAGCCCTGGAAGAA |
| | | GTGCTGTCCTGGCTGCTGTCTGCTGAGGATACACTGCAGGCTCAGGGTGA |
| | | AATCAGCAATGATGTGGAAGTGGTCAAGGACCAGTTTCACACCCATGAGG |
| | | GCTACATGATGGACCTGACAGCCCACCAGGGCAGAGTGGGAAATATCCTG |
| | | CAGCTGGGCTCCAAGCTGATTGGCACAGGCAAGCTGTCTGAGGATGAAGA |
| | | GACAGAGGTGCAAGAGCAGATGAACCTGCTGAACAGCAGATGGGAGTGTC |
| | | TGAGAGTGGCCAGCATGGAAAAGCAGAGCAACCTGCACAGAGTGCTCATG |
| | | GACCTGCAGAATCAGAAACTGAAAGAACTGAATGACTGGCTGACCAAGAC |
| | | AGAAGAAAGGACTAGGAAGATGGAAGAGGAACCTCTGGGACCAGACCTGG |
| | | AAGATCTGAAAAGACAGGTGCAGCAGCATAAGGTGCTGCAAGAGGACCTT |
| | | GAGCAAGAGCAAGTCAGAGTGAACAGCCTGACACACATGGTGGTGGTTGT |
| | | GGATGAGTCCTCTGGGGATCATGCCACAGCTGCTCTGGAAGAACAGCTGA |
| | | AGGTGCTGGGAGACAGATGGGCCAACATCTGTAGGTGGACAGAGGATAGA |
| | | TGGGTGCTGCTCCAGGACATTCTGCTGAAGTGGCAGAGACTGACAGAGGA |
| | | ACAGTGCCTGTTTTCTGCCTGGCTCTCTGAGAAAGAGGATGCTGTCAACA |
| | | AGATCCATACCACAGGCTTCAAGGATCAGAATGAGATGCTCAGCTCCCTG |
| | | CAGAAACTGGCTGTGCTGAAGGCTGACCTGGAAAAGAAAAAGCAGTCCAT |
| | | GGGCAAGCTCTACAGCCTGAAGCAGGACCTGCTGTCTACCCTGAAGAACA |
| | | AGTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCCA |
| | | GCAACCTGATCTTGCCCCTGGCCTGACCACAATTGGAGCCTCTCCAACAC |
| | | AGACTGTGACCCTGGTTACCCAGCCAGTGGTCACCAAAGAGACAGCCATC |
| | | AGCAAACTGGAAATGCCCAGCTCTCTGATGCTGGAAGTCCCCACACTGGA |
| | | AAGGCTGCAAGAACTTCAAGAGGCCACAGATGAGCTGGACCTGAAGCTGA |
| | | GACAGGCTGAAGTGATCAAAGGCAGCTGGCAGCCAGTTGGGGACCTGCTC |
| | | ATTGATAGCCTGCAGGACCATCTGGAAAAAGTGAAAGCCCTGAGGGGAGA |
| | | GATTGCCCCTCTGAAAGAAAATGTGTCCCATGTGAATGACCTGGCCAGAC |
| | | AGCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATAG |
| | | AGTCAGGCAGCTGCATGAGGCCCACAGAGATTTTGGACCAGCCAGCCAGC |
| | | ACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGA |
| | | TCACCCCAAGATGACAGAGCTGTACCAGAGTCTGGCAGACCTCAACAATG |
| | | TCAGATTCAGTGCCTACAGGACTGCCATGAAGCTCAGAAGGCTCCAGAAA |
| | | GCTCTGTGCCTGGACCTGCTTTCCCTGAGTGCAGCTTGTGATGCCCTGGA |
| | | CCAGCACAATCTGAAGCAGAATGACCAGCCTATGGACATCCTCCAGATCA |
| | | TCAACTGCCTCACCACCATCTATGATAGGCTGGAACAAGAGCACAACAAT |
| | | CTGGTCAATGTGCCCCTGTGTGTGGACATGTGCCTGAATTGGCTGCTGAA |
| | | TGTGTATGACACAGGCAGAACAGGCAGGATCAGAGTCCTGTCCTTCAAGA |
| | | CAGGCATCATCTCCCTGTGCAAAGCCCACTTGGAGGACAAGTACAGATAC |
| | | CTGTTCAAGCAAGTGGCCTCCAGCACAGGCTTTTGTGACCAGAGAAGGCT |
| | | GGGCCTGCTCCTGCATGACAGCATTCAGATCCCTAGACAGCTGGGAGAAG |
| | | TGGCTTCCTTTGGAGGCAGCAATATTGAGCCATCAGTCAGGTCCTGTTTT |
| | | CAGTTTGCCAACAACAAGCCTGAGATTGAGGCTGCCCTGTTCCTGGACTG |
| | | GATGAGACTTGAGCCTCAGAGCATGGTCTGGCTGCCTGTGCTTCATAGAG |
| | | TGGCTGCTGCTGAGACTGCCAAGCACCAGGCCAAGTGCAACATCTGCAAA |
| | | GAGTGCCCCATCATTGGCTTCAGATACAGATCCCTGAAGCACTTCAACTA |
| | | TGATATCTGCCAGAGCTGCTTCTTTAGTGGCAGGGTTGCCAAGGGCCACA |
| | | AAATGCACTACCCCATGGTGGAATACTGCACCCCAACAACCTCTGGGGAA |
| | | GATGTTAGAGACTTTGCCAAGGTGCTGAAAAACAAGTTCAGGACCAAGAG |
| | | ATACTTTGCTAAGCACCCCAGAATGGGCTACCTGCCTGTCCAGACAGTGC |
| | | TTGAGGGTGACAACATGGAAACCCCTGTGACACTGATCAATTTCTGGCCA |
| | | GTGGACTCTGCCCCTGCCTCAAGTCCACAGCTGTCCCATGATGACACCCA |
| | | CAGCAGAATTGAGCACTATGCCTCCAGACTGGCAGAGATGGAAAACAGCA |
| | | ATGGCAGCTACCTGAATGATAGCATCAGCCCCAATGAGAGCATTGATGAT |
| | | GAGCATCTGCTGATCCAGCACTACTGTCAGTCCCTGAACCAGGACTCTCC |
| | | ACTGAGCCAGCCTAGAAGCCCTGCTCAGATCCTGATCAGCCTTGAGTCTG |
| | | AGGAAAGGGGAGAGCTGGAAAGAATCCTGGCAGATCTTGAGGAAGAGAAC |
| | | AGAAACCTGCAGGCAGAGTATGACAGGCTCAAACAGCAGCATGAGCACAA |
| | | GGGACTGAGCCCTCTGCCTTCTCCTCCTGAAATGATGCCCACCTCTCCAC |
| | | AGTCTCCAAGGTGATGA |
| DYS3 | 92 | ATGCTTTGGTGGGAAGAGGTGGAAGATTGCTATGAGAGGGAAGATGTGCA |
| | | GAAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCTG |
| | | CTGGATCTGCTGGAAGGCCTGACAGGCCAGAAGCTGCCTAAAGAGAAGGG |
| | | CAGCACAAGAGTGCATGCCCTGAACAATGTGAACAAGGCCCTGAGAGTGC |
| | | TGCAGAACAACAATGTGGACCTGGTCAATATTGGCAGCACAGACATTGTG |
| | | GATGGCAACCACAAGCTGACCCTGGGCCTGATCTGGAACATCATCCTGCA |
| | | CTGGCAAGTGAAGAATGTGATGAAGAACATCATGGCTGGCCTGCAGCAGA |
| | | CCAACTCTGAGAAGATCCTGCTGAGCTGGGTCAGACAGAGCACCAGAAAC |
| | | TACCCTCAAGTGAATGTGATCAACTTCACCACCTCTTGGAGTGATGGACT |
| | | GGCCCTGAATGCCCTGATCCACAGCCACAGACCTGACCTGTTTGACTGGA |
| | | ACTCTGTTGTGTGCCAGCAGTCTGCCACACAGAGACTGGAACATGCCTTC |
| | | AACATTGCCAGATACCAGCTGGGAATTGAGAAACTGCTGGACCCTGAGGA |
| | | TGTGGACACCACCTATCCTGACAAGAAATCCATCCTCATGTACATCACCA |
| | | GCCTGTTCCAGGTGCTGCCCCAGCAAGTGTCCATTGAGGCCATTCAAGAG |
| | | GTTGAGATGCTGCCCAGACCTCCTAAAGTGACCAAAGAGGAACACTTCCA |
| | | GCTGCACCACCAGATGCACTACTCTCAGCAGATCACAGTGTCTCTGGCCC |
| | | AGGGATATGAGAGAACAAGCAGCCCCAAGCCTAGGTTCAAGAGCTATGCC |
| | | TACACACAGGCTGCCTATGTGACCACATCTGACCCCACAAGAAGCCCATT |
| | | TCCAAGCCAGCATCTGGAAGCCCCTGAGGACAAGAGCTTTGGCAGCAGCC |
| | | TGATGGAATCTGAAGTGAACCTGGATAGATACCAGACAGCCCTGGAAGAA |
| | | GTGCTGTCCTGGCTGCTGTCTGCTGAGGATACACTGCAGGCTCAGGGTGA |
| | | AATCAGCAATGATGTGGAAGTGGTCAAGGACCAGTTTCACACCCATGAGG |
| | | GCTACATGATGGACCTGACAGCCCACCAGGGCAGAGTGGGAAATATCCTG |
| | | CAGCTGGGCTCCAAGCTGATTGGCACAGGCAAGCTGTCTGAGGATGAAGA |
| | | GACAGAGGTGCAAGAGCAGATGAACCTGCTGAACAGCAGATGGGAGTGTC |
| | | TGAGAGTGGCCAGCATGGAAAAGCAGAGCAACCTGCACAGAGTGCTCATG |
| | | GACCTGCAGAATCAGAAACTGAAAGAACTGAATGACTGGCTGACCAAGAC |
| | | AGAAGAAAGGACTAGGAAGATGGAAGAGGAACCTCTGGGACCAGACCTGG |
| | | AAGATCTGAAAAGACAGGTGCAGCAGCATAAGGTGCTGCAAGAGGACCTT |
| | | GAGCAAGAGCAAGTCAGAGTGAACAGCCTGACACACATGGTGGTGGTTGT |
| | | GGATGAGTCCTCTGGGGATCATGCCACAGCTGCTCTGGAAGAACAGCTGA |
| | | AGGTGCTGGGAGACAGATGGGCCAACATCTGTAGGTGGACAGAGGATAGA |
| | | TGGGTGCTGCTCCAGGACATTCTGCTGAAGTGGCAGAGACTGACAGAGGA |
| | | ACAGTGCCTGTTTTCTGCCTGGCTCTCTGAGAAAGAGGATGCTGTCAACA |
| | | AGATCCATACCACAGGCTTCAAGGATCAGAATGAGATGCTCAGCTCCCTG |
| | | CAGAAACTGGCTGTGCTGAAGGCTGACCTGGAAAAGAAAAAGCAGTCCAT |
| | | GGGCAAGCTCTACAGCCTGAAGCAGGACCTGCTGTCTACCCTGAAGAACA |
| | | AGTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCCA |
| | | GCAACCTGATCTTGCCCCTGGCCTGACCACAATTGGAGCCTCTCCAACAC |
| | | AGACTGTGACCCTGGTTACCCAGCCAGTGGTCACCAAAGAGACAGCCATC |
| | | AGCAAACTGGAAATGCCCAGCTCTCTGATGCTGGAAGTCCCCACACTGGA |
| | | AAGGCTGCAAGAACTTCAAGAGGCCACAGATGAGCTGGACCTGAAGCTGA |
| | | GACAGGCTGAAGTGATCAAAGGCAGCTGGCAGCCAGTTGGGGACCTGCTC |
| | | ATTGATAGCCTGCAGGACCATCTGGAAAAAGTGAAAGCCCTGAGGGGAGA |
| | | GATTGCCCCTCTGAAAGAAAATGTGTCCCATGTGAATGACCTGGCCAGAC |
| | | AGCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATAG |
| | | AGTCAGGCAGCTGCATGAGGCCCACAGAGATTTTGGACCAGCCAGCCAGC |
| | | ACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGA |
| | | TCACCCCAAGATGACAGAGCTGTACCAGAGTCTGGCAGACCTCAACAATG |
| | | TCAGATTCAGTGCCTACAGGACTGCCATGAAGCTCAGAAGGCTCCAGAAA |
| | | GCTCTGTGCCTGGACCTGCTTTCCCTGAGTGCAGCTTGTGATGCCCTGGA |
| | | CCAGCACAATCTGAAGCAGAATGACCAGCCTATGGACATCCTCCAGATCA |
| | | TCAACTGCCTCACCACCATCTATGATAGGCTGGAACAAGAGCACAACAAT |
| | | CTGGTCAATGTGCCCCTGTGTGTGGACATGTGCCTGAATTGGCTGCTGAA |
| | | TGTGTATGACACAGGCAGAACAGGCAGGATCAGAGTCCTGTCCTTCAAGA |
| | | CAGGCATCATCTCCCTGTGCAAAGCCCACTTGGAGGACAAGTACAGATAC |
| | | CTGTTCAAGCAAGTGGCCTCCAGCACAGGCTTTTGTGACCAGAGAAGGCT |
| | | GGGCCTGCTCCTGCATGACAGCATTCAGATCCCTAGACAGCTGGGAGAAG |
| | | TGGCTTCCTTTGGAGGCAGCAATATTGAGCCATCAGTCAGGTCCTGTTTT |
| | | CAGTTTGCCAACAACAAGCCTGAGATTGAGGCTGCCCTGTTCCTGGACTG |
| | | GATGAGACTTGAGCCTCAGAGCATGGTCTGGCTGCCTGTGCTTCATAGAG |
| | | TGGCTGCTGCTGAGACTGCCAAGCACCAGGCCAAGTGCAACATCTGCAAA |
| | | GAGTGCCCCATCATTGGCTTCAGATACAGATCCCTGAAGCACTTCAACTA |
| | | TGATATCTGCCAGAGCTGCTTCTTTAGTGGCAGGGTTGCCAAGGGCCACA |
| | | AAATGCACTACCCCATGGTGGAATACTGCACCCCAACAACCTCTGGGGAA |
| | | GATGTTAGAGACTTTGCCAAGGTGCTGAAAAACAAGTTCAGGACCAAGAG |
| | | ATACTTTGCTAAGCACCCCAGAATGGGCTACCTGCCTGTCCAGACAGTGC |
| | | TTGAGGGTGACAACATGGAAACC |
| DYS5 | 93 | ATGCTTTGGTGGGAAGAGGTGGAAGATTGCTATGAGAGGGAAGATGTGCA |
| | | GAAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCTG |
| | | CTGGATCTGCTGGAAGGCCTGACAGGCCAGAAGCTGCCTAAAGAGAAGGG |
| | | CAGCACAAGAGTGCATGCCCTGAACAATGTGAACAAGGCCCTGAGAGTGC |
| | | TGCAGAACAACAATGTGGACCTGGTCAATATTGGCAGCACAGACATTGTG |
| | | GATGGCAACCACAAGCTGACCCTGGGCCTGATCTGGAACATCATCCTGCA |
| | | CTGGCAAGTGAAGAATGTGATGAAGAACATCATGGCTGGCCTGCAGCAGA |
| | | CCAACTCTGAGAAGATCCTGCTGAGCTGGGTCAGACAGAGCACCAGAAAC |
| | | TACCCTCAAGTGAATGTGATCAACTTCACCACCTCTTGGAGTGATGGACT |
| | | GGCCCTGAATGCCCTGATCCACAGCCACAGACCTGACCTGTTTGACTGGA |
| | | ACTCTGTTGTGTGCCAGCAGTCTGCCACACAGAGACTGGAACATGCCTTC |
| | | AACATTGCCAGATACCAGCTGGGAATTGAGAAACTGCTGGACCCTGAGGA |
| | | TGTGGACACCACCTATCCTGACAAGAAATCCATCCTCATGTACATCACCA |
| | | GCCTGTTCCAGGTGCTGCCCCAGCAAGTGTCCATTGAGGCCATTCAAGAG |
| | | GTTGAGATGCTGCCCAGACCTCCTAAAGTGACCAAAGAGGAACACTTCCA |
| | | GCTGCACCACCAGATGCACTACTCTCAGCAGATCACAGTGTCTCTGGCCC |
| | | AGGGATATGAGAGAACAAGCAGCCCCAAGCCTAGGTTCAAGAGCTATGCC |
| | | TACACACAGGCTGCCTATGTGACCACATCTGACCCCACAAGAAGCCCATT |
| | | TCCAAGCCAGCATCTGGAAGCCCCTGAGGACAAGAGCTTTGGCAGCAGCC |
| | | TGATGGAATCTGAAGTGAACCTGGATAGATACCAGACAGCCCTGGAAGAA |
| | | GTGCTGTCCTGGCTGCTGTCTGCTGAGGATACACTGCAGGCTCAGGGTGA |
| | | AATCAGCAATGATGTGGAAGTGGTCAAGGACCAGTTTCACACCCATGAGG |
| | | GCTACATGATGGACCTGACAGCCCACCAGGGCAGAGTGGGAAATATCCTG |
| | | CAGCTGGGCTCCAAGCTGATTGGCACAGGCAAGCTGTCTGAGGATGAAGA |
| | | GACAGAGGTGCAAGAGCAGATGAACCTGCTGAACAGCAGATGGGAGTGTC |
| | | TGAGAGTGGCCAGCATGGAAAAGCAGAGCAACCTGCACAGAGTGCTCATG |
| | | GACCTGCAGAATCAGAAACTGAAAGAACTGAATGACTGGCTGACCAAGAC |
| | | AGAAGAAAGGACTAGGAAGATGGAAGAGGAACCTCTGGGACCAGACCTGG |
| | | AAGATCTGAAAAGACAGGTGCAGCAGCATAAGGTGCTGCAAGAGGACCTT |
| | | GAGCAAGAGCAAGTCAGAGTGAACAGCCTGACACACATGGTGGTGGTTGT |
| | | GGATGAGTCCTCTGGGGATCATGCCACAGCTGCTCTGGAAGAACAGCTGA |
| | | AGGTGCTGGGAGACAGATGGGCCAACATCTGTAGGTGGACAGAGGATAGA |
| | | TGGGTGCTGCTCCAGGACATTCTGCTGAAGTGGCAGAGACTGACAGAGGA |
| | | ACAGTGCCTGTTTTCTGCCTGGCTCTCTGAGAAAGAGGATGCTGTCAACA |
| | | AGATCCATACCACAGGCTTCAAGGATCAGAATGAGATGCTCAGCTCCCTG |
| | | CAGAAACTGGCTGTGCTGAAGGCTGACCTGGAAAAGAAAAAGCAGTCCAT |
| | | GGGCAAGCTCTACAGCCTGAAGCAGGACCTGCTGTCTACCCTGAAGAACA |
| | | AGTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCCA |
| | | GCAACCTGATCTTGCCCCTGGCCTGACCACAATTGGAGCCTCTCCAACAC |
| | | AGACTGTGACCCTGGTTACCCAGCCAGTGGTCACCAAAGAGACAGCCATC |
| | | AGCAAACTGGAAATGCCCAGCTCTCTGATGCTGGAAGTCCCCACACTGGA |
| | | AAGGCTGCAAGAACTTCAAGAGGCCACAGATGAGCTGGACCTGAAGCTGA |
| | | GACAGGCTGAAGTGATCAAAGGCAGCTGGCAGCCAGTTGGGGACCTGCTC |
| | | ATTGATAGCCTGCAGGACCATCTGGAAAAAGTGAAAGCCCTGAGGGGAGA |
| | | GATTGCCCCTCTGAAAGAAAATGTGTCCCATGTGAATGACCTGGCCAGAC |
| | | AGCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATAG |
| | | AGTCAGGCAGCTGCATGAGGCCCACAGAGATTTTGGACCAGCCAGCCAGC |
| | | ACTTTCTGTCTACCTCTGTGCAAGGCCCCTGGGAGAGAGCTATCTCTCCT |
| | | AACAAGGTGCCCTACTACATCAACCATGAGACACAGACCACCTGTTGGGA |
| | | TCACCCCAAGATGACAGAGCTGTACCAGAGTCTGGCAGACCTCAACAATG |
| | | TCAGATTCAGTGCCTACAGGACTGCCATGAAGCTCAGAAGGCTCCAGAAA |
| | | GCTCTGTGCCTGGACCTGCTTTCCCTGAGTGCAGCTTGTGATGCCCTGGA |
| | | CCAGCACAATCTGAAGCAGAATGACCAGCCTATGGACATCCTCCAGATCA |
| | | TCAACTGCCTCACCACCATCTATGATAGGCTGGAACAAGAGCACAACAAT |
| | | CTGGTCAATGTGCCCCTGTGTGTGGACATGTGCCTGAATTGGCTGCTGAA |
| | | TGTGTATGACACAGGCAGAACAGGCAGGATCAGAGTCCTGTCCTTCAAGA |
| | | CAGGCATCATCTCCCTGTGCAAAGCCCACTTGGAGGACAAGTACAGATAC |
| | | CTGTTCAAGCAAGTGGCCTCCAGCACAGGCTTTTGTGACCAGAGAAGGCT |
| | | GGGCCTGCTCCTGCATGACAGCATTCAGATCCCTAGACAGCTGGGAGAAG |
| | | TGGCTTCCTTTGGAGGCAGCAATATTGAGCCATCAGTCAGGTCCTGTTTT |
| | | CAGTTTGCCAACAACAAGCCTGAGATTGAGGCTGCCCTGTTCCTGGACTG |
| | | GATGAGACTTGAGCCTCAGAGCATGGTCTGGCTGCCTGTGCTTCATAGAG |
| | | TGGCTGCTGCTGAGACTGCCAAGCACCAGGCCAAGTGCAACATCTGCAAA |
| | | GAGTGCCCCATCATTGGCTTCAGATACAGATCCCTGAAGCACTTCAACTA |
| | | TGATATCTGCCAGAGCTGCTTCTTTAGTGGCAGGGTTGCCAAGGGCCACA |
| | | AAATGCACTACCCCATGGTGGAATACTGCACCCCAACAACCTCTGGGGAA |
| | | GATGTTAGAGACTTTGCCAAGGTGCTGAAAAACAAGTTCAGGACCAAGAG |
| | | ATACTTTGCTAAGCACCCCAGAATGGGCTACCTGCCTGTCCAGACAGTGC |
| | | TTGAGGGTGACAACATGGAAACCCCTGTGACACTGATCAATTTCTGGCCA |
| | | GTGGACTCTGCCCCTGCCTCAAGTCCACAGCTGTCCCATGATGACACCCA |
| | | CAGCAGAATTGAGCACTATGCCTCCAGACTGGCAGAGATGGAAAACAGCA |
| | | ATGGCAGCTACCTGAATGATAGCATCAGCCCCAATGAGAGCATTGATGAT |
| | | GAGCATCTGCTGATCCAGCACTACTGTCAGTCCCTGAACCAGGACTCTCC |
| | | ACTGAGCCAGCCTAGAAGCCCTGCTCAGATCCTGATCAGCCTTGAGTCTT |
| | | GATGA |

### 5.3.2.1 Codon Optimization and CpG Depletion

In one aspect the nucleotide sequence encoding the microdystrophin cassette is modified by codon optimization and CpG dinucleotide and CpG island depletion. Immune response against microdystrophin transgene is a concern for human clinical application, as evidenced in the first Duchenne Muscular Dystrophy (DMD) gene therapy clinical trials and in several adeno-associated vial (AAV)-minidystrophin gene therapy in canine models [Mendell, J. R., et al., Dystrophin immunity in Duchenne's muscular dystrophy. N Engl J Med, 2010. 363(15): p. 1429-37; and Kornegay, J. N., et al., Widespread muscle expression of an AAV9 human mini-dystrophin vector after intravenous injection in neonatal dystrophin-deficient dogs. Mol Ther, 2010. 18(8): p. 1501-8].

In embodiments, the microdystrophin cassette is human codon-optimized with CpG depletion. Codon-optimized and CpG depleted nucleotide sequences may be designed by any method known in the art, including for example, by Thermo Fisher Scientific GeneArt Gene Synthesis tools utilizing GeneOptimizer (Waltham, MA USA)). Nucleotide sequences SEQ ID NOs: 91, 92, 93 described herein represent codon-optimized and CpG depleted sequences.

Provided are microdystrophin transgenes that have reduced numbers of CpG dinucleotide sequences and, as a result, have reduced number of CpG islands. In certain embodiments, the microdystrophin nucleotide sequence has fewer than two (2) CpG islands, or one (1) CpG island or zero (0) CpG islands. In embodiments, provided are microdystrophin transgenes having fewer than 2, or 1 CpG islands, or 0 CpG islands that have reduced immunogenicity, as measured by anti-drug antibody titer compared to a microdystrophin transgene having more than 2 CpG islands. In certain embodiments, the microdystrophin nucleotide sequence consisting essentially of SEQ ID NO: 91, 92, or 93 has zero (0) CpG islands. In other embodiments, the microdystrophin transgene nucleotide sequence consisting essentially of a microdystrophin gene operably linked to a promoter, wherein the microdystrophin consists of SEQ ID NO: 91, 92, or 93, has less than two (2) CpG islands. In still other embodiments, the microdystrophin transgene nucleotide sequence consisting essentially of a microdystrophin gene operably linked to a promoter, wherein the microdystrophin consists of SEQ ID NO: 91, 92, or 93, has one (1) CpG island.

### 5.3.3 Microdystrophin Transgene Constructs

Provided for use in the methods disclosed herein are microdystrophin transgene constructs and artificial rAAV genomes. The transgenes comprise nucleotide sequences encoding microdystrophins disclosed herein operably linked to transcriptional regulatory sequences, including promoters, that promote expression in muscle cells and other regulatory sequences that promote expression of the microdystrophin. The transgenes are flanked by AAV ITR sequences.

In some embodiments, the rAAV genome comprises a vector comprising the following components: (1) AAV inverted terminal repeats that flank an expression cassette; (2) regulatory control elements, such as a) promoter/enhancers, b) a poly A signal, and c) optionally an intron; and (3) nucleic acid sequences coding for the microdystrophin, for example as in Table 8. In a specific embodiment, the constructs described herein comprise the following components: (1) AAV2 or AAV8 inverted terminal repeats (ITRs) that flank the expression cassette; (2) control elements, which include a muscle-specific Spc5-12 promoter and a small poly A signal; and (3) transgene providing (e.g., coding for) a nucleic acid encoding microdystrophin as described herein, including the microdystrophin coding sequence of the RGX-DYS1 transgene (SEQ ID NO:91) or the RGX-DYS5 transgene (SEQ ID NO:93). In a specific embodiment, the constructs described herein comprise the following components: (1) AAV2 or AAV8 ITRs that flank the expression cassette; (2) control elements, which include a) the muscle-specific Spc5-12 promoter, b) a small poly A signal; and (3) microdystrophin cassette, which includes from the N-terminus to the C-terminus, ABD1-H1-R1-R2-R3-H3-R24-H4-CR-CT, wherein CT comprises at least the portion of the CT comprising an α1-syntrophin binding site, including the CT having an amino acid sequence of SEQ ID NO:48 or 49. In a specific embodiment, the constructs described herein comprise the following components: (1) AAV2 or AAV8 ITRs that flank the expression cassette; (2) control elements, which include a) the muscle-specific Spc5-12 promoter, b) an intron (e.g., VH4) and c) a small poly A signal; and (3) microdystrophin cassette, which includes from the N-terminus to the C-terminus ABD1-H1-R1-R2-R3-H3-R24-H4-CR-CT, wherein the CT comprises at least the portion of the CT comprising an α1-syntrophin binding site, including the CT having an amino acid sequence of SEQ ID NO:48 or 49, ABD1 being directly coupled to VH4.

In a specific embodiment, the constructs described herein comprise the following components: (1) AAV2 ITRs that flank the expression cassette; (2) control elements, which include a promoter, such as the muscle-specific Spc5-12 promoter (or modified Spc5-12 promoter SPc5v1 or SPc5v2 (SEQ ID NOs: 127 or 128), and b) a small poly A signal; and (3) the nucleic acid encoding an AUF1. In some embodiments, constructs described herein comprising AAV ITRs flanking an AUF1 expression cassette, which includes one or more of the AUF1 sequences disclosed herein.

In a specific embodiment, the constructs described herein comprise the following components: (1) AAV2 ITRs that flank the expression cassette; (2) control elements, which include the muscle-specific Spc5-12 promoter (or modified Spc5-12 promoter SPc5v1 or SPc5v2 (SEQ ID Nos: 127 or 128)), and b) a small poly A signal; and (3) the nucleic acid encoding the RGX-DYS1 microdystrophin having an amino acid sequence of SEQ ID NO: 52, including encoded by a nucleotide sequence of SEQ ID NO:91. In a specific embodiment, the constructs described herein comprise the following components: (1) AAV2 ITRs that flank the expression cassette; (2) control elements, which include the muscle-specific Spc5-12 promoter, and b) a small poly A signal; and (3) the nucleic acid encoding the RXG-DYS5 microdystrophin having an amino acid sequence of SEQ ID NO:54, including encoded by a nucleotide sequence of SEQ ID NO:93. In some embodiments, constructs described herein comprising AAV ITRs flanking a microdystrophin expression cassette, which includes from the N-terminus to the C-terminus ABD1-H1-R1-R2-R3-H2-R24-H4-CR-CT, wherein the CT comprises at least the portion of the CT comprising an α1-syntrophin binding site, including the CT having an amino acid sequence of SEQ ID NO:48 or 49, can be between 4000 nt and 5000 nt in length. In some embodiments, such constructs are less than 4900 nt, 4800 nt, 4700 nt, 4600 nt, 4500 nt, 4400 nt, or 4300 nt in length.

Some nucleic acid embodiments of the present disclosure comprise rAAV vectors encoding microdystrophin comprising or consisting of a nucleotide sequence of SEQ ID NO: 94, 95, or 96 provided in Table 9 below. In various embodiments, an rAAV vector comprising a nucleotide sequence that has at least 50%, at least 60%, at least 70%, at least 75%, at least 80%, at least 85%, at least 90%, at least 95%, at least 98% or at least 99% sequence identity to the nucleotide sequence of SEQ ID NO: 94, 95, or 96 or the reverse complement thereof and encodes a rAAV vector suitable for expression of a therapeutically effective microdystrophin in muscle cells. In embodiments, the constructs having the nucleotide sequence of SEQ ID NO: 94, 95 or 96 are in a recombinant rAAV8 or recombinant AAV9 particle.

TABLE 9
RGX-DYS cassette nucleotide sequences

| Structure | SEQ ID | Nucleic Acid Sequence |
|---|---|---|
| RGX-DYS1 | 94 | ctgcgcgctcgctcgctcactgaggccgcccgggcaaagcccgg |
| (ITR to ITR) | | gcgtcgggcgacctttggtcgcccggcctcagtgagcgagcgag |
| 4734 bp | | cgcgcagagagggagtggccaactccatcactaggggttcctCA |
| ITRs shown in | | TATGcagggtaatggggatcctCTAGAGGCCGTCCGCCCTCGGC |
| lower case | | ACCATCCTCACGACACCCAAATATGGCGACGGGTGAGGAATGGT |
| | | GGGGAGTTATTTTTAGAGCGGTGAGGAAGGTGGGCAGGCAGCAG |
| | | GTGTTGGCGCTCTAAAAATAACTCCCGGGAGTTATTTTTAGAGC |
| | | GGAGGAATGGTGGACACCCAAATATGGCGACGGTTCCTCACCCG |
| | | TCGCCATATTTGGGTGTCCGCCCTCGGCCGGGGCCGCATTCCTG |
| | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGCGgAATTCGCCACCATGCTTTGGTGGGAAGAGGTGGAAGATT |
| | | GCTATGAGAGGGAAGATGTGCAGAAGAAAACCTTCACCAAATGG |
| | | GTCAATGCCCAGTTCAGCAAGTTTGGCAAGCAGCACATTGAGAA |
| | | CCTGTTCAGTGACCTGCAGGATGGCAGAAGGCTGCTGGATCTGC |
| | | TGGAAGGCCTGACAGGCCAGAAGCTGCCTAAAGAGAAGGGCAGC |
| | | ACAAGAGTGCATGCCCTGAACAATGTGAACAAGGCCCTGAGAGT |
| | | GCTGCAGAACAACAATGTGGACCTGGTCAATATTGGCAGCACAG |
| | | ACATTGTGGATGGCAACCACAAGCTGACCCTGGGCCTGATCTGG |
| | | AACATCATCCTGCACTGGCAAGTGAAGAATGTGATGAAGAACAT |
| | | CATGGCTGGCCTGCAGCAGACCAACTCTGAGAAGATCCTGCTGA |
| | | GCTGGGTCAGACAGAGCACCAGAAACTACCCTCAAGTGAATGTG |
| | | ATCAACTTCACCACCTCTTGGAGTGATGGACTGGCCCTGAATGC |
| | | CCTGATCCACAGCCACAGACCTGACCTGTTTGACTGGAACTCTG |
| | | TTGTGTGCCAGCAGTCTGCCACACAGAGACTGGAACATGCCTTC |
| | | AACATTGCCAGATACCAGCTGGGAATTGAGAAACTGCTGGACCC |
| | | TGAGGATGTGGACACCACCTATCCTGACAAGAAATCCATCCTCA |
| | | TGTACATCACCAGCCTGTTCCAGGTGCTGCCCCAGCAAGTGTCC |
| | | ATTGAGGCCATTCAAGAGGTTGAGATGCTGCCCAGACCTCCTAA |
| | | AGTGACCAAAGAGGAACACTTCCAGCTGCACCACCAGATGCACT |
| | | ACTCTCAGCAGATCACAGTGTCTCTGGCCCAGGGATATGAGAGA |
| | | ACAAGCAGCCCCAAGCCTAGGTTCAAGAGCTATGCCTACACACA |
| | | GGCTGCCTATGTGACCACATCTGACCCCACAAGAAGCCCATTTC |
| | | CAAGCCAGCATCTGGAAGCCCCTGAGGACAAGAGCTTTGGCAGC |
| | | AGCCTGATGGAATCTGAAGTGAACCTGGATAGATACCAGACAGC |
| | | CCTGGAAGAAGTGCTGTCCTGGCTGCTGTCTGCTGAGGATACAC |
| | | TGCAGGCTCAGGGTGAAATCAGCAATGATGTGGAAGTGGTCAAG |
| | | GACCAGTTTCACACCCATGAGGGCTACATGATGGACCTGACAGC |
| | | CCACCAGGGCAGAGTGGGAAATATCCTGCAGCTGGGCTCCAAGC |
| | | TGATTGGCACAGGCAAGCTGTCTGAGGATGAAGAGACAGAGGTG |
| | | CAAGAGCAGATGAACCTGCTGAACAGCAGATGGGAGTGTCTGAG |
| | | AGTGGCCAGCATGGAAAAGCAGAGCAACCTGCACAGAGTGCTCA |
| | | TGGACCTGCAGAATCAGAAACTGAAAGAACTGAATGACTGGCTG |
| | | ACCAAGACAGAAGAAAGGACTAGGAAGATGGAAGAGGAACCTCT |
| | | GGGACCAGACCTGGAAGATCTGAAAAGACAGGTGCAGCAGCATA |
| | | AGGTGCTGCAAGAGGACCTTGAGCAAGAGCAAGTCAGAGTGAAC |
| | | AGCCTGACACACATGGTGGTGGTTGTGGATGAGTCCTCTGGGGA |
| | | TCATGCCACAGCTGCTCTGGAAGAACAGCTGAAGGTGCTGGGAG |
| | | ACAGATGGGCCAACATCTGTAGGTGGACAGAGGATAGATGGGTG |
| | | CTGCTCCAGGACATTCTGCTGAAGTGGCAGAGACTGACAGAGGA |
| | | ACAGTGCCTGTTTTCTGCCTGGCTCTCTGAGAAAGAGGATGCTG |
| | | TCAACAAGATCCATACCACAGGCTTCAAGGATCAGAATGAGATG |
| | | CTCAGCTCCCTGCAGAAACTGGCTGTGCTGAAGGCTGACCTGGA |
| | | AAAGAAAAAGCAGTCCATGGGCAAGCTCTACAGCCTGAAGCAGG |
| | | ACCTGCTGTCTACCCTGAAGAACAAGTCTGTGACCCAGAAAACT |
| | | GAGGCCTGGCTGGACAACTTTGCTAGATGCTGGGACAACCTGGT |
| | | GCAGAAGCTGGAAAAGTCTACAGCCCAGATCAGCCAGCAACCTG |
| | | ATCTTGCCCCTGGCCTGACCACAATTGGAGCCTCTCCAACACAG |
| | | ACTGTGACCCTGGTTACCCAGCCAGTGGTCACCAAAGAGACAGC |
| | | CATCAGCAAACTGGAAATGCCCAGCTCTCTGATGCTGGAAGTCC |
| | | CCACACTGGAAAGGCTGCAAGAACTTCAAGAGGCCACAGATGAG |
| | | CTGGACCTGAAGCTGAGACAGGCTGAAGTGATCAAAGGCAGCTG |
| | | GCAGCCAGTTGGGGACCTGCTCATTGATAGCCTGCAGGACCATC |
| | | TGGAAAAAGTGAAAGCCCTGAGGGGAGAGATTGCCCCTCTGAAA |
| | | GAAAATGTGTCCCATGTGAATGACCTGGCCAGACAGCTGACCAC |
| | | ACTGGGAATCCAGCTGAGCCCCTACAACCTGAGCACCCTTGAGG |
| | | ACCTGAACACCAGGTGGAAGCTCCTCCAGGTGGCAGTGGAAGAT |
| | | AGAGTCAGGCAGCTGCATGAGGCCCACAGAGATTTTGGACCAGC |
| | | CAGCCAGCACTTTCTGTCTACCTCTGTGCAAGGCCCCTGGGAGA |
| | | GAGCTATCTCTCCTAACAAGGTGCCCTACTACATCAACCATGAG |
| | | ACACAGACCACCTGTTGGGATCACCCCAAGATGACAGAGCTGTA |
| | | CCAGAGTCTGGCAGACCTCAACAATGTCAGATTCAGTGCCTACA |
| | | GGACTGCCATGAAGCTCAGAAGGCTCCAGAAAGCTCTGTGCCTG |
| | | GACCTGCTTTCCCTGAGTGCAGCTTGTGATGCCCTGGACCAGCA |
| | | CAATCTGAAGCAGAATGACCAGCCTATGGACATCCTCCAGATCA |
| | | TCAACTGCCTCACCACCATCTATGATAGGCTGGAACAAGAGCAC |
| | | AACAATCTGGTCAATGTGCCCCTGTGTGTGGACATGTGCCTGAA |
| | | TTGGCTGCTGAATGTGTATGACACAGGCAGAACAGGCAGGATCA |
| | | GAGTCCTGTCCTTCAAGACAGGCATCATCTCCCTGTGCAAAGCC |
| | | CACTTGGAGGACAAGTACAGATACCTGTTCAAGCAAGTGGCCTC |
| | | CAGCACAGGCTTTTGTGACCAGAGAAGGCTGGGCCTGCTCCTGC |
| | | ATGACAGCATTCAGATCCCTAGACAGCTGGGAGAAGTGGCTTCC |
| | | TTTGGAGGCAGCAATATTGAGCCATCAGTCAGGTCCTGTTTTCA |
| | | GTTTGCCAACAACAAGCCTGAGATTGAGGCTGCCCTGTTCCTGG |
| | | ACTGGATGAGACTTGAGCCTCAGAGCATGGTCTGGCTGCCTGTG |
| | | CTTCATAGAGTGGCTGCTGCTGAGACTGCCAAGCACCAGGCCAA |
| | | GTGCAACATCTGCAAAGAGTGCCCCATCATTGGCTTCAGATACA |
| | | GATCCCTGAAGCACTTCAACTATGATATCTGCCAGAGCTGCTTC |
| | | TTTAGTGGCAGGGTTGCCAAGGGCCACAAAATGCACTACCCCAT |
| | | GGTGGAATACTGCACCCCAACAACCTCTGGGGAAGATGTTAGAG |
| | | ACTTTGCCAAGGTGCTGAAAAACAAGTTCAGGACCAAGAGATAC |
| | | TTTGCTAAGCACCCCAGAATGGGCTACCTGCCTGTCCAGACAGT |
| | | GCTTGAGGGTGACAACATGGAAACCCCTGTGACACTGATCAATT |
| | | TCTGGCCAGTGGACTCTGCCCCTGCCTCAAGTCCACAGCTGTCC |
| | | CATGATGACACCCACAGCAGAATTGAGCACTATGCCTCCAGACT |
| | | GGCAGAGATGGAAAACAGCAATGGCAGCTACCTGAATGATAGCA |
| | | TCAGCCCCAATGAGAGCATTGATGATGAGCATCTGCTGATCCAG |
| | | CACTACTGTCAGTCCCTGAACCAGGACTCTCCACTGAGCCAGCC |
| | | TAGAAGCCCTGCTCAGATCCTGATCAGCCTTGAGTCTGAGGAAA |
| | | GGGGAGAGCTGGAAAGAATCCTGGCAGATCTTGAGGAAGAGAAC |
| | | AGAAACCTGCAGGCAGAGTATGACAGGCTCAAACAGCAGCATGA |
| | | GCACAAGGGACTGAGCCCTCTGCCTTCTCCTCCTGAAATGATGC |
| | | CCACCTCTCCACAGTCTCCAAGGTGATGACTCGAGAGGCCTAAT |
| | | AAAGAGCTCAGATGCATCGATCAGAGTGTGTTGGTTTTTTGTGT |
| | | GCCAGGGTAATGGGCTAGCTGCGGCCGCaggaacccctagtgat |
| | | ggagttggccactccctctctgcgcgctcgctcgctcactgagg |
| | | ccgggcgaccaaaggtcgcccgacgcccgggctttgcccgggcg |
| | | gcctcagtgagcgagcgagcgcgcag |
| RGX-DYS3 | 95 | ctgcgcgctcgctcgctcactgaggccgcccgggcaaagcccgg |
| (ITR to ITR) | | gcgtcgggcgacctttggtcgcccggcctcagtgagcgagcgag |
| 4364 bp) | | cgcgcagagagggagtggccaactccatcactaggggttcctCA |
| ITRs shown in | | TATGcagggtaatggggatcctCTAGAGGCCGTCCGCCCTCGGC |
| lower case | | ACCATCCTCACGACACCCAAATATGGCGACGGGTGAGGAATGGT |
| | | GGGGAGTTATTTTTAGAGCGGTGAGGAAGGTGGGCAGGCAGCAG |
| | | GTGTTGGCGCTCTAAAAATAACTCCCGGGAGTTATTTTTAGAGC |
| | | GGAGGAATGGTGGACACCCAAATATGGCGACGGTTCCTCACCCG |
| | | TCGCCATATTTGGGTGTCCGCCCTCGGCCGGGGCCGCATTCCTG |
| | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGGTGAGTATCTCAGGGATCCAGACATGGGGATATGGGAGGTGC |
| | | CTCTGATCCCAGGGCTCACTGTGGGTCTCTCTGTTCACAGGAAT |
| | | TCGCCACCATGCTTTGGTGGGAAGAGGTGGAAGATTGCTATGAG |
| | | AGGGAAGATGTGCAGAAGAAAACCTTCACCAAATGGGTCAATGC |
| | | CCAGTTCAGCAAGTTTGGCAAGCAGCACATTGAGAACCTGTTCA |
| | | GTGACCTGCAGGATGGCAGAAGGCTGCTGGATCTGCTGGAAGGC |
| | | CTGACAGGCCAGAAGCTGCCTAAAGAGAAGGGCAGCACAAGAGT |
| | | GCATGCCCTGAACAATGTGAACAAGGCCCTGAGAGTGCTGCAGA |
| | | ACAACAATGTGGACCTGGTCAATATTGGCAGCACAGACATTGTG |
| | | GATGGCAACCACAAGCTGACCCTGGGCCTGATCTGGAACATCAT |
| | | CCTGCACTGGCAAGTGAAGAATGTGATGAAGAACATCATGGCTG |
| | | GCCTGCAGCAGACCAACTCTGAGAAGATCCTGCTGAGCTGGGTC |
| | | AGACAGAGCACCAGAAACTACCCTCAAGTGAATGTGATCAACTT |
| | | CACCACCTCTTGGAGTGATGGACTGGCCCTGAATGCCCTGATCC |
| | | ACAGCCACAGACCTGACCTGTTTGACTGGAACTCTGTTGTGTGC |
| | | CAGCAGTCTGCCACACAGAGACTGGAACATGCCTTCAACATTGC |
| | | CAGATACCAGCTGGGAATTGAGAAACTGCTGGACCCTGAGGATG |
| | | TGGACACCACCTATCCTGACAAGAAATCCATCCTCATGTACATC |
| | | ACCAGCCTGTTCCAGGTGCTGCCCCAGCAAGTGTCCATTGAGGC |
| | | CATTCAAGAGGTTGAGATGCTGCCCAGACCTCCTAAAGTGACCA |
| | | AAGAGGAACACTTCCAGCTGCACCACCAGATGCACTACTCTCAG |
| | | CAGATCACAGTGTCTCTGGCCCAGGGATATGAGAGAACAAGCAG |
| | | CCCCAAGCCTAGGTTCAAGAGCTATGCCTACACACAGGCTGCCT |
| | | ATGTGACCACATCTGACCCCACAAGAAGCCCATTTCCAAGCCAG |
| | | CATCTGGAAGCCCCTGAGGACAAGAGCTTTGGCAGCAGCCTGAT |
| | | GGAATCTGAAGTGAACCTGGATAGATACCAGACAGCCCTGGAAG |
| | | AAGTGCTGTCCTGGCTGCTGTCTGCTGAGGATACACTGCAGGCT |
| | | CAGGGTGAAATCAGCAATGATGTGGAAGTGGTCAAGGACCAGTT |
| | | TCACACCCATGAGGGCTACATGATGGACCTGACAGCCCACCAGG |
| | | GCAGAGTGGGAAATATCCTGCAGCTGGGCTCCAAGCTGATTGGC |
| | | ACAGGCAAGCTGTCTGAGGATGAAGAGACAGAGGTGCAAGAGCA |
| | | GATGAACCTGCTGAACAGCAGATGGGAGTGTCTGAGAGTGGCCA |
| | | GCATGGAAAAGCAGAGCAACCTGCACAGAGTGCTCATGGACCTG |
| | | CAGAATCAGAAACTGAAAGAACTGAATGACTGGCTGACCAAGAC |
| | | AGAAGAAAGGACTAGGAAGATGGAAGAGGAACCTCTGGGACCAG |
| | | ACCTGGAAGATCTGAAAAGACAGGTGCAGCAGCATAAGGTGCTG |
| | | CAAGAGGACCTTGAGCAAGAGCAAGTCAGAGTGAACAGCCTGAC |
| | | ACACATGGTGGTGGTTGTGGATGAGTCCTCTGGGGATCATGCCA |
| | | CAGCTGCTCTGGAAGAACAGCTGAAGGTGCTGGGAGACAGATGG |
| | | GCCAACATCTGTAGGTGGACAGAGGATAGATGGGTGCTGCTCCA |
| | | GGACATTCTGCTGAAGTGGCAGAGACTGACAGAGGAACAGTGCC |
| | | TGTTTTCTGCCTGGCTCTCTGAGAAAGAGGATGCTGTCAACAAG |
| | | ATCCATACCACAGGCTTCAAGGATCAGAATGAGATGCTCAGCTC |
| | | CCTGCAGAAACTGGCTGTGCTGAAGGCTGACCTGGAAAAGAAAA |
| | | AGCAGTCCATGGGCAAGCTCTACAGCCTGAAGCAGGACCTGCTG |
| | | TCTACCCTGAAGAACAAGTCTGTGACCCAGAAAACTGAGGCCTG |
| | | GCTGGACAACTTTGCTAGATGCTGGGACAACCTGGTGCAGAAGC |
| | | TGGAAAAGTCTACAGCCCAGATCAGCCAGCAACCTGATCTTGCC |
| | | CCTGGCCTGACCACAATTGGAGCCTCTCCAACACAGACTGTGAC |
| | | CCTGGTTACCCAGCCAGTGGTCACCAAAGAGACAGCCATCAGCA |
| | | AACTGGAAATGCCCAGCTCTCTGATGCTGGAAGTCCCCACACTG |
| | | GAAAGGCTGCAAGAACTTCAAGAGGCCACAGATGAGCTGGACCT |
| | | GAAGCTGAGACAGGCTGAAGTGATCAAAGGCAGCTGGCAGCCAG |
| | | TTGGGGACCTGCTCATTGATAGCCTGCAGGACCATCTGGAAAAA |
| | | GTGAAAGCCCTGAGGGGAGAGATTGCCCCTCTGAAAGAAAATGT |
| | | GTCCCATGTGAATGACCTGGCCAGACAGCTGACCACACTGGGAA |
| | | TCCAGCTGAGCCCCTACAACCTGAGCACCCTTGAGGACCTGAAC |
| | | ACCAGGTGGAAGCTCCTCCAGGTGGCAGTGGAAGATAGAGTCAG |
| | | GCAGCTGCATGAGGCCCACAGAGATTTTGGACCAGCCAGCCAGC |
| | | ACTTTCTGTCTACCTCTGTGCAAGGCCCCTGGGAGAGAGCTATC |
| | | TCTCCTAACAAGGTGCCCTACTACATCAACCATGAGACACAGAC |
| | | CACCTGTTGGGATCACCCCAAGATGACAGAGCTGTACCAGAGTC |
| | | TGGCAGACCTCAACAATGTCAGATTCAGTGCCTACAGGACTGCC |
| | | ATGAAGCTCAGAAGGCTCCAGAAAGCTCTGTGCCTGGACCTGCT |
| | | TTCCCTGAGTGCAGCTTGTGATGCCCTGGACCAGCACAATCTGA |
| | | AGCAGAATGACCAGCCTATGGACATCCTCCAGATCATCAACTGC |
| | | CTCACCACCATCTATGATAGGCTGGAACAAGAGCACAACAATCT |
| | | GGTCAATGTGCCCCTGTGTGTGGACATGTGCCTGAATTGGCTGC |
| | | TGAATGTGTATGACACAGGCAGAACAGGCAGGATCAGAGTCCTG |
| | | TCCTTCAAGACAGGCATCATCTCCCTGTGCAAAGCCCACTTGGA |
| | | GGACAAGTACAGATACCTGTTCAAGCAAGTGGCCTCCAGCACAG |
| | | GCTTTTGTGACCAGAGAAGGCTGGGCCTGCTCCTGCATGACAGC |
| | | ATTCAGATCCCTAGACAGCTGGGAGAAGTGGCTTCCTTTGGAGG |
| | | CAGCAATATTGAGCCATCAGTCAGGTCCTGTTTTCAGTTTGCCA |
| | | ACAACAAGCCTGAGATTGAGGCTGCCCTGTTCCTGGACTGGATG |
| | | AGACTTGAGCCTCAGAGCATGGTCTGGCTGCCTGTGCTTCATAG |
| | | AGTGGCTGCTGCTGAGACTGCCAAGCACCAGGCCAAGTGCAACA |
| | | TCTGCAAAGAGTGCCCCATCATTGGCTTCAGATACAGATCCCTG |
| | | AAGCACTTCAACTATGATATCTGCCAGAGCTGCTTCTTTAGTGG |
| | | CAGGGTTGCCAAGGGCCACAAAATGCACTACCCCATGGTGGAAT |
| | | ACTGCACCCCAACAACCTCTGGGGAAGATGTTAGAGACTTTGCC |
| | | AAGGTGCTGAAAAACAAGTTCAGGACCAAGAGATACTTTGCTAA |
| | | GCACCCCAGAATGGGCTACCTGCCTGTCCAGACAGTGCTTGAGG |
| | | GTGACAACATGGAAACCTGATGAGTCGACAGGCCTAATAAAGAG |
| | | CTCAGATGCATCGATCAGAGTGTGTTGGTTTTTTGTGTG |
| | | GCTAGCTGCGGCCGCaggaacccctagtgatggagttggccact |
| | | ccctctctgcgcgctcgctcgctcactgaggccgggcgaccaaa |
| | | ggtcgcccgacgcccgggctttgcccgggcggcctcagtgagcg |
| | | agcgagcgcgcag |
| RGX-DYS5 | 96 | ctgcgcgctcgctcgctcactgaggccgcccgggcaaagcccgg |
| (ITR to ITR) | | gcgtcgggcgacctttggtcgcccggcctcagtgagcgagcgag |
| 4560 bp | | cgcgcagagagggagtggccaactccatcactaggggttcctCA |
| ITRs shown in | | TATGcagggtaatggggatcctCTAGAGGCCGTCCGCCCTCGGC |
| lower case | | ACCATCCTCACGACACCCAAATATGGCGACGGGTGAGGAATGGT |
| | | GGGGAGTTATTTTTAGAGCGGTGAGGAAGGTGGGCAGGCAGCAG |
| | | GTGTTGGCGCTCTAAAAATAACTCCCGGGAGTTATTTTTAGAGC |
| | | GGAGGAATGGTGGACACCCAAATATGGCGACGGTTCCTCACCCG |
| | | TCGCCATATTTGGGTGTCCGCCCTCGGCCGGGGCCGCATTCCTG |
| | | GGGGCCGGGCGGTGCTCCCGCCCGCCTCGATAAAAGGCTCCGGG |
| | | GCCGGCGGCGGCCCACGAGCTACCCGGAGGAGCGGGAGGCGCCA |
| | | AGCGGAATTCGCCACCATGCTTTGGTGGGAAGAGGTGGAAGATT |
| | | GCTATGAGAGGGAAGATGTGCAGAAGAAAACCTTCACCAAATGG |
| | | GTCAATGCCCAGTTCAGCAAGTTTGGCAAGCAGCACATTGAGAA |
| | | CCTGTTCAGTGACCTGCAGGATGGCAGAAGGCTGCTGGATCTGC |
| | | TGGAAGGCCTGACAGGCCAGAAGCTGCCTAAAGAGAAGGGCAGC |
| | | ACAAGAGTGCATGCCCTGAACAATGTGAACAAGGCCCTGAGAGT |
| | | GCTGCAGAACAACAATGTGGACCTGGTCAATATTGGCAGCACAG |
| | | ACATTGTGGATGGCAACCACAAGCTGACCCTGGGCCTGATCTGG |
| | | AACATCATCCTGCACTGGCAAGTGAAGAATGTGATGAAGAACAT |
| | | CATGGCTGGCCTGCAGCAGACCAACTCTGAGAAGATCCTGCTGA |
| | | GCTGGGTCAGACAGAGCACCAGAAACTACCCTCAAGTGAATGTG |
| | | ATCAACTTCACCACCTCTTGGAGTGATGGACTGGCCCTGAATGC |
| | | CCTGATCCACAGCCACAGACCTGACCTGTTTGACTGGAACTCTG |
| | | TTGTGTGCCAGCAGTCTGCCACACAGAGACTGGAACATGCCTTC |
| | | AACATTGCCAGATACCAGCTGGGAATTGAGAAACTGCTGGACCC |
| | | TGAGGATGTGGACACCACCTATCCTGACAAGAAATCCATCCTCA |
| | | TGTACATCACCAGCCTGTTCCAGGTGCTGCCCCAGCAAGTGTCC |
| | | ATTGAGGCCATTCAAGAGGTTGAGATGCTGCCCAGACCTCCTAA |
| | | AGTGACCAAAGAGGAACACTTCCAGCTGCACCACCAGATGCACT |
| | | ACTCTCAGCAGATCACAGTGTCTCTGGCCCAGGGATATGAGAGA |
| | | ACAAGCAGCCCCAAGCCTAGGTTCAAGAGCTATGCCTACACACA |
| | | GGCTGCCTATGTGACCACATCTGACCCCACAAGAAGCCCATTTC |
| | | CAAGCCAGCATCTGGAAGCCCCTGAGGACAAGAGCTTTGGCAGC |
| | | AGCCTGATGGAATCTGAAGTGAACCTGGATAGATACCAGACAGC |
| | | CCTGGAAGAAGTGCTGTCCTGGCTGCTGTCTGCTGAGGATACAC |
| | | TGCAGGCTCAGGGTGAAATCAGCAATGATGTGGAAGTGGTCAAG |
| | | GACCAGTTTCACACCCATGAGGGCTACATGATGGACCTGACAGC |
| | | CCACCAGGGCAGAGTGGGAAATATCCTGCAGCTGGGCTCCAAGC |
| | | TGATTGGCACAGGCAAGCTGTCTGAGGATGAAGAGACAGAGGTG |
| | | CAAGAGCAGATGAACCTGCTGAACAGCAGATGGGAGTGTCTGAG |
| | | AGTGGCCAGCATGGAAAAGCAGAGCAACCTGCACAGAGTGCTCA |
| | | TGGACCTGCAGAATCAGAAACTGAAAGAACTGAATGACTGGCTG |
| | | ACCAAGACAGAAGAAAGGACTAGGAAGATGGAAGAGGAACCTCT |
| | | GGGACCAGACCTGGAAGATCTGAAAAGACAGGTGCAGCAGCATA |
| | | AGGTGCTGCAAGAGGACCTTGAGCAAGAGCAAGTCAGAGTGAAC |
| | | AGCCTGACACACATGGTGGTGGTTGTGGATGAGTCCTCTGGGGA |
| | | TCATGCCACAGCTGCTCTGGAAGAACAGCTGAAGGTGCTGGGAG |
| | | ACAGATGGGCCAACATCTGTAGGTGGACAGAGGATAGATGGGTG |
| | | CTGCTCCAGGACATTCTGCTGAAGTGGCAGAGACTGACAGAGGA |
| | | ACAGTGCCTGTTTTCTGCCTGGCTCTCTGAGAAAGAGGATGCTG |
| | | TCAACAAGATCCATACCACAGGCTTCAAGGATCAGAATGAGATG |
| | | CTCAGCTCCCTGCAGAAACTGGCTGTGCTGAAGGCTGACCTGGA |
| | | AAAGAAAAAGCAGTCCATGGGCAAGCTCTACAGCCTGAAGCAGG |
| | | ACCTGCTGTCTACCCTGAAGAACAAGTCTGTGACCCAGAAAACT |
| | | GAGGCCTGGCTGGACAACTTTGCTAGATGCTGGGACAACCTGGT |
| | | GCAGAAGCTGGAAAAGTCTACAGCCCAGATCAGCCAGCAACCTG |
| | | ATCTTGCCCCTGGCCTGACCACAATTGGAGCCTCTCCAACACAG |
| | | ACTGTGACCCTGGTTACCCAGCCAGTGGTCACCAAAGAGACAGC |
| | | CATCAGCAAACTGGAAATGCCCAGCTCTCTGATGCTGGAAGTCC |
| | | CCACACTGGAAAGGCTGCAAGAACTTCAAGAGGCCACAGATGAG |
| | | CTGGACCTGAAGCTGAGACAGGCTGAAGTGATCAAAGGCAGCTG |
| | | GCAGCCAGTTGGGGACCTGCTCATTGATAGCCTGCAGGACCATC |
| | | TGGAAAAAGTGAAAGCCCTGAGGGGAGAGATTGCCCCTCTGAAA |
| | | GAAAATGTGTCCCATGTGAATGACCTGGCCAGACAGCTGACCAC |
| | | ACTGGGAATCCAGCTGAGCCCCTACAACCTGAGCACCCTTGAGG |
| | | ACCTGAACACCAGGTGGAAGCTCCTCCAGGTGGCAGTGGAAGAT |
| | | AGAGTCAGGCAGCTGCATGAGGCCCACAGAGATTTTGGACCAGC |
| | | CAGCCAGCACTTTCTGTCTACCTCTGTGCAAGGCCCCTGGGAGA |
| | | GAGCTATCTCTCCTAACAAGGTGCCCTACTACATCAACCATGAG |
| | | ACACAGACCACCTGTTGGGATCACCCCAAGATGACAGAGCTGTA |
| | | CCAGAGTCTGGCAGACCTCAACAATGTCAGATTCAGTGCCTACA |
| | | GGACTGCCATGAAGCTCAGAAGGCTCCAGAAAGCTCTGTGCCTG |
| | | GACCTGCTTTCCCTGAGTGCAGCTTGTGATGCCCTGGACCAGCA |
| | | CAATCTGAAGCAGAATGACCAGCCTATGGACATCCTCCAGATCA |
| | | TCAACTGCCTCACCACCATCTATGATAGGCTGGAACAAGAGCAC |
| | | AACAATCTGGTCAATGTGCCCCTGTGTGTGGACATGTGCCTGAA |
| | | TTGGCTGCTGAATGTGTATGACACAGGCAGAACAGGCAGGATCA |
| | | GAGTCCTGTCCTTCAAGACAGGCATCATCTCCCTGTGCAAAGCC |
| | | CACTTGGAGGACAAGTACAGATACCTGTTCAAGCAAGTGGCCTC |
| | | CAGCACAGGCTTTTGTGACCAGAGAAGGCTGGGCCTGCTCCTGC |
| | | ATGACAGCATTCAGATCCCTAGACAGCTGGGAGAAGTGGCTTCC |
| | | TTTGGAGGCAGCAATATTGAGCCATCAGTCAGGTCCTGTTTTCA |
| | | GTTTGCCAACAACAAGCCTGAGATTGAGGCTGCCCTGTTCCTGG |
| | | ACTGGATGAGACTTGAGCCTCAGAGCATGGTCTGGCTGCCTGTG |
| | | CTTCATAGAGTGGCTGCTGCTGAGACTGCCAAGCACCAGGCCAA |
| | | GTGCAACATCTGCAAAGAGTGCCCCATCATTGGCTTCAGATACA |
| | | GATCCCTGAAGCACTTCAACTATGATATCTGCCAGAGCTGCTTC |
| | | TTTAGTGGCAGGGTTGCCAAGGGCCACAAAATGCACTACCCCAT |
| | | GGTGGAATACTGCACCCCAACAACCTCTGGGGAAGATGTTAGAG |
| | | ACTTTGCCAAGGTGCTGAAAAACAAGTTCAGGACCAAGAGATAC |
| | | TTTGCTAAGCACCCCAGAATGGGCTACCTGCCTGTCCAGACAGT |
| | | GCTTGAGGGTGACAACATGGAAACCCCTGTGACACTGATCAATT |
| | | TCTGGCCAGTGGACTCTGCCCCTGCCTCAAGTCCACAGCTGTCC |
| | | CATGATGACACCCACAGCAGAATTGAGCACTATGCCTCCAGACT |
| | | GGCAGAGATGGAAAACAGCAATGGCAGCTACCTGAATGATAGCA |
| | | TCAGCCCCAATGAGAGCATTGATGATGAGCATCTGCTGATCCAG |
| | | CACTACTGTCAGTCCCTGAACCAGGACTCTCCACTGAGCCAGCC |
| | | TAGAAGCCCTGCTCAGATCCTGATCAGCCTTGAGTCTTGATGAG |
| | | TCGACAGGCCTAATAAAGAGCTCAGATGCATCGATCAGAGTGTG |
| | | TTGGTTTTTTGTGTGGCTAGCTGCGGCCGCaggaacccctagtg |
| | | atggagttggccactccctctctgcgcgctcgctcgctcactga |
| | | ggccgggcgaccaaaggtcgcccgacgcccgggctttgcccggg |
| | | cggcctcagtgagcgagcgagcgcgcag |
| SpcV1- | 129 | AGAGGCCGTCCGCCCTCGGCACCATCCTCACGACACCCAAATATGGCGA |
| Micro- | | CGGGTGAGGAATGGTGGGGAGTTATTTTTAGAGCGGTGAGGAAGGTGGG |
| dystrophin | | CAGGCAGCAGGTGTTGGCGCTCCATATTTGGCGGGAGTTATTTTTAGAG |
| (μDys1) | | CGGAGGAATGGTGGACACCCAAATATGGCGACGGTTCCTCACCCGTCGC |
| nucleotide | | TAAAAATAACTCCGTGTCCGCCCTCGGCCGGGGCCGCATTCCTGGGGGC |
| | | CGGGCGGTGCTCCCGCCCGCCTCGATAAAAGGCTCCGGGGCCGGCGGCG |
| | | GCCCACGAGCTACCCGGAGGAGCGGGAGGCGCCAAGCGgAATTCGCCAC |
| | | CATGCTTTGGTGGGAAGAGGTGGAAGATTGCTATGAGAGGGAAGATGTG |
| | | CAGAAGAAAACCTTCACCAAATGGGTCAATGCCCAGTTCAGCAAGTTTG |
| | | GCAAGCAGCACATTGAGAACCTGTTCAGTGACCTGCAGGATGGCAGAAG |
| | | GCTGCTGGATCTGCTGGAAGGCCTGACAGGCCAGAAGCTGCCTAAAGAG |
| | | AAGGGCAGCACAAGAGTGCATGCCCTGAACAATGTGAACAAGGCCCTGA |
| | | GAGTGCTGCAGAACAACAATGTGGACCTGGTCAATATTGGCAGCACAGA |
| | | CATTGTGGATGGCAACCACAAGCTGACCCTGGGCCTGATCTGGAACATC |
| | | ATCCTGCACTGGCAAGTGAAGAATGTGATGAAGAACATCATGGCTGGCC |
| | | TGCAGCAGACCAACTCTGAGAAGATCCTGCTGAGCTGGGTCAGACAGAG |
| | | CACCAGAAACTACCCTCAAGTGAATGTGATCAACTTCACCACCTCTTGG |
| | | AGTGATGGACTGGCCCTGAATGCCCTGATCCACAGCCACAGACCTGACC |
| | | TGTTTGACTGGAACTCTGTTGTGTGCCAGCAGTCTGCCACACAGAGACT |
| | | GGAACATGCCTTCAACATTGCCAGATACCAGCTGGGAATTGAGAAACTG |
| | | CTGGACCCTGAGGATGTGGACACCACCTATCCTGACAAGAAATCCATCC |
| | | TCATGTACATCACCAGCCTGTTCCAGGTGCTGCCCCAGCAAGTGTCCAT |
| | | TGAGGCCATTCAAGAGGTTGAGATGCTGCCCAGACCTCCTAAAGTGACC |
| | | AAAGAGGAACACTTCCAGCTGCACCACCAGATGCACTACTCTCAGCAGA |
| | | TCACAGTGTCTCTGGCCCAGGGATATGAGAGAACAAGCAGCCCCAAGCC |
| | | TAGGTTCAAGAGCTATGCCTACACACAGGCTGCCTATGTGACCACATCT |
| | | GACCCCACAAGAAGCCCATTTCCAAGCCAGCATCTGGAAGCCCCTGAGG |
| | | ACAAGAGCTTTGGCAGCAGCCTGATGGAATCTGAAGTGAACCTGGATAG |
| | | ATACCAGACAGCCCTGGAAGAAGTGCTGTCCTGGCTGCTGTCTGCTGAG |
| | | GATACACTGCAGGCTCAGGGTGAAATCAGCAATGATGTGGAAGTGGTCA |
| | | AGGACCAGTTTCACACCCATGAGGGCTACATGATGGACCTGACAGCCCA |
| | | CCAGGGCAGAGTGGGAAATATCCTGCAGCTGGGCTCCAAGCTGATTGGC |
| | | ACAGGCAAGCTGTCTGAGGATGAAGAGACAGAGGTGCAAGAGCAGATGA |
| | | ACCTGCTGAACAGCAGATGGGAGTGTCTGAGAGTGGCCAGCATGGAAAA |
| | | GCAGAGCAACCTGCACAGAGTGCTCATGGACCTGCAGAATCAGAAACTG |
| | | AAAGAACTGAATGACTGGCTGACCAAGACAGAAGAAAGGACTAGGAAGA |
| | | TGGAAGAGGAACCTCTGGGACCAGACCTGGAAGATCTGAAAAGACAGGT |
| | | GCAGCAGCATAAGGTGCTGCAAGAGGACCTTGAGCAAGAGCAAGTCAGA |
| | | GTGAACAGCCTGACACACATGGTGGTGGTTGTGGATGAGTCCTCTGGGG |
| | | ATCATGCCACAGCTGCTCTGGAAGAACAGCTGAAGGTGCTGGGAGACAG |
| | | ATGGGCCAACATCTGTAGGTGGACAGAGGATAGATGGGTGCTGCTCCAG |
| | | GACATTCTGCTGAAGTGGCAGAGACTGACAGAGGAACAGTGCCTGTTTT |
| | | CTGCCTGGCTCTCTGAGAAAGAGGATGCTGTCAACAAGATCCATACCAC |
| | | AGGCTTCAAGGATCAGAATGAGATGCTCAGCTCCCTGCAGAAACTGGCT |
| | | GTGCTGAAGGCTGACCTGGAAAAGAAAAAGCAGTCCATGGGCAAGCTCT |
| | | ACAGCCTGAAGCAGGACCTGCTGTCTACCCTGAAGAACAAGTCTGTGAC |
| | | CCAGAAAACTGAGGCCTGGCTGGACAACTTTGCTAGATGCTGGGACAAC |
| | | CTGGTGCAGAAGCTGGAAAAGTCTACAGCCCAGATCAGCCAGCAACCTG |
| | | ATCTTGCCCCTGGCCTGACCACAATTGGAGCCTCTCCAACACAGACTGT |
| | | GACCCTGGTTACCCAGCCAGTGGTCACCAAAGAGACAGCCATCAGCAAA |
| | | CTGGAAATGCCCAGCTCTCTGATGCTGGAAGTCCCCACACTGGAAAGGC |
| | | TGCAAGAACTTCAAGAGGCCACAGATGAGCTGGACCTGAAGCTGAGACA |
| | | GGCTGAAGTGATCAAAGGCAGCTGGCAGCCAGTTGGGGACCTGCTCATT |
| | | GATAGCCTGCAGGACCATCTGGAAAAAGTGAAAGCCCTGAGGGGAGAGA |
| | | TTGCCCCTCTGAAAGAAAATGTGTCCCATGTGAATGACCTGGCCAGACA |
| | | GCTGACCACACTGGGAATCCAGCTGAGCCCCTACAACCTGAGCACCCTT |
| | | GAGGACCTGAACACCAGGTGGAAGCTCCTCCAGGTGGCAGTGGAAGATA |
| | | GAGTCAGGCAGCTGCATGAGGCCCACAGAGATTTTGGACCAGCCAGCCA |
| | | GCACTTTCTGTCTACCTCTGTGCAAGGCCCCTGGGAGAGAGCTATCTCT |
| | | CCTAACAAGGTGCCCTACTACATCAACCATGAGACACAGACCACCTGTT |
| | | GGGATCACCCCAAGATGACAGAGCTGTACCAGAGTCTGGCAGACCTCAA |
| | | CAATGTCAGATTCAGTGCCTACAGGACTGCCATGAAGCTCAGAAGGCTC |
| | | CAGAAAGCTCTGTGCCTGGACCTGCTTTCCCTGAGTGCAGCTTGTGATG |
| | | CCCTGGACCAGCACAATCTGAAGCAGAATGACCAGCCTATGGACATCCT |
| | | CCAGATCATCAACTGCCTCACCACCATCTATGATAGGCTGGAACAAGAG |
| | | CACAACAATCTGGTCAATGTGCCCCTGTGTGTGGACATGTGCCTGAATT |
| | | GGCTGCTGAATGTGTATGACACAGGCAGAACAGGCAGGATCAGAGTCCT |
| | | GTCCTTCAAGACAGGCATCATCTCCCTGTGCAAAGCCCACTTGGAGGAC |
| | | AAGTACAGATACCTGTTCAAGCAAGTGGCCTCCAGCACAGGCTTTTGTG |
| | | ACCAGAGAAGGCTGGGCCTGCTCCTGCATGACAGCATTCAGATCCCTAG |
| | | ACAGCTGGGAGAAGTGGCTTCCTTTGGAGGCAGCAATATTGAGCCATCA |
| | | GTCAGGTCCTGTTTTCAGTTTGCCAACAACAAGCCTGAGATTGAGGCTG |
| | | CCCTGTTCCTGGACTGGATGAGACTTGAGCCTCAGAGCATGGTCTGGCT |
| | | GCCTGTGCTTCATAGAGTGGCTGCTGCTGAGACTGCCAAGCACCAGGCC |
| | | AAGTGCAACATCTGCAAAGAGTGCCCCATCATTGGCTTCAGATACAGAT |
| | | CCCTGAAGCACTTCAACTATGATATCTGCCAGAGCTGCTTCTTTAGTGG |
| | | CAGGGTTGCCAAGGGCCACAAAATGCACTACCCCATGGTGGAATACTGC |
| | | ACCCCAACAACCTCTGGGGAAGATGTTAGAGACTTTGCCAAGGTGCTGA |
| | | AAAACAAGTTCAGGACCAAGAGATACTTTGCTAAGCACCCCAGAATGGG |
| | | CTACCTGCCTGTCCAGACAGTGCTTGAGGGTGACAACATGGAAACCCCT |
| | | GTGACACTGATCAATTTCTGGCCAGTGGACTCTGCCCCTGCCTCAAGTC |
| | | CACAGCTGTCCCATGATGACACCCACAGCAGAATTGAGCACTATGCCTC |
| | | CAGACTGGCAGAGATGGAAAACAGCAATGGCAGCTACCTGAATGATAGC |
| | | ATCAGCCCCAATGAGAGCATTGATGATGAGCATCTGCTGATCCAGCACT |
| | | ACTGTCAGTCCCTGAACCAGGACTCTCCACTGAGCCAGCCTAGAAGCCC |
| | | TGCTCAGATCCTGATCAGCCTTGAGTCTGAGGAAAGGGGAGAGCTGGAA |
| | | AGAATCCTGGCAGATCTTGAGGAAGAGAACAGAAACCTGCAGGCAGAGT |
| | | ATGACAGGCTCAAACAGCAGCATGAGCACAAGGGACTGAGCCCTCTGCC |
| | | TTCTCCTCCTGAAATGATGCCCACCTCTCCACAGTCTCCAAGGTGATGA |
| SpcV1-μDys1 | 130 | CTGCGCGCTCGCTCGCTCACTGAGGCCGCCCGGGCAAAGCCCGGGCGTC |
| transgene | | GGGCGACCTTTGGTCGCCCGGCCTCAGTGAGCGAGCGAGCGCGCAGAGA |
| cassette (ITR | | GGGAGTGGCCAACTCCATCACTAGGGGTTCCTCATATGCAGGGTAATGG |
| to ITR) | | GGATCCTCTAGAGGCCGTCCGCCCTCGGCACCATCCTCACGACACCCAA |
| | | ATATGGCGACGGGTGAGGAATGGTGGGGAGTTATTTTTAGAGCGGTGAG |
| | | GAAGGTGGGCAGGCAGCAGGTGTTGGCGCTCCATATTTGGCGGGAGTTA |
| | | TTTTTAGAGCGGAGGAATGGTGGACACCCAAATATGGCGACGGTTCCTC |
| | | ACCCGTCGCTAAAAATAACTCCGTGTCCGCCCTCGGCCGGGGCCGCATT |
| | | CCTGGGGGCCGGGCGGTGCTCCCGCCCGCCTCGATAAAAGGCTCCGGGG |
| | | CCGGCGGCGGCCCACGAGCTACCCGGAGGAGCGGGAGGCGCCAAGCGGA |
| | | ATTCGCCACCATGCTTTGGTGGGAAGAGGTGGAAGATTGCTATGAGAGG |
| | | GAAGATGTGCAGAAGAAAACCTTCACCAAATGGGTCAATGCCCAGTTCA |
| | | GCAAGTTTGGCAAGCAGCACATTGAGAACCTGTTCAGTGACCTGCAGGA |
| | | TGGCAGAAGGCTGCTGGATCTGCTGGAAGGCCTGACAGGCCAGAAGCTG |
| | | CCTAAAGAGAAGGGCAGCACAAGAGTGCATGCCCTGAACAATGTGAACA |
| | | AGGCCCTGAGAGTGCTGCAGAACAACAATGTGGACCTGGTCAATATTGG |
| | | CAGCACAGACATTGTGGATGGCAACCACAAGCTGACCCTGGGCCTGATC |
| | | TGGAACATCATCCTGCACTGGCAAGTGAAGAATGTGATGAAGAACATCA |
| | | TGGCTGGCCTGCAGCAGACCAACTCTGAGAAGATCCTGCTGAGCTGGGT |
| | | CAGACAGAGCACCAGAAACTACCCTCAAGTGAATGTGATCAACTTCACC |
| | | ACCTCTTGGAGTGATGGACTGGCCCTGAATGCCCTGATCCACAGCCACA |
| | | GACCTGACCTGTTTGACTGGAACTCTGTTGTGTGCCAGCAGTCTGCCAC |
| | | ACAGAGACTGGAACATGCCTTCAACATTGCCAGATACCAGCTGGGAATT |
| | | GAGAAACTGCTGGACCCTGAGGATGTGGACACCACCTATCCTGACAAGA |
| | | AATCCATCCTCATGTACATCACCAGCCTGTTCCAGGTGCTGCCCCAGCA |
| | | AGTGTCCATTGAGGCCATTCAAGAGGTTGAGATGCTGCCCAGACCTCCT |
| | | AAAGTGACCAAAGAGGAACACTTCCAGCTGCACCACCAGATGCACTACT |
| | | CTCAGCAGATCACAGTGTCTCTGGCCCAGGGATATGAGAGAACAAGCAG |
| | | CCCCAAGCCTAGGTTCAAGAGCTATGCCTACACACAGGCTGCCTATGTG |
| | | ACCACATCTGACCCCACAAGAAGCCCATTTCCAAGCCAGCATCTGGAAG |
| | | CCCCTGAGGACAAGAGCTTTGGCAGCAGCCTGATGGAATCTGAAGTGAA |
| | | CCTGGATAGATACCAGACAGCCCTGGAAGAAGTGCTGTCCTGGCTGCTG |
| | | TCTGCTGAGGATACACTGCAGGCTCAGGGTGAAATCAGCAATGATGTGG |
| | | AAGTGGTCAAGGACCAGTTTCACACCCATGAGGGCTACATGATGGACCT |
| | | GACAGCCCACCAGGGCAGAGTGGGAAATATCCTGCAGCTGGGCTCCAAG |
| | | CTGATTGGCACAGGCAAGCTGTCTGAGGATGAAGAGACAGAGGTGCAAG |
| | | AGCAGATGAACCTGCTGAACAGCAGATGGGAGTGTCTGAGAGTGGCCAG |
| | | CATGGAAAAGCAGAGCAACCTGCACAGAGTGCTCATGGACCTGCAGAAT |
| | | CAGAAACTGAAAGAACTGAATGACTGGCTGACCAAGACAGAAGAAAGGA |
| | | CTAGGAAGATGGAAGAGGAACCTCTGGGACCAGACCTGGAAGATCTGAA |
| | | AAGACAGGTGCAGCAGCATAAGGTGCTGCAAGAGGACCTTGAGCAAGAG |
| | | CAAGTCAGAGTGAACAGCCTGACACACATGGTGGTGGTTGTGGATGAGT |
| | | CCTCTGGGGATCATGCCACAGCTGCTCTGGAAGAACAGCTGAAGGTGCT |
| | | GGGAGACAGATGGGCCAACATCTGTAGGTGGACAGAGGATAGATGGGTG |
| | | CTGCTCCAGGACATTCTGCTGAAGTGGCAGAGACTGACAGAGGAACAGT |
| | | GCCTGTTTTCTGCCTGGCTCTCTGAGAAAGAGGATGCTGTCAACAAGAT |
| | | CCATACCACAGGCTTCAAGGATCAGAATGAGATGCTCAGCTCCCTGCAG |
| | | AAACTGGCTGTGCTGAAGGCTGACCTGGAAAAGAAAAAGCAGTCCATGG |
| | | GCAAGCTCTACAGCCTGAAGCAGGACCTGCTGTCTACCCTGAAGAACAA |
| | | GTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCC |
| | | AGCAACCTGATCTTGCCCCTGGCCTGACCACAATTGGAGCCTCTCCAAC |
| | | ACAGACTGTGACCCTGGTTACCCAGCCAGTGGTCACCAAAGAGACAGCC |
| | | ATCAGCAAACTGGAAATGCCCAGCTCTCTGATGCTGGAAGTCCCCACAC |
| | | TGGAAAGGCTGCAAGAACTTCAAGAGGCCACAGATGAGCTGGACCTGAA |
| | | GCTGAGACAGGCTGAAGTGATCAAAGGCAGCTGGCAGCCAGTTGGGGAC |
| | | CTGCTCATTGATAGCCTGCAGGACCATCTGGAAAAAGTGAAAGCCCTGA |
| | | GGGGAGAGATTGCCCCTCTGAAAGAAAATGTGTCCCATGTGAATGACCT |
| | | GGCCAGACAGCTGACCACACTGGGAATCCAGCTGAGCCCCTACAACCTG |
| | | AGCACCCTTGAGGACCTGAACACCAGGTGGAAGCTCCTCCAGGTGGCAG |
| | | TGGAAGATAGAGTCAGGCAGCTGCATGAGGCCCACAGAGATTTTGGACC |
| | | AGCCAGCCAGCACTTTCTGTCTACCTCTGTGCAAGGCCCCTGGGAGAGA |
| | | GCTATCTCTCCTAACAAGGTGCCCTACTACATCAACCATGAGACACAGA |
| | | CCACCTGTTGGGATCACCCCAAGATGACAGAGCTGTACCAGAGTCTGGC |
| | | AGACCTCAACAATGTCAGATTCAGTGCCTACAGGACTGCCATGAAGCTC |
| | | AGAAGGCTCCAGAAAGCTCTGTGCCTGGACCTGCTTTCCCTGAGTGCAG |
| | | CTTGTGATGCCCTGGACCAGCACAATCTGAAGCAGAATGACCAGCCTAT |
| | | GGACATCCTCCAGATCATCAACTGCCTCACCACCATCTATGATAGGCTG |
| | | GAACAAGAGCACAACAATCTGGTCAATGTGCCCCTGTGTGTGGACATGT |
| | | GCCTGAATTGGCTGCTGAATGTGTATGACACAGGCAGAACAGGCAGGAT |
| | | CAGAGTCCTGTCCTTCAAGACAGGCATCATCTCCCTGTGCAAAGCCCAC |
| | | TTGGAGGACAAGTACAGATACCTGTTCAAGCAAGTGGCCTCCAGCACAG |
| | | GCTTTTGTGACCAGAGAAGGCTGGGCCTGCTCCTGCATGACAGCATTCA |
| | | GATCCCTAGACAGCTGGGAGAAGTGGCTTCCTTTGGAGGCAGCAATATT |
| | | GAGCCATCAGTCAGGTCCTGTTTTCAGTTTGCCAACAACAAGCCTGAGA |
| | | TTGAGGCTGCCCTGTTCCTGGACTGGATGAGACTTGAGCCTCAGAGCAT |
| | | GGTCTGGCTGCCTGTGCTTCATAGAGTGGCTGCTGCTGAGACTGCCAAG |
| | | CACCAGGCCAAGTGCAACATCTGCAAAGAGTGCCCCATCATTGGCTTCA |
| | | GATACAGATCCCTGAAGCACTTCAACTATGATATCTGCCAGAGCTGCTT |
| | | CTTTAGTGGCAGGGTTGCCAAGGGCCACAAAATGCACTACCCCATGGTG |
| | | GAATACTGCACCCCAACAACCTCTGGGGAAGATGTTAGAGACTTTGCCA |
| | | AGGTGCTGAAAAACAAGTTCAGGACCAAGAGATACTTTGCTAAGCACCC |
| | | CAGAATGGGCTACCTGCCTGTCCAGACAGTGCTTGAGGGTGACAACATG |
| | | GAAACCCCTGTGACACTGATCAATTTCTGGCCAGTGGACTCTGCCCCTG |
| | | CCTCAAGTCCACAGCTGTCCCATGATGACACCCACAGCAGAATTGAGCA |
| | | CTATGCCTCCAGACTGGCAGAGATGGAAAACAGCAATGGCAGCTACCTG |
| | | AATGATAGCATCAGCCCCAATGAGAGCATTGATGATGAGCATCTGCTGA |
| | | TCCAGCACTACTGTCAGTCCCTGAACCAGGACTCTCCACTGAGCCAGCC |
| | | TAGAAGCCCTGCTCAGATCCTGATCAGCCTTGAGTCTGAGGAAAGGGGA |
| | | GAGCTGGAAAGAATCCTGGCAGATCTTGAGGAAGAGAACAGAAACCTGC |
| | | AGGCAGAGTATGACAGGCTCAAACAGCAGCATGAGCACAAGGGACTGAG |
| | | CCCTCTGCCTTCTCCTCCTGAAATGATGCCCACCTCTCCACAGTCTCCA |
| | | AGGTGATGACTCGAGAGGCCTAATAAAGAGCTCAGATGCATCGATCAGA |
| | | GTGTGTTGGTTTTTTGTGTGCCAGGGTAATGGGCTAGCTGCGGCCGCAG |
| | | GAACCCCTAGTGATGGAGTTGGCCACTCCCTCTCTGCGCGCTCGCTCGC |
| | | TCACTGAGGCCGGGCGACCAAAGGTCGCCCGACGCCCGGGCTTTGCCCG |
| | | GGCGGCCTCAGTGAGCGAGCGAGCGCGCAG |
| SpcV2- | 131 | GGCCGTCCGCCCTCGGCACCATCCTCACGACACCCAAATATGGCGACGG |
| Micro- | | GTGAGGAATGGTGGGGAGTTATTTTTAGAGCGGTGAGGAAGGTGGGCAG |
| dystrophin | | GCAGCAGGTGTTGGGGGAGTTATTTTTAGAGCGGGGAGTTATTTTTAGA |
| (μDys1) | | GCGGAGGAATGGTGGACACCCAAATATGGCGACGGTTCCTCACGGACAC |
| nucleotide | | CCAAATATGGCGACGGGCCCTCGGCCGGGGCCGCATTCCTGGGGGCCGG |
| | | GCGGTGCTCCCGCCCGCCTCGATAAAAGGCTCCGGGGCCGGCGGCGGCC |
| | | CACGAGCTACCCGGAGGAGCGGGAGGCGCCAAGCGGAATTCGCCACCAT |
| | | GCTTTGGTGGGAAGAGGTGGAAGATTGCTATGAGAGGGAAGATGTGCAG |
| | | AAGAAAACCTTCACCAAATGGGTCAATGCCCAGTTCAGCAAGTTTGGCA |
| | | AGCAGCACATTGAGAACCTGTTCAGTGACCTGCAGGATGGCAGAAGGCT |
| | | GCTGGATCTGCTGGAAGGCCTGACAGGCCAGAAGCTGCCTAAAGAGAAG |
| | | GGCAGCACAAGAGTGCATGCCCTGAACAATGTGAACAAGGCCCTGAGAG |
| | | TGCTGCAGAACAACAATGTGGACCTGGTCAATATTGGCAGCACAGACAT |
| | | TGTGGATGGCAACCACAAGCTGACCCTGGGCCTGATCTGGAACATCATC |
| | | CTGCACTGGCAAGTGAAGAATGTGATGAAGAACATCATGGCTGGCCTGC |
| | | AGCAGACCAACTCTGAGAAGATCCTGCTGAGCTGGGTCAGACAGAGCAC |
| | | CAGAAACTACCCTCAAGTGAATGTGATCAACTTCACCACCTCTTGGAGT |
| | | GATGGACTGGCCCTGAATGCCCTGATCCACAGCCACAGACCTGACCTGT |
| | | TTGACTGGAACTCTGTTGTGTGCCAGCAGTCTGCCACACAGAGACTGGA |
| | | ACATGCCTTCAACATTGCCAGATACCAGCTGGGAATTGAGAAACTGCTG |
| | | GACCCTGAGGATGTGGACACCACCTATCCTGACAAGAAATCCATCCTCA |
| | | TGTACATCACCAGCCTGTTCCAGGTGCTGCCCCAGCAAGTGTCCATTGA |
| | | GGCCATTCAAGAGGTTGAGATGCTGCCCAGACCTCCTAAAGTGACCAAA |
| | | GAGGAACACTTCCAGCTGCACCACCAGATGCACTACTCTCAGCAGATCA |
| | | CAGTGTCTCTGGCCCAGGGATATGAGAGAACAAGCAGCCCCAAGCCTAG |
| | | GTTCAAGAGCTATGCCTACACACAGGCTGCCTATGTGACCACATCTGAC |
| | | CCCACAAGAAGCCCATTTCCAAGCCAGCATCTGGAAGCCCCTGAGGACA |
| | | AGAGCTTTGGCAGCAGCCTGATGGAATCTGAAGTGAACCTGGATAGATA |
| | | CCAGACAGCCCTGGAAGAAGTGCTGTCCTGGCTGCTGTCTGCTGAGGAT |
| | | ACACTGCAGGCTCAGGGTGAAATCAGCAATGATGTGGAAGTGGTCAAGG |
| | | ACCAGTTTCACACCCATGAGGGCTACATGATGGACCTGACAGCCCACCA |
| | | GGGCAGAGTGGGAAATATCCTGCAGCTGGGCTCCAAGCTGATTGGCACA |
| | | GGCAAGCTGTCTGAGGATGAAGAGACAGAGGTGCAAGAGCAGATGAACC |
| | | TGCTGAACAGCAGATGGGAGTGTCTGAGAGTGGCCAGCATGGAAAAGCA |
| | | GAGCAACCTGCACAGAGTGCTCATGGACCTGCAGAATCAGAAACTGAAA |
| | | GAACTGAATGACTGGCTGACCAAGACAGAAGAAAGGACTAGGAAGATGG |
| | | AAGAGGAACCTCTGGGACCAGACCTGGAAGATCTGAAAAGACAGGTGCA |
| | | GCAGCATAAGGTGCTGCAAGAGGACCTTGAGCAAGAGCAAGTCAGAGTG |
| | | AACAGCCTGACACACATGGTGGTGGTTGTGGATGAGTCCTCTGGGGATC |
| | | ATGCCACAGCTGCTCTGGAAGAACAGCTGAAGGTGCTGGGAGACAGATG |
| | | GGCCAACATCTGTAGGTGGACAGAGGATAGATGGGTGCTGCTCCAGGAC |
| | | ATTCTGCTGAAGTGGCAGAGACTGACAGAGGAACAGTGCCTGTTTTCTG |
| | | CCTGGCTCTCTGAGAAAGAGGATGCTGTCAACAAGATCCATACCACAGG |
| | | CTTCAAGGATCAGAATGAGATGCTCAGCTCCCTGCAGAAACTGGCTGTG |
| | | CTGAAGGCTGACCTGGAAAAGAAAAAGCAGTCCATGGGCAAGCTCTACA |
| | | GCCTGAAGCAGGACCTGCTGTCTACCCTGAAGAACAAGTCTGTGACCCA |
| | | GAAAACTGAGGCCTGGCTGGACAACTTTGCTAGATGCTGGGACAACCTG |
| | | GTGCAGAAGCTGGAAAAGTCTACAGCCCAGATCAGCCAGCAACCTGATC |
| | | TTGCCCCTGGCCTGACCACAATTGGAGCCTCTCCAACACAGACTGTGAC |
| | | CCTGGTTACCCAGCCAGTGGTCACCAAAGAGACAGCCATCAGCAAACTG |
| | | GAAATGCCCAGCTCTCTGATGCTGGAAGTCCCCACACTGGAAAGGCTGC |
| | | AAGAACTTCAAGAGGCCACAGATGAGCTGGACCTGAAGCTGAGACAGGC |
| | | TGAAGTGATCAAAGGCAGCTGGCAGCCAGTTGGGGACCTGCTCATTGAT |
| | | AGCCTGCAGGACCATCTGGAAAAAGTGAAAGCCCTGAGGGGAGAGATTG |
| | | CCCCTCTGAAAGAAAATGTGTCCCATGTGAATGACCTGGCCAGACAGCT |
| | | GACCACACTGGGAATCCAGCTGAGCCCCTACAACCTGAGCACCCTTGAG |
| | | GACCTGAACACCAGGTGGAAGCTCCTCCAGGTGGCAGTGGAAGATAGAG |
| | | TCAGGCAGCTGCATGAGGCCCACAGAGATTTTGGACCAGCCAGCCAGCA |
| | | CTTTCTGTCTACCTCTGTGCAAGGCCCCTGGGAGAGAGCTATCTCTCCT |
| | | AACAAGGTGCCCTACTACATCAACCATGAGACACAGACCACCTGTTGGG |
| | | ATCACCCCAAGATGACAGAGCTGTACCAGAGTCTGGCAGACCTCAACAA |
| | | TGTCAGATTCAGTGCCTACAGGACTGCCATGAAGCTCAGAAGGCTCCAG |
| | | AAAGCTCTGTGCCTGGACCTGCTTTCCCTGAGTGCAGCTTGTGATGCCC |
| | | TGGACCAGCACAATCTGAAGCAGAATGACCAGCCTATGGACATCCTCCA |
| | | GATCATCAACTGCCTCACCACCATCTATGATAGGCTGGAACAAGAGCAC |
| | | AACAATCTGGTCAATGTGCCCCTGTGTGTGGACATGTGCCTGAATTGGC |
| | | TGCTGAATGTGTATGACACAGGCAGAACAGGCAGGATCAGAGTCCTGTC |
| | | CTTCAAGACAGGCATCATCTCCCTGTGCAAAGCCCACTTGGAGGACAAG |
| | | TACAGATACCTGTTCAAGCAAGTGGCCTCCAGCACAGGCTTTTGTGACC |
| | | AGAGAAGGCTGGGCCTGCTCCTGCATGACAGCATTCAGATCCCTAGACA |
| | | GCTGGGAGAAGTGGCTTCCTTTGGAGGCAGCAATATTGAGCCATCAGTC |
| | | AGGTCCTGTTTTCAGTTTGCCAACAACAAGCCTGAGATTGAGGCTGCCC |
| | | TGTTCCTGGACTGGATGAGACTTGAGCCTCAGAGCATGGTCTGGCTGCC |
| | | TGTGCTTCATAGAGTGGCTGCTGCTGAGACTGCCAAGCACCAGGCCAAG |
| | | TGCAACATCTGCAAAGAGTGCCCCATCATTGGCTTCAGATACAGATCCC |
| | | TGAAGCACTTCAACTATGATATCTGCCAGAGCTGCTTCTTTAGTGGCAG |
| | | GGTTGCCAAGGGCCACAAAATGCACTACCCCATGGTGGAATACTGCACC |
| | | CCAACAACCTCTGGGGAAGATGTTAGAGACTTTGCCAAGGTGCTGAAAA |
| | | ACAAGTTCAGGACCAAGAGATACTTTGCTAAGCACCCCAGAATGGGCTA |
| | | CCTGCCTGTCCAGACAGTGCTTGAGGGTGACAACATGGAAACCTGATGA |
| SpcV2-μDys | 132 | CTGCGCGCTCGCTCGCTCACTGAGGCCGCCCGGGCAAAGCCCGGGCGTC |
| transgene | | GGGCGACCTTTGGTCGCCCGGCCTCAGTGAGCGAGCGAGCGCGCAGAGA |
| cassette (ITR | | GGGAGTGGCCAACTCCATCACTAGGGGTTCCTCATATGCAGGGTAATGG |
| to ITR) | | GGATCCTCTAGAGGCCGTCCGCCCTCGGCACCATCCTCACGACACCCAA |
| | | ATATGGCGACGGGTGAGGAATGGTGGGGAGTTATTTTTAGAGCGGTGAG |
| | | GAAGGTGGGCAGGCAGCAGGTGTTGGGGGAGTTATTTTTAGAGCGGGGA |
| | | GTTATTTTTAGAGCGGAGGAATGGTGGACACCCAAATATGGCGACGGTT |
| | | CCTCACGGACACCCAAATATGGCGACGGGCCCTCGGCCGGGGCCGCATT |
| | | CCTGGGGGCCGGGCGGTGCTCCCGCCCGCCTCGATAAAAGGCTCCGGGG |
| | | CCGGCGGCGGCCCACGAGCTACCCGGAGGAGCGGGAGGCGCCAAGCGGA |
| | | ATTCGCCACCATGCTTTGGTGGGAAGAGGTGGAAGATTGCTATGAGAGG |
| | | GAAGATGTGCAGAAGAAAACCTTCACCAAATGGGTCAATGCCCAGTTCA |
| | | GCAAGTTTGGCAAGCAGCACATTGAGAACCTGTTCAGTGACCTGCAGGA |
| | | TGGCAGAAGGCTGCTGGATCTGCTGGAAGGCCTGACAGGCCAGAAGCTG |
| | | CCTAAAGAGAAGGGCAGCACAAGAGTGCATGCCCTGAACAATGTGAACA |
| | | AGGCCCTGAGAGTGCTGCAGAACAACAATGTGGACCTGGTCAATATTGG |
| | | CAGCACAGACATTGTGGATGGCAACCACAAGCTGACCCTGGGCCTGATC |
| | | TGGAACATCATCCTGCACTGGCAAGTGAAGAATGTGATGAAGAACATCA |
| | | TGGCTGGCCTGCAGCAGACCAACTCTGAGAAGATCCTGCTGAGCTGGGT |
| | | CAGACAGAGCACCAGAAACTACCCTCAAGTGAATGTGATCAACTTCACC |
| | | ACCTCTTGGAGTGATGGACTGGCCCTGAATGCCCTGATCCACAGCCACA |
| | | GACCTGACCTGTTTGACTGGAACTCTGTTGTGTGCCAGCAGTCTGCCAC |
| | | ACAGAGACTGGAACATGCCTTCAACATTGCCAGATACCAGCTGGGAATT |
| | | GAGAAACTGCTGGACCCTGAGGATGTGGACACCACCTATCCTGACAAGA |
| | | AATCCATCCTCATGTACATCACCAGCCTGTTCCAGGTGCTGCCCCAGCA |
| | | AGTGTCCATTGAGGCCATTCAAGAGGTTGAGATGCTGCCCAGACCTCCT |
| | | AAAGTGACCAAAGAGGAACACTTCCAGCTGCACCACCAGATGCACTACT |
| | | CTCAGCAGATCACAGTGTCTCTGGCCCAGGGATATGAGAGAACAAGCAG |
| | | CCCCAAGCCTAGGTTCAAGAGCTATGCCTACACACAGGCTGCCTATGTG |
| | | ACCACATCTGACCCCACAAGAAGCCCATTTCCAAGCCAGCATCTGGAAG |
| | | CCCCTGAGGACAAGAGCTTTGGCAGCAGCCTGATGGAATCTGAAGTGAA |
| | | CCTGGATAGATACCAGACAGCCCTGGAAGAAGTGCTGTCCTGGCTGCTG |
| | | TCTGCTGAGGATACACTGCAGGCTCAGGGTGAAATCAGCAATGATGTGG |
| | | AAGTGGTCAAGGACCAGTTTCACACCCATGAGGGCTACATGATGGACCT |
| | | GACAGCCCACCAGGGCAGAGTGGGAAATATCCTGCAGCTGGGCTCCAAG |
| | | CTGATTGGCACAGGCAAGCTGTCTGAGGATGAAGAGACAGAGGTGCAAG |
| | | AGCAGATGAACCTGCTGAACAGCAGATGGGAGTGTCTGAGAGTGGCCAG |
| | | CATGGAAAAGCAGAGCAACCTGCACAGAGTGCTCATGGACCTGCAGAAT |
| | | CAGAAACTGAAAGAACTGAATGACTGGCTGACCAAGACAGAAGAAAGGA |
| | | CTAGGAAGATGGAAGAGGAACCTCTGGGACCAGACCTGGAAGATCTGAA |
| | | AAGACAGGTGCAGCAGCATAAGGTGCTGCAAGAGGACCTTGAGCAAGAG |
| | | CAAGTCAGAGTGAACAGCCTGACACACATGGTGGTGGTTGTGGATGAGT |
| | | CCTCTGGGGATCATGCCACAGCTGCTCTGGAAGAACAGCTGAAGGTGCT |
| | | GGGAGACAGATGGGCCAACATCTGTAGGTGGACAGAGGATAGATGGGTG |
| | | CTGCTCCAGGACATTCTGCTGAAGTGGCAGAGACTGACAGAGGAACAGT |
| | | GCCTGTTTTCTGCCTGGCTCTCTGAGAAAGAGGATGCTGTCAACAAGAT |
| | | CCATACCACAGGCTTCAAGGATCAGAATGAGATGCTCAGCTCCCTGCAG |
| | | AAACTGGCTGTGCTGAAGGCTGACCTGGAAAAGAAAAAGCAGTCCATGG |
| | | GCAAGCTCTACAGCCTGAAGCAGGACCTGCTGTCTACCCTGAAGAACAA |
| | | GTCTGTGACCCAGAAAACTGAGGCCTGGCTGGACAACTTTGCTAGATGC |
| | | TGGGACAACCTGGTGCAGAAGCTGGAAAAGTCTACAGCCCAGATCAGCC |
| | | AGCAACCTGATCTTGCCCCTGGCCTGACCACAATTGGAGCCTCTCCAAC |
| | | ACAGACTGTGACCCTGGTTACCCAGCCAGTGGTCACCAAAGAGACAGCC |
| | | ATCAGCAAACTGGAAATGCCCAGCTCTCTGATGCTGGAAGTCCCCACAC |
| | | TGGAAAGGCTGCAAGAACTTCAAGAGGCCACAGATGAGCTGGACCTGAA |
| | | GCTGAGACAGGCTGAAGTGATCAAAGGCAGCTGGCAGCCAGTTGGGGAC |
| | | CTGCTCATTGATAGCCTGCAGGACCATCTGGAAAAAGTGAAAGCCCTGA |
| | | GGGGAGAGATTGCCCCTCTGAAAGAAAATGTGTCCCATGTGAATGACCT |
| | | GGCCAGACAGCTGACCACACTGGGAATCCAGCTGAGCCCCTACAACCTG |
| | | AGCACCCTTGAGGACCTGAACACCAGGTGGAAGCTCCTCCAGGTGGCAG |
| | | TGGAAGATAGAGTCAGGCAGCTGCATGAGGCCCACAGAGATTTTGGACC |
| | | AGCCAGCCAGCACTTTCTGTCTACCTCTGTGCAAGGCCCCTGGGAGAGA |
| | | GCTATCTCTCCTAACAAGGTGCCCTACTACATCAACCATGAGACACAGA |
| | | CCACCTGTTGGGATCACCCCAAGATGACAGAGCTGTACCAGAGTCTGGC |
| | | AGACCTCAACAATGTCAGATTCAGTGCCTACAGGACTGCCATGAAGCTC |
| | | AGAAGGCTCCAGAAAGCTCTGTGCCTGGACCTGCTTTCCCTGAGTGCAG |
| | | CTTGTGATGCCCTGGACCAGCACAATCTGAAGCAGAATGACCAGCCTAT |
| | | GGACATCCTCCAGATCATCAACTGCCTCACCACCATCTATGATAGGCTG |
| | | GAACAAGAGCACAACAATCTGGTCAATGTGCCCCTGTGTGTGGACATGT |
| | | GCCTGAATTGGCTGCTGAATGTGTATGACACAGGCAGAACAGGCAGGAT |
| | | CAGAGTCCTGTCCTTCAAGACAGGCATCATCTCCCTGTGCAAAGCCCAC |
| | | TTGGAGGACAAGTACAGATACCTGTTCAAGCAAGTGGCCTCCAGCACAG |
| | | GCTTTTGTGACCAGAGAAGGCTGGGCCTGCTCCTGCATGACAGCATTCA |
| | | GATCCCTAGACAGCTGGGAGAAGTGGCTTCCTTTGGAGGCAGCAATATT |
| | | GAGCCATCAGTCAGGTCCTGTTTTCAGTTTGCCAACAACAAGCCTGAGA |
| | | TTGAGGCTGCCCTGTTCCTGGACTGGATGAGACTTGAGCCTCAGAGCAT |
| | | GGTCTGGCTGCCTGTGCTTCATAGAGTGGCTGCTGCTGAGACTGCCAAG |
| | | CACCAGGCCAAGTGCAACATCTGCAAAGAGTGCCCCATCATTGGCTTCA |
| | | GATACAGATCCCTGAAGCACTTCAACTATGATATCTGCCAGAGCTGCTT |
| | | CTTTAGTGGCAGGGTTGCCAAGGGCCACAAAATGCACTACCCCATGGTG |
| | | GAATACTGCACCCCAACAACCTCTGGGGAAGATGTTAGAGACTTTGCCA |
| | | AGGTGCTGAAAAACAAGTTCAGGACCAAGAGATACTTTGCTAAGCACCC |
| | | CAGAATGGGCTACCTGCCTGTCCAGACAGTGCTTGAGGGTGACAACATG |
| | | GAAACCTGATGAGTCGACAGGCCTAATAAAGAGCTCAGATGCATCGATC |
| | | AGAGTGTGTTGGTTTTTTGTGTGGCTAGCTGCGGCCGCAGGAACCCCTA |
| | | GTGATGGAGTTGGCCACTCCCTCTCTGCGCGCTCGCTCGCTCACTGAGG |
| | | CCGGGCGACCAAAGGTCGCCCGACGCCCGGGCTTTGCCCGGGCGGCCTC |
| | | AGTGAGCGAGCGAGCGCGCAG |

### 5.4. Regulatory Elements

The expression cassettes, rAAV genomes and rAAV vectors disclosed herein comprise transgenes encoding either AUF1 or a microdystrophin operably linked to regulatory elements, including promoter elements, and, optionally, enhancer elements and/or introns, to enhance or facilitate expression of the transgene. In some embodiments, the rAAV vector also includes such regulatory control elements known to one skilled in the art to influence the expression of the RNA and/or protein products encoded by nucleic acids (transgenes) within target cells of the subject. Regulatory control elements and may be tissue-specific, that is, active (or substantially more active or significantly more active) only in the target cell/tissue.

### 5.4.1 Promoters

### 5.4.1.1 Tissue-Specific Promoters

In specific embodiments, the expression cassette of an AAV vector comprises a regulatory sequence, such as a promoter, operably linked to the transgene that allows for expression in target tissues. The promoter may be a muscle promoter. In certain embodiments, the promoter is a muscle-specific promoter. The phrase “muscle-specific”, “muscle-selective” or “muscle-directed” refers to nucleic acid elements that have adapted their activity in muscle cells or tissue due to the interaction of such elements with the intracellular environment of the muscle cells. Such muscle cells may include myocytes, myotubes, cardiomyocytes, and the like. Specialized forms of myocytes with distinct properties such as cardiac, skeletal, and smooth muscle cells are included. Various therapeutics may benefit from muscle-specific expression of a transgene. In particular, gene therapies that treat various forms of muscular dystrophy delivered to and enabling high transduction efficiency in muscle cells have the added benefit of directing expression of the transgene in the cells where the transgene is most needed. Cardiac tissue may also benefit from muscle-directed expression of the transgene. Muscle-specific promoters may be operably linked to the transgenes of the invention.

Adeno-associated viral (AAV) vectors disclosed herein comprise a muscle cell-specific promoter operatively linked to the nucleic acid encoding the AUF1 and/or the microdystrophin or therapeutic protein for treatment of a dystrophinopathy. In some embodiments, the muscle cell-specific promoter mediates cell-specific and/or tissue-specific expression of an AUF1 protein or fragment thereof. The promoter may be a mammalian promoter. For example, the promoter may be selected from the group consisting of a human promoter, a murine promoter, a porcine promoter, a feline promoter, a canine promoter, an ovine promoter, a non-human primate promoter, an equine promoter, a bovine promoter, and the like.

In some embodiments, the muscle cell-specific promoter is one of a muscle creatine kinase (MCK) promoter, a syn100 promoter, a creatine kinase (CK) 6 promoter, a creatine kinase (CK) 7 promoter, a dMCK promoter, a tMCK promoter, a smooth muscle 22 (SM22) promoter, a myo-3 promoter, a Spc5-12 promoter, a creatine kinase (CK) 8 promoter, a creatine kinase (CK) 8e promoter, a creatine kinase (CK) 9 promoter, a U6 promoter, a H1 promoter, a desmin promoter, a Pitx3 promoter, a skeletal alpha-actin promoter, a MHCK7 promoter, and a Sp-301 promoter. Suitable muscle cell-specific promoter sequences are well known in the art and exemplary promoters are provided in Table 10 below (Malerba et al., “PABPN1 Gene Therapy for Oculopharyngeal Muscular Dystrophy,” Nat. Commun. 8:14848 (2017); Wang et al., “Construction and Analysis of Compact Muscle-Specific Promoters for AAV Vectors,” Gene. Ther. 15:1489-1499 (2008); Piekarowicz et al., “A Muscle Hybrid Promoter as a Novel Tool for Gene Therapy,” Mol. Ther. Methods Clin. Dev. 15:157-169 (2019); Salva et al., “Design of Tissue-Specific Regulatory Cassettes for High-Level rAAV-Mediated Expression in Skeletal and Cardiac Muscle,” Mol. Ther. 15(2):320-329 (2007); Lui et al., “Synthetic Promoter for Efficient and Muscle-Specific Expression of Exogenous Genes,” Plasmid 106:102441 (2019), Li, X. et al. “Synthetic muscle promoters: activities exceeding naturally occurring regulatory sequences” 1999, Nature Biotechnology 17:241-245; Liu Y L, et al. “Therapeutic levels of factor IX expression using a muscle-specific promoter and adeno-associated virus serotype 1 vector.” Hum Gene Ther 2004; 15: 783-792; Draghia-Akli R, et al. “Myogenic expression of an injectable protease-resistant growth hormone-releasing hormone augments long-term growth in pigs.” Nat Biotechnol 1999; 17: 1179-1183; Hagstrom J N, et al. “Improved muscle-derived expression of human coagulation factor IX from a skeletal actin/CMV hybrid enhancer/promoter.” Blood 2000; 95: 2536-2542; Li J, et al. “rAAV vector-mediated sarcogylcan gene transfer in a hamster model for limb girdle muscular dystrophy.” Gene Therapy 1999; 6: 74-82; Wang, B. et al. “Construction and analysis of compact muscle-specific promoters for AAV vectors” Gene Therapy 2008, 15:1489-1499; and Qiao, C. et al. “Muscle and Heart Function Restoration in a Limb Girdle Muscular Dystrophy 21 (LGMD2I) Mouse Model by Systemic FKRP Gene Delivery” Mol Ther. 2014, 22(11): 1890-1899, which are hereby incorporated by reference in their entirety.).

TABLE 10
Promoter Sequences

| | | SEQ |
|---|---|---|
| | | ID |
| Promoter | Sequence* | NO: |
| Human | AGCCAGCCTCAGTTTCCCCTCCACTCAGTCCCTAGGAGGAAGGGGCGCCC | 97 |
| muscle | AAGCGCGGGTTTCTGGGGTTAGACTGCCCTCCATTGCAATTGGTCCTTCT | |
| creatine | CCCGGCCTCTGCTTCCTCCAGCTCACAGGGTATCTGCTCCTCCTGGAGCC | |
| kinase | ACACCTTGGTTCCCCGAGGTGCCGCTGGGACTCGGGTAGGGGTGAGGGCC | |
| (MCK) | CAGGGGGCACAGGGGGAGCCGAGGGCCACAGGAAGGGCTGGTGGCTGAAG | |
| | GAGACTCAGGGGCCAGGGGACGGTGGCTTCTACGTGCTTGGGACGTTCCC | |
| | AGCCACCGTCCCATGTTCCCGGCGGGGGGCCAGCTGTCCCCACCGCCAGC | |
| | CCAACTCAGCACTTGGTCAGGGTATCAGCTTGGTGGGGGGGCGTGAGCCC | |
| | AGCCCCTGGGGCGGCTCAGCCCATACAAGGCCATGGGGCTGGGCGCAAAG | |
| | CATGCCTGGGTTCAGGGTGGGTATGGTGCGGGAGCAGGGAGGTGAGAGGC | |
| | TCAGCTGCCCTCCAGAACTCCTCCCTGGGGACAACCCCTCCCAGCCAATA | |
| | GCACAGCCTAGGTCCCCCTATATAAGGCCACGGCTGCTGGCCCTTCCTTT | |
| | (NCBI sequence ID No. 1158) | |
| Human | CTGAGGCTCAGGGCTAGCTCGCCCATAGACATACATGGCAGGCAGGCTTT | 98 |
| desmin | GGCCAGGATCCCTCCGCCTGCCAGGCGTCTCCCTGCCCTCCCTTCCTGCC | |
| | TAGAGACCCCCACCCTCAAGCCTGGCTGGTCTTTGCCTGAGACCCAAACC | |
| | TCTTCGACTTCAAGAGAATATTTAGGAACAAGGTGGTTTAGGGCCTTTCC | |
| | TGGGAACAGGCCTTGACCCTTTAAGAAATGACCCAAAGTCTCTCCTTGAC | |
| | CAAAAAGGGGACCCTCAAACTAAAGGGAAGCCTCTCTTCTGCTGTCTCCC | |
| | CTGACCCCACTCCCCCCCACCCCAGGACGAGGAGATAACCAGGGCTGAAA | |
| | GAGGCCCGCCTGGGGGCTGCAGACATGCTTGCTGCCTGCCCTGGCGAAGG | |
| | ATTGGCAGGCTTGCCCGTCACAGGACCCCCGCTGGCTGACTCAGGGGCGC | |
| | AGGCCTCTTGCGGGGGAGCTGGCCTCCCCGCCCCCACGGCCACGGGCCGC | |
| | CCTTTCCTGGCAGGACAGCGGGATCTTGCAGCTGTCAGGGGAGGGGAGGC | |
| | GGGGGCTGATGTCAGGAGGGATACAAATAGTGCCGACGGCTGGGGGCCCT | |
| | (NCBI sequence ID No. 1674) | |
| Human | ctgcagacatgcttgctgcctgccctggcgtgccctggcgaggcttgccgtcacagga | 99 |
| desmin 2 | cccccgctggctgactcaggggcgcaggctcttgcgggggagctggcctcccgccccc | |
| | acggccacgggccctttcctggcaggacagcgggatcttgcagctgtcaggggagggg | |
| | atgacgggggactgatgtcaggaggggatacaaatagtgccgaacaaggaccggatta | |
| | gatctacc | |
| Human | GGAGTTCCAGGGGCGTAAAGGAGAGGGAGTTCGCCTTCCTTCCCTTCCTG | 100 |
| skeletal | AGACTCAGGAGTGACTGCTTCTCCAATCCTCCCAAGCCCACCACTCCACA | |
| muscle | CGACTCCCTCTTCCCGGTAGTCGCAAGTGGGAGTTTGGGGATCTGAGCAA | |
| alpha | AGAACCCGAAGAGGAGTTGAAATATTGGAAGTCAGCAGTCAGGCACCTTC | |
| actin | CCGAGCGCCCAGGGCGCTCAGAGTGGACATGGTTGGGGAGGCCTTTGGGA | |
| acta 1 | CAGGTGCGGTTCCCGGAGCGCAGGCGCACACATGCACCCACCGGCGAACG | |
| | CGGTGACCCTCGCCCCACCCCATCCCCTCCGGCGGGCAACTGGGTCGGGT | |
| | CAGGAGGGGCAAACCCGCTAGGGAGACACTCCATATACGGCCCGGCCCGC | |
| | GTTACCTGGGACCGGGCCAACCCGCTCCTTCTTTGGTCAACGCAGGGGAC | |
| | CCGGGCGGGGGCCCAGGCCGCGAACCGGCCGAGGGAGGGGGCTCTAGTGC | |
| | CCAACACCCAAATATGGCTCGAGAAGGGCAGCGACATTCCTGCGGGGTGG | |
| | CGCGGAGGGAATGCCCGCGGGCTATATAAAACCTGAGCAGAGGGACAAGC | |
| | (NCBI sequence ID No. 58) | |
| Mouse | AGAAACCTGTGGTCTAGAGGCGGGGGGGGGCCGATGGAGGCAACGCACGC | 101 |
| muscle | CCCCGCAGGCGCCCAGGCCACGCCCTCTGCCGCAGCATTCGGTGAAACCT | |
| creatine | GCGTTCCGAGAACTTCTGAAAACTTTATCTGGGGGCCTTCGAGAAGGCTC | |
| kinase | AGACAGTAAGGGTGCATGCTGCCAATCCTGAGGAGCTGAGTTCGATCCCT | |
| (MCK) | GAGACCTTCAGGGTGGACAGAGACGGACTCCCACATGTTGTTTTCTGACT | |
| | TCTACATGTGTCCAGTCATACATACACAAATATGGAATAAACAGATGGCT | |
| | CATCAGGTAAGAGTGCTGGCTGCTTTTGCAGAGGACCCAGGTTCGATTTC | |
| | CAGAACCCACATGTCGGCTCAAAATCATCTGTAATTCCAGTTCCAGGGAG | |
| | ATCCAGCACTTTCTTCCAGGGCCTCCACAGACACACATAAAATAAAGATA | |
| | AAAATCTCCAAAAAATATTGTTTTAATAATTACAACCTGAAGACCTTGCA | |
| | CAACTATTCCTGGCTGAGAAGATGGTAAGGGCGCTAGCTGCCAAGCTTGA | |
| | CAGCCTGAGTTTCATCTCCAAGAACCATGAAAACTGACTCCTGGGAATTA | |
| | (NCBI sequence ID No. 12715) | |
| Mouse | GGAAGCAGAAGGCCAACATTCCTCCCAAGGGAAACTGAGGCTCAGAGTTA | 102 |
| desmin | AAACCCAGGTATCAGTGATATGCATGTGCCCCGGCCAGGGTCACTCTCTG | |
| | ACTAACCGGTACCTACCCTACAGGCCTACCTAGAGACTCTTTTGAAAGGA | |
| | TGGTAGAGACCTGTCCGGGCTTTGCCCACAGTCGTTGGAAACCTCAGCAT | |
| | TTTCTAGGCAACTTGTGCGAATAAAACACTTCGGGGGTCCTTCTTGTTCA | |
| | TTCCAATAACCTAAAACCTCTCCTCGGAGAAAATAGGGGGCCTCAAACAA | |
| | ACGAAATTCTCTAGCCCGCTTTCCCCAGGATAAGGCAGGCATCCAAATGG | |
| | AAAAAAAGGGGCCGGCCGGGGGTCTCCTGTCAGCTCCTTGCCCTGTGAAA | |
| | CCCAGCAGGCCTGCCTGTCTTCTGTCCTCTTGGGGCTGTCCAGGGGCGCA | |
| | GGCCTCTTGCGGGGGAGCTGGCCTCCCCGCCCCCTCGCCTGTGGCCGCCC | |
| | TTTTCCTGGCAGGACAGAGGGATCCTGCAGCTGTCAGGGGAGGGGCGCCG | |
| | GGGGGTGATGTCAGGAGGGCTACAAATAGTGCAGACAGCTAAGGGGCTCC | |
| | (NCBI sequence ID No. 13346) | |
| Mouse | GGGGTGATGTGTGTCAGATCTCTGGATTGGGGGAGCTTCAAAGTGGGAAA | 103 |
| skeletal | GAAAATGGAGTTCAAATGTGGGGCTTATTTTCCATCCCTACCTGGAGCCC | |
| muscle | ATGACTCCTCCCGGCTCACCTGACCACAGGGCTACCTCCCCTGAGCTTAA | |
| alpha | GCATCAAGGCTTAGTAGTCTGAGTTAAGdAACCCATAAATGGGGTGCATT | |
| actin | GTGGCAGGTCAGCAATCGTGTGTCCAGGTGGGCAGAACTGGGGAGACCTT | |
| acta 1 | TCAAACAGGTAAATCTTGGGAAGTACAGACCAGCAGTCTGCAAAGCAGTG | |
| | ACCTTTGGCCCAGCACAGCCCTTCCGTGAGCCTTGGAGCCAGTTGGGAGG | |
| | GGCAGACAGCTGGGGATACTCTCCATATACGGCCTGGTCCGGTCCTAGCT | |
| | ACCTGGGCCAGGGCCAGTCCTCTCCTTCTTTGGTCAGTGCAGGAGACCCG | |
| | GGCGGGGACCCAGGCTGAGAACCAGCCGAAGGAAGGGACTCTAGTGCCCG | |
| | ACACCCAAATATGGCTTGGGAAGGGCAGCAACATTCCTTCGGGGCGGTGT | |
| | GGGGAGAGCTCCCGGGACTATATAAAAACCTGTGCAAGGGGACAGGCGGT | |
| | C | |
| MCK7 | CTAGAAGCTGCATGTCTAAGCTAGACCCTTCAGATTAAAAATAACTGAGG | 104 |
| | TAAGGGCCTGGGTAGGGGAGGTGGTGTGAGACGCTCCTGTCTCTCCTCTA | |
| | TCTGCCCATCGGCCCTTTGGGGAGGAGGAATGTGCCCAAGGACTAAAAAA | |
| | AGGCCATGGAGCCAGAGGGGCGAGGGCAACAGACCTTTCATGGGCAAACC | |
| | TTGGGGCCCTGCTGTCTAGCATGCCCCACTACGGGTCTAGGCTGCCCATG | |
| | TAAGGAGGCAAGGCCTGGGGACACCCGAGATGCCTGGTTATAATTAACCC | |
| | AGACATGTGGCTGCCCCCCCCCCCCCAACACCTGCTGCCTCTAAAAATAA | |
| | CCCTGTCCCTGGTGGATCCCCTGCATGCGAAGATCTTCGAACAAGGCTGT | |
| | GGGGGACTGAGGGCAGGCTGTAACAGGCTTGGGGGCCAGGGCTTATACGT | |
| | GCCTGGGACTCCCAAAGTATTACTGTTCCATGTTCCCGGCGAAGGGCCAG | |
| | CTGTCCCCCGCCAGCTAGACTCAGCACTTAGTTTAGGAACCAGTGAGCAA | |
| | GTCAGCCCTTGGGGCAGCCCATACAAGGCCATGGGGCTGGGCAAGCTGCA | |
| | CGCCTGGGTCCGGGGTGGGCACGGTGCCCGGGCAACGAGCTGAAAGCTCA | |
| | TCTGCTCTCAGGGGCCCCTCCCTGGGGACAGCCCCTCCTGGCTAGTCACA | |
| | CCCTGTAGGCTCCTCTATATAACCCAGGGGCACAGGGGCTGCCCTCATTC | |
| | TACCACCACCTCCACAGCAC | |
| truncated | CCACTACGGG TCTAGGCTGC CCATGTAAGG AGGCAAGGCC | 105 |
| MCK | TGGGGACACC CGAGATGCCT GGTTATAATT AACCCCAACA | |
| (tMCK) | CCTGCTGCCC CCCCCCCCCC AACACCTGCT GCCTGAGCCT | |
| | GAGCGGTTAC CCCACCCCGG TGCCTGGGTC TTAGGCTCTG | |
| | TACACCATGG AGGAGAAGCT CGCTCTAAAA ATAACCCTGT | |
| | CCCTGGTGGA TCCACTACGG GTCTATGCTG CCCATGTAAG | |
| | GAGGCAAGGC CTGGGGACAC CCGAGATGCC TGGTTATAAT | |
| | TAACCCCAAC ACCTGCTGCC CCCCCCCCCC CAACACCTGC | |
| | TGCCTGAGCC TGAGCGGTTA CCCCACCCCG GTGCCTGGGT | |
| | CTTAGGCTCT GTACACCATG GAGGAGAAGC TCGCTCTAAA | |
| | AATAACCCTG TCCCTGGTGG ACCACTACGG GTCTAGGCTG | |
| | CCCATGTAAG GAGGCAAGGC CTGGGGACAC CCGAGATGCC | |
| | TGGTTATAAT TAACCCCAAC ACCTGCTGCC CCCCCCCCCC | |
| | AACACCTGCT GCCTGAGCCT GAGCGGTTAC CCCACCCCGG | |
| | TGCCTGGGTC TTAGGCTCTG TACACCATGG AGGAGAAGCT | |
| | CGCTCTAAAA ATAACCCTGT CCCTGGTCCT CCCTGGGGAC | |
| | AGCCCCTCCT GGCTAGTCAC ACCCTGTAGG CTCCTCTATA | |
| | TAACCCAGGG GCACAGGGGC TGCCCCCGGG TCAC | |
| Spc5-12 | CGAGCTCCACCGCGGTGGCGGCCGTCCGCCCTCGGCACCATCCTCACGAC | 106 |
| (1) | ACCCAAATATGGCGACGGGTGAGGAATGGTGGGGAGTTATTTTTAGAGCG | |
| | GTGAGGAAGGTGGGCAGGCAGCAGGTGTTGGCGCTCTAAAAATAACTCCC | |
| | GGGAGTTATTTTTAGAGCGGAGGAATGGTGGACACCCAAATATGGCGACG | |
| | GTTCCTCACCCGTCGCCATATTTGGGTGTCCGCCCTCGGCCGGGGCCGCA | |
| | TTCCTGGGGGCCGGGCGGTGCTCCCGCCCGCCTCGATAAAAGGCTCCGGG | |
| | GCCGGCGGCGGCCCACGAGCTACCCGGAGGAGCGGGAGGCGCCAAGCTCT | |
| | AGAACTAGTGGATCCCCCGGGCTGCAGGAATTC | |
| Spc5-12 | GGCCGTCCGCCCTCGGCACCATCCTCACGACACCCAAATATGGCGACGGG | 18 |
| | TGAGGAATGGTGGGGAGTTATTTTTAGAGCGGTGAGGAAGGTGGGCAGGC | |
| | AGCAGGTGTTGGCGCTCTAAAAATAACTCCCGGGAGTTATTTTTAGAGCG | |
| | GAGGAATGGTGGACACCCAAATATGGCGACGGTTCCTCACCCGTCGCCAT | |
| | ATTTGGGTGTCCGCCCTCGGCCGGGGCCGCATTCCTGGGGGCCGGGCGGT | |
| | GCTCCCGCCCGCCTCGATAAAAGGCTCCGGGGCCGGCGGCGGCCCACGAG | |
| | CTACCCGGAGGAGCGGGAGGCGCCAAGC | |
| CK8 | CCACTACGGGTTTAGGCTGCCCATGTAAGGAGGCAAGGCCTGGGGACACC | 107 |
| | CGAGATGCCTGGTTATAATTAACCCAGACATGTGGCTGCCCCCCCCCCCC | |
| | CCAACACCTGCTGCCTCTAAAAATAACCCTGTCCCTGGTGGATCCCACTA | |
| | CGGGTTTAGGCTGCCCATGTAAGGAGGCAAGGCCTGGGGACACCCGAGAT | |
| | GCCTGGTTATAATTAACCCAGACATGTGGCTGCCCCCCCCCCCCCCAACA | |
| | CCTGCTGCCTCTAAAAATAACCCTGTCCCTGGTGGATCCCACTACGGGTT | |
| | TAGGCTGCCCATGTAAGGAGGCAAGGCCTGGGGACACCCGAGATGCCTGG | |
| | TTATAATTAACCCAGACATGTGGCTGCCCCCCCCCCCCCCAACACCTGCT | |
| | GCCTCTAAAAATAACCCTGTCCCTGGTGGATCCCCTGCATGCGAAGATCT | |
| | TCGAACAAGGCTGTGGGGGACTGAGGGCAGGCTGTAACAGGCTTGGGGGC | |
| | CAGGGCTTATACGTGCCTGGGACTCCCAAAGTATTACTGTTCCATGTTCC | |
| | CGGCGAAGGGCCAGCTGTCCCCCGCCAGCTAGACTCAGCACTTAGTTTAG | |
| | GAACCAGTGAGCAAGTCAGCCCTTGGGGCAGCCCATACAAGGCCATGGGG | |
| | CTGGGCAAGCTGCACGCCTGGGTCCGGGGTGGGCACGGTGCCCGGGCAAC | |
| | GAGCTGAAAGCTCATCTGCTCTCAGGGGCCCCTCCCTGGGGACAGCCCCT | |
| | CCTGGCTAGTCACACCCTGTAGGCTCCTCTATATAACCCAGGGGCACAGG | |
| | GGCTGCCCTCATTCTACCACCACCTCCACAGCACAGACAGACACTCAGGA | |
| | GCCAGCCAGCGTCGA | |
| CAG | GACATTGATTATTGACTAGTTATTAATAGTAATCAATTACGGGGTCATTA | 108 |
| | GTTCATAGCCCATATATGGAGTTCCGCGTTACATAACTTACGGTAAATGG | |
| | CCCGCCTGGCTGACCGCCCAACGACCCCCGCCCATTGACGTCAATAATGA | |
| | CGTATGTTCCCATAGTAACGCCAATAGGGACTTTCCATTGACGTCAATGG | |
| | GTGGAGTATTTACGGTAAACTGCCCACTTGGCAGTACATCAAGTGTATCA | |
| | TATGCCAAGTACGCCCCCTATTGACGTCAATGACGGTAAATGGCCCGCCT | |
| | GGCATTATGCCCAGTACATGACCTTATGGGACTTTCCTACTTGGCAGTAC | |
| | ATCTACGTATTAGTCATCGCTATTACCATGGTCGAGGTGAGCCCCACGTT | |
| | CTGCTTCACTCTCCCCATCTCCCCCCCCTCCCCACCCCCAATTTTGTATT | |
| | TATTTATTTTTTAATTATTTTGTGCAGCGATGGGGGCGGGGGGGGGGGGG | |
| | GGGCGCGCGCCAGGCGGGGCGGGGCGGGGCGAGGGGCGGGGCGGGGCGAG | |
| | GCGGAGAGGTGCGGCGGCAGCCAATCAGAGCGGCGCGCTCCGAAAGTTTC | |
| | CTTTTATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGCTCCGC | |
| | CGCCGCCTCGCGCCGCCCGCCCCGGCTCTGACTGACCGCGTTACTCCCAC | |
| | AGGTGAGCGGGCGGGACGGCCCTTCTCCTCCGGGCTGTAATTAGCGCTTG | |
| | GTTTAATGACGGCTTGTTTCTTTTCTGTGGCTGCGTGAAAGCCTTGAGGG | |
| | GCTCCGGGAGGGCCCTTTGTGCGGGGGGAGCGGCTCGGGGGGTGCGTGCG | |
| | TGTGTGTGTGCGTGGGGAGCGCCGCGTGCGGCTCCGCGCTGCCCGGCGGC | |
| | TGTGAGCGCTGCGGGCGCGGCGCGGGGCTTTGTGCGCTCCGCAGTGTGCG | |
| | CGAGGGGAGCGCGGCCGGGGGCGGTGCCCCGCGGTGCGGGGGGGGCTGCG | |
| | AGGGGAACAAAGGCTGCGTGCGGGGTGTGTGCGTGGGGGGGTGAGCAGGG | |
| | GGTGTGGGCGCGTCGGTCGGGCTGCAACCCCCCCTGCACCCCCCTCCCCG | |
| | AGTTGCTGAGCACGGCCCGGCTTCGGGTGCGGGGCTCCGTACGGGGCGTG | |
| | GCGCGGGGCTCGCCGTGCCGGGCGGGGGGTGGCGGCAGGTGGGGGTGCCG | |
| | GGCGCGGCGGGGCCGCCTCGGGCCGGGGAGGGCTCGGGGGAGGGGCGCGG | |
| | CGGCCCCCGGAGCGCCGGCGGCTGTCGAGGCGCGGCGAGCCGCAGCCATT | |
| | GCCTTTTATGGTAATCGTGCGAGAGGGCGCAGGGACTTCCTTTGTCCCAA | |
| | ATCTGTGCGGAGCCGAAATCTGGGAGGCGCCGCCGCACCCCCTCTAGCGG | |
| | GCGCGGGGCGAAGCGGTGCGGCGCCGGCAGGAAGGAAATGGGCGGGGAGG | |
| | GCCTTCGTGCGTCGCCGCGCCGCCGTCCCCTTCTCCCTCTCCAGCCTCGG | |
| | GGCTGTCCGCGGGGGGACGGCTGCCTTCGGGGGGGACGGGGCAGGGGGG | |
| | GTTCGGCTTCTGGCGTGTGACCGGCGGCTCTAGAGCCTCTGCTAACCATG | |
| | TTCATGCCTTCTTCTTTTTCCTACAGCTCCTGGGCAACGTGCTGGTTATT | |
| | GTGCTGTCTCATCATTTTGGCAAAG | |
| mU1a | ATGGAGGCGGTACTATGTAGATGAGAATTCAGGAGCAAACTGGGAAAAGC | 109 |
| | AACTGCTTCCAAATATTTGTGATTTTTACAGTGTAGTTTTGGAAAAACTC | |
| | TTAGCCTACCAATTCTTCTAAGTGTTTTAAAATGTGGGAGCCAGTACACA | |
| | TGAAGTTATAGAGTGTTTTAATGAGGCTTAAATATTTACCGTAACTATGA | |
| | AATGCTACGCATATCATGCTGTTCAGGCTCCGTGGCCACGCAACTCATAC | |
| | T | |
| EF-1α | GGGCAGAGCGCACATCGCCCACAGTCCCCGAGAAGTTGGGGGGAGGGGTC | 110 |
| | GGCAATTGAACGGGTGCCTAGAGAAGGTGGCGCGGGGTAAACTGGGAAAG | |
| | TGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCC | |
| | GCCAGAACACAG | |
| Spc | AGAGGCCGTCCGCCCTCGGCACCATCCTCACGACACCCAAATATGGCGACGGGTG | 127 |
| Version 1 | AGGAATGGTGGGGAGTTATTTTTAGAGCGGTGAGGAAGGTGGGCAGGCAGCAGGT | |
| ((Spc5v1) | GTTGGCGCTCCATATTTGGCGGGAGTTATTTTTAGAGCGGAGGAATGGTGGACAC | |
| mutant of | CCAAATATGGCGACGGTTCCTCACCCGTCGCTAAAAATAACTCCGTGTCCGCCCT | |
| Spc5-12) | CGGCCGGGGCCGCATTCCTGGGGGCCGGGCGGTGCTCCCGCCCGCCTCGATAAAA | |
| | GGCTCCGGGGCCGGCGGCGGCCCACGAGCTACCCGGAGGAGCGGGAGGCGCCAAG | |
| | CGGAA | |
| Spc | GGCCGTCCGCCCTCGGCACCATCCTCACGACACCCAAATATGGCGACGGGTGAGG | 128 |
| Version 2 | AATGGTGGGGAGTTATTTTTAGAGCGGTGAGGAAGGTGGGCAGGCAGCAGGTGTT | |
| ((Spc5v2) | GGGGGAGTTATTTTTAGAGCGGGGAGTTATTTTTAGAGCGGAGGAATGGTGGACA | |
| mutant of | CCCAAATATGGCGACGGTTCCTCACGGACACCCAAATATGGCGACGGGCCCTCGG | |
| Spc5-12) | CCGGGGCCGCATTCCTGGGGGCCGGGCGGTGCTCCCGCCCGCCTCGATAAAAGGC | |
| | TCCGGGGCCGGCGGCGGCCCACGAGCTACCCGGAGGAGCGGGAGGCGCCAAGC | |

In some embodiments, the muscle cell-specific promoter is a muscle creatine-kinase (“MCK”) promoter. The muscle creatine kinase (MCK) gene is highly active in all striated muscles. Creatine kinase plays an important role in the regeneration of ATP within contractile and ion transport systems. It allows for muscle contraction when neither glycolysis nor respiration is present by transferring a phosphate group from phosphocreatine to ADP to form ATP. There are four known isoforms of creatine kinase: brain creatine kinase (CKB), muscle creatine kinase (MCK), and two mitochondrial forms (CKMi). MCK is the most abundant non-mitochondrial mRNA that is expressed in all skeletal muscle fiber types and is also highly active in cardiac muscle. The MCK gene is not expressed in myoblasts, but becomes transcriptionally active when myoblasts commit to terminal differentiation into myocytes. MCK gene regulatory regions display striated muscle-specific activity and have been extensively characterized in vivo and in vitro. The major known regulatory regions in the MCK gene include a muscle-specific enhancer located approximately 1.1 kb 5′ of the transcriptional start site in mouse and a 358-bp proximal promoter. Additional sequences that modulate MCK expression are distributed over 3.3 kb region 5′ of the transcriptional start site and in the 3.3-kb first intron. Mammalian MCK regulatory elements, including human and mouse promoter and enhancer elements, are described in Hauser et al., “Analysis of Muscle Creatine Kinase Regulatory Elements in Recombinant Adenoviral Vectors,” Mol. Therapy 2:16-25 (2000), which is hereby incorporated by reference in its entirety. Suitable muscle creatine kinase (MCK) promoters include, without limitation, a wild type MCK promoter, a dMCK promoter, and a tMCK promoter (Wang et al., “Construction and Analysis of Compact Muscle-Specific Promoters for AAV Vectors,” Gene Ther. 15(22):1489-1499 (2008), which is hereby incorporated by reference in its entirety).

In some embodiments, the muscle-specific promoter is selected from an Spc5-12 promoter (SEQ ID NO: 18 or 106)(including a modified Spc5-12 promoter SPc5v1 or SPc5v2 (SEQ ID NO: 127 or 128, respectively), a muscle creatine kinase myosin light chain (MLC) promoter, a myosin heavy chain (MHC) promoter, a desmin promoter (human—SEQ ID NO: 98), a MCK7 promoter (SEQ ID NO: 104), a CK6 promoter, a CK8 promoter (SEQ ID NO: 107), a MCK promoter (or a truncated form thereof) (SEQ ID NO: 105 or 21), an alpha actin promoter, a beta actin promoter, an gamma actin promoter, an E-syn promoter, a cardiac troponin C promoter, a troponin I promoter, a myoD gene family promoter, or a muscle-selective promoter residing within intron 1 of the ocular form of Pitx3.

Synthetic promoter c5-12 (Li, X. et al. Nature Biotechnology Vol. 17, pp. 241-245, MARCH 1999), known as the Spc5-12 promoter, has been shown to have cell type restricted expression, specifically muscle-cell specific expression. At less than 350 bp in length, the Spc5-12 promoter is smaller in length than most endogenous promoters, which can be advantageous when the length of the nucleic acid encoding the therapeutic protein is relatively long.

Alternatively, the promoter may be a constitutive promoter, for example, the CB7 promoter. Additional promoters include: cytomegalovirus (CMV) promoter, Rous sarcoma virus (RSV) promoter, MMT promoter, EF-1 alpha promoter (SEQ ID NO: 110), UB6 promoter, chicken beta-actin promoter, CAG promoter (SEQ ID NO: 108). In some embodiments, particularly where it may be desirable to turn off transgene expression, an inducible promoter is used, e.g., hypoxia-inducible or rapamycin-inducible promoter.

### 5.4.2 Introns

Certain gene expression cassettes further include an intron, for example, 5′ of the AUF1 or microdystrophin coding sequence which may enhance proper splicing and, thus, transgene expression. Accordingly, in some embodiments, an intron is coupled to the 5′ end of a sequence encoding an AUF1 or microdystrophin protein. In certain embodiments, the intron is less than 100 nucleotides in length.

In embodiments, the intron is a VH4 intron. The VH4 intron nucleic acid can comprise SEQ ID NO: 111 as shown in Table 11 below.

TABLE 11
Nucleotide sequences for different introns

| | SEQ | |
|---|---|---|
| Structure | ID | Sequence |
| VH4 | 111 | GTGAGTATCTCAGGGATCCAGACATGGGGATA |
| intron | | TGGGAGGTGCCTCTGATC |
| | | CCAGGGCTCACTGTGGGTCTCTCTGTTCACAG |
| Chimeric | 112 | GTAAGTATCAAGGTTACAAGACAGGTTTAAGG |
| intron | | AGACCAATAGAAACTGGGCTTGTCGAGACAGA |
| | | GAAGACTCTTGCGTTTCTGATAGGCACCTATT |
| | | GGTCTTACTGACATCCACTTTGCCTTTCTCTC |
| | | CACAG |
| SV40 | 113 | GTAAGTTTAGTCTTTTTGTCTTTTATTTCAGG |
| intron | | TCCCGGATCCGGTGGTGGTGCAAATCAAAGAA |
| | | CTGCTCCTCAGTGGATGTTGCCTTTACTTCTA |
| | | G |
| ß- | 138 | GTAAGTATCAAGGTTACAAGACAGGTTTAAGG |
| globin/ | | AGACCAATAGAAACTGGGCTTGTCGAGACAGA |
| Ig Intron | | GAAGACTCTTGCGTTTCTGATAGGCACCTATT |
| | | GGTCTTACTGACATCCACTTTGCCTTTCT |

In other embodiments, the intron is a chimeric intron derived from human β-globin and Ig heavy chain (also known as β-globin splice donor/immunoglobulin heavy chain splice acceptor intron, or β-globin/IgG chimeric intron) (Table 11, SEQ ID NO: 112). Other introns well known to the skilled person may be employed, such as the chicken 0-actin intron, minute virus of mice (MVM) intron, human factor IX intron (e.g., FIX truncated intron 1), β-globin splice donor/immunoglobulin heavy chain splice acceptor intron (Table 11, SEQ ID NO: 138), adenovirus splice donor/immunoglobulin splice acceptor intron, SV40 late splice donor/splice acceptor (19S/16S) intron (Table 11, SEQ ID NO: 113).

### 5.4.3 Other Regulatory Elements

Another aspect of the present disclosure relates to expression cassettes comprising a polyadenylation (polyA) site downstream of the coding region of the microdystrophin transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene, the bovine growth hormone (BPH) gene, the human growth hormone (hGH) gene, and the synthetic polyA (SPA) site. Exemplary polyA signal sequences useful in the constructs described herein are provided in Table 2 supra.

Also provided are constructs comprising a Woodchuck Hepatitis Virus Posttranscriptional Regulatory Element (WPRE) which may enhance transgene expression. The WPRE element may be inserted into 3′ untranslated regions of the transgene 5′ of the polyadenylation signal sequence. See, e.g., Zufferey et al, J. Virol. 73:2886-2892 (1999), which is hereby incorporated by reference in its entirety. In particular embodiments, the WPRE element has a nucleotide sequence of SEQ ID NO: 24 (see Table 2 supra).

Other elements that may be included in the construct are filler or stuffer sequences that may be incorporated particularly at the 5′ and 3′ ends between the ITR sequences and the expression cassette sequences to optimized the length of nucleic acid between the ITR sequences to improve packaging efficiency. An SV40 polyadenylation sequence positioned adjacent to an ITR sequence (can insulate transgene transcription from interference from the ITRs. Exemplary stuffer sequences and the SV40 polyA sequence are provided in Table 2, supra. Alternative polyA sequences and stuffer sequences are known in the art, see e.g. Table 12.

Nucleic acids comprising a stuffer (or filler) polynucleotide sequence extend the transgene size of any heterologous gene, for example an AUF1 gene of Table 2 or 3. In some embodiments, a stuffer (or filler) polynucleotide sequence comprises SEQ ID NO:26 or 27. In some embodiments, a stuffer (or filler) polynucleotide sequence comprises SEQ ID NO:139-143, or a fragment of SEQ ID NO:X139-143 (see Table 12) between 1-10, 10-20, 20-30, 30-40, 40-50, 50-60, 60-75, 75-100, 100-150, 150-200, 200-250, 250-300, 300-400, 400-500, 500-600, 600-750, 750-1,000, 1,000-1,500, 1,500-1,601, nucleotides in length. In other embodiments, the stuffer polynucleotide comprises a nucleic acid sequence SEQ ID NO:139, SEQ ID NO:140, SEQ ID NO141, SEQ ID NO:142, or SEQ ID NO:X143 (see Table 12), or a fragment or fragments thereof.

In some embodiments, the stuffer polynucleotide sequence has a length that when combined with the heterologous gene sequence, the total combined length of the heterologous gene sequence and stuffer polynucleotide sequence is between about 2.4-5.2 kb, or between about 3.1-4.7 kb. The transgene may comprise any one of the genes or nucleic acids encoding a therapeutic AUF1 gene listed in, but not limited to, Tables 2 and 3.

In the case of stuffer sequences, and enhancer sequences such as introns, the nucleic acid sequences are operably linked to the transgene in a contiguous, or substantially contiguous manner. Where necessary, operably linked may refer to joining a coding region and a non-coding region, or two coding regions in a contiguous manner, e.g. in reading frame. In some instances, for example enhancers which may function when separated from the promoter by several kilobases, such as intronic sequences and stuffer sequences, these regulatory sequences may be operably linked while not directly contiguous with a downstream or upstream promoter and/or heterologous gene.

TABLE 12

| Short description | Nucleotide sequence |
|---|---|
| Non-coding stuffer | ATAGTCTATCCAGGTTGAGCATCCTGCTGGTGGTTACAAGAAACTGTT |
| sequence 1602 bp | TGAAACTGTGGAGGAACTGTCCTCGCCGCTCACAGCTCATGTAACAGG |
| SEQ ID NO: 139 | CAGGATCCCCCTCTGGCTCACCGGCAGTCTCCTTCGATGTGGGCCAGG |
| | ACTCTTTGAAGTTGGATCTGAGCCATTTTACCACCTGTTTGATGGGCA |
| | AGCCCTCCTGCACAAGTTTGACTTTAAAGAAGGACATGTCACATACCA |
| | CAGAAGGTTCATCCGCACTGATGCTTACGTACGGGCAATGACTGAGAA |
| | AAGGATCGTCATAACAGAATTTGGCACCTGTGCTTTCCCAGATCCCTG |
| | CAAGAATATATTTTCCAGGTTTTTTTCTTACTTTCGAGGAGTAGAGGT |
| | TACTGACAATTGCCCTTGTTAATGTCTACCCAGTGGGGGAAGATTACT |
| | ACGCTTGCACAGAGACCAACTTTATTACAAAGATTAATCCAGAGACCT |
| | TGGAGACAATTAAGCAGGTTGATCTTTGCAACTAAGTCTCTGTCAATG |
| | GGGCCACTGCTCACCCCCACATTGAAAATGATGGAACCGTTTACAATA |
| | TTGGTAATTGCTTTGGAAAAAATTTTTCAATTGCCTACAACATTGTAA |
| | AGATCCCACCACTGCAAGCAGACAAGGAAGATCCAATAAGCAAGTCAG |
| | AGATCGTTGTACAATTCCCCTGCAGTGACCGATTCAAGCCATCTTACG |
| | TTCATAGTTTTGGTCTGACTCCCAACTATATCGTTTTTGTGGAGACAC |
| | CAGTCAAAATTAACCTGTTCAAGTTCCTTTCTTCATGGAGTCTTTGGG |
| | GAGCCAACTACATGGATTGTTTTGAGTCCAATGAAACCATGGGGTTTG |
| | GCTTCATATTGCTGACAAAAAAAGGAAAAAGTACCTCAATAATAAATA |
| | CAGAACTTCTCCTTTCAACCTCTTCCATCACATCAACACCTATGAAGA |
| | CAATGGGTTTCTGATTGTGGATCTCTGCTGCTGGAAAGGATTTGAGTT |
| | TGTTTATAATTACTTATATTTAGCCAATTTACGTGAGAACTGGGAAGA |
| | GGTGAAAAAAAATGCCAGAAAGGCTCCCCAACCTGAAGTTAGGAGATA |
| | TGTACTTCCTTTGAATATTGACAAGGCTGACACAGGCAAGAATTTAGT |
| | CAGCTCCCCAATACAACTGCCACTGCAATTCTGTGCAGTGACGAGACT |
| | ATCTGGCTGGAGCCTGAAGTTCTCTTTTCAGGGCCTCGTCAAGCATTT |
| | GAGTTTCCTCAAATCAATTACCAGAAGTATTGTGGGAAACCTTACACA |
| | TATGCGTATGGACTTGGCTTGAATCACTTTGTTCCAGATAGGCTCTGT |
| | AAGCTGAATGTCAAAACTAAAGAAACTTGGGTTTGGCAAGAGCCTGAT |
| | TCATACCCATCAGAACCCATCTTTGTTTCTCACCCAGATGCCTTGGAA |
| | GAAGATGATGGTGTAGTTCTGAGTGTGGTGGTGAGCCCAGGAGCAGGA |
| | CAAAAGCCTGCTTATCTCCTGATTCTGAATGCCAAGGACTTAAGTGAA |
| | GTTGCCCGGGCTGAAGTGGAGATTAACATCCCTGTCACCTTTCATGGA |
| | CTGTTCAAAAAATCTTGA |
| Non-coding stuffer | CGAGTTTAATTGGTTTATAGAACTCTTCAAACAAATTAAACCAAAAAT |
| sequence 596 bp | TTCAATGCCAAGAAAGGGTCTTTAAAACGAAATTACAGAAGGACCAAA |
| SEQ ID NO: 140 | TGATAAGGAAGAAAAATGCAGAGATAAAAGTAATATCAATTAGGATCA |
| | TAAGCTACTTATTATCAATGAAAAGTAACAGAAACATAGATGCTGCAG |
| | AAATCTTCTGAGGAGTAGCTTCAACGCCTCAGGGTGTGGACAATGTAT |
| | TCAGCATAGAGGTCCCTGTAATGGGGATATCAGAATCCAGAGTTGCTT |
| | TAATGTTACAAACTAAAAAAGATGTAAGAGAGTTTGGTTCTTGATAAA |
| | GAAACAGAGGCTTACATTGAGTACTGGATAGCTTCAACCGCAGACTCA |
| | GATGGCAGAAAATCATTCACTGCAACTTCCTTGTTCTCGTTTTTCTTG |
| | TCTGTAAGATATTAGAGTTAAAGGGAAAAACTAATACTTGTTGAGAGA |
| | TCAATAGAGATGAATAAGGAGGAACACTGAAGAAAAAGGATACAGTCT |
| | TCGAAGAAACGACGGATTTCAGAGAGACGGTGAGGAGGAAGTTCTTTG |
| | ATGTCAGTGTAGTGCTTATA |
| Non-coding stuffer | CGAGTTTAATTGGTTTATAGAACTCTTCAAACAAATTAAACCAAAAAT |
| sequence 1096 | TTCAATGCCAAGAAAGGGTCTTTAAAACGAAATTACAGAAGGACCAAA |
| SEQ ID NO: 141 | TGATAAGGAAGAAAAATGCAGAGATAAAAGTAATATCAATTAGGATCA |
| | TAAGCTACTTATTATCAATGAAAAGTAACAGAAACATAGATGCTGCAG |
| | AAATCTTCTGAGGAGTAGCTTCAACGCCTCAGGGTGTGGACAATGTAT |
| | TCAGCATAGAGGTCCCTGTAATGGGGATATCAGAATCCAGAGTTGCTT |
| | TAATGTTACAAACTAAAAAAGATGTAAGAGAGTTTGGTTCTTGATAAA |
| | GAAACAGAGGCTTACATTGAGTACTGGATAGCTTCAACCGCAGACTCA |
| | GATGGCAGAAAATCATTCACTGCAACTTCCTTGTTCTCGTTTTTCTTG |
| | TCTGTAAGATATTAGAGTTAAAGGGAAAAACTAATACTTGTTGAGAGA |
| | TCAATAGAGATGAATAAGGAGGAACACTGAAGAAAAAGGATACAGTCT |
| | TCGAAGAAACGACGGATTTCAGAGAGACGGTGAGGAGGAAGTTCTTTG |
| | ATGTCAGTGTAGTGCTTATATTCAGGATCATCAACACACACTGCAATG |
| | ATCTTGTCATCTTTTTCACCCTAAAATTACAGCGCCAAAAATACAAGA |
| | TTGGAGTACAAGACCATTTAAACTGACCTAAAGGATTAGAGTAAGAGA |
| | AAAAAAAAACAGAGTCTTTTCATTGATCAAGTTTAGGTTTTACCTGGT |
| | CAATCATAGGCATTAATCCAATGGCTCTGGCACGCAGAAAACAACCCG |
| | GAAGCACAGGTTCCTACACAAAGATAATAATATATATTTGAAATACAA |
| | AAAATTGGTGCAAATAGTATAGGGATAATATGAGAAAGAAAGAAAGAG |
| | TAATACCTGCATGATGACTAAGACATCAATGGGGTCATTGTCTTCACA |
| | CAATGTGCGAGGAACAAAACCATAGTTGTGAGGGTACACAACTGATGA |
| | GTAGAGAATACGATCAACCTGAATGAGAGATATCAAACTTGTTGAGAT |
| | TGATTTTGCTATAAGAAAACCATTCATATAAAAAATAAAA |
| Non-coding stuffer | CGAGTTTAATTGGTTTATAGAACTCTTCAAACAAATTAAACCAAAAAT |
| sequence 1596 bp | TTCAATGCCAAGAAAGGGTCTTTAAAACGAAATTACAGAAGGACCAAA |
| SEQ ID NO: 142 | TGATAAGGAAGAAAAATGCAGAGATAAAAGTAATATCAATTAGGATCA |
| | TAAGCTACTTATTATCAATGAAAAGTAACAGAAACATAGATGCTGCAG |
| | AAATCTTCTGAGGAGTAGCTTCAACGCCTCAGGGTGTGGACAATGTAT |
| | TCAGCATAGAGGTCCCTGTAATGGGGATATCAGAATCCAGAGTTGCTT |
| | TAATGTTACAAACTAAAAAAGATGTAAGAGAGTTTGGTTCTTGATAAA |
| | GAAACAGAGGCTTACATTGAGTACTGGATAGCTTCAACCGCAGACTCA |
| | GATGGCAGAAAATCATTCACTGCAACTTCCTTGTTCTCGTTTTTCTTG |
| | TCTGTAAGATATTAGAGTTAAAGGGAAAAACTAATACTTGTTGAGAGA |
| | TCAATAGAGATGAATAAGGAGGAACACTGAAGAAAAAGGATACAGTCT |
| | TCGAAGAAACGACGGATTTCAGAGAGACGGTGAGGAGGAAGTTCTTTG |
| | ATGTCAGTGTAGTGCTTATATTCAGGATCATCAACACACACTGCAATG |
| | ATCTTGTCATCTTTTTCACCCTAAAATTACAGCGCCAAAAATACAAGA |
| | TTGGAGTACAAGACCATTTAAACTGACCTAAAGGATTAGAGTAAGAGA |
| | AAAAAAAAACAGAGTCTTTTCATTGATCAAGTTTAGGTTTTACCTGGT |
| | CAATCATAGGCATTAATCCAATGGCTCTGGCACGCAGAAAACAACCCG |
| | GAAGCACAGGTTCCTACACAAAGATAATAATATATATTTGAAATACAA |
| | AAAATTGGTGCAAATAGTATAGGGATAATATGAGAAAGAAAGAAAGAG |
| | TAATACCTGCATGATGACTAAGACATCAATGGGGTCATTGTCTTCACA |
| | CAATGTGCGAGGAACAAAACCATAGTTGTGAGGGTACACAACTGATGA |
| | GTAGAGAATACGATCAACCTGAATGAGAGATATCAAACTTGTTGAGAT |
| | TGATTTTGCTATAAGAAAACCATTCATATAAAAAATAAACTTTGTTCT |
| | CATCTAACCTTGATGAGTCCTGTCTTTTTGTCAAGCTCGTATTTGACC |
| | TTGCTTCCTTTAGTGATCTCAACAACCTAATAATCATCCAAAGATAAA |
| | ATGATTAGAGAATCTAATAACAACATACTCTGTTTAGAACAAAGAGTA |
| | GGAAAAAACTTACCACATTGAAAATCTGTGGAGCTCCAGGTCCTAGAT |
| | AGATTATCCAGACTTATGATTTAGTAACAGAATACAAAAGTATGAAAT |
| | CAAAAAGTAGCATGTTTAGAATGATTTATATACCAATCTCAAGATCAT |
| | GCCATGGATGAGCAGCTACGGATCTTCTTGACAAGGATGAGAGAATCC |
| | TCTCGTTAAGACGAGGAGCTGGTCGCTGCAGCCTCTGGTTATCTTTAG |
| | TTTCTTCACTCATCTGTCAAAATCAGAACGTTTCATCACTCATTGATA |
| | TTGACTGAATCTAACATCATAACCCTAATTGGCAGAGAGAGAATCAAT |
| | CGAATCAAGAGA |
| Non-coding stuffer | CGAGTTTAATTGGTTTATAGAACTCTTCAAACAAATTAAACCAAAAAT |
| sequence 2002 bp | TTCAATGCCAAGAAAGGGTCTTTAAAACGAAATTACAGAAGGACCAAA |
| SEQ ID NO: 143 | TGATAAGGAAGAAAAATGCAGAGATAAAAGTAATATCAATTAGGATCA |
| | TAAGCTACTTATTATCAATGAAAAGTAACAGAAACATAGATGCTGCAG |
| | AAATCTTCTGAGGAGTAGCTTCAACGCCTCAGGGTGTGGACAATGTAT |
| | TCAGCATAGAGGTCCCTGTAATGGGGATATCAGAATCCAGAGTTGCTT |
| | TAATGTTACAAACTAAAAAAGATGTAAGAGAGTTTGGTTCTTGATAAA |
| | GAAACAGAGGCTTACATTGAGTACTGGATAGCTTCAACCGCAGACTCA |
| | GATGGCAGAAAATCATTCACTGCAACTTCCTTGTTCTCGTTTTTCTTG |
| | TCTGTAAGATATTAGAGTTAAAGGGAAAAACTAATACTTGTTGAGAGA |
| | TCAATAGAGATGAATAAGGAGGAACACTGAAGAAAAAGGATACAGTCT |
| | TCGAAGAAACGACGGATTTCAGAGAGACGGTGAGGAGGAAGTTCTTTG |
| | ATGTCAGTGTAGTGCTTATATTCAGGATCATCAACACACACTGCAATG |
| | ATCTTGTCATCTTTTTCACCCTAAAATTACAGCGCCAAAAATACAAGA |
| | TTGGAGTACAAGACCATTTAAACTGACCTAAAGGATTAGAGTAAGAGA |
| | AAAAAAAAACAGAGTCTTTTCATTGATCAAGITTAGGTTTTACCTGGT |
| | CAATCATAGGCATTAATCCAATGGCTCTGGCACGCAGAAAACAACCCG |
| | GAAGCACAGGTTCCTACACAAAGATAATAATATATATTTGAAATACAA |
| | AAAATTGGTGCAAATAGTATAGGGATAATATGAGAAAGAAAGAAAGAG |
| | TAATACCTGCATGATGACTAAGACATCAATGGGGTCATTGTCTTCACA |
| | CAATGTGCGAGGAACAAAACCATAGTTGTGAGGGTACACAACTGATGA |
| | GTAGAGAATACGATCAACCTGAATGAGAGATATCAAACTTGTTGAGAT |
| | TGATTTTGCTATAAGAAAACCATTCATATAAAAAATAAACTTTGTTCT |
| | CATCTAACCTTGATGAGTCCTGTCTTTTTGTCAAGCTCGTATTTGACC |
| | TTGCTTCCTTTAGTGATCTCAACAACCTAATAATCATCCAAAGATAAA |
| | ATGATTAGAGAATCTAATAACAACATACTCTGTTTAGAACAAAGAGTA |
| | GGAAAAAACTTACCACATTGAAAATCTGTGGAGCTCCAGGTCCTAGAT |
| | AGATTATCCAGACTTATGATTTAGTAACAGAATACAAAAGTATGAAAT |
| | CAAAAAGTAGCATGTTTAGAATGATTTATATACCAATCTCAAGATCAT |
| | GCCATGGATGAGCAGCTACGGATCTTCTTGACAAGGATGAGAGAATCC |
| | TCTCGTTAAGACGAGGAGCTGGTCGCTGCAGCCTCTGGTTATCTTTAG |
| | TTTCTTCACTCATCTGTCAAAATCAGAACGTTTCATCACTCATTGATA |
| | TTGACTGAATCTAACATCATAACCCTAATTGGCAGAGAGAGAATCAAT |
| | CGAATCAAGAGTATTAAATGGAAAAAGCGAATCAAGACCCCACAAGGG |
| | AAAACAATCCTTAAAGCAGACTTGAGATCGATCATACCCAAATTATGG |
| | ATTCATATATTGTTAACGTATCGATTACTGAAAAGATGTATACCAAAT |
| | CTGTTCACTTTTTCTCTATAGACTCGATGGATGATTGAGATTTGAAGC |
| | AACAAAATACCCAGAAGATTAAACATGGAAAAGCATCAAACTTTGATG |
| | ATCTTAGAACGATGACAAAAGAAAAAAAAACGTACCTTTGGATCGAAA |
| | CGAAACAGCCGATTGTTGTTTTCTTTATCGCAAGGATGATGAAGAAAC |
| | TTTGGGAGAGAAACAAGTGAAGCCCGTTGGTCTAGCAAGTGATTGTAA |
| | AATGTATATATGAGTCACCACCGAGATATACGGA |

### 5.4.4 Reporter Genes

In some embodiments, the disclosed gene cassettes, and thus the adeno-associated viral vectors, comprise a nucleic acid molecule encoding a reporter protein. The reporter protein may be selected from the group consisting of, e.g., P3-galactosidase, chloramphenicol acetyl transferase, luciferase, and fluorescent proteins.

In certain embodiments, the reporter protein is a fluorescent protein. Suitable fluorescent proteins include, without limitation, green fluorescent proteins (e.g., GFP, GFP-2, tagGFP, turboGFP, EGFP, Emerald, Azami Green, Monomeric Azami Green, CopGFP, AceGFP, ZsGreenl), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, mRasberry, mStrawberry, Jred), and orange fluorescent proteins (mOrange, mKO, Kusabira-Orange, Monomeric Kusabira-Orange, mTangerine, tdTomato), or any other suitable fluorescent protein. In certain embodiments, the reporter protein is a fluorescent protein selected from the group consisting of green fluorescent protein (GFP), enhanced green fluorescent protein (EGFP), and yellow fluorescent protein (YFP).

In some embodiments, the reporter protein is luciferase. As used herein, the term “luciferase” refers to members of a class of enzymes that catalyze reactions that result in production of light. Luciferases have been identified in and cloned from a variety of organisms including fireflies, click beetles, sea pansy (Renilla), marine copepods, and bacteria among others. Examples of luciferases that may be used as reporter proteins include, e.g., Renilla (e.g., Renilla reniformis) luciferase, Gaussia (e.g., Gaussia princeps) luciferase), Metridia luciferase, firefly (e.g., Photinus pyralis luciferase), click beetle (e.g., Pyrearinus termitilluminans) luciferase, deep sea shrimp (e.g., Oplophorus gracilirostris) luciferase). Luciferase reporter proteins include both naturally occurring proteins and engineered variants designed to have one or more altered properties relative to the naturally occurring protein, such as increased photostability, increased pH stability, increased fluorescence or light output, reduced tendency to dimerize, oligomerize, aggregate or be toxic to cells, an altered emission spectrum, and/or altered substrate utilization.

### 5.4.5 Viral Vectors

The AUF1 and microdystrophin transgenes disclosed herein can be included in an AAV vector for gene therapy administration to a human subject. In some embodiments, recombinant AAV (rAAV) vectors can comprise an AAV viral capsid and a viral or artificial genome comprising an expression cassette flanked by AAV inverted terminal repeats (ITRs) wherein the expression cassette comprises an AUF1 or microdystrophin transgene, operably linked to one or more regulatory sequences that control expression of the transgene in human muscle cells to express and deliver the AUF1 protein or the microdystrophin as the case may be. The provided methods are suitable for use in the production of any isolated recombinant AAV particles for delivery of an AUF1 protein or microdystrophin described herein, in the production of a composition comprising any isolated recombinant AAV particles encoding an AUF1 protein or a microdystrophin, or in the method for treating a disease or disorder amenable for treatment with an AUF1 protein or a combination of an AUF1 protein and a microdystrophin in a subject in need thereof comprising the administration of any isolated recombinant AAV particles encoding an AUF1 protein or a combination (including administered separately) of an rAAV particle encoding an AUF1 protein and an rAAV particle encoding a microdystrophin described herein. As such, the rAAV can be of any serotype, variant, modification, hybrid, or derivative thereof, known in the art, or any combination thereof (collectively referred to as “serotype”). In particular embodiments, the AAV serotype has a tropism for muscle tissue (including skeletal muscle, cardiac muscle or smooth muscle).

In some embodiments, rAAV particles have a capsid protein from an AAV8 serotype. In other embodiments, rAAV particles have a capsid protein from an AAV9 serotype. In particular, provided are AUF1 constructs of vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, which have nucleotide sequences of SEQ ID NO:31 to 36 in an rAAV particle having an AAV8 capsid. Further provided for use in methods disclosed herein are the RGX-DYS1 construct in an rAAV particle having an AAV8 capsid and the RGX-DYS1 construct in an rAAV particle having an AAV9 capsid. Also provided are the RGX-DYS5 construct in an rAAV particle having an AAV8 capsid and the RGX-DYS5 construct in an rAAV particle having an AAV9 capsid.

In some embodiments, the rAAV particles comprise a capsid protein from an AAV capsid serotype selected from the group consisting of AAV1, AAV2, AAV3, AAV4, AAV5, AAV6, AAV7, AAV8, AAV9, AAV10, AAV11, AAV2i8 or AAV2.5 serotype or alternatively may be an AAVrh.8, AAVrh.10, AAVrh.43, AAVrh.74, AAVhu.37, AAVAAV.hu31, or AAVhu.32 serotype.

In some embodiments, rAAV particles comprise a capsid protein that is a derivative, modification, or pseudotype of AAV8 capsid protein. In some embodiments, rAAV particles comprise a capsid protein that has a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV8 capsid protein (SEQ ID NO: 114) (Table 13). In some embodiments, rAAV particles comprise a capsid protein that has a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein (SEQ ID NO: 115) (Table 13). In some embodiments, rAAV particles comprise a capsid protein that has capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV1, AAV2, AAV3, AAV4, AAV5, AAV6, AAV7, AAV8, AAV9, AAV10, AAV11, AAV2i8, AAV2.5, AAVrh.8, AAVrh.10, AAVrh.43, AAVrh.74 (SEQ ID NO: 119 and 120), AAVhu.37 (SEQ ID NO: 116), AAVAAV.hu31 (SEQ ID NO: 117), or AAVhu.32 (SEQ ID NO: 118) serotype capsid protein (see Table 13).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety). Self-complementary vectors may include a mutant ITR sequence, for example, the mutant 5′ ITR sequence in Table 2.

In additional embodiments, rAAV particles comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV2, AAV3, AAV4, AAV5, AAV6, AAV7, AAV8, AAV9, AAV10, AAV11, AAV2i8, AAV2.5, AAVrh.8, AAVrh.10, AAVrh.43, AAVrh.74, AAVhu.37, AAVAAV.hu31, or AAVhu.32), in particular AAV8. In additional embodiments, rAAV particles comprise a pseudotyped rAAV particle containing AAV8 capsid protein. In some embodiments, the pseudotyped rAAV8 particle is an rAAV2/8 pseudotyped particle. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In some embodiments, the rAAV particles comprise an AAV capsid protein chimeric of AAV8 capsid protein and one or more AAV capsid proteins from an AAV serotype selected from AAV1, AAV2, AAV3, AAV4, AAV5, AAV6, AAV7, AAV8, AAV9, AAV10, AAV11, AAV2i8, AAV2.5, AAVrh.8, AAVrh.10, AAVrh.43, AAVrh.74, AAVhu.37, AAVAAV.hu31, or AAVhu.32.

In some embodiments the rAAV particles comprises a Clade A, B, E, or F AAV capsid protein. In some embodiments, the rAAV particles comprises a Clade F AAV capsid protein. In some embodiments the rAAV particles comprises a Clade E AAV capsid protein.

Table 13 below provides examples of amino acid sequences for an AAV8, AAV9, AAV.rh74, AAV.hu31, AAVhu.32, and AAV.hu37 capsid proteins. Exemplary ITR sequences are provided in Table 2.

TABLE 13

| | SEQ | |
|---|---|---|
| Structure | ID | Sequence |
| AAV8 | 114 | MAADGYLPDW LEDNLSEGIR EWWALKPGAP |
| Capsid | | KPKANQQKQD DGRGLVLPGY KYLGPFNGLD |
| | | KGEPVNAADA AALEHDKAYD QQLQAGDNPY |
| | | LRYNHADAEF QERLQEDTSF GGNLGRAVFQ |
| | | AKKRVLEPLG LVEEGAKTAP GKKRPVEPSP |
| | | QRSPDSSTGI GKKGQQPARK RLNFGQTGDS |
| | | ESVPDPQPLG EPPAAPSGVG PNTMAAGGGA |
| | | PMADNNEGAD GVGSSSGNWH CDSTWLGDRV |
| | | ITTSTRTWAL PTYNNHLYKQ ISNGTSGGAT |
| | | NDNTYFGYST PWGYFDFNRF HCHFSPRDWQ |
| | | RLINNNWGFR PKRLSFKLFN IQVKEVTQNE |
| | | GTKTIANNLT STIQVFTDSE YQLPYVLGSA |
| | | HQGCLPPFPA DVFMIPQYGY LTLNNGSQAV |
| | | GRSSFYCLEY FPSQMLRTGN NFQFTYTFED |
| | | VPFHSSYAHS QSLDRLMNPL IDQYLYYLSR |
| | | TQTTGGTANT QTLGFSQGGP NTMANQAKNW |
| | | LPGPCYRQQR VSTTTGQNNN SNFAWTAGTK |
| | | YHLNGRNSLA NPGIAMATHK DDEERFFPSN |
| | | GILIFGKQNA ARDNADYSDV MLTSEEEIKT |
| | | TNPVATEEYG IVADNLQQQN TAPQIGTVNS |
| | | QGALPGMVWQ NRDVYLQGPI WAKIPHTDGN |
| | | FHPSPLMGGF GLKHPPPQIL IKNTPVPADP |
| | | PTTFNQSKLN SFITQYSTGQ VSVEIEWELQ |
| | | KENSKRWNPE IQYTSNYYKS TSVDFAVNTE |
| | | GVYSEPRPIG TRYLTRNL |
| AAV9 | 115 | MAADGYLPDW LEDNLSEGIR EWWALKPGAP |
| Capsid | | QPKANQQHQD NARGLVLPGY KYLGPGNGLD |
| | | KGEPVNAADA AALEHDKAYD QQLKAGDNPY |
| | | LKYNHADAEF QERLKEDTSF GGNLGRAVFQ |
| | | AKKRLLEPLG LVEEAAKTAP GKKRPVEQSP |
| | | QEPDSSAGIG KSGAQPAKKR LNFGQTGDTE |
| | | SVPDPQPIGE PPAAPSGVGS LTMASGGGAP |
| | | VADNNEGADG VGSSSGNWHC DSQWLGDRVI |
| | | TTSTRTWALP TYNNHLYKQI SNSTSGGSSN |
| | | DNAYFGYSTP WGYFDFNRFH CHFSPRDWQR |
| | | LINNNWGFRP KRLNFKLFNI QVKEVTDNNG |
| | | VKTIANNLTS TVQVFTDSDY QLPYVLGSAH |
| | | EGCLPPFPAD VFMIPQYGYL TLNDGSQAVG |
| | | RSSFYCLEYF PSQMLRTGNN FQFSYEFENV |
| | | PFHSSYAHSQ SLDRLMNPLI DQYLYYLSKT |
| | | INGSGQNQQT LKFSVAGPSN MAVQGRNYIP |
| | | GPSYRQQRVS TTVTQNNNSE FAWPGASSWA |
| | | LNGRNSLMNP GPAMASHKEG EDRFFPLSGS |
| | | LIFGKQGTGR DNVDADKVMI TNEEEIKTTN |
| | | PVATESYGQV ATNHQSAQAQ AQTGWVQNQG |
| | | ILPGMVWQDR DVYLQGPIWA KIPHTDGNFH |
| | | PSPLMGGFGM KHPPPQILIK NTPVPADPPT |
| | | AFNKDKLNSF ITQYSTGQVS VEIEWELQKE |
| | | NSKRWNPEIQ YTSNYYKSNN VEFAVNTEGV |
| | | YSEPRPIGTR YLTRNL |
| hu.37 | 116 | MAADGYLPDW LEDNLSEGIR EWWDLKPGAP |
| Capsid | | KPKANQQKQD DGRGLVLPGY KYLGPFNGLD |
| | | KGEPVNAADA AALEHDKAYD QQLKAGDNPY |
| | | LRYNHADAEF QERLQEDTSF GGNLGRAVFQ |
| | | AKKRVLEPLG LVEEAAKTAP GKKRPVEPSP |
| | | QRSPDSSTGI GKKGQQPAKK RLNFGQTGDS |
| | | ESVPDPQPIG EPPAGPSGLG SGTMAAGGGA |
| | | PMADNNEGAD GVGSSSGNWH CDSTWLGDRV |
| | | ITTSTRTWAL PTYNNHLYKQ ISNGTSGGST |
| | | NDNTYFGYST PWGYFDFNRF HCHFSPRDWQ |
| | | RLINNNWGFR PKRLSFKLFN IQVKEVTQNE |
| | | GTKTIANNLT STIQVFTDSE YQLPYVLGSA |
| | | HQGCLPPFPA DVFMIPQYGY LTLNNGSQAV |
| | | GRSSFYCLEY FPSQMLRTGN NFEFSYTFED |
| | | VPFHSSYAHS QSLDRLMNPL IDQYLYYLSR |
| | | TQSTGGTQGT QQLLFSQAGP ANMSAQAKNW |
| | | LPGPCYRQQR VSTTLSQNNN SNFAWTGATK |
| | | YHLNGRDSLV NPGVAMATHK DDEERFFPSS |
| | | GVLMFGKQGA GRDNVDYSSV MLTSEEEIKT |
| | | TNPVATEQYG VVADNLQQTN TGPIVGNVNS |
| | | QGALPGMVWQ NRDVYLQGPI WAKIPHTDGN |
| | | FHPSPLMGGF GLKHPPPQIL IKNTPVPADP |
| | | PTTFSQAKLA SFITQYSTGQ VSVEIEWELQ |
| | | KENSKRWNPE IQYTSNYYKS TNVDFAVNTE |
| | | GTYSEPRPIG TRYLTRNL |
| hu.31 | 117 | MAADGYLPDW LEDTLSEGIR QWWKLKPGPP |
| Capsid | | PPKPAERHKD DSRGLVLPGY KYLGPGNGLD |
| | | KGEPVNAADA AALEHDKAYD QQLKAGDNPY |
| | | LKYNHADAEF QERLKEDTSF GGNLGRAVFQ |
| | | AKKRLLEPLG LVEEAAKTAP GKKRPVEQSP |
| | | QEPDSSAGIG KSGSQPAKKK LNFGQTGDTE |
| | | SVPDPQPIGE PPAAPSGVGS LTMASGGGAP |
| | | VADNNEGADG VGSSSGNWHC DSQWLGDRVI |
| | | TTSTRTWALP TYNNHLYKQI SNSTSGGSSN |
| | | DNAYFGYSTP WGYFDFNRFH CHFSPRDWQR |
| | | LINNNWGFRP KRLNFKLFNI QVKEVTDNNG |
| | | VKTIANNLTS TVQVFTDSDY QLPYVLGSAH |
| | | EGCLPPFPAD VFMIPQYGYL TLNDGGQAVG |
| | | RSSFYCLEYF PSQMLRTGNN FQFSYEFENV |
| | | PFHSSYAHSQ SLDRLMNPLI DQYLYYLSKT |
| | | INGSGQNQQT LKFSVAGPSN MAVQGRNYIP |
| | | GPSYRQQRVS TTVTQNNNSE FAWPGASSWA |
| | | LNGRNSLMNP GPAMASHKEG EDRFFPLSGS |
| | | LIFGKQGTGR DNVDADKVMI TNEEEIKTTN |
| | | PVATESYGQV ATNHQSAQAQ AQTGWVQNQG |
| | | ILPGMVWQDR DVYLQGPIWA KIPHTDGNFH |
| | | PSPLMGGFGM KHPPPQILIK NTPVPADPPT |
| | | AFNKDKLNSF ITQYSTGQVS VEIEWELQKE |
| | | NSKRWNPEIQ YTSNYYKSNN VEFAVSTEGV |
| | | YSEPRPIGTR YLTRNL |
| hu.32 | 118 | MAADGYLPDW LEDTLSEGIR QWWKLKPGPP |
| Capsid | | PPKPAERHKD DSRGLVLPGY KYLGPGNGLD |
| | | KGEPVNAADA AALEHDKAYD QQLKAGDNPY |
| | | LKYNHADAEF QERLKEDTSF GGNLGRAVFQ |
| | | AKKRLLEPLG LVEEAAKTAP GKKRPVEQSP |
| | | QEPDSSAGIG KSGSQPAKKK LNFGQTGDTE |
| | | SVPDPGQPIG EPPAAPSGVG SLTMASGGGA |
| | | PVADNNEGAD GVGSSSGNWH CDSQWLGDRV |
| | | ITTSTRTWAL PTYNNHLYKQ ISNSTSGGSS |
| | | NDNAYFGYST PWGYFDFNRF HCHFSPRDWQ |
| | | RLINNNWGFR PKRLNFKLFN IQVKEVTDNN |
| | | GVKTIANNLT STVQVFTDSD YQLPYVLGSA |
| | | HEGCLPPFPA DVFMIPQYGY LTLNDGSQAV |
| | | GRSSFYCLEY FPSQMLRTGN NFQFSYEFEN |
| | | VPFHSSYAHS QSLDRLMNPL IDQYLYYLSK |
| | | TINGSGQNQQ TLKFSVAGPS NMAVQGRNYI |
| | | PGPSYRQQRV STTVTQNNNS EFAWPGASSW |
| | | ALNGRNSLMN PGPAMASHKE GEDRFFPLSG |
| | | SLIFGKQGTG RDNVDADKVM ITNEEEIKTT |
| | | NPVATESYGQ VATNHQSAQA QAQTGWVQNQ |
| | | GILPGMVWQD RDVYLQGPIW AKIPHTDGNF |
| | | HPSPLMGGFG MKHPPPQILI KNTPVPADPP |
| | | TAFNKDKLNS FITQYSTGQV SVEIEWELQK |
| | | ENSKRWNPEI QYTSNYYKSN NVEFAVNTEG |
| | | VYSEPRPIGT RYLTRNL |
| Rh.74 | 119 | MAADGYLPD WLEDNLSEG IREWWDLKP |
| version | | GAPKPKANQ QKQDNGRGL VLPGYKYLG |
| 1 | | PFNGLDKGE PVNAADAAA LEHDKAYDQ |
| | | QLQAGDNPY LRYNHADAE FQERLQEDT |
| | | SFGGNLGRA VFQAKKRVL EPLGLVESP |
| | | VKTAPGKKR PVEPSPQRS PDSSTGIGK |
| | | KGQQPAKKR LNFGQTGDS ESVPDPQPI |
| | | GEPPAGPSG LGSGTMAAG GGAPMADNN |
| | | EGADGVGSS SGNWHCDST WLGDRVITT |
| | | STRTWALPT YNNHLYKQI SNGTSGGST |
| | | NDNTYFGYS TPWGYFDFN RFHCHFSPR |
| | | DWQRLINNN WGFRPKRLN FKLFNIQVK |
| | | EVTQNEGTK TIANNLIST IQVFTDSEY |
| | | QLPYVLGSA HQGCLPPFP ADVFMIPQY |
| | | GYLTLNNGS QAVGRSSFY CLEYFPSQM |
| | | LRTGNNFEF SYNFEDVPF HSSYAHSQS |
| | | LDRLMNPLI DQYLYYLSR TQSTGGTAG |
| | | TQQLLFSQA GPNNMSAQA KNWLPGPCY |
| | | RQQRVSTTL SQNNNSNFA WTGATKYHL |
| | | NGRDSLVNP GVAMATHKD DEERFFPSS |
| | | GVLMFGKQG AGKDNVDYS SVMLTSEEE |
| | | IKTTNPVAT EQYGVVADN LQQQNAAPI |
| | | VGAVNSQGA LPGMVWQNR DVYLQGPIW |
| | | AKIPHTDGN FHPSPLMGG FGLKHPPPQ |
| | | ILIKNTPVP ADPPTTFNQ AKLASFITQ |
| | | YSTGQVSVE IEWELQKEN SKRWNPEIQ |
| | | YTSNYYKST NVDFAVNTE GTYSEPRPI |
| | | GTRYLTRNL |
| Rh.74 | 120 | MAADGYLPD WLEDNLSEG IREWWDLKP |
| version | | GAPKPKANQ QKQDNGRGL VLPGYKYLG |
| 2 | | PFNGLDKGE PVNAADAAA LEHDKAYDQ |
| | | QLQAGDNPY LRYNHADAE FQERLQEDT |
| | | SFGGNLGRA VFQAKKRVL EPLGLVESP |
| | | VKTAPGKKR PVEPSPQRS PDSSTGIGK |
| | | KGQQPAKKR LNFGQTGDS ESVPDPQPI |
| | | GEPPAAPSG VGPNTMAAG GGAPMADNN |
| | | EGADGVGSS SGNWHCDST WLGDRVITT |
| | | STRTWALPT YNNHLYKQI SNGTSGGST |
| | | NDNTYFGYS TPWGYFDFN RFHCHFSPR |
| | | DWQRLINNN WGFRPKRLN FKLFNIQVK |
| | | EVTQNEGTK TIANNLTST IQVFTDSEY |
| | | QLPYVLGSA HQGCLPPFP ADVFMIPQY |
| | | GYLTLNNGS QAVGRSSFY CLEYFPSQM |
| | | LRTGNNFEF SYNFEDVPF HSSYAHSQS |
| | | LDRLMNPLI DQYLYYLSR TQSTGGTAG |
| | | TQQLLFSQA GPNNMSAQA KNWLPGPCY |
| | | RQQRVSTTL SQNNNSNFA WTGATKYHL |
| | | NGRDSLVNP GVAMATHKD DEERFFPSS |
| | | GVLMFGKQG AGKDNVDYS SVMLTSEEE |
| | | IKTTNPVAT EQYGVVADN LQQQNAAPI |
| | | VGAVNSQGA LPGMVWQNR DVYLQGPIW |
| | | AKIPHTDGN FHPSPLMGG FGLKHPPPQ |
| | | ILIKNTPVP ADPPTTFNQ AKLASFITQ |
| | | YSTGQVSVE IEWELQKEN SKRWNPEIQ |
| | | YTSNYYKST NVDFAVNTE GTYSEPRPI |
| | | GTRYLTRNL |

5.4.6 Methods of Making rAAV Particles

Another aspect of the present invention involves making molecules disclosed herein. In some embodiments, a molecule according to the invention is made by providing a nucleotide comprising the nucleic acid sequence encoding any of the capsid protein molecules herein; and using a packaging cell system to prepare corresponding rAAV particles with capsid coats made up of the capsid protein. Such capsid proteins are described in Section 5.6.5, supra. In some embodiments, the nucleic acid sequence encodes a sequence having at least 60%, 70%, 80%, 85%, 90%, or 95%, including 96%, 97%, 98%, 99% or 99.9%, identity to the sequence of a capsid protein molecule described herein. In some embodiments, the nucleic acid encodes a sequence having at least 60%, 70%, 80%, 85%, 90%, or 95%, including 96%, 97%, 98%, 99% or 99.9%, identity to the sequence of the AAV8 capsid protein, while retaining (or substantially retaining) biological function of the AAV8 capsid protein. In some embodiments, the nucleic acid encodes a sequence having at least 60%, 70%, 80%, 85%, 90%, or 95%, including 96%, 97%, 98%, 99% or 99.9%, identity to the sequence of the AAV9 capsid protein, while retaining (or substantially retaining) biological function of the AAV9 capsid protein

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In embodiments, the cap and rep genes are provided by a packaging cell and not present in the viral genome.

In some embodiments, the nucleic acid encoding the engineered capsid protein is cloned into an AAV Rep-Cap plasmid in place of the existing capsid gene. When introduced together into host cells, this plasmid helps package an rAAV genome into the engineered capsid protein as the capsid coat. Packaging cells can be any cell type possessing the genes necessary to promote AAV genome replication, capsid assembly, and packaging.

Numerous cell culture-based systems are known in the art for production of rAAV particles, any of which can be used to practice a method disclosed herein. The cell culture-based systems include transfection, stable cell line production, and infectious hybrid virus production systems which include, but are not limited to, adenovirus-AAV hybrids, herpesvirus-AAV hybrids and baculovirus-AAV hybrids. rAAV production cultures for the production of rAAV virus particles require: (1) suitable host cells, including, for example, human-derived cell lines, mammalian cell lines, or insect-derived cell lines; (2) suitable helper virus function, provided by wild type or mutant adenovirus (such as temperature-sensitive adenovirus), herpes virus, baculovirus, or a plasmid construct providing helper functions; (3) AAV rep and cap genes and gene products; (4) a transgene (such as a therapeutic transgene) flanked by AAV ITR sequences and optionally regulatory elements; and (5) suitable media and media components (nutrients) to support cell growth/survival and rAAV production.

Nonlimiting examples of host cells include: A549, WEHI, 10T1/2, BHK, MDCK, COS1, COS7, BSC 1, BSC 40, BMT 10, VERO, W138, HeLa, HEK293 and their derivatives (HEK293T cells, HEK293F cells), Saos, C2C12, L, HT1080, HepG2, primary fibroblast, hepatocyte, myoblast cells, CHO cells or CHO-derived cells, or insect-derived cell lines such as SF-9 (e.g. in the case of baculovirus production systems). For a review, see Aponte-Ubillus et al., 2018, Appl. Microbiol. Biotechnol. 102:1045-1054, which is incorporated by reference herein in its entirety for manufacturing techniques.

In one aspect, provided herein is a method of producing rAAV particles, comprising (a) providing a cell culture comprising an insect cell; (b) introducing into the cell one or more baculovirus vectors encoding at least one of: i. an rAAV genome to be packaged, ii. an AAV rep protein sufficient for packaging, and iii. an AAV cap protein sufficient for packaging; (c) adding to the cell culture sufficient nutrients and maintaining the cell culture under conditions that allow production of the rAAV particles. In some embodiments, the method comprises using a first baculovirus vector encoding the rep and cap genes and a second baculovirus vector encoding the rAAV genome. In some embodiments, the method comprises using a baculovirus encoding the rAAV genome and an insect cell expressing the rep and cap genes. In some embodiments, the method comprises using a baculovirus vector encoding the rep and cap genes and the rAAV genome. In some embodiments, the insect cell is an Sf-9 cell. In some embodiments, the insect cell is an Sf-9 cell comprising one or more stably integrated heterologous polynucleotide encoding the rep and cap genes.

In some embodiments, a method disclosed herein uses a baculovirus production system. In some embodiments the baculovirus production system uses a first baculovirus encoding the rep and cap genes and a second baculovirus encoding the rAAV genome. In some embodiments the baculovirus production system uses a baculovirus encoding the rAAV genome and a host cell expressing the rep and cap genes. In some embodiments the baculovirus production system uses a baculovirus encoding the rep and cap genes and the rAAV genome. In some embodiments, the baculovirus production system uses insect cells, such as Sf-9 cells.

A skilled artisan is aware of the numerous methods by which AAV rep and cap genes, AAV helper genes (e.g., adenovirus E1a gene, E1b gene, E4 gene, E2a gene, and VA gene), and rAAV genomes (comprising one or more genes of interest flanked by ITRs) can be introduced into cells to produce or package rAAV. The phrase “adenovirus helper functions” refers to a number of viral helper genes expressed in a cell (as RNA or protein) such that the AAV grows efficiently in the cell. The skilled artisan understands that helper viruses, including adenovirus and herpes simplex virus (HSV), promote AAV replication and certain genes have been identified that provide the essential functions, e.g. the helper may induce changes to the cellular environment that facilitate such AAV gene expression and replication. In some embodiments of a method disclosed herein, AAV rep and cap genes, helper genes, and rAAV genomes are introduced into cells by transfection of one or more plasmid vectors encoding the AAV rep and cap genes, helper genes, and rAAV genome. In some embodiments of a method disclosed herein, AAV rep and cap genes, helper genes, and rAAV genomes can be introduced into cells by transduction with viral vectors, for example, rHSV vectors encoding the AAV rep and cap genes, helper genes, and rAAV genome. In some embodiments of a method disclosed herein, one or more of AAV rep and cap genes, helper genes, and rAAV genomes are introduced into the cells by transduction with an rHSV vector. In some embodiments, the rHSV vector encodes the AAV rep and cap genes. In some embodiments, the rHSV vector encodes the helper genes. In some embodiments, the rHSV vector encodes the rAAV genome. In some embodiments, the rHSV vector encodes the AAV rep and cap genes. In some embodiments, the rHSV vector encodes the helper genes and the rAAV genome. In some embodiments, the rHSV vector encodes the helper genes and the AAV rep and cap genes.

In one aspect, provided herein is a method of producing rAAV particles, comprising (a) providing a cell culture comprising a host cell; (b) introducing into the cell one or more rHSV vectors encoding at least one of: i. an rAAV genome to be packaged, ii. helper functions necessary for packaging the rAAV particles, iii. an AAV rep protein sufficient for packaging, and iv. an AAV cap protein sufficient for packaging; (c) adding to the cell culture sufficient nutrients and maintaining the cell culture under conditions that allow production of the rAAV particles. In some embodiments, the rHSV vector encodes the AAV rep and cap genes. In some embodiments, the rHSV vector encodes helper functions. In some embodiments, the rHSV vector comprises one or more endogenous genes that encode helper functions. In some embodiments, the rHSV vector comprises one or more heterogeneous genes that encode helper functions. In some embodiments, the rHSV vector encodes the rAAV genome. In some embodiments, the rHSV vector encodes the AAV rep and cap genes. In some embodiments, the rHSV vector encodes helper functions and the rAAV genome. In some embodiments, the rHSV vector encodes helper functions and the AAV rep and cap genes. In some embodiments, the cell comprises one or more stably integrated heterologous polynucleotide encoding the rep and cap genes.

In one aspect, provided herein is a method of producing rAAV particles, comprising (a) providing a cell culture comprising a mammalian cell; (b) introducing into the cell one or more polynucleotides encoding at least one of: i. an rAAV genome to be packaged, ii. helper functions necessary for packaging the rAAV particles, iii. an AAV rep protein sufficient for packaging, and iv. an AAV cap protein sufficient for packaging; (c) adding to the cell culture sufficient nutrients and maintaining the cell culture under conditions that allow production of the rAAV particles. In some embodiments, the helper functions are encoded by adenovirus genes. In some embodiments, the mammalian cell comprises one or more stably integrated heterologous polynucleotide encoding the rep and cap genes.

Molecular biology techniques to develop plasmid or viral vectors encoding the AAV rep and cap genes, helper genes, and/or rAAV genome are commonly known in the art. In some embodiments, AAV rep and cap genes are encoded by one plasmid vector. In some embodiments, AAV helper genes (e.g., adenovirus E1a gene, E1b gene, E4 gene, E2a gene, and VA gene) are encoded by one plasmid vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one plasmid vector. In some embodiments, one or more helper genes are stably expressed by the host cell, and one or more helper genes are introduced into the cell by transfection by one plasmid vector. In some embodiments, the helper genes are stably expressed by the host cell. In some embodiments, AAV rep and cap genes are encoded by one viral vector. In some embodiments, AAV helper genes (e.g., adenovirus E1a gene, E1b gene, E4 gene, E2a gene, and VA gene) are encoded by one viral vector. In some embodiments, the E1a gene or E1b gene is stably expressed by the host cell, and the remaining AAV helper genes are introduced into the cell by transfection by one viral vector. In some embodiments, the E1a gene and E1b gene are stably expressed by the host cell, and the E4 gene, E2a gene, and VA gene are introduced into the cell by transfection by one viral vector. In some embodiments, one or more helper genes are stably expressed by the host cell, and one or more helper genes are introduced into the cell by transfection by one viral vector. In some embodiments, the AAV rep and cap genes, the adenovirus helper functions necessary for packaging, and the rAAV genome to be packaged are introduced to the cells by transfection with one or more polynucleotides, e.g., vectors. In some embodiments, a method disclosed herein comprises transfecting the cells with a mixture of three polynucleotides: one encoding the cap and rep genes, one encoding adenovirus helper functions necessary for packaging (e.g., adenovirus E1a gene, E1b gene, E4 gene, E2a gene, and VA gene), and one encoding the rAAV genome to be packaged. In some embodiments, the AAV cap gene is an AAV8 cap gene. In some embodiments, the AAV cap gene is an AAV1, AAV2, AAV3, AAV4, AAV5, AAV6, AAV7, AAV8, AAV9, AAV10, AAV11, AAV2i8, AAV2.5, AAVrh.8, AAVrh.10, AAVrh.43, AAVrh.74, AAVhu.37, AAVAAV.hu31, or AAVhu.32 cap gene. In some embodiments, the vector encoding the rAAV genome to be packaged comprises a gene of interest flanked by AAV ITRs. In certain embodiments, the ITR sequences are AAV2 ITR sequences and include 5′ and 3′ sequences of SEQ ID NO: 28 and 29, respectively, as set forth in Table 2.

Any combination of vectors can be used to introduce AAV rep and cap genes, AAV helper genes, and rAAV genome to a cell in which rAAV particles are to be produced or packaged. In some embodiments of a method disclosed herein, a first plasmid vector encoding an rAAV genome comprising a gene of interest flanked by AAV inverted terminal repeats (ITRs), a second vector encoding AAV rep and cap genes, and a third vector encoding helper genes can be used. In some embodiments, a mixture of the three vectors is co-transfected into a cell. In some embodiments, a combination of transfection and infection is used by using both plasmid vectors as well as viral vectors.

In some embodiments, one or more of rep and cap genes, and AAV helper genes are constitutively expressed by the cells and does not need to be transfected or transduced into the cells. In some embodiments, the cell constitutively expresses rep and/or cap genes. In some embodiments, the cell constitutively expresses one or more AAV helper genes. In some embodiments, the cell constitutively expresses E1a. In some embodiments, the cell comprises a stable transgene encoding the rAAV genome.

In some embodiments, AAV rep, cap, and helper genes (e.g., E1a gene, E1b gene, E4 gene, E2a gene, or VA gene) can be of any AAV serotype. In some embodiments, AAV rep and cap genes for the production of a rAAV particle are from different serotypes. For example, the rep gene is from AAV2 whereas the cap gene is from AAV8.

In some embodiments, the rep gene is from AAV1, AAV2, AAV3, AAV4, AAV5, AAV6, AAV7, AAV8, AAV9, AAV10, AAV11, AAV2i8, AAV2.5, AAVrh.8, AAVrh.10, AAVrh.43, AAVrh.74, AAVhu.37, AAVAAV.hu31, or AAVhu.32 or other AAV serotypes (e.g., a hybrid serotype harboring sequences from more than one serotype). In other embodiments, the rep and the cap genes are from the same serotype. In still other embodiments, the rep and the cap genes are from the same serotype, and the rep gene comprises at least one modified protein domain or modified promoter domain. In certain embodiments, the at least one modified domain comprises a nucleotide sequence of a serotype that is different from the capsid serotype. The modified domain within the rep gene may be a hybrid nucleotide sequence consisting fragments different serotypes.

Hybrid rep genes provide improved packaging efficiency of rAAV particles, including packaging of a viral genome comprising a microdystrophin transgene greater than 4 kb, greater than 4.1 kb, greater than 4.2 kB, greater than 4.3 kb, greater than 4.4 kB, greater than 4.5 kb, or greater than 4.6 kb. AAV rep genes consist of nucleic acid sequences that encode the non-structural proteins needed for replication and production of virus. Transcription of the rep gene initiates from the p5 or p19 promoters to produce two large (Rep78 and Rep68) and two small (Rep52 and Rep40) nonstructural Rep proteins, respectively. Additionally, Rep78/68 domain contains a DNA-binding domain that recognizes specific ITR sequences within the ITR. All four Rep proteins have common helicase and ATPase domains that function in genome replication and/or encapsidation (Maurer A C, 2020, DOI: 10.1089/hum.2020.069). Transcription of the cap gene initiates from a p40 promoter, which sequence is within the C-terminus of the rep gene, and it has been suggested that other elements in the rep gene may induce p40 promoter activity. The p40 promoter domain includes transcription factor binding elements EF1A, MLTF, and ATF, Fos/Jun binding elements (AP-1), Sp1-like elements (Sp1 and GGT), and the TATA element (Pereira and Muzyczka, Journal of Virology, June 1997, 71(6):4300-4309). In some embodiments, the rep gene comprises a modified p40 promoter. In some embodiments, the p40 promoter is modified at any one or more of the EF1A binding element, MLTF binding element, ATF binding element, Fos/Jun binding elements (AP-1), Sp1-like elements (Sp1 or GGT), or the TATA element. In other embodiments, the rep gene is of serotype 1, 3, 4, 5, 6, 7, 8, 9, 10, 11, 12, 13, 14, 15, 16, rh8, rh10, rh20, rh39, rh.74, RHM4-1, or hu37, and the portion or element of the p40 promoter domain is modified to serotype 2. In still other embodiments, the rep gene is of serotype 8 or 9, and the portion or element of the p40 promoter domain is modified to serotype 2.

ITRs contain A and A′ complimentary sequences, B and B′ complimentary sequences, and C and C′ complimentary sequences; and the D sequence is contiguous with the ssDNA genome. The complimentary sequences of the ITRs form hairpin structures by self-annealing (Berns K I. The Unusual Properties of the AAV Inverted Terminal Repeat. Hum Gene Ther 2020). The D sequence contains a Rep Binding Element (RBE) and a terminal resolution site (TRS), which together constitute the AAV origin of replication. The ITRs are also required as packaging signals for genome encapsidation following replication. In some embodiments, the ITR sequences and the cap genes are from the same serotype, except that one or more of the A and A′ complimentary sequences, B and B′ complimentary sequences, C and C′ complimentary sequences, or the D sequence may be modified to contain sequences from a different serotype than the capsid. In some embodiments, the modified ITR sequences are from the same serotype as the rep gene. In other embodiments, the ITR sequences and the cap genes are from different serotypes, except that one or more of the ITR sequences selected from A and A′ complimentary sequences, B and B′ complimentary sequences, C and C′ complimentary sequences, or the D sequence are from the same serotype as the capsid (cap gene), and one or more of the ITR sequences are from the same serotype as the rep gene.

In some embodiments, the rep and the cap genes are from the same serotype, and the rep gene comprises a modified Rep78 domain, DNA binding domain, endonuclease domain, ATPase domain, helicase domain, p5 promoter domain, Rep68 domain, p5 promoter domain, Rep52 domain, p19 promoter domain, Rep40 domain or p40 promoter domain. In other embodiments, the rep and the cap genes are from the same serotype, and the rep gene comprises at least one protein domain or promoter domain from a different serotype. In one embodiment, an rAAV comprises a transgene flanked by AAV2 ITR sequences, an AAV8 cap, and a hybrid AAV2/8 rep. In another embodiment, the AAV2/8 rep comprises serotype 8 rep except for the p40 promoter domain or a portion thereof is from serotype 2 rep. In other embodiments, the AAV2/8 rep comprises serotype 2 rep except for the p40 promoter domain or a portion thereof is from serotype 8 rep. In some embodiments, more than two serotypes may be utilized to construct a hybrid rep/cap plasmid.

Any suitable method known in the art may be used for transfecting a cell may be used for the production of rAAV particles according to a method disclosed herein. In some embodiments, a method disclosed herein comprises transfecting a cell using a chemical based transfection method. In some embodiments, the chemical-based transfection method uses calcium phosphate, highly branched organic compounds (dendrimers), cationic polymers (e.g., DEAE dextran or polyethylenimine (PEI)), lipofection. In some embodiments, the chemical-based transfection method uses cationic polymers (e.g., DEAE dextran or polyethylenimine (PEI)). In some embodiments, the chemical-based transfection method uses polyethylenimine (PEI). In some embodiments, the chemical-based transfection method uses DEAE dextran. In some embodiments, the chemical-based transfection method uses calcium phosphate.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

Provided are host cell lines for production of the rAAV particles containing the constructs encoding the rAUF1 proteins as disclosed herein, including the constructs of SEQ ID Nos: 31 to 36 (spc-hu-opti-AUF1-CpG(−), tMCK-huAUF1, spc5-12-hu-opti-AUF1-WPRE, ss-CK7-hu-AUF1, spc-hu-AUF1-no-intron, and D(+)-CK7AUF1, respectively) or containing the constructs encoding microdystrophin proteins, SEQ ID NO: 94 or 96 (RGX-DYS1 or RGX-DYS5).

In preferred embodiments, the rAAVs provide transgene delivery vectors that can be used in therapeutic and prophylactic applications, as discussed in more detail below.

Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

### 5.5. Therapeutic Utility

Provided are methods of testing of the infectivity of a recombinant vector disclosed herein, for example rAAV particles. For example, the infectivity of recombinant gene therapy vectors in muscle cells can be tested in C2C12 myoblasts. Several muscle or heart cell lines may be utilized, including but not limited to T0034 (human), L6 (rat), MM14 (mouse), P19 (mouse), G-7 (mouse), G-8 (mouse), QM7 (quail), H9c2(2-1) (rat), Hs 74.Ht (human), and Hs 171.Ht (human) cell lines. Vector copy numbers may be assessed using polymerase chain reaction techniques and level of microdystrophin expression may be tested by measuring levels of microdystrophin mRNA in the cells.

### Animal Models

The efficacy of a viral vector containing a transgene encoding an AUF1 protein or microdystrophin as described herein may be tested by administering to an animal model to replace mutated dystrophin, for example, by using the mdx mouse and/or the golden retriever muscular dystrophy (GRMD) model and to assess the biodistribution, expression and therapeutic effect of the transgene expression. The therapeutic effect may be assessed, for example, by assessing change in muscle strength in the animal receiving the transgene. Animal models using larger mammals as well as nonmammalian vertebrates and invertebrates can also be used to assess pre-clinical therapeutic efficacy of a vector described herein. Accordingly, provided are compositions and methods for therapeutic administration comprising a dose of an AUF1 or microdystrophin encoding vector disclosed herein in an amount demonstrated to be effective according to the methods for assessing therapeutic efficacy disclosed here either alone or in combination with a second therapeutic described herein.

### Murine Models

The efficacy of gene therapy vectors alone or in combination with the second therapeutics disclosed herein may be assessed in murine models of DMD. The mdx mouse model (Yucel, N., et al, Humanizing the mdx mouse model of DMD: the long and the short of it, Regenerative Medicine volume 3, Article number: 4 (2018)), carries a nonsense mutation in exon 23, resulting in an early termination codon and a truncated protein (mdx). Mdx mice have 3-fold higher blood levels of pyruvate kinase activity compared to littermate controls. Like the human DMD disease, mdx skeletal muscles exhibit active myofiber necrosis, cellular infiltration, a wide range of myofiber sizes and numerous centrally nucleated regenerating myofibers. This phenotype is enhanced in the diaphragm, which undergoes progressive degeneration and myofiber loss resulting in an approximately 5-fold reduction in muscle isometric strength. Necrosis and regeneration in hind-limb muscles peaks around 3-4 weeks of age, but plateaus thereafter. In mdx mice and mdx mice crossed onto other mouse backgrounds (for example DBA/2J), a mild but significant decrease in cardiac ejection fraction is observed (Van Westering, Molecules 2015, 20, 8823-8855). Such DMD model mice with cardiac functional defects may be used to assess the cardioprotective effects or improvement or maintenance of cardiac function or attenuation of cardiac dysfunction of the gene therapy vectors described herein alone or in combination with the second therapeutics disclosed herein.

### Cardiac Function

Assessment of efficacy on cardiac function can be measured in mice, including mdx mice. To measure the blood pressure (BP) mice are sedated using 1.5% isofluorane with constant monitoring of the plane of anesthesia and maintenance of the body temperature at 36.5-37.58 C. The heart rate is maintained at 450-550 beats/min. A BP cuff is placed around the tail, and the tail is then placed in a sensor assembly for noninvasive BP monitoring during anesthesia. Ten consecutive BP measurements are taken. Qualitative and quantitative measurements of tail BP, including systolic pressure, diastolic pressure and mean pressure, are made offline using analytic software. See, for example, Wehling-Henricks et al, Human Molecular Genetics, 2005, Vol. 14, No. 14; Uaesoontrachoon et al, Human Molecular Genetics, 2014, Vol. 23, No. 12.

To monitor ECG wave heights and interval durations in awake, freely moving mice, radio telemetry devices are used. Transmitter units are implanted in the peritoneal cavity of anesthetized mice and the two electrical leads are secured near the apex of the heart and the right acromion in a lead II orientation. Mice are housed singly in cages over antenna receivers connected to a computer system for data recording. Unfiltered ECG data is collected for 10 seconds each hour for 35 days. The first 7 days of data are discarded to allow for recovery from the surgical procedure and ensure any effects of anesthesia has subsided. Data waveforms and parameters are analyzed with the DSI analysis packages (ART 3.01 and Physiostat 4.01) and measurements are compiled and averaged to determine heart rates, ECG wave heights and interval durations. Raw ECG waveforms are scanned for arrhythmias by two independent observers.

Picro-Sirius red staining is performed to measure the degree of fibrosis in the heart of trial mice. In brief, at the end of trial, directly following euthanasia, the heart muscle is removed and fixed in 10% formalin for later processing. The heart is sectioned and paraffin sections are deparaffinized in xylene followed by nuclear staining with Weigert's hematoxylin for 8 min. They are then washed and then stained with Picro-Sirius red (0.5 g of Sirius red F3B, saturated aqueous solution of picric acid) for an additional 30 min. The sections are cleared in three changes of xylene and mounted in Permount. Five random digital images are taken using an Eclipse E800 (Nikon, Japan) microscope, and blinded analysis is done using Image J (NIH). Blood samples are taken via cardiac puncture when the animals are euthanized, and the serum collected is used for the measurement of muscle CK levels.

### Canine

Most canine studies are conducted in the golden retriever muscular dystrophy (GRMD) model (Korneygay, J. N., et al, The golden retriever model of Duchenne muscular dystrophy. Skelet Muscle. 2017; 7: 9, which is incorporated by reference in its entirety). Dogs with GRMD are afflicted with a progressive, fatal disease with skeletal and cardiac muscle phenotypes and selective muscle involvement—a severe phenotype that more closely mirrors that of DMD. GRMD dogs carry a single nucleotide change that leads to exon skipping and an out-of-frame DMD transcript. Phenotypic features in dogs include elevation of serum CK, CRDs on EMG, and histopathologic evidence of grouped muscle fiber necrosis and regeneration. Phenotypic variability is frequently observed in GRMD, as in humans. GRMD dogs develop paradoxical muscle hypertrophy which seems to play a role in the phenotype of affected dogs, with stiffness at gait, decreased joint range of motion, and trismus being common features. Objective biomarkers to evaluate disease progression include tetanic flexion, tibiotarsal joint angle, % eccentric contraction decrement, maximum hip flexion angle, pelvis angle, cranial sartorius circumference, and quadriceps femoris weight.

### 5.6. Methods of Combination Treatment

Provided are methods of treating human subjects for any muscular dystrophy disease (dystrophinopathy) that can be treated by providing a functional AUF1, as disclosed herein, in combination with a second therapeutic, wherein the second therapeutic can treat a dystrophinopathy disease or ameliorate one or more symptoms thereof. DMD is the most common of such disease, and the gene therapy vectors that express AUF1 provided herein can be administered in combination with a second therapeutic described herein to treat a dystrophinopathy, including DMD, Becker muscular dystrophy (BMD), myotonic muscular dystrophy (Steinert's disease), Facioscapulohumeral disease (FSHD), limb-girdle muscular dystrophy, X-linked dilated cardiomyopathy, or oculopharyngeal muscular dystrophy. In some aspects, the combination therapy is a combination of any one of the AUF1 gene therapy vectors disclosed herein with any one of the microdystrophin gene therapy vectors disclosed herein.

In embodiments, the methods of combination treatment provide for the treatment of Duchenne muscular dystrophy in human subjects in need thereof. In embodiments, the methods of combination treatment provide for the treatment of Becker muscular dystrophy in human subjects in need thereof. In embodiments, the methods of combination treatment provide for the treatment of X-linked dilated cardiomyopathy in human subjects in need thereof. In embodiments, the methods of combination treatment provide for the treatment of limb girdle muscular dystrophy (LGMD) in human subjects in need thereof.

In embodiments, the methods of treating human subjects provide a first gene therapy vector comprising a genome comprising a transgene encoding p37^{AUF1} In embodiments, the methods of treating human subjects provide a first gene therapy vector comprising a genome comprising a transgene encoding p40^{AUF1}. In embodiments, the methods of treating human subjects provide a first gene therapy vector comprising a genome comprising a transgene encoding p42^{AUF1}. In embodiments, the methods of treating human subjects provide a first gene therapy vector comprising a genome comprising a transgene encoding p45^{AUF1}. In embodiments, provided are methods of treating human subjects with gene therapy vectors with two or more AUF1 isoforms, i.e., a combination of p37AUF1, p40AUF1, p42AUF1, and/or p45AUF1.

In embodiments, the methods of treating human subjects comprise a first therapeutic comprising an rAAV particle comprising a nucleic acid molecule encoding an AUF1 protein, or functional fragment thereof, operably coupled to a muscle creatine kinase (MCK) promoter. In embodiments, the methods of treating human subjects comprise a first therapeutic comprising an rAAV particle comprising a nucleic acid molecule encoding an AUF1 protein, or functional fragment thereof, operably coupled to a syn100 promoter.

In embodiments, the methods of treating human subjects comprise a first therapeutic comprising an rAAV particle comprising a nucleic acid molecule encoding an AUF1 protein, or functional fragment thereof, operably coupled to a CK6 promoter.

In embodiments, the methods of treating human subjects comprise a first therapeutic comprising an rAAV particle comprising a nucleic acid molecule encoding an AUF1 protein, or functional fragment thereof, operably coupled to a CK7 promoter. In embodiments, the methods of treating human subjects comprise a first therapeutic comprising an rAAV particle comprising a nucleic acid molecule encoding an AUF1 protein, or functional fragment thereof, operably coupled to a CK8 promoter. In embodiments, the methods of treating human subjects comprise a first therapeutic comprising an rAAV particle comprising a nucleic acid molecule encoding an AUF1 protein, or functional fragment thereof, operably coupled to a CK9 promoter. In embodiments, the methods of treating human subjects comprise a first therapeutic comprising an rAAV particle comprising a nucleic acid molecule encoding an AUF1 protein, or functional fragment thereof, operably coupled to a dMCK promoter. In embodiments, the methods of treating human subjects comprise a first therapeutic comprising an rAAV particle comprising a nucleic acid molecule encoding an AUF1 protein, or functional fragment thereof, operably coupled to a tMCK promoter.

In embodiments, the methods of treating human subjects comprise a first therapeutic comprising an rAAV particle comprising a nucleic acid molecule encoding an AUF1 protein, or functional fragment thereof, operably coupled to a smooth muscle 22 (SM22) promoter. In embodiments, the methods of treating human subjects comprise a first therapeutic comprising an rAAV particle comprising a nucleic acid molecule encoding an AUF1 protein, or functional fragment thereof, operably coupled to a myo-3 promoter. In embodiments, the methods of treating human subjects comprise a first therapeutic comprising an rAAV particle comprising a nucleic acid molecule encoding an AUF1 protein, or functional fragment thereof, operably coupled to a Spc5-12 promoter. In embodiments, the methods of treating human subjects comprise a first therapeutic comprising an rAAV particle comprising a nucleic acid molecule encoding an AUF1 protein, or functional fragment thereof, operably coupled to a creatine kinase (CK) 8e promoter. In embodiments, the methods of treating human subjects comprise a first therapeutic comprising an rAAV particle comprising a nucleic acid molecule encoding an AUF1 protein, or functional fragment thereof, operably coupled to a U6 promoter.

In embodiments, the methods of treating human subjects comprise a first therapeutic comprising an rAAV particle comprising a nucleic acid molecule encoding an AUF1 protein, or functional fragment thereof, operably coupled to a H1 promoter. In embodiments, the methods of treating human subjects comprise a first therapeutic comprising an rAAV particle comprising a nucleic acid molecule encoding an AUF1 protein, or functional fragment thereof, operably coupled to a desmin promoter. In embodiments, the methods of treating human subjects comprise a first therapeutic comprising an rAAV particle comprising a nucleic acid molecule encoding an AUF1 protein, or functional fragment thereof, operably coupled to a Pitx3 promoter. In embodiments, the methods of treating human subjects comprise a first therapeutic comprising an rAAV particle comprising a nucleic acid molecule encoding an AUF1 protein, or functional fragment thereof, operably coupled to a skeletal alpha-actin promoter. In embodiments, the methods of treating human subjects comprise a first therapeutic comprising an rAAV particle comprising a nucleic acid molecule encoding an AUF1 protein, or functional fragment thereof, operably coupled to a MHCK7 promoter. In embodiments, the methods of treating human subjects comprise a first therapeutic comprising an rAAV particle comprising a nucleic acid molecule encoding an AUF1 protein, or functional fragment thereof, operably coupled to a Sp-301 promoter.

In embodiments, the methods of treating human subjects utilize AUF1 gene therapy constructs that have been codon-optimized. In embodiments, the methods of treating human subjects utilize AUF1 gene therapy constructs that have been CpG depleted. In embodiments, the AUF1 gene therapy constructs of the methods have the nucleotide sequences of SEQ ID NO: 31. In embodiments, the AUF1 gene therapy constructs of the methods have the nucleotide sequences of SEQ ID NO: 36.

In embodiments, the methods of treating human subjects comprise a first therapeutic comprising an rAAV particle having the nucleotide sequence of SEQ ID NO: 31 (spc-hu-opti-AUF1-CpG(−)). In embodiments, the methods of treating human subjects comprise a first therapeutic comprising an rAAV particle having the nucleotide sequence of SEQ ID NO: 32 (tMCK-huAUF1). In embodiments, the methods of treating human subjects comprise a first therapeutic comprising an rAAV particle having the nucleotide sequence of SEQ ID NO: 33 (spc5-12-hu-opti-AUF1-WPRE). In embodiments, the methods of treating human subjects comprise a first therapeutic comprising an rAAV particle having the nucleotide sequence of SEQ ID NO: 34 (ss-CK7-hu-AUF1). In embodiments, the methods of treating human subjects comprise a first therapeutic comprising an rAAV particle having the nucleotide sequence of SEQ ID NO: 35 (spc-hu-AUF1-no-intron). In embodiments, the methods of treating human subjects comprise a first therapeutic comprising an rAAV particle having the nucleotide sequence of SEQ ID NO: 36 (D(+)-CK7AUF1).

In embodiments, the methods of treating human subjects utilize AAV8 gene therapy vectors. In embodiments, the methods of treating human subjects utilize AAV9 gene therapy vectors. In embodiments, the methods of treating human subjects utilize AAV having a capsid that is at least 95% identical to SEQ ID NO:114 (AAV8 capsid). In embodiments, the methods of treating human subjects utilize AAV having a capsid that is at least 95% identical to SEQ ID NO:115 (AAV9 capsid). In embodiments, the methods of treating human subjects utilize AAV having a capsid that is at least 95% identical to SEQ ID NO: 118 (AAVhu 32 capsid).

Disclosed are methods of treating a dystrophinopathy in a subject in need thereof, comprising administering to the subject a therapeutically effective amount (either alone or when administered with the second therapeutic) of a first therapeutic and a therapeutically effective amount (either alone or when administered with the first therapeutic) second therapeutic which is different from said first therapeutic, wherein the first therapeutic is a first rAAV particle comprising a nucleic acid molecule encoding an AUF1 protein, or functional fragment thereof, operatively coupled to a muscle cell-specific promoter. In embodiments, the rAAV particle comprises a construct having the nucleotide sequence of one of SEQ ID Nos: 31 to 36 (spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively), including where the rAAV is an AAV8 serotype or an AAV9 serotype.

In embodiments, the second therapeutic is a microdystrophin pharmaceutical composition, including an AAV vector particle comprising a microdystrophin construct, including DYS1, DYS3 or DYS5 (SEQ ID NO: 94, 95 or 96, respectively), including where the rAAV is an AAV8 serotype or an AAV9 serotype.

In certain embodiments, the AUF1 gene therapy product and the microdystrophin gene therapy product are delivered at the same time or are delivered within 1 hour, 2 hours, 3 hours, 4 hours, 6 hours, 12 hours, 1 day, 2 days, 3 days, 4 days, 5 days, 6 days, 7 days, 8 days, 9 days, 10 days, 11 days, 12 days, 13 days or 2 weeks, 3 weeks or 4 weeks of each other, including that the second product is administered prior to any immune response against the first gene therapy product. In other embodiments, the AUF1 gene therapy product and the microdystrophin gene therapy product are delivered simultaneously or are delivered within 1 hour, 2 hours or 3 hours, including that the second product is administered prior to any immune response against the first gene therapy product. In still other embodiments, the AUF1 gene therapy product and the microdystrophin gene therapy product both comprise an AAV vector of the same serotype and are delivered simultaneously or are delivered no more than 1 hour apart.

In other embodiments, the second therapeutic is a mutation suppression therapy, an exon skipping therapy, a steroid therapy, an immunosuppressive/anti-inflammatory therapy, any therapy that treats one or more symptoms of the dystrophinopathy, as disclosed herein in more detail or any combination thereof. Alternatively, a therapeutic is administered in addition to the AUF1 gene therapy vector and the microdystrophin gene therapy vector, as a third therapeutic, which may be a mutation suppression therapy, an exon skipping therapy, a steroid therapy, an immunosuppressive/anti-inflammatory therapy, any therapy that treats one or more symptoms of the dystrophinopathy, as disclosed herein in more detail or any combination thereof. Dosing for each second therapeutic can be any of the known doses for administering each of the second therapeutics.

In some embodiments, the second therapeutic (or third therapeutic as the case may be) can be administered to alleviate or further alleviate one or more symptoms or characteristics of dystrophinopathies which may be assessed by any of, but not limited to, the following assays on the subject: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, improvement of the quality of life. Each of these assays is known to the skilled person. As an example, the publication of Manzur et al. (Manzur A Y et al, (2008), Glucocorticoid corticosteroids for Duchenne muscular dystrophy (review), Wiley publishers, The Cochrane collaboration.) gives an extensive explanation of each of these assays. For each of these assays, as soon as a detectable improvement or prolongation of a parameter measured in an assay has been found, it may indicate that one or more symptoms of Duchenne Muscular Dystrophy has been alleviated in an individual using a method of the invention. Detectable improvement or prolongation may be a statistically significant improvement or prolongation as described in Hodgetts et al (Hodgetts S., et al, (2006), Neuromuscular Disorders, 16: 591-602.2006). Alternatively, the alleviation of one or more symptom(s) of Duchenne Muscular Dystrophy may be assessed by measuring an improvement of a muscle fiber function, integrity and/or survival as later defined herein.

A treatment in a method according to the invention may have a duration of at least one week, at least one month, at least several months, at least one year, at least 2, 3, 4, 5, 6 years or more. The frequency of administration of any of the second therapeutics, including those not delivered by gene therapy and described herein may depend on several parameters such as the age of the patient, the type of mutation, the number of molecules (dose), the formulation of said molecule. The frequency may be ranged between at least once in a two weeks, or three weeks or four weeks or five weeks or a longer time period.

The first therapeutic and second therapeutic, and optionally a third or even further therapeutics can be administered to an individual in any order. When more than one second therapeutic (e.g., a third therapeutic) is administered those can also be administer in any order relevant to each other and to the first therapeutic. In one embodiment, said therapeutics are administered simultaneously (meaning that said therapeutics are administered within 10 hours, including within one hour). In another embodiment, said therapeutics are administered sequentially. In some aspects, administration of the first and second therapeutic can occur within 7, 10, or 14 days of each other. In some aspects, simultaneous administration can mean the first and second therapeutic are formulated together in a single composition or each can be formulated by itself. In some aspects, a third therapeutic is administered concurrently with the first and/or second therapeutic, or is administered at a separate time, including on a regular dosing schedule, such as daily, weekly, or monthly.

In some embodiments, the first and second therapeutics provide a synergistic therapeutic effect with respect to one or more clinical end points in the treatment of a dystrophinopathy in a subject, in particular, where the therapeutic effect is greater than the additive therapeutic effects of the first and second therapeutics when administered alone. In some embodiments, the first and second therapeutics provide a synergistic effect in that the therapeutics result in improvements in different sets of clinical endpoints such that the therapeutic benefit of the combination is greater than the therapeutic benefit of each therapeutic individually.

In some embodiments, when a third or further therapeutics are administered, the first, second and third therapeutics provide a synergistic therapeutic effect with respect to one or more clinical end points in the treatment of a dystrophinopathy in a subject, in particular, where the therapeutic effect is greater than the additive therapeutic effects of the first, second and third therapeutics when administered alone. In some embodiments, the first, second and third therapeutics provide a synergistic effect in that the therapeutics result in improvements in different sets of clinical endpoints such that the therapeutic benefit of the combination is greater than the therapeutic benefit of each therapeutic individually.

### 5.6.1 Microdystrophin Therapy in a Combination Therapy

Disclosed are methods of treating a dystrophinopathy in a subject in need thereof, comprising administering to the subject a first therapeutic and a second therapeutic, wherein the first therapeutic is an rAAV vector comprising a transgene encoding a AUF1 disclosed herein and the second therapeutic is a gene therapy vector, including an rAAV gene therapy vector encoding a microdystrophin as disclosed herein.

In some embodiments, the transgene that encodes a microdystrophin protein consists of dystrophin domains arranged from amino-terminus to the carboxy terminus: ABD-H1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site.

In some embodiments, the CT comprises or consists of the proximal 194 amino acids of the C-terminus of dystrophin or at least the proximal portion of the C-terminus encoding human dystrophin amino acid residues 3361-3554 of SEQ ID NO: 51 (UniProtKB-P11532) or at least the proximal portion of the C-terminus encoded by exons 70 to 74 and the first 36 amino acids of the amino acid sequence encoded by the nucleotide sequence of exon 75.

In some embodiments, the microdystrophin protein has the amino acid sequence of the microdystrophin encoded by DYS1, DYS3 or DYS5 (SEQ ID NO: 52, 53, or 54). Alternatively, the microdystrophin protein has an amino acid sequence of one of SEQ ID NO: 133 to 137. In some embodiments, the microdystrophin protein is encoded by the nucleic acid sequence of SEQ ID NO: 91, 92, or 93. In embodiments, the nucleic acid sequence coding for the microdystrophin is operably linked to regulatory sequences, including promoters as listed in Table 10 and other regulatory elements, for example, as in Table 2 or 11. In certain embodiments, the rAAV has a recombinant genome having the nucleotide sequence of SEQ ID NO: 94, 95 or 96 (RGX-DYS-1, RGX-DYS-3, or RGX-DYS-5) or alternatively SpcV1-μDys1 (SEQ ID NO: 130) or SpcV2-μDys1 (SEQ ID NO: 132). In specific embodiment, the rAAV is an AAV8 serotype, AAV9 serotype, or AAVhu.32 or any other serotype, including with a tropism for muscle cells, as disclosed in Section 5.4.5, supra.

In other embodiments, the microdystrophin gene therapy is SGT-001, serotype AAV9, rAAVrh74.MHCK7.micro-dystrophin, SRP-9001 (see, Willcocks et al. “Assessment of rAAVrh.74.MHCK7.micro-dystrophin Gene Therapy Using Magnetic Resonance Imaging in Children with Duchenne Muscular Dystrophy” JAMA Network Open 2021 4:e2031851, which is incorporated herein by reference); GNT-004 (Le Guiner et al. “Long-term microdystrophin gene therapy is effective in a canine model of Duchenne muscular dystrophy” Nat Commun 8, 16105 (2017), which is incorporated herein by reference); or Pfizer PF-06939926 (AAV9 mini-dystrophin) or any other mini-dystrophin or micro-dystrophin construct.

In some embodiments, the therapeutically effective amount of the rAAV particle encoding the microdystrophin is administered intravenously or intramuscularly at dose of 2×10¹³ to 1×10¹⁵ genome copies/kg.

In certain embodiments, the first therapeutic is an rAAV particle comprises a construct having the nucleotide sequence of one of SEQ ID Nos: 31 to 36 (spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively), including where the rAAV is an AAV8 serotype or an AAV9 serotype, and the second therapeutic is an rAAV particle which has a recombinant genome having the nucleotide sequence of SEQ ID NO: 94, 95 or 96 (DYS-1, DYS-3, or DYS-5), including where the rAAV is an AAV8 serotype or is an AAV9 serotype. In embodiments, the ratio of the rAAV particle having a transgene encoding AUF1 and the rAAV particle having a transgene encoding the microdystrophin is 1:1, 1:2, 1:4, 1:5; 1:10, 1:50, 1:100 or 1:1000. Alternatively, the ratio of the AUF1 gene therapy vector and the microdystrophin gene therapy vector is 0.5:1, 0.25:1, 0.2:1, or 0.1:1.

### 5.6.2 Mutation Suppression Therapy

Disclosed are methods of treating a dystrophinopathy in a subject in need thereof, comprising administering to the subject a first therapeutic and a second therapeutic, wherein the first therapeutic is an rAAV vector comprising a transgene encoding a AUF1 disclosed herein and the second therapeutic is a mutation suppression therapy. In embodiments, a combination of the rAAV encoding AUF1, the rAAV encoding the microdystrophin and the mutation suppression therapeutic (as a third therapeutic) is administered to treat or ameliorate the symptoms of the dystrophinopathy of the subject.

In some embodiments, the second therapeutic (or third therapeutic) is ataluren. In some embodiments, ataluren is administered orally. In some embodiments, ataluren can be administered in a dose of 10 mg/kg/day to 200 mg/kg/day. In some embodiments, ataluren can be administered in a dose of 40 mg/kg. For example, the dosing can be 10 mg/kg in the morning, 10 mg/kg at midday, and 20 mg/kg in the evening. The length of time for ataluren administration can be weeks, months, or years. In some embodiments, treatment resulted in increased ability to walk/run longer distances and/or increased ability to climb stairs compared to pre-treatment levels.

In some embodiments, the second therapeutic (or third therapeutic is gentamicin. In some embodiments, gentamicin is administered intravenously. In some embodiments, gentamicin can be administered in a dose of 3 mg/kg/day to 25 mg/kg/day. In some embodiments, gentamicin can be administered in a dose of 7.5 mg/kg/day. The length of time for ataluren administration can be weeks, months, or years. In some embodiments, treatment resulted in increased hearing, kidney function and/or muscle strength compared to pre-treatment levels.

In some embodiments, the mutation suppressor therapy is a nonsense suppressor mutation. For example, the subject can have a nonsense mutation and the second therapeutic enables a ribosome to read through a premature nonsense mutation.

Nonsense suppressor therapies can be of two general classes. A first class includes compounds that disrupt codon-anticodon recognition during protein translation in a eukaryotic cell, so as to promote readthrough of a nonsense codon. These agents can act by, for example, binding to a ribosome so as to affect its activity in initiating translation or promoting polypeptide chain elongation, or both. For example, nonsense suppressor agents of this class can act by binding to rRNA (e.g., by reducing binding affinity to 18S rRNA). A second class are those that provide the eukaryotic translational machinery with a tRNA that provides for incorporation of an amino acid in a polypeptide where the mRNA normally encodes a stop codon, e.g., suppressor tRNAs.

### 5.6.3 Exon Skipping Therapy

Disclosed are methods of treating a dystrophinopathy in a subject in need thereof, comprising administering to the subject a first therapeutic and a second therapeutic, wherein the first therapeutic is an rAAV comprising a transgene encoding an AUF1 disclosed herein and the second therapeutic is an exon skipping therapy (or the third therapeutic is an exon skipping therapy and the second therapeutic is a microdystrophin gene therapy vector). In some embodiments, the exon skipping therapy is an antisense oligonucleotide. In embodiments, a combination of the rAAV encoding AUF1, the rAAV encoding the microdystrophin and the exon skipping therapeutic (as a third therapeutic) is administered to treat or ameliorate the symptoms of the dystrophinopathy of the subject.

In some embodiments, a subject is first identified as being amenable to treatment with an exon skipping therapy.

Exon skipping refers to the induction in a cell of a mature mRNA that does not contain a particular exon that is normally present therein. Exon skipping is achieved by providing a cell expressing the pre-mRNA of said mRNA with a molecule (i.e. exon skipping therapy) capable of interfering with sequences such as, for example, the splice donor or splice acceptor sequence that are both required for allowing the enzymatic process of splicing, or a molecule (i.e. exon skipping therapy) that is capable of interfering with an exon inclusion signal required for recognition of a stretch of nucleotides as an exon to be included in the mRNA. The term pre-mRNA refers to a non-processed or partly processed precursor mRNA that is synthesized from a DNA template in the cell nucleus by transcription.

In some embodiments, a subject treated with the exon skipping therapy means that at least 1%, 10%, 20%, 30%, 40%, 50%, 60%, 70%, 80%, 90% or more of the DMD mRNA in one or more (muscle) cells of the subject will not contain said exon.

In some embodiments, the exon skipping therapy results in skipping of one or more exons of dystrophin. In some embodiments, one or more of exons 1-60 can be skipped. In some embodiments, one or more of exons 2, 43, 44, 45, 50, 51, 52, 53, or 55 of the human dystrophin gene can be skipped to express a form of dystrophin protein.

In some embodiments, the exon skipping therapy results in skipping exon 45. For example, in some embodiments, the exon skipping therapy can be casimersen. In some embodiments, casimersen can be administered intravenously. In some embodiments, administration can be daily, weekly, or monthly. In some embodiments, the length of treatment can be weeks, months or years. In some embodiments, casimersen can be administered in a dose of 10 mg/kg to 200 mg/kg. In some embodiments, casimersen can be administered in a dose of 30 mg/kg. For example, administration can be once weekly via intravenous (IV) infusions of 30 mg/kg. In some embodiments, the exon skipping therapy can be SRP-5045. In some embodiments, the exon skipping therapy can be DS-5141B. In some embodiments, DS-5141B can be administered subcutaneously. In some embodiments, administration can be daily, weekly, or monthly. In some embodiments, the length of treatment can be weeks, months or years. In some embodiments, DS-5141B can be administered in a dose of 0.1 mg/kg to 20 mg/kg. In some embodiments, DS-5141B can be administered in a dose of 2 mg/kg or 6 mg/kg. For example, administration can be subcutaneously once a week for 2 weeks at a dose of 2 to 6 mg/kg/week.

In some embodiments, the exon skipping therapy results in skipping exon 50. For example, in some embodiments, the exon skipping therapy can be SRP-5050. In some embodiments, SRP-5050 can be administered intravenously or subcutaneously. In some embodiments, administration can be daily, weekly, or monthly. In some embodiments, the length of treatment can be weeks, months or years. SRP-5050 is part of a peptide phosphorodiamidate morpholino oligomer (PPMO) technology that includes a cell-penetrating peptide that is conjugated to an oligomer backbone with the goal of increasing cellular uptake in the muscle tissue. In some embodiments, the PPMO technology used herein is similar to that described in Tsoumpra et al. EBioMedicine 45(2019):630-645 and/or Guidotti et al. Trends in Pharmacological Sciences, vol 38, issue 4, 406-424, 2017, both of which are incorporated herein by reference in their entirety.

In some embodiments, the exon skipping therapy results in skipping exon 51. For example, in some embodiments, the exon skipping therapy can be eteplirsen. In some embodiments, the exon skipping therapy can be SRP-5051. SRP-5050 is part of the PPMO technology that includes a cell-penetrating peptide that is conjugated to an oligomer backbone with the goal of increasing cellular uptake in the muscle tissue. In some embodiments, SRP-5051 can be administered intravenously. In some embodiments, administration can be daily, weekly, or monthly. In some embodiments, the length of treatment can be weeks, months or years. In some embodiments, SRP-5051 can be administered in a dose of 1 mg/kg to 200 mg/kg. In some embodiments, SRP-5051 can be administered in a dose of 4 mg/kg to 40 mg/kg. For example, administration can be once monthly via intravenous (IV) infusion at a dose of 20 mg/kg.

In some embodiments, the exon skipping therapy results in skipping exon 53. For example, in some embodiments, the exon skipping therapy can be golodirsen. In some embodiments, golodirsen can be administered intravenously. In some embodiments, administration can be daily, weekly, or monthly. In some embodiments, the length of treatment can be weeks, months or years. In some embodiments, golodirsen can be administered in a dose of 10 mg/kg/day to 200 mg/kg/day. In some embodiments, golodirsen can be administered in a dose of 30 mg/kg. For example, administration can be once weekly via intravenous (IV) infusions of 30 mg/kg.

In some embodiments, the exon skipping therapy can be SRP-5053. SRP-5053 is part of the PPMO technology that includes a cell-penetrating peptide that is conjugated to an oligomer backbone with the goal of increasing cellular uptake in the muscle tissue. In some embodiments, SRP-5053 can be administered intravenously or subcutaneously. In some embodiments, administration can be daily, weekly, or monthly. In some embodiments, the length of treatment can be weeks, months or years.

In some embodiments, the exon skipping therapy can be viltolarsen. In some embodiments, viltolarsen can be administered intravenously. In some embodiments, administration can be daily, weekly, or monthly. In some embodiments, the length of treatment can be weeks, months or years. In some embodiments, viltolarsen can be administered in a dose of 10 mg/kg to 200 mg/kg. In some embodiments, viltolarsen can be administered in a dose of 80 mg/kg. For example, administration can be once weekly via intravenous (IV) infusions of 80 mg/kg.

In some embodiments, the exon skipping therapy results in skipping exon 52. For example, in some embodiments, the exon skipping therapy can be SRP-5052. SRP-5052 is part of the PPMO technology that includes a cell-penetrating peptide that is conjugated to an oligomer backbone with the goal of increasing cellular uptake in the muscle tissue. In some embodiments, SRP-5052 can be administered intravenously or subcutaneously. In some embodiments, administration can be daily, weekly, or monthly. In some embodiments, the length of treatment can be weeks, months or years.

In some embodiments, the exon skipping therapy results in skipping exon 44. For example, in some embodiments, the exon skipping therapy can be SRP-5044. SRP-5044 is part of the PPMO technology that includes a cell-penetrating peptide that is conjugated to an oligomer backbone with the goal of increasing cellular uptake in the muscle tissue. In some embodiments, SRP-5044 can be administered intravenously or subcutaneously. In some embodiments, administration can be daily, weekly, or monthly. In some embodiments, the length of treatment can be weeks, months or years.

In some embodiments, the exon skipping therapy can be NS-089/NCNP-02. In some embodiments, NS-089/NCNP-02 can be administered intravenously. In some embodiments, administration can be daily, weekly, or monthly. In some embodiments, the length of treatment can be weeks, months or years. In some embodiments, NS-089/NCNP-02 can be administered in a dose of 0.5 mg/kg to 200 mg/kg. In some embodiments, NS-089/NCNP-02 can be administered in a dose of 1.62 mg/kg, 10 mg/kg, 40 mg/kg, or 80 mg/kg. For example, administration can be once weekly via intravenous (IV) infusions of 1.62 mg/kg, 10 mg/kg, 40 mg/kg, or 80 mg/kg.

In some embodiments, the exon skipping therapy results in skipping exon 2. For example, in some embodiments, the exon skipping therapy can be scAAV9.U7.ACCA. scAAV9.U7.ACCA is an AAV9 vector carrying U7snRNA to treat a duplicate of exon 2. In some embodiments, scAAV9.U7.ACCA can be administered intravenously. In some embodiments, administration can be daily, weekly, or monthly. In some embodiments, the length of treatment can be weeks, months or years. In some embodiments, scAAV9.U7.ACCA can be administered in a dose of 1×10¹² viral genomes/kilogram (vg/kg) to 1×10¹⁵ vg/kg. In some embodiments, NS-089/NCNP-02 can be administered in a dose of 3×10¹³ vg/kg to 8×10¹³ vg/kg. For example, administration can be once daily, weekly, monthly or yearly via intravenous (IV) infusions of 3×10¹³ vg/kg or 8×10¹³ vg/kg.

In some embodiments, the second therapeutic can be a combination of two or more of the exon skipping therapies described herein. For example, in some embodiments, the exon skipping therapy can be a combination of casimersen and golodiresen or casimersen, eteplirsen, and golodiresen.

### 5.6.4 Steroid Therapy

Disclosed are methods of treating a dystrophinopathy in a subject in need thereof, comprising administering to the subject a first therapeutic and a second therapeutic, wherein the first therapeutic is an rAAV comprising a transgene encoding a AUF1 disclosed herein and the second therapeutic is a steroid therapy. In some embodiments, the steroid therapy is a glucocorticoid steroid. In embodiments, a combination of the rAAV encoding AUF1, the rAAV encoding the microdystrophin and the steroid therapy (as a third therapeutic) is administered to treat or ameliorate the symptoms of the dystrophinopathy of the subject.

In some embodiments, the steroid therapy is prednisone, deflazacort, Vamorolone, or Spironolactone, or a combination thereof. Spironolactone is an aldosterone antagonist and although may not be considered a steroid, it is used in a similar manner to steroids and is often compared to corticosteroids.

In some embodiments, the daily dose of prednisone is 0.2 mg/kg/day to 10 mg/kg/day. In some embodiments, the daily dose of prednisone is 0.75 mg/kg/day. In some embodiments, the daily dose of deflazacort is 0.2 mg/kg/day to 40 mg/kg/day. In some embodiments, the daily dose of deflazacort is 0.9 mg/kg/day. In some embodiments, the daily dose of Vamorolone is 0.5 mg/kg to 40 mg/kg. In some embodiments, the daily dose of Vamorolone is 2 mg/kg, 6 mg/kg or 20 mg/kg. In some embodiments, the daily dose of Spironolactone is 5 mg to 40 mg. In some embodiments, the daily dose of Spironolactone is 12.5 mg or 25 mg.

The steroid dose can be increased or decreased based on growth, weight, and other side effects experienced. In some embodiments, dosing can be either daily or high dose weekends. For example, inn some embodiments, doses of twice weekly can go up to 250 mg/day of prednisone or 300 mg/day of deflazacort. In some embodiments, dosing can be 10 days on, 10 days off, etc.

### 5.6.5 Immunosuppressive/Anti-Inflammatory Therapy

Disclosed are methods of treating a dystrophinopathy in a subject in need thereof, comprising administering to the subject a first therapeutic and a second therapeutic, wherein the first therapeutic is an rAAV comprising a transgene encoding an AUF1 disclosed herein and the second therapeutic is an immunosuppressive or anti-inflammatory therapy. In embodiments, a combination of the rAAV encoding AUF1, the rAAV encoding the microdystrophin and the immunosuppressive/anti-inflammatory therapeutic (as a third therapeutic) is administered to treat or ameliorate the symptoms of the dystrophinopathy of the subject.

In some embodiments, the immunosuppressive or anti-inflammatory therapy is edasalonexent.

In some embodiments, the immunosuppressive or anti-inflammatory therapy is canakinumab. Canakinumab is a monoclonal antibody, targeting IL1b, which is a cytokine that plays a role in inflammation and immune responses. In some embodiments, canakinumab can be administered subcutaneously. In some embodiments, administration can be daily, weekly, or monthly. In some embodiments, the length of treatment can be weeks, months or years. In some embodiments, canakinumab can be administered in a dose of 0.5 mg/kg to 20 mg/kg. In some embodiments, canakinumab can be administered in a dose of 2 mg/kg or 4 mg/kg. For example, administration can be a single dose via subcutaneous injection of 2 or 4 mg/kg.

In some embodiments, the immunosuppressive or anti-inflammatory therapy is pamrevlumab. Pamrevlumab is an antibody therapy designed to block the activity of connective tissue growth factor (CTGF), a pro-inflammatory protein that promotes fibrosis (scarring) and is found at unusually high levels in the muscles of people with DMD. Fibrosis is a hallmark of muscular dystrophies, contributing to muscle weakness and injury, including to cardiac muscle. In some embodiments, inhibition of connective tissue growth factor (CTGF) by pamrevlumab could result in decreased fibrosis in muscles leading to increased muscle function. In some embodiments, Pamrevlumab can be administered intravenously. In some embodiments, administration can be daily, weekly, or monthly. In some embodiments, the length of treatment can be weeks, months or years. In some embodiments, Pamrevlumab can be administered in a dose of 10 mg/kg to 200 mg/kg. In some embodiments, Pamrevlumab can be administered in a dose of 35 mg/kg. For example, administration can be every two weeks via intravenous (IV) infusions of 35 mg/kg.

In some embodiments, the immunosuppressive or anti-inflammatory therapy is imlifidase. Imlifidase is an enzyme that rapidly cleaves IgG antibodies, thereby suppressing the immune response against AAVs. Thus, once the immune response against AAVs has been suppressed, gene therapy treatments using an AAV vector can be used more efficiently. In some embodiments, imlifidase can be administered intravenously. In some embodiments, administration can be daily, weekly, or monthly. In some embodiments, the length of treatment can be weeks, months or years. In some embodiments, imlifidase can be administered in a dose of 0.1 mg/kg to 10 mg/kg. In some embodiments, imlifidase can be administered in a dose of 0.25 mg/kg. For example, administration can a single dose via intravenous (IV) infusions of 0.25 mg/kg.

5.6.6 Therapies that Treat One or More Symptoms of the Dystrophinopathy

Disclosed are methods of treating a dystrophinopathy in a subject in need thereof, comprising administering to the subject a first therapeutic and a second therapeutic, wherein the first therapeutic is an rAAV comprising a transgene encoding a AUF1 disclosed herein and the second therapeutic is a therapy that treats one or more symptoms of the dystrophinopathy. In some embodiments, a therapy that treats one or more symptoms of the dystrophinopathy can also include any of the mutation suppression therapies, exon skipping therapies, steroid therapies, and immunosuppressive/anti-inflammatory therapies described herein. In embodiments, a combination of the rAAV encoding AUF1, the rAAV encoding the microdystrophin and therapy that treats one or more symptoms of the dystrophinopathy (as a third therapeutic) is administered to treat or ameliorate the symptoms of the dystrophinopathy of the subject.

In some embodiments, the one or more symptoms of the dystrophinopathy is decreased muscle mass and/or strength, wherein the second therapeutic improves muscle mass and/or strength. For example, the second therapeutic can be spironolactone (same as described for steroid therapy), Follistatin, SERCA2a, EDG-5506, Tamoxifen, Givinostat, ASP0367, or a combination thereof.

In some embodiments, follistatin or follistatin variants can be used as the second therapeutic. In some embodiments, follistatin can be administered as a gene therapy in a viral vector such as AAV.

In some embodiments, SERCA2a can be used as the second therapeutic (or a third therapeutic). In some embodiments, SERCA2a can be administered as a gene therapy in a viral vector such as AAV. In some embodiments, SERCA2a can be administered intravenously. In some embodiments, administration can be daily, weekly, or monthly. In some embodiments, the length of treatment can be weeks, months or years. In some embodiments, 1×10¹¹ to 1×10¹⁴ vg is administered. In some embodiments, 6×10¹² vg is administered.

EDG-5506 is a small molecule therapy that can stabilize skeletal muscle fibers (muscles under voluntary control) and protect them from damage during contractions. In some embodiments, SERCA2a can be administered orally. In some embodiments, administration can be daily, weekly, or monthly. In some embodiments, the length of treatment can be weeks, months or years.

In some embodiments, the second therapeutic (or third therapeutic) is tamoxifen. In some embodiments, tamoxifen can be administered orally. In some embodiments, administration can be daily, weekly, or monthly. In some embodiments, the length of treatment can be weeks, months or years. In some embodiments, tamoxifen can be administered in a dose of 0.1 mg/kg to 20 mg/kg. In some embodiments, tamoxifen can be administered in a dose of 0.6 mg/kg. In some embodiments, tamoxifen can be administered in a dose of 5 mg to 100 mg. For example, administration can be a single oral dose of 0.6 mg/kg daily.

In some embodiments, Givinostat is a molecule that inhibits enzymes called histone deacetylases (HDACs) that turn off gene expression and can reduce a muscle's ability to regenerate. By inhibiting HDACs, givinostat may reduce fibrosis and the death of muscle cells while also enabling muscles to regenerate. In some embodiments, Givinostat is administered via oral suspension. In some embodiments, administration can be daily, weekly, or monthly. In some embodiments, the length of treatment can be weeks, months or years. In some embodiments, Givinostat can be administered in a dose of 1 mg/ml to 100 mg/ml. In some embodiments, Givinostat can be administered in a dose of 10 mg/ml. For example, administration can be twice daily via oral suspension of 10 mg/ml.

In some embodiments, ASP0367 is used turn on the PPAR delta (δ) pathway. The PPAR-δ pathway regulates mitochondria by turning on different genes in the cell. When the pathway is on, the mitochondria use fatty acids more often and more mitochondria are made. Using more fatty acids for energy results in increased energy production. Thus, ASP0367 is a mitochondrial-directed medicine for the treatment of DMD, which is designed to treat DMD by increasing fatty acid oxidation and mitochondrial biogenesis in muscle cells.

In some embodiments, the second therapeutic (or third therapeutic) is a cell based therapy. For example, the cell based therapy is one or more myoblasts. In some embodiments, the myoblast cell based therapy is as described in NCT02196467. In some embodiments, 1-500 million myoblasts can be transplanted per centimeter cube in the Extensor carpi radialis of one of the patient's forearms, resuspended in saline. More specifically, 30 million myoblasts can be transplanted per centimeter cube can be transplanted.

In some embodiments, the cell based therapy is CAP-1002 and can improve respiratory, cardiac and upper limb function. Thus, in some embodiments, the cell based therapy is a cardiosphere derived cell.

In some embodiments, the one or more symptoms of the dystrophinopathy is a symptom related to a cardiac condition. In some embodiments, the cardiac condition is cardiomyopathy, decreased cardiac function, fibrosis in the heart, or a combination thereof. Thus, in some embodiments, the second therapeutic (or third therapeutic) is Ifetroban, Bisoprolol fumarate, Eplerenone, or a combination thereof.

Ifetroban is a potent and selective thromboxane receptor antagonist. In some embodiments ifetroban can stop important molecular signals that mediate inflammation and fibrosis (tissue scaring) mechanisms in the heart, triggered by the loss of dystrophin protein—the hallmark feature of DMD. In some embodiments, ifetroban is administered orally. In some embodiments, administration can be daily, weekly, or monthly. In some embodiments, the length of treatment can be weeks, months or years. In some embodiments, ifetroban can be administered in a dose of 50 mg to 400 mg. In some embodiments, ifetroban can be administered in a dose of 200 mg. For example, administration can be once daily via capsule—four 50 mg capsules. In some embodiments, Bisoprolol is administered at a dose of 0.05 mg/kg to 20 mg/kg. In some embodiments, Bisoprolol is administered at a dose of 0.2 mg/kg. In some embodiments, Bisoprolol is administered at a dose of 1.25 mg every 24 hr and the subject is monitored for heart rate, blood pressure, and other heart related symptoms. The bisoprolol dose can be increased 1.25 mg progressively until a daily dose of 0.2 mg/kg or the maximum tolerated dose (he rest heart rate <75 bpm and systolic blood pressure <90 mmHg) is achieved. Dosing can be increased with an assessment of the subject's heart rate, blood pressure, symptoms and ECG.

In some embodiments, eplerenone is administered orally. In some embodiments, administration can be daily, weekly, or monthly. In some embodiments, the length of treatment can be weeks, months or years. In some embodiments, eplerenone can be administered in a dose of 10 mg to 200 mg. In some embodiments, eplerenone can be administered in a dose of 25 mg. For example, administration can be once daily via capsule in a single 25 mg capsule.

In some embodiments, the one or more symptoms of the dystrophinopathy is a respiratory symptom. Thus, the second therapeutic (or third therapeutic) can be Idebenone. In some embodiments, Idebenone can be administered orally. In some embodiments, administration can be daily, weekly, or monthly. In some embodiments, the length of treatment can be weeks, months or years. In some embodiments, Idebenone can be administered in a dose of 250 mg/day to 2000 mg/day. In some embodiments, Idebenone can be administered in a dose of 900 mg/day. For example, administration can be three times a day, orally, wherein each oral administration is two tablets each of 150 mg. In some embodiments, the second therapeutic (or third therapeutic) is orthopedic management, endocrinologic management, gastrointestinal management, urologic management, or a combination thereof. In some embodiments, the second therapeutic (or third therapeutic) is transcutaneous electrical nerve stimulation (TENS). TENS can increase muscle strength, increase range of joint motions and/or improve sleep. In some embodiments, the TENS is applied using VECTTOR system. The VT-200, or VECTTOR system, delivers electrical stimulation via electrodes on the acupuncture points of a subject's feet/legs and hands/arms to provide symptomatic relief of chronic intractable pain and/or management of post-surgical pain. In some embodiments, nerve stimulator treatment (e.g. TENS) can be administered one time, two times, three times, four times, five times or more daily.

### 5.6.7 Therapeutically Effective Dosages

Disclosed are methods of treatment of human patients (e.g. subjects) amenable to treatment with an rAAV encoding a functional AUF1 and a second therapeutic, including an rAAV encoding a microdystrophin, effective to treat or ameliorate one or more symptoms of a dystrophinopathy, by peripheral, including intravenous, administration. In some aspects, a patient/subject amenable to treatment with the rAAV encoding an AUF1 is a patient having a dystrophinopathy (e.g. DMD or BMD).

In some aspects, the first therapeutic is an rAAV particle, including an AAV8 serotype or an AAV9 serotype, containing a construct encoding a AUF1 and administration of an rAAV particle containing a construct encoding a AUF1 as described herein, including the constructs having nucleotide sequences of SEQ ID NO:31 to 36 (spc-hu-opti-AUF1-CpG(−), tMCK-huAUF1, spc5-12-hu-opti-AUF1-WPRE, ss-CK7-hu-AUF1, spc-hu-AUF1-no-intron, and D(+)-CK7AUF1, respectively), can occur at a dosage of 2×10¹³ to 1×10¹⁵, including a dose of 2×10¹⁴ vg/kg. Doses can range from 1×10⁸ vector genomes per kg (vg/kg) to 1×10¹⁵ vg/kg. In some embodiments, the dose can be 2×10¹³, 3×10¹³, 1×10¹⁴, 3×10¹⁴, 5×10¹⁴ vg/kg. In some embodiments, the dose can be 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ vg/kg in combination with the second therapeutic.

In some aspects, the second therapeutic is an rAAV particle containing a construct encoding a microdystrophin and administration of an rAAV particle containing a construct encoding a microdystrophin described herein, including constructs having a nucleotide sequence of SEQ ID NO: 94, 95 or 96 (serotype AAV8 or AAV9) can occur at a dosage of 2×10¹³ to 1×10¹⁵, including a dose of 2×10¹⁴ vg/kg. Doses can range from 1×10⁸ vector genomes per kg (vg/kg) to 1×10¹⁵ vg/kg. In some embodiments, the dose can be 2×10¹³, 3×10¹³, 1×10¹⁴, 3×10¹⁴, 5×10¹⁴ vg/kg. In some embodiments, the dose can be 1×10¹⁴, 1.1×10¹⁴, 1.2×10¹⁴, 1.3×10¹⁴, 1.4×10¹⁴, 1.5×10¹⁴, 1.6×10¹⁴, 1.7×10¹⁴, 1.8×10¹⁴, 1.9×10¹⁴, 2×10¹⁴, 2.1×10¹⁴, 2.2×10¹⁴, 2.3×10¹⁴, 2.4×10¹⁴, 2.5×10¹⁴, 2.6×10¹⁴, 2.7×10¹⁴, 2.8×10¹⁴, 2.9×10¹⁴, or 3×10¹⁴ vg/kg.

In certain aspects, the ratio of the AUF1 gene therapy vector and the microdystrophin gene therapy vector is 1:1, 1:2, 1:4, 1:5; 1:10, 1:50, 1:100 or 1:1000. Alternatively, the ratio of the AUF1 gene therapy vector and the microdystrophin gene therapy vector is 0.5:1, 0.25:1, 0.2:1, or 0.1:1.

Therapeutically effective dosages are administered as a single dosage (for example, simultaneously in a single composition or separate compositions) or within 1 hour, 2 hours, 3 hours, 4 hours, 12 hours, 1 day, 2 day, 3, days, 4 days, 5 days, 6 days, 7 days, or 2 weeks. In embodiments, the first therapeutic, the AUF1 gene therapy vector is administered prior to the second therapeutic, the microdystrophin gene therapy vector. In some embodiments, the first therapeutic, the AUF1 gene therapy vector, is administered subsequent to the second gene therapy vector, the microdystrophin gene therapy vector. If the second therapeutic is not a gene therapy or if a third therapeutic (or even further therapeutics) are administered which are not gene therapy vectors, it may be administered in multiple doses during the course of a treatment regimen (i.e., days, weeks, months, etc.) and may be administered before or after the first (and/or the second) therapeutic or both before and after the first (and or second) gene therapy vector.

The dosages are therapeutically effective, which can be assessed at appropriate times after the administration, including 12 weeks, 26 weeks, 52 weeks or more, and include assessments for improvement or amelioration of symptoms and/or biomarkers of the dystrophinopathy as known in the art and detailed herein. Recombinant vectors used for delivering the transgene encoding AUF1 and microdystrophin are described herein. Such vectors should have a tropism for human muscle cells (including skeletal muscle, smooth muscle and/or cardiac muscle) and can include non-replicating rAAV, particularly those bearing an AAV8 capsid. The recombinant vectors, including vectors having the construct spc-hu-opti-AUF1-CpG(−), tMCK-huAUF1, spc5-12-hu-opti-AUF1-WPRE, ss-CK7-hu-AUF1, spc-hu-AUF1-no-intron, and D(+)-CK7AUF1 (see FIG. 1), for AUF1 expression and RGX-DYS1 or RGX-DYS5 for microdystrophin can be administered in any manner such that the recombinant vector enters the muscle tissue, including by introducing the recombinant vector into the bloodstream, including intravenous administration.

Subjects to whom such gene therapy is administered can be those responsive to gene therapy mediated delivery of AUF1, including in combination with gene therapy mediated delivery of microdystrophin, to muscles. In particular embodiments, the methods encompass treating patients who have been diagnosed with DMD or other muscular dystrophy disease, such as, Becker muscular dystrophy (BMD), myotonic muscular dystrophy (Steinert's disease), Facioscapulohumeral disease (FSHD), limb-girdle muscular dystrophy, X-linked dilated cardiomyopathy, or oculopharyngeal muscular dystrophy, or have one or more symptoms associated therewith, and identified as responsive to treatment with microdystrophin, or considered a good candidate for therapy with gene mediated delivery of microdystrophin. In specific embodiments, the patients have previously been treated with synthetic version of dystrophin and have been found to be responsive to one or more of synthetic versions of dystrophin. To determine responsiveness, the synthetic version of dystrophin (e.g., produced in human cell culture, bioreactors, etc.) may be administered directly to the subject.

Therapeutically effective doses of any such recombinant vector should be administered in any manner such that the recombinant vector enters the muscle (e.g., skeletal muscle or cardiac muscle), including by introducing the recombinant vector into the bloodstream. In specific embodiments, the vector is administered subcutaneously, intramuscularly or intravenously. The expression of the transgene product results in delivery and maintenance of the transgene product in the muscle.

Pharmaceutical compositions suitable for intravenous, intramuscular, or subcutaneous administration comprise a suspension of the recombinant AAV comprising any of the transgenes disclosed herein in a formulation buffer comprising a physiologically compatible aqueous buffer. The formulation buffer can comprise one or more of a polysaccharide, a surfactant, polymer, or oil. The disclosed pharmaceutical compositions can comprise any of the microdystrophins, particularly the rAAV vectors comprising a transgene encoding AUF1 or the microdystrophins, disclosed herein and can be used in the disclosed methods.

The disclosed methods of treatment can result in one of many endpoints indicative of therapeutic efficacy described herein. In some embodiments, the endpoints can be monitored 6 weeks, 12 weeks, 24 weeks, 30 weeks, 36 weeks, 42 weeks, 48 weeks, 1 year, 2 years, 3 years, 4 years or 5 years after the administration of a rAAV particle comprising a transgene that encodes AUF1.

In some embodiments, creatine kinase activity can be used as an endpoint for therapeutic efficacy of the methods of treatment and administration disclosed herein. The creatine kinase activity can decrease in the subject relative to the level (of creatine kinase activity) prior to said administration. In some embodiments, the creatine kinase activity can decrease in the subject relative to the level (of creatine kinase activity) in the subject prior to treatment or relative to the level (of creatine kinase activity) in a non-treated subject having a dystrophinopathy (for example, a reference level identified in a natural history study). The creatine kinase activity measured in the human subject after administration of a rAAV with a transgene encoding AUF1, including in combination with an rAAV with a transgene encoding a microdystrophin, can be to a control value which can be the creatine kinase activity in the subject prior to administration, creatine kinase activity in a subject with a dystrophinopathy that has not be treated, creatine kinase activity in a subject that does not have a dystrophinopathy, creatine kinase activity in a standard. In some embodiments, administration results in a decrease in creatine kinase activity, which can be a decrease of 1000 to 10,000 units/liter compared to a control or the value measured in the subject amount prior to administration of the therapeutic. In some embodiments, an amount of 1000, 2000, 3000, 4000, or 5000 units/liter in the after-administration endpoint is indicative of a decrease.

In some embodiments, reduction in lesions in a gastrocnemius muscle (or other muscle) can be used as an endpoint measure for therapeutic efficacy for the methods of treatment and administration disclosed herein. The lesions in a gastrocnemius muscle can decrease in the subject relative to the level (of lesions in the gastrocnemius muscle) prior to administration of the therapeutics. In some embodiments, the lesions in the gastrocnemius muscle can decrease in the subject relative to the level (of lesions in the gastrocnemius muscle) in a non-treated subject having a dystrophinopathy. The comparison of lesions in the gastrocnemius muscle can be to a standard, wherein the standard is a number or set of numbers that represent the lesions in a subject that does not have a dystrophinopathy or the lesions in a non-treated subject having a dystrophinopathy. Thus, in some embodiments, the comparison of lesions in the gastrocnemius muscle after administration of a therapeutic can be to a control subject. The control can be the lesions in the gastrocnemius muscle in the subject prior to administration lesions in the gastrocnemius muscle in a subject with a dystrophinopathy that has not be treated, lesions in the gastrocnemius muscle in a subject that does not have a dystrophinopathy, or lesions in the gastrocnemius muscle in a standard.

In some embodiments, the lesions in the gastrocnemius muscle of the subject are assessed using magnetic resonance imaging (MRI). MRI can be a good tool for imagine muscles, ligaments, and tendons, therefore, muscle disorders can be detected and/or characterized using MRI. In some embodiments, administration of therapeutics disclosed herein results in a decrease of lesions in gastrocnemius muscle after administration is about 1-100%, 2-50%, or 3-10% compared a control, for example, compared to the lesions in the gastrocnemius muscle of the subject prior to said administration. For example, a subject treated with a rAAV with a transgene encoding AUF1, including in combination with an rAAV encoding a microdystrophin can have 1, 5, 10, 15, 20, 25, 30, 35, 40, 45, or 50% or greater decrease in lesions compared to a control.

In some embodiments, gastrocnemius muscle volume (or muscle volume of any other muscle) can be used as an endpoint for treatment efficacy. The gastrocnemius muscle volume can decrease in the subject relative to the level (of gastrocnemius muscle volume) prior to said administration of rAAV with a transgene encoding AUF1. In some embodiments, the gastrocnemius muscle volume can decrease in the subject relative to the level (of gastrocnemius muscle volume) in a subject that does not have a dystrophinopathy. In some embodiments, the gastrocnemius muscle volume can decrease in the subject relative to the level (of gastrocnemius muscle volume) in a non-treated subject having a dystrophinopathy. The comparison of gastrocnemius muscle volume can be to a standard, wherein the standard is a number or set of numbers that represent the volume in a subject that does not have a dystrophinopathy or the volume in a non-treated subject having a dystrophinopathy. Thus, in some embodiments, the comparison of gastrocnemius muscle volume after administration of the therapeutics disclosed herein can be to a control. The control can be the gastrocnemius muscle volume in the subject prior to administration, gastrocnemius muscle volume in a subject with a dystrophinopathy that has not be treated, gastrocnemius muscle volume in a subject that does not have a dystrophinopathy, or gastrocnemius muscle volume in a standard.

In some embodiments, the gastrocnemius muscle volume of the subject can be assessed using MRI. In some embodiments, the administration results in a decrease in gastrocnemius muscle volume of about 1-100%, 2-50%, or 3-20% compared a control, for example, compared to the gastrocnemius muscle volume prior to said administration. In some embodiments, a decrease of gastrocnemius muscle volume after administration of a rAAV comprising a transgene that encodes AUF1, including in combination with an rAAV comprising a transgene encoding a microdystrophin, can be about 2-400 mm³, 5-200 mm³, or 20-100 mm³ compared a control. For example, a subject treated with a rAAV with a transgene encoding AUF1, including in combination with an rAAV comprising a transgene encoding a microdystrophin, can have 10, 20, 30, 40, 50, 60, 70, 80, 90, 100, 110, 120, 130, 140, or 150 mm³ or greater decrease in gastrocnemius muscle volume compared to a control.

In some embodiments, a fat fraction of muscle can be used as an endpoint for therapeutic efficacy of the methods of administering rAAV therapeutics disclosed herein. The muscle can be muscles in the pelvic girdle and thigh (gluteus maximus, adductor magnus, rectus femoris, vastus lateralis, vastus medialis, biceps femoris, semitendinosus, and gracilis). The fat fraction of muscle can decrease in the subject relative to the level (of fat fraction of muscle) prior to said administration of rAAV with a transgene encoding AUF1, including in combination with an rAAV comprising a transgene encoding a microdystrophin, as disclosed herein. In some embodiments, the fat fraction of muscle can decrease in the subject relative to the level (of fat fraction of muscle) in a non-treated subject having a dystrophinopathy. The comparison of fat fraction of muscle can be to a standard, wherein the standard is a number or set of numbers that represent the amount or percent of fat fraction of muscle in a subject that does not have a dystrophinopathy or the amount or percent in a non-treated subject having a dystrophinopathy. Thus, in some embodiments, the comparison of fat fraction of muscle after administration of a rAAV with a transgene encoding an AUF1, including in combination with an rAAV comprising a transgene encoding a microdystrophin, can be to a control. The control can be the fat fraction of muscle in the subject prior to administration, fat fraction of muscle in a subject with a dystrophinopathy that has not be treated, fat fraction of muscle in a subject that does not have a dystrophinopathy, or fat fraction of muscle of a standard.

In some embodiments, the fat fraction of muscle of the subject are assessed using magnetic resonance imaging (MRI). In some embodiments, provided are methods of treating a dystrophinopathy, including DMD and BMD, by peripheral, including intravenous administration of an rAAV vector containing a AUF1 construct, including a microdystrophin construct disclosed herein, results in a decrease of fat fraction of muscle after administration can be about 1-100%, 2-50%, or 3-10% compared a control, for example, compared to the fat fraction of muscle prior to said administration. For example, a subject so administered can have 1, 5, 10, 15, 20, 25, 30, 35, 40, 45, or 50% or greater decrease in fat fraction of muscle compared to a control.

In some embodiments, gait score can be used as an endpoint for treatment. The gait score can be about −1 to 2 after administration. In some embodiments, the North Star Ambulatory Assessment (NSAA) can be used as an endpoint for treatment. The NSAA of the treated subject can be compared to NSAA prior to administration. The NSAA of the treated subject can be compared to NSAA in a subject that does not have a dystrophinopathy. The NSAA of the treated subject can be compared to a non-treated subject having a dystrophinopathy. The NSAA of the treated subject can be compared to a standard, wherein the standard is a score or set of scores that represent the NSAA in a subject that does not have a dystrophinopathy or the NSAA in a non-treated subject having a dystrophinopathy. In some embodiments, the NSAA of the subject treated compared to the NSAA score prior to said administration or compared to any of the NSAA comparisons described above. In some embodiments, the increase can be from 0 to 1, 0 to 2 or from 1 to 2.

### 5.6.8 Cardiac Output

Although skeletal muscle syriptoms are considered the defining characteristic of DMD, patients most commonly die of respiratory or cardiac failure. DMD patients develop dilated cardiomyopathy (DCM) due to the absence of dystrophin in cardiomyocytes, which is required for contractile function. This leads to an influx of extracellular calcium, triggering protease activation, cardionyocyte death, tissue necrosis, and inflammation, ultimately leading to accumulation of fat and fibrosis. This process first affects the left ventricle (LV), which is responsible for pumping blood to most of the body and is thicker and therefore experiences a greater workload. Atrophic cardiomyocytes exhibit a loss of striations, vacuolization, fragmentation, and nuclear degeneration. Functionally, atrophy and scarring leads to structural instability and hypokinesis of the LV, ultimately progressing to general DCM. DMD may be associated with various ECG changes like sinus tachycardia, reduction of circadian index, decreased heart rate variability, short PR interval, right ventricular hypertrophy, S-T segment depression and prolonged QTc.

Gene therapy treatment provided herein can slow or arrest the progression of DMD and other dystrophinopathies, particularly to reduce the progression of or attenuate cardiac dysfunction and/or maintain or improve cardiac function. Efficacy may be monitored by periodic evaluation of signs and symptoms of cardiac involvement or heart failure that are appropriate for the age and disease stage of the trial population, using serial electrocardiograms, and serial noninvasive imaging studies (e.g., echocardiography or cardiac magnetic resonance imaging (CMR)). CMR may be used to monitor changes from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), inflammation, and fibrosis. ECG may be used to monitor conduction abnormalities and arrythmias. In particular, ECG may be used to assess normalization of the PR interval, R waves in V1, Q waves in V6, ventricular repolarization, QS waves in inferior and/or upper lateral wall, conduction disturbances in right bundle branch, QT C, and QRS.

Therapeutic methods disclosed herein can improve or maintain cardiac function or slow the loss of cardiac function, for example, by preventing reductions in decreasing LVEF below 45% and/or normalization of function (LVFS≥28%) as measured by serial electrocardiograms, and/or serial noninvasive imaging studies (e.g., echocardiography or cardiac magnetic resonance imaging (CMR)). Measurements may be compared to an untreated control or to the subject prior to treatment. Alternatively, treatment as disclosed herein results in an improvement in cardiac function or reduction in the loss of cardiac function as assessed by monitoring changes from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), inflammation, and fibrosis. ECG may be used to monitor conduction abnormalities and arrythmias. In particular, ECG may be used to assess normalization of the PR interval, R waves in V1, Q waves in V6, ventricular repolarization, QS waves in inferior and/or upper lateral wall, conduction disturbances in right bundle branch, QT C, and QRS.

In some embodiments, cardiac function and/or pulmonary function can be used as an endpoint for assessment of therapeutic efficacy of the administration. The cardiac function and/or pulmonary function can improve or increase in the subject relative to the level (of cardiac function and/or pulmonary function) prior to said administration. In some embodiments, the cardiac function and/or pulmonary function can improve or increase in the subject relative to the level (of cardiac function and/or pulmonary function) in a subject that does not have a dystrophinopathy. In some embodiments, the cardiac function and/or pulmonary function can decrease in the subject relative to the level (of cardiac function and/or pulmonary function) in a non-treated subject having a dystrophinopathy. The comparison of cardiac function and/or pulmonary function can be to a standard, wherein the standard is a number or set of numbers that represent the cardiac function and/or pulmonary function in a subject that does not have a dystrophinopathy or the cardiac function and/or pulmonary function in a non-treated subject having a dystrophinopathy. Thus, in some embodiments, the comparison of cardiac function and/or pulmonary function after administration can be to a control. The control can be the cardiac function and/or pulmonary function in the subject prior to administration, cardiac function and/or pulmonary function in a subject with a dystrophinopathy that has not be treated, cardiac function and/or pulmonary function in a subject that does not have a dystrophinopathy, cardiac function and/or pulmonary function in a standard.

In some embodiments, an improvement or increase in cardiac function and/or pulmonary function is 1 to 100% compared to a control, for example, compared to the subject prior to administration. In some embodiments, cardiac function can be measured using impedance, electric activities, and calcium handling.

### 5.6.9 Patient Primary Endpoints

The efficacy of the compositions, including the dosage of the composition, and methods described herein may be assessed in clinical evaluation of subjects being treated. Patient primary endpoints may include monitoring the change from baseline in forced vital capacity (FVC), forced expiratory volume (FEV1), maximum inspiratory pressure (MIP), maximum expiratory pressure (MEP), peak expiratory flow (PEF), peak cough flow, left ventricular ejection fraction (LVEF), left ventricular fractional shortening (LVFS), change from baseline in the NSAA, change from baseline in the Performance of Upper Limp (PUL) score, and change from baseline in the Brooke Upper Extremity Scale score (Brooke score), change from baseline in grip strength, pinch strength, change in cardiac fibrosis score by MRI, change in upper arm (bicep) muscle fat and fibrosis assessed by MRI, measurement of leg strength using a dynamometer, walk test 6-minutes, walk test 10-minutes, walk analysis—3D recording of walking, change in utrophin membrane staining via quantifiable imaging of immunostained biopsy sections, and a change in regenerating fibers by measuring (via muscle biopsy) a combination of fiber size and neonatal myosin positivity. See, for example, Mazzone E et al, North Star Ambulatory Assessment, 6-minute walk test and timed items in ambulant boys with Duchenne muscular dystrophy. Neuromuscular Disorders 20 (2010) 712-716.; Abdelrahim Abdrabou Sadek, et al, Evaluation of cardiac functions in children with Duchenne Muscular Dystrophy: A prospective case-control study. Electron Physician (2017) November; 9(11): 5732-5739; Magrath, P. et al, Cardiac MRI biomarkers for Duchenne muscular dystrophy. BIOMARKERS IN MEDICINE (2018) VOL. 12, NO. 11.; Pane, M. et al, Upper limb function in Duchenne muscular dystrophy: 24 month longitudinal data. PLoS One. 2018 Jun. 20; 13(6):e0199223.

5.7. Methods of Treatment with AUF1 Gene Therapy Constructs

Advancing age and sedentary life-style promotes significant muscle loss that becomes largely irreversible with advancing age, including very severe muscle loss and atrophy with age (sarcopenia). Sarcopenia and age-related muscle loss is a significant source of morbidity and mortality in the aging and the elderly population. Only physical exercise is considered an effective strategy to improve muscle maintenance and function, but it must begin well before the onset of disease. In addition, traumatic muscle injury can resulting in lasting muscle loss and debilitation. There are few effective therapeutic options. AUF1 skeletal muscle gene transfer: (1) strongly enhances exercise endurance in middle-aged (12 month; equivalent to approximately 38 to 47 year old humans) and old mice (18 months; equivalent to about 56 years of age humans) to even older mice (24 months, equivalent to approximately 70 year or older) to levels of performance displayed by young mice (3 months old; equivalent to late teens, early 20's in humans) (see, e.g., Flurkey, Currer, and Harrison, 2007. ‘The mouse in biomedical research.’ in James G. Fox (ed.), American College of Laboratory Animal Medicine series (Elsevier, AP: Amsterdam; Boston, which is incorporated by reference herein in its entirety) (2) stimulates both fast and slow muscle, but specifically specifies slow muscle lineage by increasing levels of expression of the gene pgc1a (Peroxisome proliferator-activated receptor gamma co-activator 1-alpha), a major activator of mitochondrial biogenesis and slow-twitch myofiber specification; (3) significantly increases skeletal muscle mass and normal muscle fiber formation in middle age and old mice in age-related muscle loss; and (4) reduces expression of established biomarkers of muscle atrophy and muscle inflammation in age-related muscle loss.

Thus, another aspect provided herein relates to a method of promoting muscle regeneration by administration of the rAAV vectors comprising a transgene encoding AUF1 as disclosed herein. Thus, provided are methods of promoting muscle regeneration in a subject in need thereof by contacting muscle cells with a therapeutically effective amount of an rAAV vector, including an AAV8 vector or an AAV9 vector, that comprises a recombinant genome comprising a nucleotide sequence encoding a human AUF1 protein, including the nucleotide sequence of SEQ ID NO; 17, operably linked to one or more regulatory sequences that promote expression of the AUF1 protein in muscle cells of the subject, flanked by ITR sequences (see Table 2 for nucleotide sequences of potential components of these recombinant genomes), and, may be one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively), under conditions effective to express exogenous AUF1 in the muscle cells to increase muscle cell mass, increase muscle cell endurance, and/or reduce serum markers of muscle atrophy. In embodiments, the method results, for example, 1 month, 2 months, 3 months, 4 months, 5 months or six months after administration to the subject, in an increase in muscle cell mass, endurance and/or reduction in serum markers of muscle atrophy by 20%, 30%, 40%, 50%, 60%, 70%, 80%, 90% or 100% or greater (or 2 fold, 3 fold or greater) relative to levels in the subject prior (for example 1 day, 1 week or 2 weeks prior) to the administration or to reference levels.

Accordingly, provided are methods of treating sarcopenia in a subject in need thereof by administering a therapeutically effective amount of an rAAV vector, including an AAV8 vector, an AAV9 vector, or an AAVhu.32 vector, that comprises a recombinant genome comprising a nucleotide sequence of SEQ ID NO: 17 encoding human AUF1 and regulatory sequences (see Table 2) and, may be one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) to the muscles of the subject. The subject is human and may be middle aged (from 40 to 50, from 45 to 55, from 50 to 60, from 55 to 65 years of age) or, alternatively, the subject may be elderly, including subjects from 65 to 75 years of age, 70 to 80 years of age, 75 to 85 years of age, 80 to 90 years of age or even older than 90 years of age and the administration of AUF1 results in increased muscle mass, muscle performance, muscle stamina and slowing or even reversal of muscle atrophy, for example, as assessed by biomarkers for muscle mass, muscle performance, muscle stamina or muscle atrophy. In embodiments, the method results in an increase in muscle cell mass, endurance and/or reduction in serum markers of muscle atrophy, for example, 1 month, 2 months, 3 months, 4 months, 5 months or six months after administration to the subject, by 20%, 30%, 40%, 50%, 60%, 70%, 80%, 90% or 100% or greater (or 2 fold, 3 fold or greater) relative to levels in the subject prior (for example 1 day, 1 week or 2 weeks prior) to the administration or to reference levels. In alternative embodiments, the subject is a non-human mammal, including dogs, cats, horses, cows, pigs, sheep, etc. and is middle aged or elderly.

The dystrophin glycoprotein complex (DGC), also known as the DAPC, supra, is a specialization of cardiac and skeletal muscle membrane. This large multicomponent complex has both mechanical stabilizing and signaling roles in mediating interactions between the cytoskeleton, membrane, and extracellular matrix. The DGC links the actin cytoskeleton to the basement membrane and is thought to provide mechanical stability to the sarcolemma (see, e.g., Petrof B J (2002) Am J Phys Med Rehabil 81, S162-5174). AUF1 increases expression or stability of one or more of the components in the DGC or that interact with the DGC, which provides stability to the sarcolemma and thereby increases or improves muscle strength and/or function.

Accordingly, disclosed are methods of stabilizing sarcolemma in a subject, including a human subject, in need thereof, said method comprising administering to the subject a pharmaceutical composition comprising a therapeutically effective amount of an rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively). These methods may be useful in the treatment of muscle degenerative diseases and disorders, such as dystrophinopathies, as described below.

β-dystroglycan, present in the DGC, forms a complex in skeletal muscle fibers and plays a role in linking dystrophin to the laminin in the extracellular matrix. The presence of the DGC helps strengthen muscle fibers and protect them from injury. Disclosed are methods of increasing β-dystroglycan in a DGC comprising administering to the subject an rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2) and, may be one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

β-sarcoglycan can also form a complex with the DGC to help stabilize and strengthen muscle. Disclosed are methods of increasing β-sarcoglycan or γ sarcoglycan in a DGC comprising administering to the subject an rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2) and, may be one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively). Also provided are methods of increasing expression of one or a combination of α-sarcoglycan, β-sarcoglycan, δ-sarcoglycan, γ-sarcoglycan, ε-Sarcoglycan, ζ-sarcoglycan, α-dystroglycan, β-dystroglycan, sarcospan, α-syntrophin, β-syntrophin, α-dystrobrevin, β-dystrobrevin, caveolin-3, or nNOS by administering to a subject an rAAV vector, including an AAV8 vector or an AAV9 vector or an AAVhu.32 vector, that comprises a recombinant genome having comprising a nucleotide sequence of SEQ ID NO: 17 encoding human AUF1 and regulatory sequences (see Table 2) and, may one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

Further provided are methods of increasing utrophin participation in DGCs in a subject in need thereof by administering to the subject an rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

A further aspect of the present application relates to a method of treating degenerative skeletal muscle loss in a subject. This method involves selecting a subject in need of treatment for skeletal muscle loss and administering to the selected subject administering to the subject an rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively), under conditions effective to cause skeletal muscle regeneration in the selected subject. For example, the administering may be effective to activate muscle stem cells, accelerate the regeneration of mature muscle fibers (myofibers), enhance expression of muscle regeneration factors, accelerate the regeneration of injured skeletal muscle, increase regeneration of slow-twitch (Type I) and/or fast-twitch (Type II) fibers), and/or restore muscle mass, muscle strength, and create normal muscle and/or improve mitochondrial oxidative capacity, muscle exercise capacity, muscle performance, stamina and resistance to fatigue in the selected subject.

In embodiments, stabilization of the sarcolemma is compared (at, for example, 1 month, 2 months, 3 months. 4 months, 5 months or 6 months after administration) to normal muscle (or reference normal or diseased muscle) or muscle of the subject prior (e.g., 2 weeks, 1 month or 2 months prior) to administration of the therapeutic (including “pre-treatment levels” being measured within 1 day, 1 week, 2 weeks or 1 month prior to therapeutic administration or other appropriate time period for assessing a baseline value), wherein the stabilization provides for 20%, 30%, 40%, 50%, 60%, 70%, 80%, 90% or 100% or greater (2 fold, 3 fold or more) reduction in markers of sarcolemma integrity, including, for example, serum creatine kinase levels, 20%, 30%, 40%, 50%, 60%, 70%, 80%, 90% or 100% or greater (2 fold, 3 fold or more) reduction in markers of muscle atrophy (for example, biomarkers as disclosed herein), 20%, 30%, 40%, 50%, 60%, 70%, 80%, 90% or 100% or greater (2 fold, 3 fold or more) increase in utrophin levels or a member of the dystrophin sarcoglycan complex, 20%, 30%, 40%, 50%, 60%, 70%, 80%, 90% or 100% or greater (2 fold, 3 fold or more) increase compared to normal muscle or muscle of the subject prior to administration of the therapeutic of muscle mass, or muscle function, or performance using methods known in the art for assessing muscle mass, muscle function or muscle performance.

In some embodiments, the subject has a degenerative muscle condition. As used herein, the term “degenerative muscle condition” refers to conditions, disorders, diseases and injuries characterized by one or more of muscle loss, muscle degeneration or wasting, muscle weakness, and defects or deficiencies in proteins associated with normal muscle function, growth or maintenance. In certain embodiments, a degenerative muscle condition is sarcopenia or cachexia. In other embodiments, a degenerative muscle condition is one or more of muscular dystrophy, muscle injury, including acute muscle injury, resulting in loss of muscle tissue, muscle atrophy, wasting or degeneration, muscle overuse, muscle disuse atrophy, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis. Thus, in some embodiments, the subject has a degenerative muscle condition selected from the group consisting of sarcopenia or myopathy.

The subject may have a muscle disorder mediated by functional AUF1 deficiency or a muscle disorder not mediated by functional AUF deficiency.

In some embodiments, the subject has an adult-onset myopathy or muscle disorder.

Accordingly, provided are methods of treating or ameliorating the symptoms of a dystrophinopathy, including DMD, Becker disease, or limb girdle muscular dystrophy, in a subject in need thereof by administering to the subject a therapeutically effective amount of a rAAV vector, including an AAV8 vector or an AAV9 vector or an AAVhu.32 vector, that comprises a recombinant genome having a nucleotide sequence of one of SEQ ID NO: 31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

In some embodiments, the administering is effective to transduce muscle cells, including skeletal muscle cells, cardiac muscle cells, and/or diaphragm muscle cells and/or provide long-term (e.g., lasting at least 1 month, 2 months, 3 months, 4 months, 5 months, 6 months, 7 months, 8 months, 9 months, 10 months, 11 months, or more) muscle cell-specific AUF1 expression in the selected subject.

In other embodiments, the administering the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is effective to (i) activate high levels of satellite cells and myoblasts; (ii) significantly increase skeletal muscle mass and normal muscle fiber formation relative to pre-treatment levels or a reference standard; and/or (iii) significantly enhanced exercise endurance in the selected subject as compared to when the administering is not carried out.

In further embodiments, the administering the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is effective to reduce (i) biomarkers of muscle atrophy and muscle cell death; (ii) inflammatory immune cell invasion in skeletal muscle (including diaphragm); and/or (iii) muscle fibrosis and necrosis in skeletal muscle (including diaphragm) in the selected subject, as compared to when the administering is not carried out.

In certain embodiments, the administering of the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is effective to (i) increase expression of endogenous utrophin in DMD muscle cells and/or (ii) suppress expression of embryonic dystrophin, a marker of muscle degeneration in DMD in the selected subject, as compared to when the administering is not carried out. In some embodiments of the methods disclosed herein, said administering of an rAAV encoding AUF1 is effective to upregulate endogenous utrophin protein expression in the selected subject, as compared to when the administering is not carried out. In some embodiments of the methods disclosed herein, said administering and rAAV encoding AUF1 is effective to upregulate endogenous utrophin protein expression in said muscle cells, as compared to when the administering is not carried out.

In some embodiments, the administering of the rAAV vector, including an AAV8 vector or an AAV9 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is effective to (i) increase normal expression of genes involved in muscle development and regeneration and/or (ii) suppress genes involved in muscle cell fibrosis, death, atrophy and muscle-expressed inflammatory cytokines in the selected subject, as compared to when the administering is not carried out.

In further embodiments, the administering does not increase muscle mass, endurance, or activate satellite cells in normal skeletal muscle (i.e., healthy skeletal muscle that does not express markers of atrophy, degeneration or loss of weight or stamina).

In some embodiments, the administering is effective to accelerate muscle gain in the selected subject, as compared to when said administering is not carried out.

In certain embodiments, the administering is effective to reduce (for example, by 20%, 30%, 40%, 50%, 60%, 70%, 80%, 90% or 100% or greater) expression of established biomarkers of muscle atrophy in a subject having degenerative skeletal muscle loss relative to the expression levels in the subject prior to therapeutic administration or a reference sample. Suitable biomarkers of muscle atrophy include, without limitation, TRIM63 and Fbxo32 mRNA. In some embodiments, the administering is effective to enhance expression of established biomarkers of muscle myoblast activation, differentiation, and muscle regeneration in the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

### Traumatic Muscle Injury

A further aspect of the present application relates to a method of preventing traumatic muscle injury in a subject. This method involves selecting a subject at risk of traumatic muscle injury and administering to the selected subject the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

Still another aspect of the present application relates to a method of treating traumatic muscle injury in a subject. This method involves selecting a subject having traumatic muscle injury and administering to the selected subject the rAAV vector, including an AAV8 vector or an AAV9 or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively).

In some embodiments of the methods disclosed herein, the subject has traumatic muscle injury. As used herein, the term “traumatic muscle injury” refers to a condition resulting from a wide variety of incidents, ranging from, e.g., everyday accidents, falls, sporting accidents, automobile accidents, to surgical resections to injuries on the battlefield, and many more. Non-limiting examples of traumatic muscle injuries include battlefield muscle injuries, auto accident-related muscle injuries, and sports-related muscle injuries.

Suitable subjects for treatment according to the methods of the present application include, without limitation, domesticated and undomesticated animals such as rodents (mouse or rat), cats, dogs, rabbits, horses, sheep, pigs, and non-human primates. In some embodiments the subject is a human subject. Exemplary human subjects include, without limitation, infants, children, adults, and elderly subjects.

In some embodiments, the subject is at risk of developing or is in need of treatment for a traumatic muscle injury selected from the group consisting of a laceration, a blunt force contusion, a shrapnel wound, a muscle pull, a muscle tear, a burn, an acute strain, a chronic strain, a weight or force stress injury, a repetitive stress injury, an avulsion muscle injury, and compartment syndrome.

In some embodiments, the subject is at risk of developing or is in need of treatment for a traumatic muscle injury that involves volumetric muscle loss (“VML”). The terms “volumetric muscle loss” or “VML” refer to skeletal muscle injuries in which endogenous mechanisms of repair and regeneration are unable to fully restore muscle function in a subject. The consequences of VML are substantial functional deficits in joint range of motion and skeletal muscle strength, resulting in life-long dysfunction and disability.

In some embodiments, the administering is carried to treat a subject having traumatic muscle injury and said administering is carried out immediately after the traumatic muscle injury (for example, within one minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 60 minutes, or any amount of time there between) of the traumatic muscle injury. In certain embodiments, said administering is carryout out within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of the traumatic muscle injury. In other embodiments, said administering is carried out within 1 day, 2 days, 3 days, 4 days, 5 days, 6 days, 7 days, 8 days, 9 days, 10 days, 11 days, 12 days, 13 days, or 14 days of the traumatic muscle injury. In further embodiments, said administering may be carried out within 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 52 weeks, or any amount of time there between of the traumatic muscle injury.

In some embodiments, the administering is effective to prevent muscle atrophy and/or muscle loss following traumatic muscle injury to the selected subject. In other embodiments, the administering is effective to activate muscle stem cells following traumatic muscle injury to the selected subject. In further embodiments, the administering is effective to accelerate the regeneration of mature muscle fibers (myofibers), enhance expression of muscle regeneration factors, accelerate the regeneration of injured muscle, increased regeneration of slow-twitch (Type I) and/or fast-twitch (Type II) fibers), and/or restore muscle mass, muscle, strength and create normal muscle following traumatic muscle injury in the selected subject.

In some embodiments, the administering is effective to accelerate muscle gain following traumatic muscle injury in the selected subject, as compared to when said administering is not carried out.

In certain embodiments, the administering is effective to reduce expression of established biomarkers of muscle atrophy following traumatic muscle injury to the selected subject. Suitable biomarkers of muscle atrophy include, without limitation, TRIM63 and Fbxo32 mRNA. In some embodiments, the administering is effective to enhance expression of established biomarkers of muscle myoblast activation, differentiation and muscle regeneration following traumatic muscle injury to the selected subject. Suitable biomarkers of muscle atrophy include, without limitation, myogenin and MyoD mRNA levels, biomarkers of myoblast activation, differentiation and muscle regeneration (Zammit, “Function of the Myogenic Regulatory Factors Myf5, MyoD, Myogenin and MRF4 in Skeletal Muscle, Satellite Cells and Regenerative Myogenesis,” Semin. Cell. Dev. Biol. 72:19-32 (2017), which is hereby incorporated by reference in its entirety).

Administering, according to the methods of the present application, may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Thus, in some embodiments, the administering is carried out intramuscularly, intravenously, subcutaneously, orally, or intraperitoneally. In specific embodiments, the administering is carried out by intramuscular injection. In some embodiments, the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is administered peripherally, including intramuscularly, intravenously or any other systemic administration method or any method that results in delivery of the rAAV to muscle cells.

In certain embodiments, the dosage of the rAAV vector, including an AAV8 vector or an AAV9 vector or an AAVhu.32 vector, that comprises a recombinant genome comprising a nucleotide sequence of SEQ ID NO: 17 encoding human AUF1 and regulatory sequences (see Table 2), including constructs having a nucleotide sequence of one of SEQ ID NO:31 to 36 (vectors spc-hu-opti-AUF1-CpG(−), tMCK-huAUF1, spc5-12-hu-opti-AUF1-WPRE, ss-CK7-hu-AUF1, spc-hu-AUF1-no-intron, or D(+)-CK7AUF1, respectively) is administered systemically, including intravenously, at 1E13 vg/kg to 1E 14, vg/kg, including a dose of 2E13 vg/kg, and may also be a dose of 3E13 vg/kg, 4E13 vg/kg, 5E13 vg/kg, 6E13 vg/kg, 7E13 vg/kg, 8E13 vg/kg, or 9E13 vg/kg.

### 6. EXAMPLES

### 6.1 Example 1: AUF1 Gene Expression Cassettes for Insertion into Cis Plasmids

Constructs for preparing rAAV8 vectors encoding p40 AUF1 were synthesized. A codon optimized, CpG depleted nucleotide sequence encoding human p40 AUF1 (SEQ ID NO: 17) was identified, synthesized and cloned into a cis plasmid. Expression cassettes were generated incorporating the opti-CpG(−) AUF1 coding sequence (SEQ ID NO: 17) using regulatory elements, the amino acid sequence of which are provided in Table 2. The constructs, spc-hu-opti-AUF1-CpG(−)(SEQ ID NO: 31), tMCK-huAUF1 (SEQ ID NO: 32), spc5-12-hu-opti-AUF1-WPRE (SEQ ID NO: 33), ss-CK7-hu-AUF1 (SEQ ID NO: 34), spc-hu-AUF1-no-intron (SEQ ID NO: 35), or D(+)-CK7AUF1 (SEQ ID NO: 36) are depicted in FIG. 1 (nucleotide sequences provided in Table 3). The constructs were introduced into cis plasmids to be used in producing rAAV, e.g. rAAV8 particles containing the recombinant genome encoding AUF1. Production methods for rAAV particles are known in the art, and for the foregoing experiments using rAAV particles (Examples 2-5), triple transfection of HEK293 cells was performed with (1) the cis plasmid (transgene (such as the therapeutic transgenes described herein) flanked by AAV ITR sequences); (2) rep/cap plasmid (AAV rep and cap genes and gene products, e.g. rep2/cap8 for AAV8); and (3) helper plasmid (suitable helper virus function, usually mutant adenovirus); then the cells cultured in suitable media and media components to support rAAV production until harvest and purification of the particles (rAAV vector).

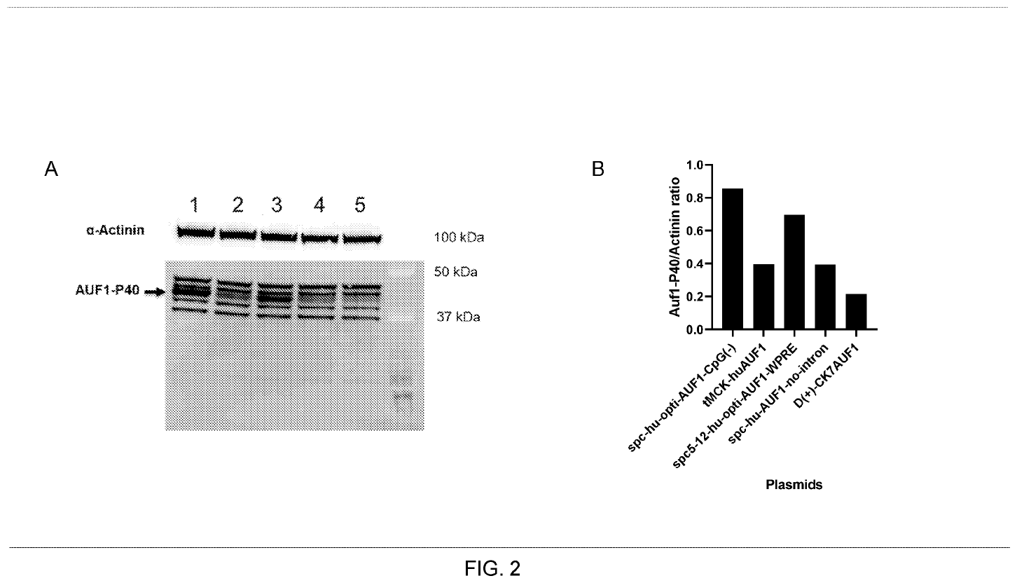

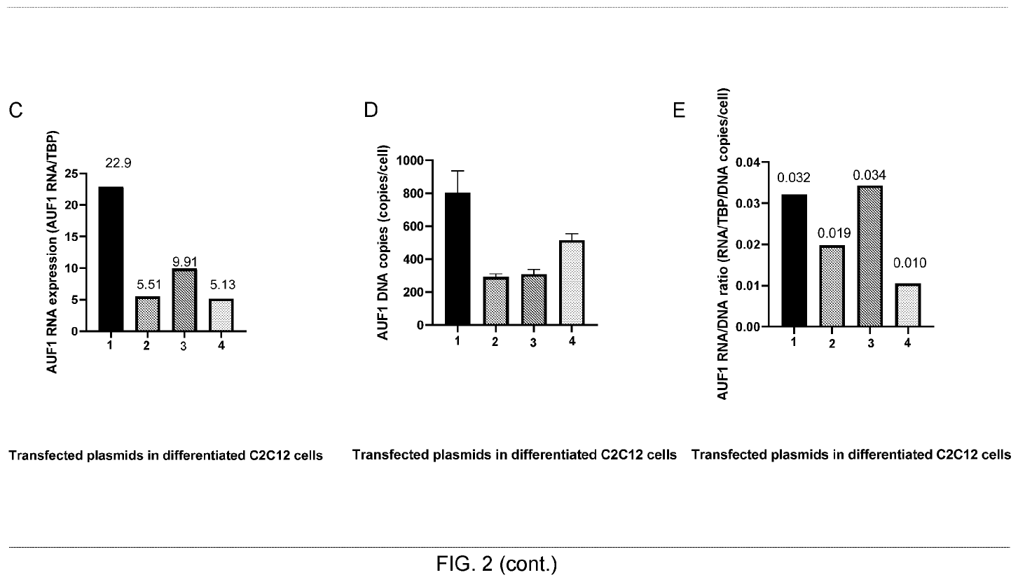

In vitro cell experiments were first performed using the cis plasmids. The cis plasmids were transfected into differentiated C2C12 cells to confirm AUF1 protein expression. The transduced cells were assayed for AUF1 expression either by immunofluorescence or western blot analysis which demonstrated expression of AUF1 (FIG. 2A-B). Briefly, Western blot analysis was performed using an anti-AUF1 antibody. Individual plasmids were transfected into a 6-well plate of C2C12 mouse myoblast with lipofectamine 3000 reagent (ThermoFisher). After overnight transfection, the transfected cells were changed to differentiation media (DMEM+2% HS). Three days after differentiation, the cells were harvested and lysed and subjected to western blot analysis. The polyclonal anti-AUF1 antibody was from Millipore Sigma (Sigma-Aldrich, 07-260, 1:1000 dilution). α-actinin (Abcam, a68167, 1:10000) was used as endogenous control to normalize protein amount.

Quantification of RNA expression and DNA copy numbers was also done by well-known method digital PCR in differentiated C2C12 myotubes after transfection of cis plasmids. The AUF1 RNA expression was expressed as a ratio of AUF1 transcripts to the endogenous control TBP (TATA-box-binding protein) transcripts. See FIG. 2C. The primers and probe sequences were listed in Table 14. The AUF1 DNA copy numbers in transfected cells was also analyzed by digital PCR. See FIG. 2D. The Naica Crystal Digital PCR system from Stilla Technologies was used for this analysis. The copies/cell was calculated as (AUF1 DNA copy numbers/endogenous control glucagon copy numbers)×2. See primers and probe used as listed in Table 14. Finally, AUF1 RNA expression normalized by DNA copy numbers was calculated and represented in FIG. 2E. It was observed that the VH4-intron increased AUF1 RNA expression in differentiated C2C12 cells by around 3-fold, and the increase was also reflected in protein level quantification. WPRE however did not appear to increase AUF1 expression in differentiated C2C12 cells.

TABLE 14
ddPCR primers and probe sequences for digital PCR

| Primer or | Sequences or |
|---|---|
| Probe name | Catalog Number |
| Hu-AUF1-dd- | GGCTTTGTGCTGTTCAAAGAAT |
| F2 | (SEQ ID NO: 121) |
| Hu-AUF1-dd- | ATGGCTTTGGCCCTCTTG |
| R2 | (SEQ ID NO: 122) |
| Hu-AUF1- | Fam-AGCTGAATGGGAAAGTG-MGB |
| Probe-Fam | (SEQ ID NO: 123) |
| Mu_Glucagon- | AAGGGACCTTTACCAGTGATGTG |
| Real-F | (SEQ ID NO: 124) |
| Mu_Glucagon- | ACTTACTCTCGCCTTCCTCGG |
| real-R | (SEQ ID NO: 125) |
| Mu-Glucagon- | Vic-cagcaaaggaattca-MGB |
| probe-Vic | (SEQ ID NO: 126) |
| TBP (20x | ThermoFisher, Mm01277042_ml |
| primers and | Tbp, Lot #: 1909605 |
| probe) | |

### 6.2 Materials and Methods for Examples 2-4

### Dexa Muscle Mass Non-Invasive Quantitative Analysis

Dual energy X-ray absorptiometry (DEXA) is used to record lean muscle mass and changes in muscle mass upon injury or age previously published (Chenette et al., “Targeted mRNA Decay by RNA Binding Protein AUF1 Regulates Adult Muscle Stem Cell Fate, Promoting Skeletal Muscle Integrity,” Cell Rep. 16(5):1379-1390 (2016), which is hereby incorporated by reference in its entirety).

### Muscle Function Tests

Grid hanging time. Mice were placed in the center of a grid, 30 cm above soft bedding to prevent injury should they fall. The grid was then inverted. Grid hanging time was measured as the amount of time mice held on before dropping off the grid. Each mouse may be analyzed twice with 5 repetitions per mouse. See also, Abbadi et al. (2021) “AUF1 Gene Transfer Increases Exercise Performance and Improves Skeletal Muscle Deficit in Adult Mice,” Molecular Therapy 22:222-236, which is incorporated by reference herein in its entirety.

Time, distance to exhaustion, and maximum speed. After 1 week of acclimation, mice were placed on a treadmill and the speed is increased by 1 m/min every 3 minutes and the slope is increased every 9 minutes by 5 cm to a maximum of 15 cm. Mice were considered to be exhausted when they stay on the electric grid more than 10 seconds. Based on their weight and running performance, work performance is calculated in Joules (J). Each mouse may be analyzed twice with 5 repetitions per mouse.

Strength by grip test: In this test, mice grasp a horizon tall grid connected to a dynamometer and are pulled backwards five times by tugging on the tail. The force applied to the grid each time before the animal loses its grip is recorded in Newtons. The average of the five tests is then normalized to the whole-body weight of each mouse. Mice are typically analyzed twice with 5 repetitions per mouse.

### Quantification of Satellite Cells

Muscles were excised and digested in collagenase type I. Cell numbers were quantified by flow cytometry gating for Sdc4⁺ CD45⁻ CD31⁻ Sca1⁻ satellite cell populations (Shefer et al., “Satellite-Cell Pool Size Does Matter: Defining the Myogenic Potency of Aging Skeletal Muscle,” Dev. Biol. 294(1):50-66 (2006) and Brack et al., “Pax7 is Back,” Skelet. Muscle 4(1):24 (2014), which are hereby incorporated by reference in their entirety).

### Muscle Fiber Type Analysis

Skeletal muscles were removed, put in OCT compound, fixed in 4% paraformaldehyde, and immunostained with antibodies to AUF1 (07-260, Millipore), slow myosin (NOQ7.5.4D, Sigma), fast myosin (MY-32, Sigma), and laminin alpha 2 membrane component (4H8-2, Sigma).

### Histological Studies and Biochemical Analysis of Muscle Tissues

Muscles were removed and frozen in OCT compound, fixed in 4% paraformaldehyde, and blocked in 3% BSA in TBS. Immunofluorescence or immunochemistry (Hematoxylin and Eosin, Masson Trichome) was performed. Fibrosis may be assessed by staining of muscle sections with Masson trichrome to visualize areas of collagen deposition and quantified using ImageJ software. Immunofluorescence images may be acquired using a Zeiss LSM 700 confocal microscope. Images and morphometric analysis (Feret diameter, Cross sectional area) are processed using ImageJ as recently described (Abbadi et al., “Muscle Development and Regeneration Controlled by AUF1-Mediated Stage-Specific Degradation of Fate-Determining Checkpoint mRNAs,” Proc. Natl. Acad. Sci. USA 116(23):11285-11290 (2019), and Abbadi et al. (2021) “AUF1 Gene Transfer Increases Exercise Performance and Improves Skeletal Muscle Deficit in Adult Mice,” Molecular Therapy 22:222-236, which are both hereby incorporated by reference in their entireties). Muscles were harvested for biochemical analysis including immunoblot, RNAseq, and RT-PCR analysis.

### Evans Blue Dye Analysis

Evans Blue dye was used as an in vivo marker of muscle damage. It identifies permeable skeletal myofibers that have become damaged (Wooddell et al., “Myofiber Damage Evaluation by Evans Blue Dye Injection,” Curr. Protoc. Mouse Biol. 1(4):463-488 (2011), and Abbadi et al. (2021) “AUF1 Gene Transfer Increases Exercise Performance and Improves Skeletal Muscle Deficit in Adult Mice,” Molecular Therapy 22:222-236, which are hereby incorporated by reference in their entireties).

### Serum Creatine Kinase (CK) Activity

Serum CK was evaluated at 37° C. by standard spectrophotometric analysis using a creatine kinase activity assay kit (abcam). The results are expressed in mU/mL.

### 6.3 Example 2: Evaluation of Combinations of AUF1 and Microdystrophin Gene Therapy Constructs in Mdx Mice

AUF1 or microdystrophin gene therapy constructs (rAAV8 particles), and a combination thereof, are evaluated for efficacy in mdx mice. At 3-4 weeks of age, mdx mice are administered i.v. (either retro-orbital or tail vein) the following AAV8 constructs:
AAV8-RGX-DYS5 (SEQ ID NO: 96) at a dose of 1E14 vg/kg and 2E14 vg/kg body weight;
AAV-8-spc-hu-opti-AUF1-CpG(−) (SEQ ID NO: 31) (or one of tMCK-huAUF1, spc5-12-hu-opti-AUF1-WPRE, ss-CK7-hu-AUF1, spc-hu-AUF1-no-intron, or D(+)-CK7AUF1 (SEQ ID Nos: 32 to 36, respectively) at a dose of 1E13 vg/kg and 1E14 vg/kg body weight;
AAV8-RGX-DYS5 (SEQ ID NO: 96) at a dose of 1E14 vg/kg and simultaneously or shortly preceding or after, but at least within one hour of, the administration of AAV8-Spc-hu-opti-AUF1-CpG(−) (SEQ ID NO: 31) (or one of tMCK-huAUF1, spc5-12-hu-opti-AUF1-WPRE, ss-CK7-hu-AUF1, spc-hu-AUF1-no-intron, or D(+)-CK7AUF1 (SEQ ID Nos: 32 to 36, respectively) at a dose of 1E14 vg/kg body weight.

Mice are sacrificed at 3, 6 and 12 months after injection and the following assessed and compared in a blinded manner:
Dexa muscle mass non-invasive quantitative analysis
Live animal muscle exercise performance function tests, such as, grip strength, grid hanging time, time and distance to exhaustion and max speed
Quantification of satellite cells
Histochemical analysis of muscle tissues using analysis for DAPC or Utrophin and Dystrophin
Gene Expression analysis for AUF1, Utrophin and micro-dystrophin by analyzing mRNA and/or protein levels.
Evans blue dye analysis
Blood and PBMC analysis for CK levels, cytokines and inflammatory markers (markers for T cells, monocytes/macrophages and C-reactive protein).
Vector biodistribution analysis.
RNAseq analysis
Gross anatomical pathology
MRI assessment for muscle size and lesions

### 6.4 Example 3: Evaluation of Combinations of AUF1 and Microdystrophin Gene Therapy Constructs in mdxlutrn Deficient Mice

AUF1 or microdystrophin gene therapy constructs (rAAV particles), and a combination thereof are evaluated for efficacy in C57BL/10 ScSn-congenic utrophin/dystrophin double mutant mice (Jackson Labs). At 3-4 weeks of age, mdxlutrn deficient mice are administered intravenously (either retro-orbital or tail vein) the following AAV8 constructs:
AAV8-RGX-DYS5 (artificial genome having a nucleotide sequence of SEQ ID NO: 96) at a dose of 1E14 vg/kg body weight;
AAV-8-spc-hu-opti-AUF1-CpG(−) (SEQ ID NO: 31) (or one of tMCK-huAUF1, spc5-12-hu-opti-AUF1-WPRE, ss-CK7-hu-AUF1, spc-hu-AUF1-no-intron, or D(+)-CK7AUF1 (artificial genomes having a nucleotide sequence of SEQ ID Nos: 32 to 36, respectively) at a dose of 1E14 vg/kg body weight;
AAV8-RGX-DYS5 (SEQ ID NO: 96) at a dose of 1E14 vg/kg and then simultaneously, or shortly preceding or after, but at least within one hour of, the administration of AAV8-Spc-hu-opti-AUF1-CpG(−) (SEQ ID NO: 31) (or one of tMCK-huAUF1, spc5-12-hu-opti-AUF1-WPRE, ss-CK7-hu-AUF1, spc-hu-AUF1-no-intron, or D(+)-CK7AUF1 (SEQ ID Nos: 32 to 36, respectively) at a dose of 1E14 vg/kg.

Mice are sacrificed at 3 months after injection and the following assessed and compared in a blinded manner:
Dexa muscle mass non-invasive quantitative analysis
Live animal muscle exercise performance function tests, such as, grip strength, grid hanging time, time and distance to exhaustion and max speed
Quantification of satellite cells
Histochemical analysis of muscle tissues
Gene Expression analysis for AUF1, Utrophin and micro-dystrophin by analyzing mRNA and/or protein levels.
Evans blue dye analysis
Blood and PBMC analysis for CK levels, cytokines and inflammatory markers (markers for T cells, monocytes/macrophages and C-reactive protein).
Vector biodistribution analysis
Survival endpoint assessment

### 6.5 Example 4: Evaluation of Combinations of AUF1 and Microdystrophin Gene Therapy Constructs in Mdx Mice

Four-week old mdx mice (C57BL/10ScSn-Dmdmdx/J from Jackson Laboratories) were injected in the retro-orbital sinus with AAV8 vectors. The AAV8-mAUF1 construct, which contains a nucleotide sequence encoding the murine p40 AUF1 isoform under the control of the tMCK promoter, was administered at 2E13 vg/kg. The AAV8-hAUF1 construct has an artificial genome of tMCK-huAUF1 (SEQ ID NO: 32 (including ITR sequences)), which contains a nucleotide sequence encoding a human p40AUF1 protein (SEQ ID NO: 17) under control of the tMCK promoter and was injected at either 2E13 vg/kg or 6E13 vg/kg as indicated. AAV8-RGX-DYS5 (AAV8 containing an RGX-DYS5 artificial genome having a nucleotide sequence of SEQ ID NO: 96 (ITR to ITR), which contains a cDNA encoding a DYS5 microdystrophin (SEQ ID NO: 93 encoding microdystrophin protein SEQ ID NO: 54) driven by an Spc5-12 promoter) was injected at 1E14 vg/kg. Combination therapies consisted of AAV8-hAUF1 injected at 2E13 or 6E13 vg/kg as indicated and AAV8-RGX-DYS5 injected at 1E14 vg/kg.

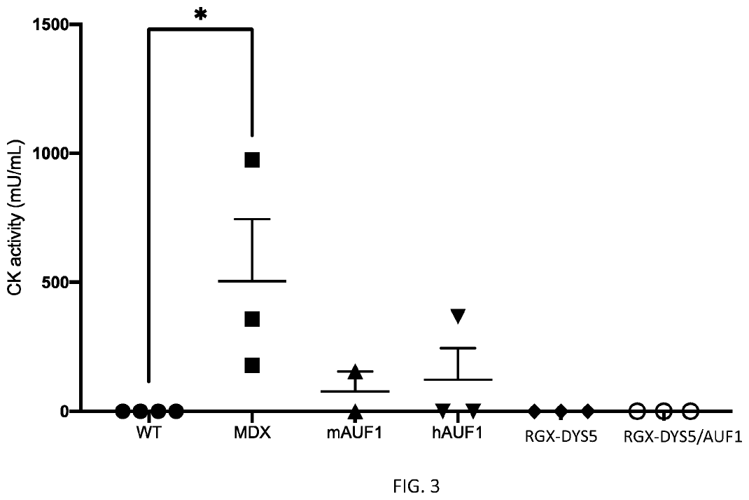

Treatment of mdx mice with AAV8 vectors encoding mAUF1 (AAV8-mAUF1) (2E13 vg/kg, ▴), hAUF1 (AAV8-tMCK-huAUF1) (2E13 vg/kg, ▾), AAV8-RGX-DYS5 (1E14 vg/kg, ♦) or a combination of AAV8-RGX-DYS5 and AAV8-hAUF1 (◯) gene therapy vectors as detailed above strongly decreased serum creatine kinase (CK, indicator of sarcolemma leakiness) levels 1 month after administration. FIG. 3. Wild type non-mdx mice (C57/B16) were used as a control. n=3 mice per treatment group. The data indicate that mdx mice treated with AAV8-RGX-DYS5 and/or AAV8-huAUF1 gene therapy have reduced muscle damage compared to untreated mdx mice. *, P<0.05 by t-test.

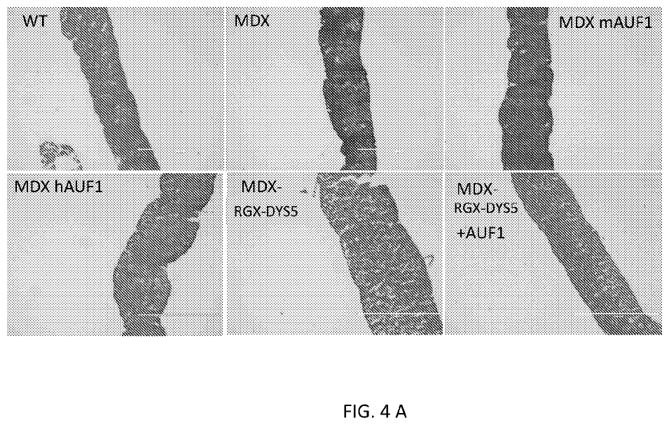

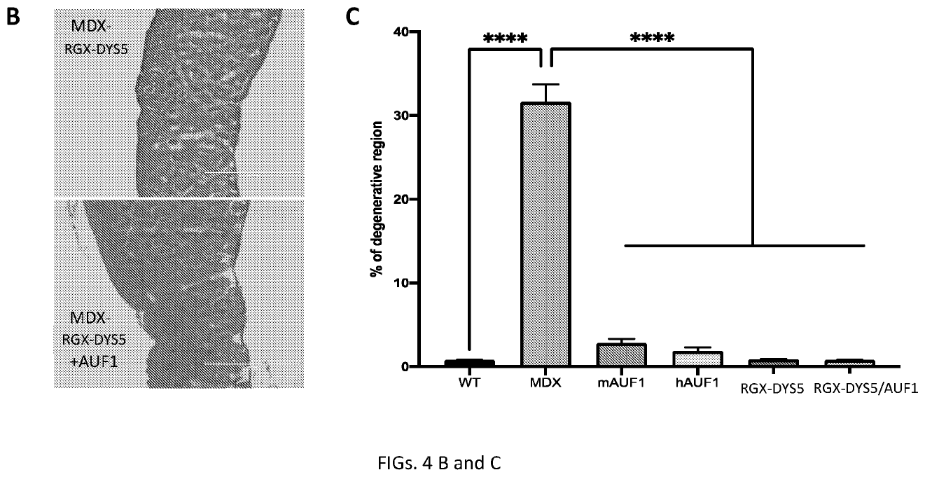

Treatment of mdx mice with a combination of AAV8-RGX-DYS5 and AAV8-huAUF1 gene therapy vectors reduces diaphragm muscle degeneration and promotes development of a larger myofiber size with healthier muscle organization than RGX-DYS5 gene therapy alone. FIG. 4A shows a low magnification image (scale bar 1000 mm) of Hematoxylin and Eosin (H&E) stain of the diaphragm muscle in treated mdx mice. FIG. 4B shows a high magnification H&E stain of the diaphragm muscle in mdx mice treated with RGX-DYS5 gene therapy alone or in combination with hAUF1 (scale bar 400 μm). FIG. 4C shows the percentage of the degenerative region of diaphragm muscle in treated mdx mice (n=3 per treatment group). ****, P<0.0001 by ANOVA.

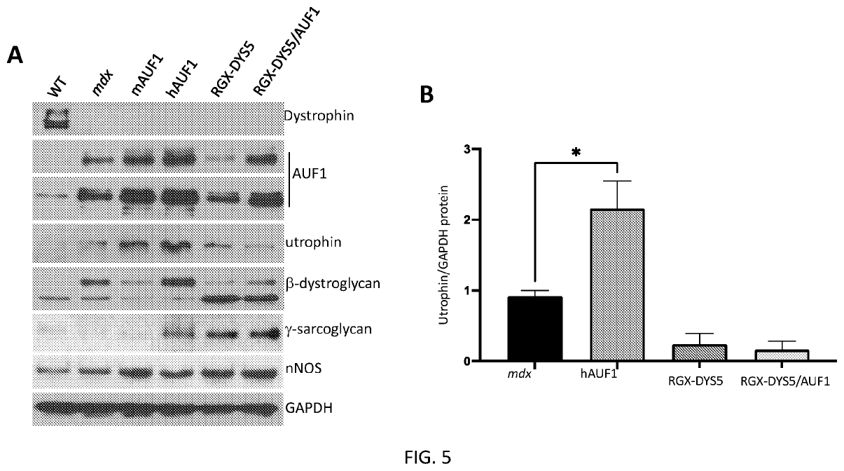

FIG. 5A is a representative immunoblot analysis (n=3 per treatment group) showing that mAUF1 and hAUF1 gene therapy increased utrophin protein levels, which is not observed in AAV8-RGX-DYS5+AAV8-mAUF1 combination gene therapy. Results also show that DAPC proteins (nNOS, γ-sarcoglycan and β-dystroglycan) are increased by hAUF1, RGX-DYS5 and combination therapy in the gastrocnemius muscle. FIG. 5B is a graph showing quantification of utrophin levels from 3 independent studies as shown in FIG. 5A. These results demonstrate that AUF1 gene transfer increases utrophin expression, which is prevented in combination therapy with RGX-DYS5, likely because efficient expression of microdystrophin from RGX-DYS5 suppresses endogenous utrophin expression. *, P<0.05 by t-test.

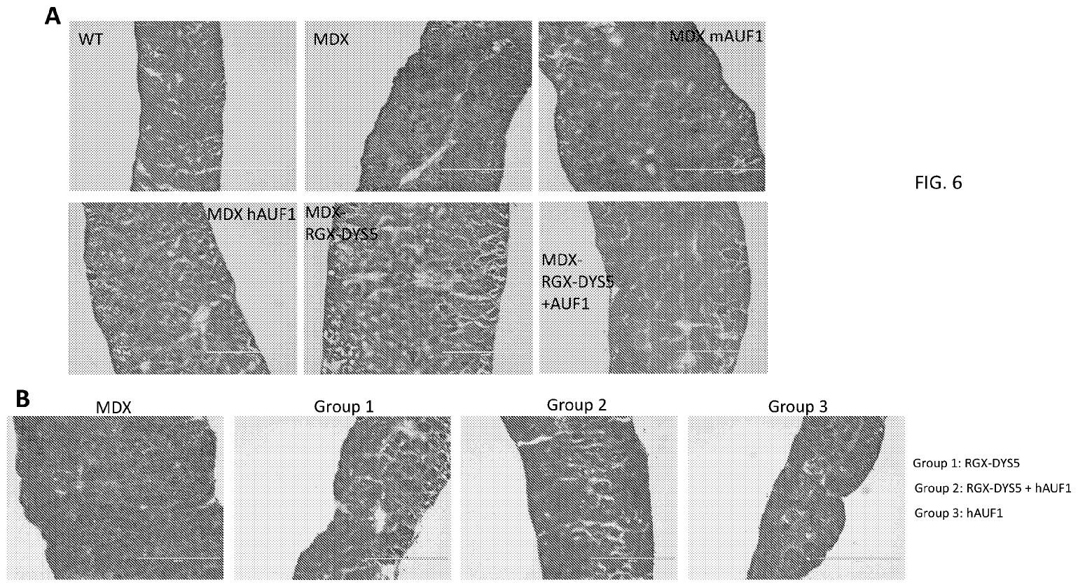

While single agent gene transfer of RGX-DYS5 or hAUF1 reduced diaphragm muscle degeneration, the combination of RGX-DYS5 plus hAUF1 gene transfer was superior at reducing diaphragm muscle degeneration. FIGS. 6A and B. Mice treated with combination AAV8-RGX-DYS5 plus AAV8-huAUF1 gene therapy developed a larger myofiber size than AAV8-RGX-DYS5 alone and had a healthier diaphragm muscle organization. FIG. 6 shows H&E staining of the diaphragm muscle in unblinded studies (A) and blinded studies (B). For blinded study in FIG. 6B, group 1 was treated with AAV8-RGX-DYS5 therapy alone, group 2 was treated with AAV8-RGX-DYS5 and AAV8-huAUF1 combination therapy and group 3 was treated with AAV8-huAUF1 therapy alone. Scale bar 400 μm. **, P<0.01; ***, P<0.001; ****, P<0.0001 by ANOVA.

Three months after administration of AAV8-mAUF1 (2E13 vg/kg), AAV8-huAUF1 (2E13 vg/kg), AAV8-RGX-DYS5 (1E14 vg/kg) or a combination of RGX-DYS5 and hAUF1 gene therapy, immunofluorescence images of diaphragm muscle were analyzed. Laminin alpha 2 was used for sarcolemma staining and DAPI was used for nucleus staining. The dystrophic phenotype found in mdx mice was most strongly reduced by a combination of RGX-DYS5 and hAUF1 gene therapy (data not shown).

Immunofluorescent imaging was also performed to analyze embryonic myosin heavy chain (eMHC) (indicative of continuous muscle regeneration), laminin alpha 2 (sarcolemma staining indicative of myofiber morphology and integrity) and DAPI (nuclei staining indicative of muscle fiber maturation). Results show that eHMC positive fibers are decreased with RGX-DYS5 treatment alone, hAUF1 treatment alone and RGX-DYS5 plus hAUF1 combination treatment of mdx mice, indicative of slowing the progression of (or progressive cycle of) muscle degeneration and regeneration, which means the myogenesis process has matured and is completed, which is not seen in the absence of hAUF1 gene transfer. However, mdx mice treated with a combination of RGX-DYS5 and hAUF1 had muscle fiber morphology most similar to WT muscle fiber morphology compared to mdx mice treated with either RGX-DYS5 or hAUF1 alone showing the superiority of the combination therapy (data not shown). n=3 mice per treatment group.

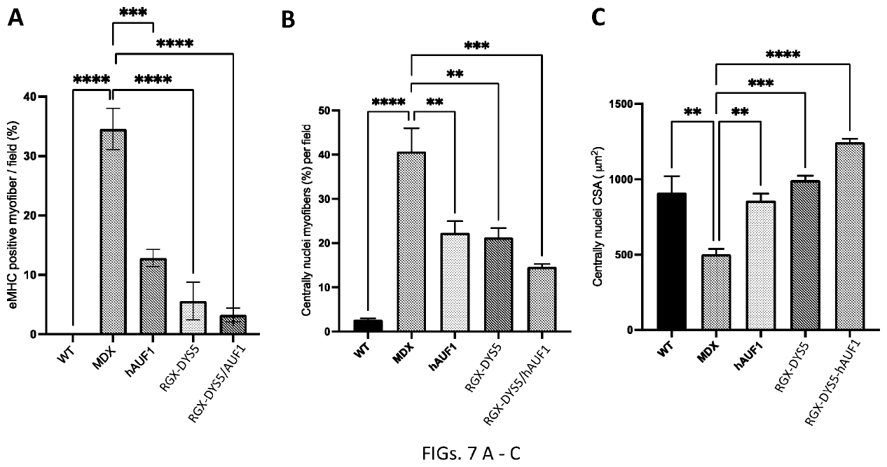

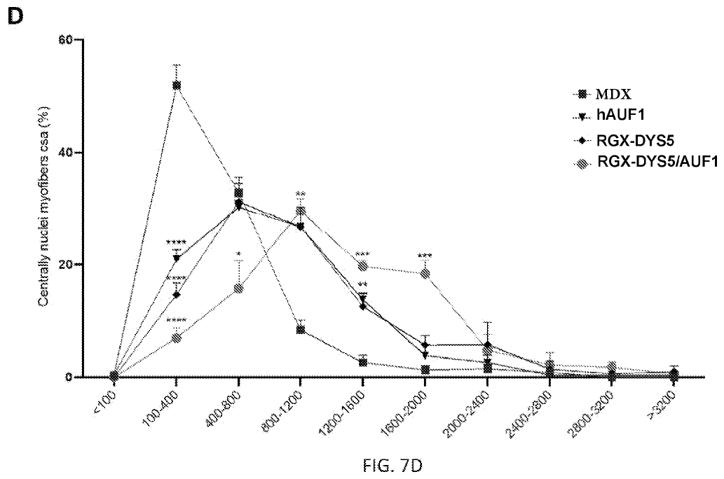

While hAUF1 and RGX-DYS5 treatment each reduces the percent of eMHC positive muscle fibers and the percent of centrally located nuclei fibers per field, it is the RGX-DYS5 plus hAUF1 combination therapy that shows the strongest increase in myofiber area (csa) and reduction in eMHC expression compared to either RGX-DYS5 or hAUF1 alone. FIG. 7A shows the quantification by image J of the percent of eMHC positive fibers in diaphragm, and the percent (FIG. 7B) and area (FIG. 7C) of central nuclei in muscle fiber. FIG. 7D shows the percentage of central nuclei myofibers CSA using multiple diaphragm muscles at different depths (layers) of muscle tissues. **, P<0.01; ***, P<0.001; ****, P<0.0001 by ANOVA.

Immunofluorescent imaging was also performed to analyze PAX7, a marker of muscle stem (satellite) cells and myoblasts. The presence of PAX7 is indicative of continuous muscle regeneration. Results show that PAX7 expression was decreased in mdx mice treated with either RGX-DYS5 or hAUF1 alone. Treatment of mdx mice with a combination of RGX-DYS5 and hAUF1 showed a greater decrease in PAX7 expression than either treatment alone, indicating that in treated mice there was a cessation of muscle regeneration (data not shown). Combination treatment also resulted in the most normal muscle morphology (data not shown).

Immunofluorescent imaging was performed to analyze β-dystroglycan and DAPI (nuclei) in diaphragm muscle and tibialis anterior (TA) muscle. Studies were conducted in a blinded manner on three mice per group. Gene transfer of hAUF1 or RGX-DYS5 alone induced a small increase in β-dystroglycan at the membrane but with strong cytoplasmic staining, indicative of incomplete membrane association (data not shown). Combination hAUF1 plus RGX-DYS5 gene transfer produced the strongest increase in β-dystroglycan membrane staining and the lowest level of cytoplasmic staining in both diaphragm and TA muscle (data not shown).

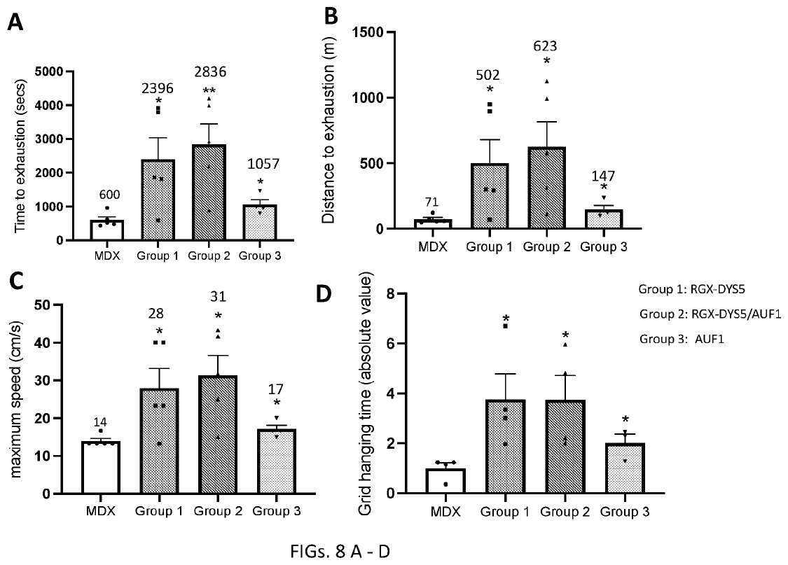

Muscle function studies were conducted on mdx mice in a blinded manner at three months post-gene transfer of AAV8-RGX-DYS5 alone, AAV8-hAUF1 (AAV8-tMCK-huAUF1) alone and AAV8-RGX-DYS5 plus AAV8-hAUF1. hAUF1 or RGX-DYS5 gene transfer increased time and distance to exhaustion (FIGS. 8A and B), maximum speed (FIG. 8C) and grid hanging time (FIG. 8D) compared to untreated mdx mice, whereas the combination therapy of RGX-DYS5 plus hAUF1 overall produced the strongest results indicative of improved muscle function and endurance. *, P<0.05; **, P<0.01 by ANOVA.

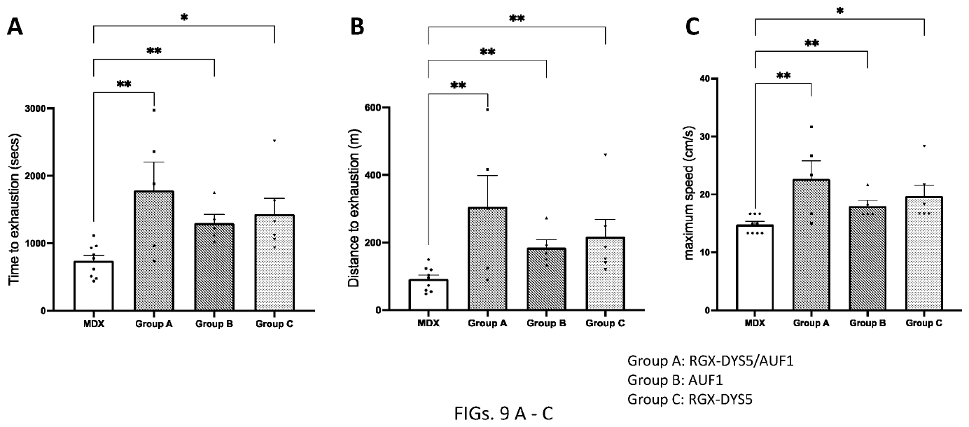

Muscle exercise function tests were carried out in a blinded manner in mdx mice treated with a higher dose of AAV8-hAUF1 (AAV8-tMCK-huAUF1) (6E13 vg/kg) at three months post-gene transfer, compared to AAV8-RGX-DYS5 at 1E14 vg/kg alone or in combination with AAV8-hAUF1 at the higher dose. Results show that the higher dose of AAV8-hAUF1 in combination with AAV8-RGX-DYS5 outperformed either gene transfer result alone, in all three tests for time to exhaustion (FIG. 9A), distance to exhaustion (FIG. 9B) and maximum speed (FIG. 9C). *, P<0.05; **, P<0.01 by ANOVA.

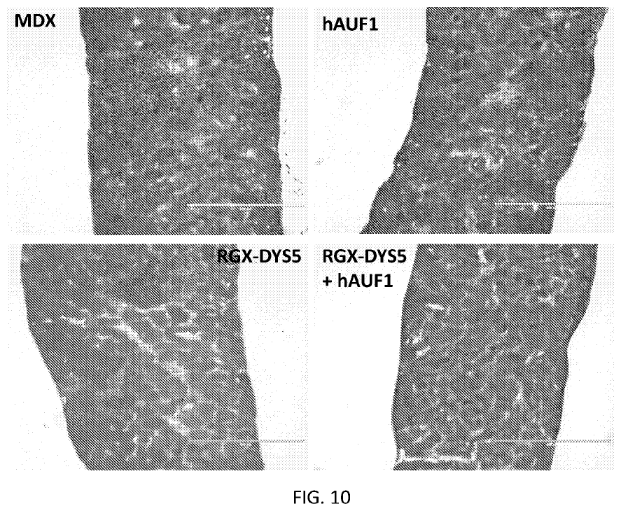

FIG. 10 shows H&E staining of mdx mouse diaphragm muscle in blinded studies at higher dose of AAV8-hAUF1 (AAV8-tMCK-huAUF1) (6E13 vg/kg) at three months post-gene transfer, compared to AAV8-RGX-DYS5 at 1E14 vg/kg alone or in combination with AAV8-hAUF1 at the higher dose. Results show that whereas single agent gene transfer of AAV8-RGX-DYS5 or AAV8-hAUF1 reduced diaphragm muscle degeneration compared to untreated mdx mouse diaphragm, the combination gene transfer of AAV8-RGX-DYS5 plus AAV8-hAUF1 at higher dose is superior. Scale bar 400 μm. Results are representative of three mice per group.

Immunofluorescence images of diaphragm muscle performed at three months post-gene transfer using a higher dose (6E13 vg/kg) of AAV8-hAUF1 (AAV8-tMCK-huAUF1). Imaging was carried out for eMHC (embryonic myosin heavy chain), indicative of continuous muscle regeneration, laminin alpha 2 for sarcolemma staining indicative of myofiber morphology and integrity, and DAPI for nuclei staining indicative of muscle fiber maturation. Wild type muscle is untreated. Results show that embryonic MHC positive fibers are decreased in RGX-DYS5 alone, hAUF1 alone and RGX-DYS5 plus hAUF1 combination gene transferred mdx muscle, indicative of greater muscle regeneration cessation, but muscle fibers demonstrate the best normal morphology in the combination treated samples (data not shown). Results are representative of three mice per condition.

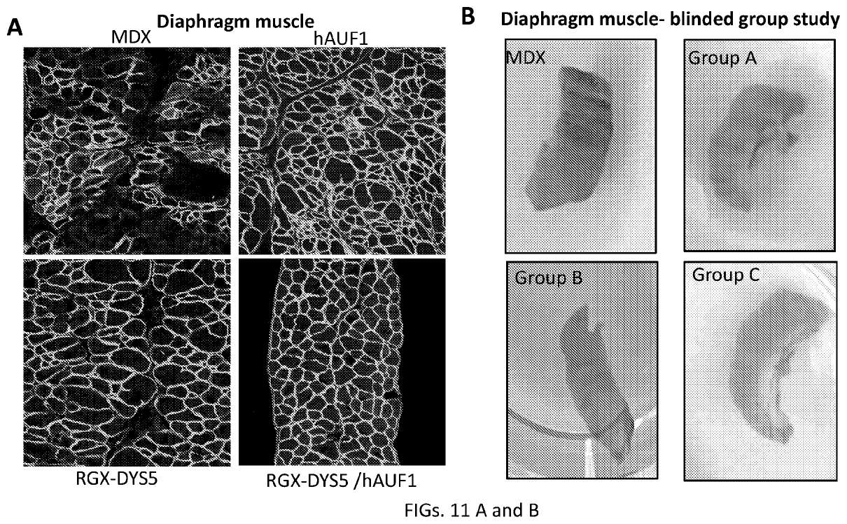

FIG. 11A shows immunofluorescence images of diaphragm muscle (Laminin a2) and FIG. 11B shows Evans blue staining (10 mg/ml IP (0.1 ml/10 gm body mass) of muscle diaphragm from blinded and unblinded studies of mdx mice at three months post-gene transfer with AAV8-hAUF1 (AAV8-tMCK-huAUF1) at high dose (6E13 vg/kg), AAV8-RGX-DYS5 at 1E14 vg/kg or combination of both. By light microscopy, Evans blue stains blue in damaged myofibers. Evans blue uptake was strongly reduced in diaphragm of mdx mice treated with hAUF1 or RGX-DYS5, but most strongly in combination gene transfer of RGX-DYS5 plus hAUF1 (FIG. 11B). Images are representative of three mice per condition.

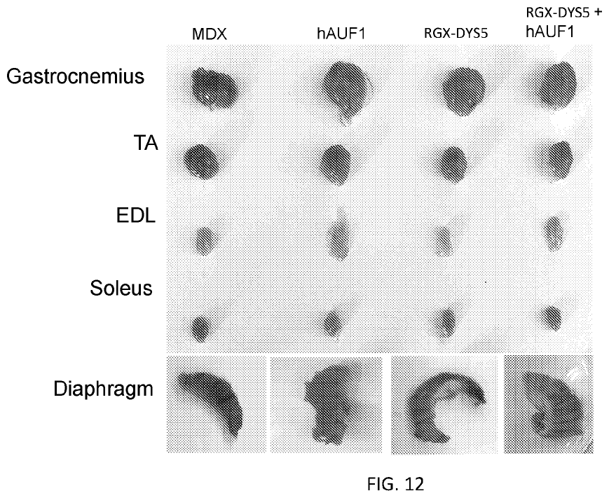

FIG. 12 shows Evans blue staining (10 mg/ml IP (0.1 ml/10 gm body mass) of muscles as indicated from blinded and unblinded studies of mdx mice at six months post-gene transfer with AAV8-hAUF1 (AAV8-tMCK-huAUF1) at high dose (6E13 vg/kg), AAV8-RGX-DYS5 at 1E14 vg/kg or combination of both. Evans blue stains blue in damaged myofibers. Evans blue uptake is strongly reduced in gastrocnemius, TA, EDL and diaphragm muscles of mdx mice treated with combination of hAUF1 plus RGX-DYS5, indicating more reduction in muscle damage than either gene transfer treatment alone. Images are representative of three mice per group.

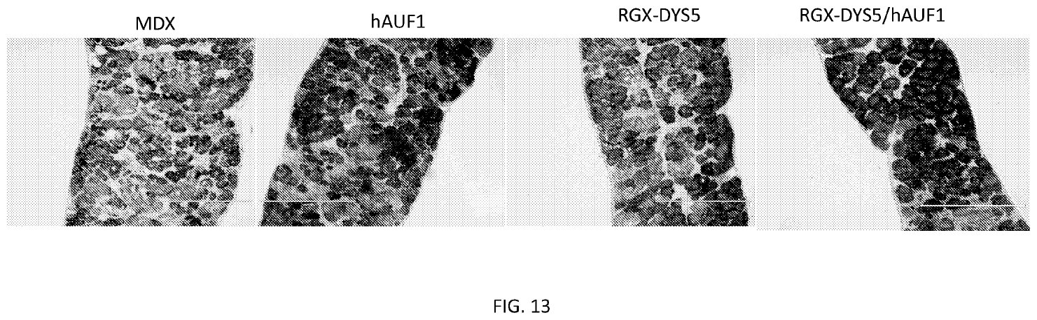

Succinate dehydrogenase (SDH) is a key mitochondrial enzyme complex composed of four subunits, and is a marker of mitochondrial activity and an index of muscle oxidative phenotype. FIG. 13 shows SDH activity staining in the diaphragm muscle of mdx mice from blinded studies at three months post-gene transfer with AAV8-hAUF1 (AAV8-tMCK-huAUF1) at higher dose (6E13 vg/kg), AAV8-RGX-DYS5 at 1E14 vg/kg or combination of both. SDH activity is increased in hAUF1 and most strongly in combination hAUF1/microdystrophin (e.g. RGX-DYS5) gene therapy. This indicates an improved the strongest improvement in mitochondrial function and respiration occurs in combination therapy treated animals. This is highly important because it is known that in mdx mice and Duchenne patients, mitochondrial dysfunction is apparent. Scale bar, 400 μm.

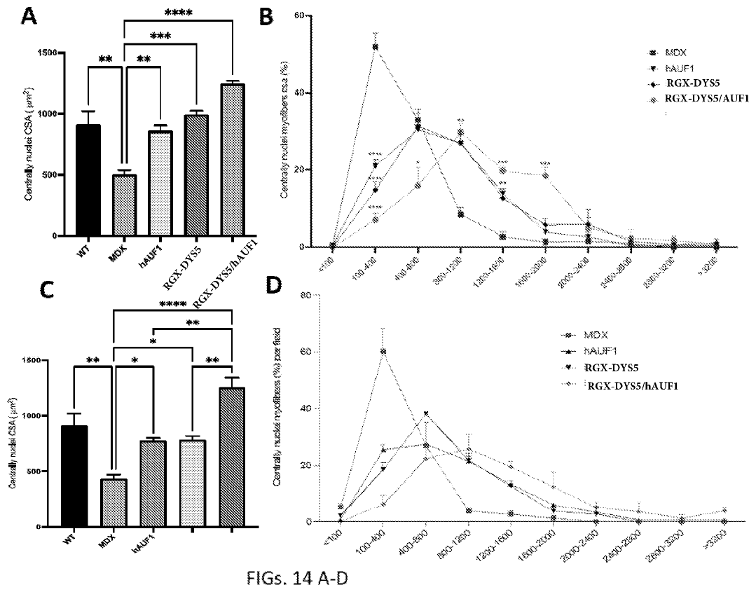

FIGS. 14 A-D shows the quantification of the percent (FIGS. 14 B and D) and area (FIGS. 14 A and C) of central nuclei in muscle fibers from mdx mice treated with either lower dose (2E13 vg/kg) AAV8-hAUF1 (AAV8-tMCK-huAUF1) and 1E14 vg/kg AAV8-RGX-DYS5 gene therapy alone or in combination (FIGS. 14 A and B) and higher dose (6E13 vg/kg) AAV8-hAUF1 and 1E14 vg/kg AAV8-RGX-DYS5 gene therapy alone or in combination (FIGS. 14 C and D). Results show that the combination gene transfer produces the strongest percent of centrally located nuclei fibers per field and the strongest increase in myofiber area (csa) compared to either RGX-DYS5 or hAUF1 alone. *, P<0.05; **, P<0.01; ***, P<0.001; ****, P<0.0001 by ANOVA.

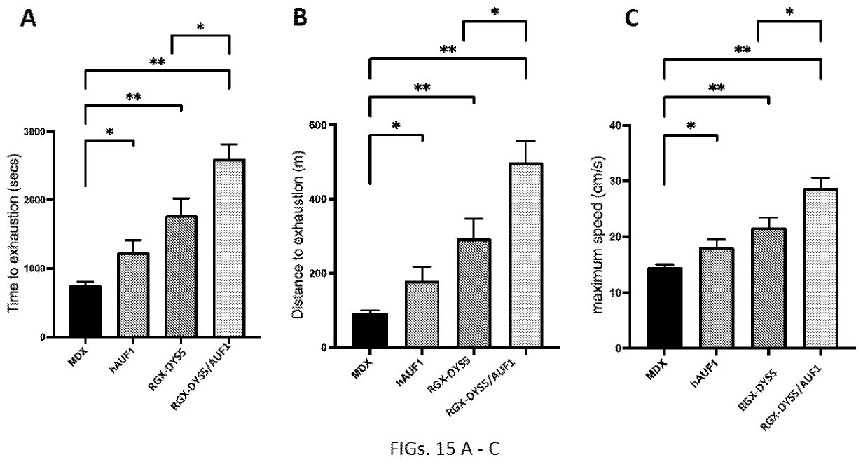

FIGS. 15 A-C shows the results of muscle exercise function tests at six months post-gene transfer in mdx mice with higher dose (6E13 vg/kg) AAV8-hAUF1 (AAV8-tMCK-huAUF1) and 1E14 vg/kg AAV8-RGX-DYS5 gene therapy alone or in combination. Muscle strength by all three tests for time to exhaustion (FIG. 15A), distance to exhaustion (FIG. 15B) and maximum speed (FIG. 15C) demonstrated the strongest improvement in the hAUF1 plus RGX-DYS5 combination gene transfer animals. *, P<0.05; **, P<0.01 by ANOVA.

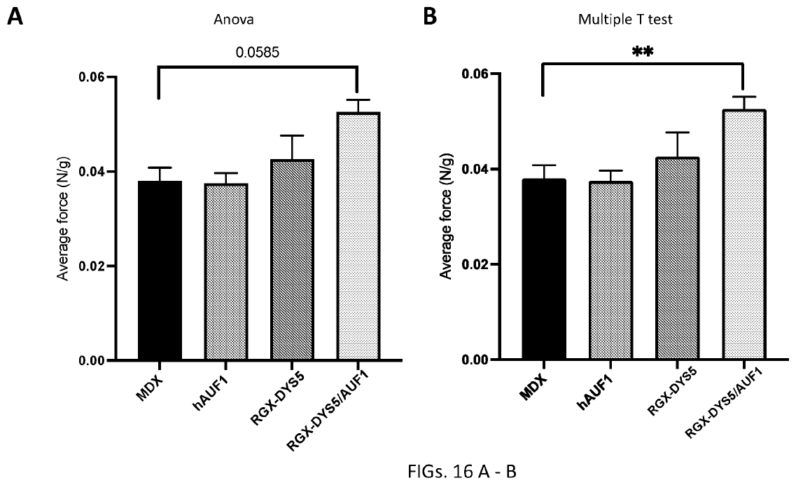

FIGS. 16 A and B show the results of muscle grip strength function tests were performed at six months post-gene transfer in mdx mice with higher dose (6E13 vg/kg) AAV8-hAUF1 (AAV8-tMCK-huAUF1) and 1E14 vg/kg AAV8-RGX-DYS5 gene therapy alone or in combination. Muscle grip strength was performed five times. The final fifth grip strength is most diagnostic of fatigued grip strength, indicative of endurance and stamina, and reported here. When analyzed two different ways by ANOVA (FIG. 16A) or multiple t-tests (FIG. 16B), the combination therapy of hAUF1 plus RGX-DYS5 demonstrated the strongest improvement in grip strength. **, P<0.01 by t-test.

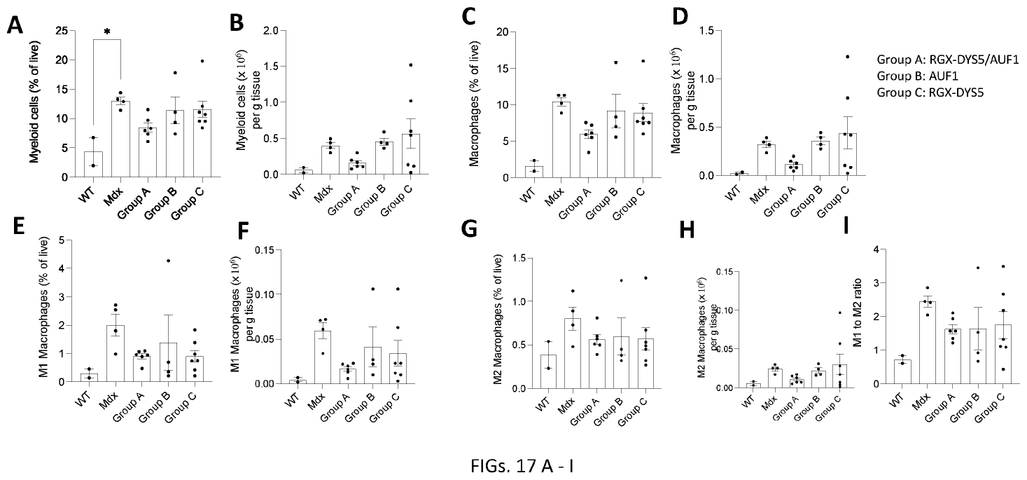

Combination treatment of mdx mice with hAUF1 plus microdystrophin (e.g., RGX-DYS5) results in greater reduction of myeloid cells, inflammatory and immune suppressive macrophages in muscle than either treatment alone, indicating greater reduction in muscle damage than either gene transfer treatment alone (FIGS. 17 A-I). Myeloid cells, total macrophages, M1 or M2 macrophages were quantified in the gastrocnemius muscle as indicated from blinded studies of mdx mice at three months post-gene transfer with AAV8-hAUF1 (AAV8-tMCK-huAUF1) at high dose (6E13 vg/kg), AAV8-RGX-DYS5 at 1E14 vg/kg or combination of both. Results indicate that Images are representative of three mice per group. *, P<0.05 by t-test.

Treatment with hAUF1 gene therapy (AAV8-tMCK-huAUF1) and a combination of microdystrophin (e.g. AAV8-RGX-DYS5) and hAUF1 gene therapy decreases the percent of muscle atrophy compared to mdx control mice. BaCl₂ was injected into the tibialis anterior muscle of mdx mice three months after gene therapy treatment. Percent atrophy was measured 7 days after BaCl₂ induction of muscle necrosis. FIG. 18. These data indicate that prophylaxis AUF1 gene transfer protects muscle from traumatic injuries in an mdx mouse model of DMD.

Injection of 1.2% of BaCl2 was performed into the tibialis anterior (TA) muscle of mdx mice at 3 months post-administration of 6E13 vg/kg AAV8-hAUF1, 1E14 vg/kg AAV8-RGX-DYS5 or combination therapy. TA muscles were harvested at 7 d post-injury from injured mdx mice and stained with H&E. Results show that uninjured TA muscles show that gene therapy improves muscle degeneration compared to untreated mdx mice (data not shown). Results also shows that prophylactic administration of hAUF1 significantly decreases muscle degeneration (darker staining) compared to mdx mice and mice that did received RGX-DYS5. Results show significant improvement in muscle fiber morphology and demonstrate clear evidence for reduced muscle necrosis and injury in hAUF1 prophylaxed TA muscle specimens (data not shown).

### Example 5: Transduction and Expression Analysis of AAV Vectors Expressing hAUF1 or hAUF1 and Microdystrophin in Mdx Mice

Three to four week old mdx mice were injected with 2E13 vg/kg of AAV8-mouse AUF1 (mAUF1) or AAV8-human AUF1 (hAUF1) vectors. Another cohort of mdx mice were injected with 1E14 vg/kg of AAV8-microdystrophin vector (RGX-DYS5) alone. A third cohort of mdx mice were injected with a combination mixture 1E14 vg/kg and 2E13 vg/kg of AAV8-microdystrophin vector (RGX-DYS5) and AAV8-hAUF1 (tMCK-huAUF1) vectors, respectively. A control (AAV8-eGFP/2E13 vg/kg dose) mdx mouse group and uninjected wild-type mouse group (C57BL/6 mice) were also included in the following experiments.

Tissues were harvested three months post injection for nucleic acid extraction and quantitation of DNA copy numbers and RNA transcripts by methods analogous to the methods described hereinabove in Example 1. In some examples, AUF1 and microdystrophin (μDys) RNA expression were calculated as a ratio of RNA transcripts to the endogenous control TBP (TATA-box-binding protein) transcripts, as previously described in Example 1.

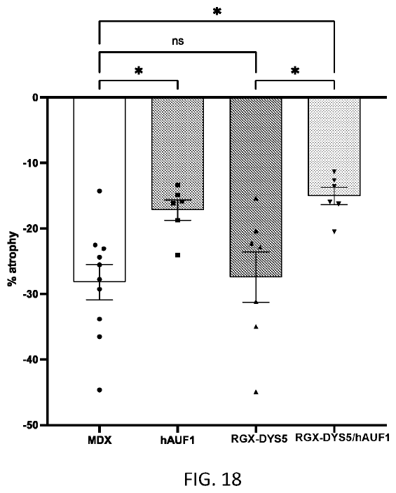

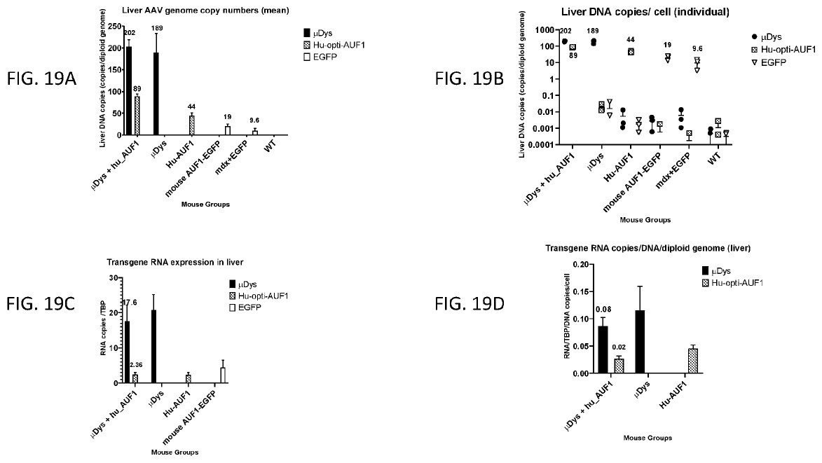

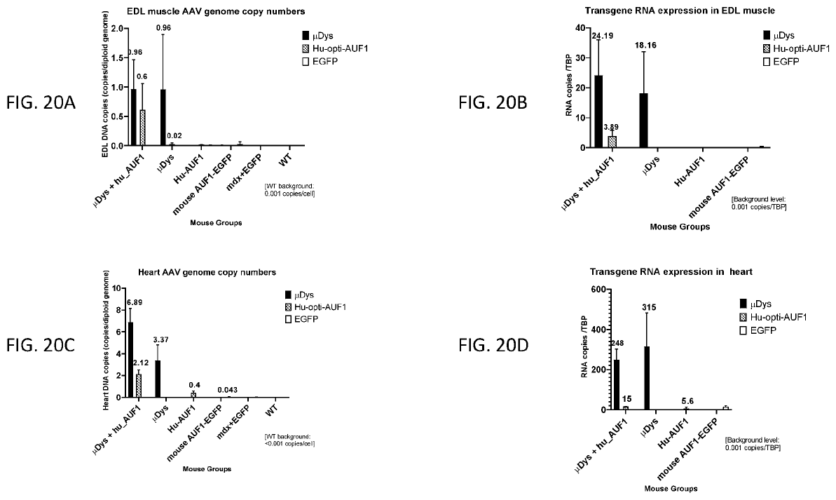

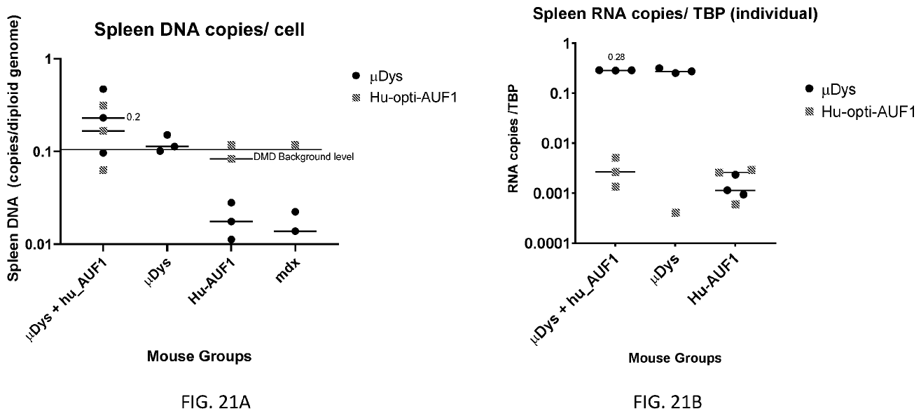

DNA copies and RNA expression of the vectors in liver and muscle (EDL and heart) tissue were assessed and results are provided in FIGS. 19A-19D and 20A-20D. As seen in these experiments, the combination of hAUF1 and microdystrophin (μDys) results in greater transduction of both transgenes, compared to the individual administration of either hAUF1 vector or μDys vector at the respective doses. See FIGS. 19A, 20A and 20C. Spleen biodistribution data confirms the increased amount of vector transduced into the tissue with respect to combination administration with both vectors (FIG. 21A).

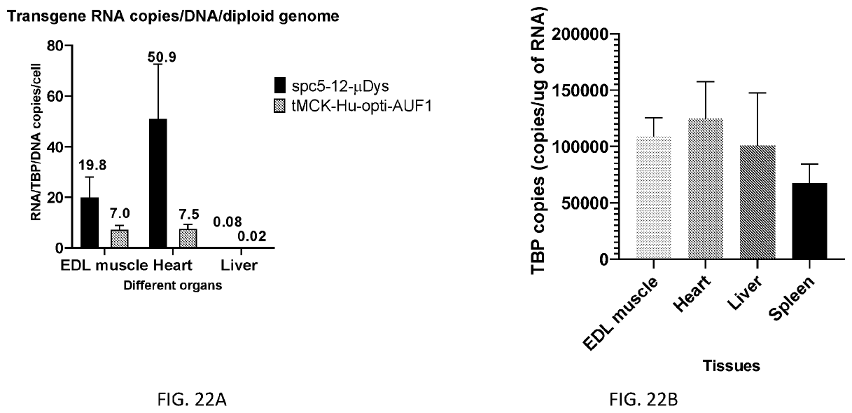

Assessing the RNA expression of hAUF1 (driven by tMCK promoter) or μDys (driven by Spc5-12 promoter) vectors in EDL, heart and liver compared to a control transcript (TBP) indicates measurable and adequate transcript levels was achieved upon administration of each of these vectors compared to an abundant mRNA endogenous to these tissues (FIGS. 22A-22B). This analysis provides a general assessment of promoter strength in each tissue.

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

1. A pharmaceutical composition for use in treating a dystrophinopathy in a subject in need thereof, wherein said pharmaceutical composition comprises a first therapeutic administered to said subject in combination with a second therapeutic which is different from said first therapeutic, wherein;
(a) the first therapeutic is a first rAAV particle comprising a nucleic acid molecule encoding an AU-rich mRNA binding factor 1 (AUF1) protein, or functional fragment thereof, operatively coupled to a muscle cell-specific promoter and flanked by inverted terminal repeat (ITR) sequences and
(b) the second therapeutic is a microdystrophin pharmaceutical composition, a mutation suppression therapy, an exon skipping therapy, a steroid therapy, an immunosuppressive/anti-inflammatory therapy, or a therapy that treats one or more symptoms of the dystrophinopathy.

2. The composition of claim 1, wherein the muscle cell-specific promoter is a muscle creatine kinase (MCK) promoter, a syn100 promoter, a CK6 promoter, a CK7 promoter, a CK8 promoter, or a CK9 promoter, a dMCK promoter, a tMCK promoter, a smooth muscle 22 (SM22) promoter, a myo-3 promoter, a Spc5-12 promoter, an Spc5V1 promoter, an Spc5V2 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter.

3. The composition of claim 2, wherein the muscle cell-specific promoter is a tMCK promoter, a Spc5-12 promoter, or a CK7 promoter.

4. The composition of claim 2, wherein the nucleic acid molecule encodes one or more of human p37^{AUF1}, p40^{AUF1}, p42^{AUF1}, or p45^{AUF1}.

5. The composition of claim 4, wherein the nucleotide sequence encoding the p40^{AUF1} protein is the nucleotide sequence of SEQ ID NO: 17.

6. (canceled)

7. The composition of claim 1, wherein the first rAAV particle comprises a recombinant genome having the nucleotide sequence of SEQ ID NO: 31 (spc-hu-opti-AUF1-CpG(−)), SEQ ID NO: 32 (tMCK-huAUF1), SEQ ID NO: 33 (Spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1).

8. The composition of claim 1, wherein the nucleic acid encoding the AUF 1 protein is a single stranded or self-complementary recombinant artificial genome.

9. The composition of claim 1, wherein the AAV has a capsid that is at least 95%, 99% or 100% identical to SEQ ID NO: 114 (AAV8 capsid), SEQ ID NO: 115 (AAV9 capsid), or SEQ ID NO: 118 (AAVhu.32).

10. The composition of claim 1, wherein the rAAV is administered at a dose of 1E13 to 1E14 vg/kg or a dose of 2E13 vg/kg.

11. The composition of claim 1, wherein the second therapeutic is a microdystrophin pharmaceutical composition.

12. The composition of claim 11, wherein the microdystrophin protein consists of dystrophin domains arranged from amino-terminus to the carboxy terminus: ABDH1-R1-R2-R3-H3-R24-H4-CR-CT, wherein ABD is an actin-binding domain of dystrophin, H1 is a hinge 1 region of dystrophin, R1 is a spectrin 1 region of dystrophin, R2 is a spectrin 2 region of dystrophin, R3 is a spectrin 3 region of dystrophin, H3 is a hinge 3 region of dystrophin, R24 is a spectrin 24 region of dystrophin, H4 is hinge 4 region of dystrophin, CR is the cysteine-rich region of dystrophin, and CT comprises at least the portion of the CT comprising an α1-syntrophin binding site.

13. The composition of claim 12, wherein the microdystrophin protein has the amino acid sequence of SEQ ID NO: 52 or SEQ ID NO: 54.

14. The composition of claim 11, wherein the microdystrophin protein has an amino acid sequence of one of SEQ ID NO: 133 to 137.

15. The composition of claim 11, wherein the microdystrophin pharmaceutical composition comprises a therapeutically effective amount of a second rAAV particle comprising an artificial genome comprising a nucleic acid that encodes the microdystrophin protein operatively coupled to a regulatory sequence that promotes expression in muscle cells, which trans gene is flanked by ITRs; and a pharmaceutically acceptable carrier.

16. The composition of claim 15, wherein the regulatory sequence comprises a muscle-specific promoter.

17. The composition of claim 16, wherein the muscle-specific promoter is a muscle creatine kinase (MCK) promoter, a syn100 promoter, a CK6 promoter, a CK7 promoter, a CK8 promoter, or a CK9 promoter, a dMCK promoter, a tMCK promoter, a smooth muscle 22 (SM22) promoter, a myo-3 promoter, a Spc5-12 promoter, an Spc5V1 promoter, an Spc5V2 promoter, a creatine kinase (CK) 8e promoter, a U6 promoter, a H1 promoter, a desmin promoter, a Pitx3 promoter, a skeletal alpha-actin promoter, a MHCK7 promoter, or a Sp-301 promoter.

18. The composition of claim 17, wherein the muscle specific promoter is Spc5-12, Spc5V1, or Spc5V2.

19. The composition of claim 15, wherein the artificial genome comprises the nucleotide sequence of SEQ ID NO: 94, 96, 130, or 132.

20. The composition of claim 15, wherein the AAV has a capsid that is at least 95%, 99% or 100% identical to SEQ ID NO: 114 (AAV8 capsid), SEQ ID NO: 115 (AAV9 capsid), or SEQ ID NO: 118 (AAVhu.32 capsid).

21. The composition of claim 15, wherein the therapeutically effective amount of the second rAAV particle is administered intravenously or intramuscularly at dose of 2×10¹³ to 1×10¹⁵ genome copies/kg.

22. The composition of claim 15, wherein the first therapeutic and the second therapeutic are administered concurrently or within 1 week or within 2 weeks of each other.

23. The composition of claim 15, wherein the ratio of the vector genomes of the first rAAV particle in the first therapeutic to the vector genomes of the second rAAV particle in the second therapeutic is 0.5 to 1; 0.25 to 1; 0.2 to 1; 0.1 to 1; 1 to 1; 1 to 2; 1 to 5; 1 to 10; 1 to 20; 1 to 100; or 1 to 1000.

24. The composition of claim 11 wherein the microdystrophin pharmaceutical composition comprises a therapeutically effective amount of SGT-001, GNT 004, rAAVrh74.MHCK7, micro-dystrophin (SRP-9001), or PF-06939926.

25-27. (canceled)

28. The composition of claim 1, wherein the dystrophinopathy is Duchenne muscular dystrophy (DMD), Becker muscular dystrophy (BMD), X-linked dilated cardiomyopathy, or limb-girdle muscular dystrophy.

29. A nucleic acid comprising a nucleotide sequence of SEQ ID NO: 17 encoding AUF1 p40.

30. A vector comprising the nucleic acid of claim 29 operably linked to a muscle cell specific promoter.

31. (canceled)

32. The vector of claim 30, wherein the muscle cell-specific promoter is a tMCK promoter, an Spc5-12 promoter, or a CK7 promoter.

33-36. (canceled)

37. The vector of claim 30 which comprises a nucleotide sequence of SEQ ID NO: 31 (spc-hu-opti-AUF1-CpG(−)), SEQ ID NO: 32 (tMCK-huAUF1), SEQ ID NO: 33 (Spc5-12-hu-opti-AUF1-WPRE), SEQ ID NO: 34 (ss-CK7-hu-AUF1), SEQ ID NO: 35 (spc-hu-AUF1-no-intron), or SEQ ID NO: 36 (D(+)-CK7AUF1).

38-57. (canceled)

58. The composition of claim 11, wherein the dystrophinopathy is Duchenne muscular dystrophy (DMD), Becker muscular dystrophy (BMD), X-linked dilated cardiomyopathy, or limb-girdle muscular dystrophy.
